(19) United States
(12) Patent Application Publication
(10) Pub. No.: US 2020/0362421 A1
(43) Pub. Date: Nov. 19, 2020

(54) CLASSIFICATION AND ACTIONABILITY INDICES FOR CANCER

(71) Applicant: LIFE TECHNOLOGIES CORPORATION, Carlsbad, CA (US)

(72) Inventors: Daniel RHODES, Ann Arbor, MI (US); Seth SADIS, Ann Arbor, MI (US); Santhoshi BANDLA, Northville, MI (US); Douglas ROSS, Burlingame, CA (US); Peter WYNGAARD, Ann Arbor, MI (US)

(21) Appl. No.: 16/863,360

(22) Filed: Apr. 30, 2020

Related U.S. Application Data
(63) Continuation-in-part of application No. 15/828,333, filed on Nov. 30, 2017, now abandoned.
Application No. 15/828,333 is a continuation of application No. 14/212,717, filed on Mar. 14, 2014, now abandoned.
Application No. 14/212,717 is a continuation-in-part of application No. 15/068,011, filed on Mar. 11, 2016, now abandoned.
Application No. 15/068,011 is a continuation of application No. 14/212,115, filed on Mar. 14, 2014, now abandoned.
(60) Provisional application No. 61/877,827, filed on Sep. 13, 2013.
(60) Provisional application No. 61/891,224, filed on Oct. 15, 2013.
(60) Provisional application No. 61/799,399, filed on Mar. 15, 2013.

(51) Int. Cl. C12Q 1/6886

(52) U.S. Cl. CPC C12Q 1/6886; C12Q 2600/158; C12Q 2600/118; C12Q 2600/106

(57) ABSTRACT

The disclosure provides compositions, kits, and methods for detecting a plurality of genes and associated variants in a sample from a subject with cancer (e.g., lung cancer). The compositions, kits, and methods include a set of oligonucleotides, typically primers and/or probes that can hybridize to identify a gene variant. The methods disclosed herein provide for a mutation status of a tumor to be determined and subsequently associated with a report comprising an actionable treatment recommendation (e.g., a report comprising an actionable treatment recommendation).

### CROSS-REFERENCE TO RELATED APPLICATIONS

This application is a continuation-in-part application of pending U.S. application Ser. No. 15/828,333, filed Nov. 30, 2017, the entire contents of which application is incorporated by reference herein, which is a continuation application of U.S. application Ser. No. 14/212,717 filed Mar. 14, 2014, now abandoned, which claims priority to U.S. Provisional Application Nos. 61/891,224 filed Oct. 15, 2013 and 61/877,827 filed Sep. 13, 2013; and this application is a continuation-in-part application of pending U.S. application Ser. No. 15/068,011, filed Mar. 11, 2016, the entire contents of which application is incorporated by reference herein, which is a continuation application of U.S. application Ser. No. 14/212,115 filed Mar. 14, 2014, now abandoned, which claims priority to U.S. Provisional Application No. 61/799,399 filed Mar. 15, 2013.

### BACKGROUND

Cancer is a broad group of diseases involving unregulated cell growth. Although the causes of cancer are diverse, our understanding of genetic alterations that are involved is increasing rapidly.

Lung cancer is the leading cause of cancer deaths among both men and women. It is a fast growing and highly fatal disease. Nearly 60% of people diagnosed with lung cancer die within one year of diagnosis and approximately 75% die within 2 years. There are two major types of lung cancer: small cell lung cancer (SCLC) and non-small cell lung cancer (NSCLC). Approximately 85% of lung cancers are NSCLC. There are 3 sub-types of NSCLC, which differ in size, shape, and biochemical make-up. Approximately 25-30% of all lung cancers are squamous cell carcinomas. Adenocarcinomas (e.g., bronchioloalveolar carcinoma) account for approximately 40% of lung cancers, and are usually found in the outer region of the lung. Large-cell undifferentiated carcinoma accounts for approximately 10-15% of all lung cancers. SCLC and NSCLC are treated very differently. SCLC is mainly treated with chemotherapy, either alone or in combination with radiation. In contrast with treatment for SCLC, surgery is the only reliable method to cure NSCLC. Lymph nodes are also removed to assess the spread of cancer. In addition to surgery, chemotherapy can be used to treat NSCLC.

A growing number of treatment regimens are available and becoming available. However, many treatment regimes are only effective against cancers (e.g., lung cancers) that have a particular genetic variation. Therefore, a test that can detect many different specific actionable genetic variations would have significant value to cancer patients (e.g., lung cancer). However, the tissue required for currently available multiple genetic variance assays far exceeds what is available from a typical tumor biopsy or resection. Furthermore, tests are not available for many of the genetic variations, and there is no tool that can recommend a treatment based on a comprehensive scan of many known, actionable genetic variations.

The disclosed compositions, kits and methods provide comprehensive genetic variance screening of a cancer (e.g., lung cancer) in a single panel utilizing a single cancer (e.g., lung cancer) sample. The genetic variants form the basis of an actionable treatment recommendation framework provided herein.

### BRIEF SUMMARY

The disclosure provides methods, compositions and kits. In one embodiment, a method to determine an actionable treatment recommendation for a subject diagnosed with lung cancer is provided. In one embodiment provided method comprises: obtaining a biological sample from the subject; detecting at least one variant using a set of probes that hybridize to and amplify EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS genes to detect at least one variant; determining, based on the at least one variant detected, an actionable treatment recommendation for the subject. In another embodiment provided method comprises: obtaining a biological sample from the subject; detecting at least one variant using a set of probes that hybridize to and amplify EGFR, ALK, ROS1, KRAS, BRAF, ERBB2, MET, RET, FGFR1, KIT/PGDFRA, PIK3CA, AKT1, BRAF, and HRAS genes to detect at least one variant; determining, based on the at least one variant detected, an actionable treatment recommendation for the subject.

In one embodiment provided method comprises: contacting a biological sample from a subject; detecting at least one variant using a set of probes that hybridize to and amplify EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, BRAF, and HRAS genes to detect at least one variant; determining, based on the at least one variant detected, an actionable treatment recommendation for the subject. In another embodiment provided method comprises: contacting a biological sample from a subject; detecting at least one variant using a set of probes that hybridize to and amplify EGFR, ALK, ROS1, KRAS, BRAF, ERBB2, MET, RET, FGFR1, KIT/PGDFRA, PIK3CA, AKT1, BRAF, and HRAS genes to detect at least one variant; determining, based on the at least one variant detected, an actionable treatment recommendation for the subject.

In another embodiment, the disclosure provides a method to determine an actionable treatment recommendation for a subject diagnosed with lung cancer, comprising: detecting in a sample from a subject, at least one variant using a set of probes that hybridize to and amplify ALK, ROS1, KRAS, BRAF, ERBB2, MET, RET, FGFR1, and KIT/PDGFRA genes to detect at least one variant, and determining, based on the at least one variant detected, an actionable treatment recommendation for the subject.

In yet other embodiments, a method to determine the likelihood of a response to a treatment in an individual afflicted with lung cancer is provided. The method comprises: determining the presence or absence of at least one gene variant in a sample obtained from the individual, wherein the at least one variant is in EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS genes, wherein the presence of at least one variant indicates the individual is likely or unlikely to respond to the treatment, wherein the treatment is selected from: crizotinib when the variant detected is an ALK fusion; ROS1 fusion (EZR, SLC34A2, CD74, and/or SDC4); MET gene amplification; EGFR tyrosine kinase inhibitor (TKI) when the variant detected is EGFR (L858R, Exon 19 del, and/or G719X); a non-EGFR TKI treatment when the variant detected is EGFR T790M; a MEK inhibitor when the variant detected is KRAS G12C/V/D/A/S/R/F, G13C, G3D and/or G12F; vermurafenib when the variant detected is BRAF V600E; an irreversible pan-erb inhibitor when the variant detected is ERBB2 exon 20 ins; and a PIC3CA inhibitor when the variant detected is PIK3CA (E545K, E545G, E545a, H1047R, E542K and/or H1047L). In other embodiments the method comprises: determining the presence or absence of at least one gene variant in a sample obtained from the individual, wherein the at least one variant is in EGFR, ALK, ROS1, KRAS, BRAF, ERBB2, MET, RET, FGFR1, KIT/PGDFRA, PIK3CA, AKT1, BRAF, and/or HRAS genes, wherein the presence of at least one variant indicates the individual is likely or unlikely to respond to the treatment, wherein the treatment is selected from: crizotinib when the variant detected is an ALK fusion; ROS1 fusion (EZR, SLC34A2, CD74, and/or SDC4); MET gene amplification; EGFR tyrosine kinase inhibitor (TKI) when the variant detected is EGFR (L858R, Exon 19 del, and/or G719X); a non-EGFR TKI treatment when the variant detected is EGFR T790M; a MEK inhibitor when the variant detected is KRAS G12C/V/D/A/S/R/F, G13C, G13D and/or G12F; vermurafenib when the variant detected is BRAF V600E; an irreversible pan-erb inhibitor when the variant detected is ERBB2 exon 20 ins; and a PIC3CA inhibitor when the variant detected is PIK3CA (E545K, E545G, E545a, H1047R, E542K and/or H1047L).

In another embodiment, the disclosure provides a method of detecting a nucleic acid variant in a sample, comprising obtaining a biological sample, amplifying at least one gene selected from EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS genes, using primers that (a) amplifying at least one variant selected from EGFR (L858R, Exon 19 del, G719X and/or T790M), KRAS (G12C/V/D/A/S/R/F, G13C, G13D and/or G12F), BRAF (L597R, D594H/N, V600E), ERBB2 exon 20 ins, PIK3CA (E545K, E545G, E545a, H1047R, and/or H1047L); and (b) detecting at least one nucleic acid variant present in the sample.

In another embodiment, the disclosure provides a method of detecting a nucleic acid variant in a sample, comprising obtaining a biological sample, amplifying at least one gene selected from EGFR, ALK, ROS1, KRAS, BRAF, ERBB2, MET, RET, FGFR1, KIT/PGDFRA, PIK3CA, AKT1, BRAF, and HRAS genes, using primers that (a) amplifying at least one variant selected from EGFR (L858R, Exon 19 del, G719X and/or T790M), KRAS (G12C/V/D/A/S/R/F, G13C, G13D and/or G12F), BRAF (L597R, D594H/N, V600E), ERBB2 exon 20 ins, PIK3CA (E545K, E545G, E545a, H1047R, and/or H1047L); and (b) detecting at least one nucleic acid variant present in the sample.

In yet embodiment, a method of treating lung adenocarcinoma in a patient is disclosed. The method comprises: testing for the presence of variants in at least one of ALK, ROS1, KRAS, BRAF, ERBB2, MET, RET, FGFR1, and KIT/PDGFRA genes in a lung tumor sample from the patient and administering a therapeutically effective amount a treatment to the patient, wherein the treatment is: Crizotinib when the variant detected is an ALK fusion, ROS1 fusion (EZR, SLC34A2, CD74, and/or SDC4), or MET gene amplification; EGFR tyrosine kinase inhibitor (TKI) when the variant detected is EGFR (L858R, Exon 19 del, and/or G719X); a MEK inhibitor when the variant detected is KRAS G12C/V/D/A/S/R/F, G13C, G3D and/or G12F; Vermurafenib when the variant detected is BRAF V600E; and an irreversible pan-erb inhibitor when the variant detected is ERBB2 exon 20 ins.

In yet another embodiment, the disclosure provides a method of identifying patients with lung cancer eligible for treatment with crizotnib, an EGFR TKI, or a treatment other than an EGFR TKI, a MEK inhibitor, vermurafenib, or an irreversible pan-erb inhibitor, comprising testing a lung tumor sample from the patient for the presence of a variant comprising an ALK fusion, ROS1 fusion (EZR, SLC34A2, CD74, and/or SDC4), EGFR (L858R, Exon 19 del, and/or T790M), KRAS (G12C/V/D/A), wherein the presence of at least one of said variants indicates the patient is eligible for treatment with at least one of said treatments.

The disclosure, in certain embodiments, also provides a kit comprising a set of probes, wherein the set of probes specifically recognize the genes EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS, and wherein the set of probes can recognize and distinguish one or more allelic variants of the genes EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS.

Certain embodiments of the disclosure further provide a composition comprising a set of probes, wherein the set of probes specifically recognize the genes EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS, and wherein the set of probes can recognize and distinguish one or more allelic variants of the genes EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS.

The disclosure, in certain embodiments, also provides a kit comprising a set of probes, wherein the set of probes specifically recognize the genes AKT1, ALK, BRAF, ERBB2, EGFR, FGFR1, HRAS, KIT, KRAS, MET, PIK3CA, RET and ROS, and wherein the set of probes can recognize and distinguish one or more allelic variants of the genes AKT1, ALK, BRAF, ERBB2, EGFR, HRAS, KRAS, MET, PIK3CA, RET and ROS.

Certain embodiments of the disclosure further provide a composition comprising a set of probes, wherein the set of probes specifically recognize the genes AKT1, ALK, BRAF, ERBB2, EGFR, FGFR1, HRAS, KIT, KRAS, MET, PIK3CA, RET and ROS, and wherein the set of probes can recognize and distinguish one or more allelic variants of the genes AKT1, ALK, BRAF, ERBB2, EGFR, HRAS, KRAS, MET, PIK3CA, RET and ROS.

In certain embodiments of the disclosure, the compositions can comprise a set of probes that specifically recognize the genes in Tables 13-17 and 19. Further, the methods and kits can comprise the identifying, detecting, and/or determining the presence of one or more of the genes, copy number variations, and/or gene fusions in Tables 13-17 and 19 These genes, copy number variations, and/or gene fusions can be associated with any type of cancer.

In yet another embodiment of the disclosure, a composition comprising a set of probes is provided, wherein the set of probes specifically recognizes driver gene alterations associated with a cancer. In certain embodiments, the driver gene alterations have associated actionability, such as evidence that the driver gene alteration is associated with a drug response. In certain embodiments, the driver gene alterations comprise one or more of the genes, copy number variations, and/or gene fusions in Tables 13-17 and 19.

In certain embodiments of the disclosure, the driver gene alterations are detected or identified by a method comprising next generation sequencing. The driver gene alterations can be associated with a cancer.

In yet another embodiment of the disclosure, the driver gene alterations detected or identified by a method comprising next generation sequencing are confirmed by a method comprising sanger sequencing or thermo cycle sequencing.

### BRIEF DESCRIPTION OF THE FIGURES

FIG. 1 a work flow, according to one embodiment of the disclosure, in which a sample is screened by NGS and a Reflex Test is conducted. A report is generated and actionability of an FDA-approved drug or additional classification with a companion diagnostic test is reported. Treatment can proceed based on the report.

FIG. 2 is workflow, according to another embodiment of the disclosure, in which a tumor sample is sequenced and a report with actionability is generated.

FIG. 3 is workflow, according to another embodiment of the disclosure, in which a tumor sample is sequenced and a report with actionability is generated.

FIG. 4 is a bioinformatics workflow in accordance with an embodiment of the disclosure, in which variants are identified and a report is generated

FIGS. 5A-5B are bioinformatics workflow according to an embodiment of the disclosure, in which a variant calls are reviewed and a report is generated.

FIG. 6 is a schematic depicting how gene content can be defined by driver analysis, according to an embodiment of the disclosure.

### DETAILED DESCRIPTION

The disclosure provides compositions, kits, and methods for detecting a plurality of genes and associated variants in a subject with cancer; and compositions, kits, and methods for detecting a plurality of genes and associated variants in a subject with lung cancer. The compositions, kits, and methods include a set of oligonucleotides, typically primers and/or probes that can hybridize to identify a gene variant. The methods disclosed herein provide for a mutation status of a tumor to be determined and subsequently associated with an actionable treatment recommendation. In certain embodiments, methods for determining a treatment and treating a subject with cancer are provided. In certain embodiments, methods for determining a treatment and treating a subject with lung cancer are provided.

An advantage of the disclosed compositions, kits, and methods is the ability to recommend an actionable treatment for a subject diagnosed with cancer, by comprehensively screening a tumor sample for a variety of mutations, including driver mutations. Driver mutations can be associated with treatment response. Therefore, by determining the driver mutation status, the disclosed methods can determine and provide an actionable treatment recommendation. This comprehensive screening is performed in a single panel and therefore can be performed utilizing a single biological sample, thus preserving valuable sample.

An advantage of the disclosed compositions, kits, and methods is the ability to recommend an actionable treatment for a subject diagnosed with lung cancer, by comprehensively screening a tumor sample for a plurality of high and/or optionally low prevalence genetic variances that are most likely to have an impact on the appropriate clinical course of action for the subject. In certain embodiments, by determining the mutation status of the disclosed combination of gene variations, the methods provide an actionable treatment recommendation for greater than 50% of lung adenocarcinoma subjects. This comprehensive screening is performed in a single panel and therefore can be performed utilizing a single biological sample, thus preserving valuable sample.

### Definitions

“Cancer” refers to a broad group of diseases involving unregulated cell growth. A large variety of cancers are known. Examples of known cancers are provided throughout the disclosure and are listed in Table 18.

“Lung cancer” refers generally to two main types of lung cancer categorized by the size and appearance of the malignant cells: non-small cell (approximately 80% of cases) and small-cell (roughly 20% of cases) lung cancer. Lung adenocarcinoma is the most common subtype of non-small cell lung cancer (NSCLC); other subtypes include squamous cell lung carcinoma, bronchioloalveolar carcinoma, large cell carcinoma, carcinoid, adenoid cystic carcinoma, cylindroma, and mucoepidermoid carcinoma. In one embodiment, lung cancers are staged according to stages I-IV, with I being an early stage and IV being the most advanced.

“Prognosis” refers, e.g., to overall survival, long term mortality, and disease free survival. In one embodiment, long term mortality refers to death within 5 years after diagnosis of lung cancer. Although prognosis within 1, 2, or 3 years is also contemplated as is a prognosis beyond 5 years.

Other forms of cancer include carcinomas, sarcomas, adenocarcinomas, lymphomas, leukemias, etc., including solid and lymphoid cancers, head and neck cancer, e.g., oral cavity, pharyngeal and tongue cancer, kidney, breast, kidney, bladder, colon, ovarian, prostate, pancreas, stomach, brain, head and neck, skin, uterine, testicular, esophagus, and liver cancer, including hepatocarcinoma, lymphoma, including non-Hodgkin's lymphomas (e.g., Burkitt's, Small Cell, and Large Cell lymphomas) and Hodgkin's lymphoma, leukemia, and multiple myeloma.

The term “marker” or “biomarker” refers to a molecule (typically protein, nucleic acid, carbohydrate, or lipid) that is expressed in the cell, expressed on the surface of a cancer cell or secreted by a cancer cell in comparison to a non-cancer cell, and which is useful for the diagnosis of cancer, for providing a prognosis, and for preferential targeting of a pharmacological agent to the cancer cell. Oftentimes, such markers are molecules that are overexpressed in a lung cancer or other cancer cell in comparison to a non-cancer cell, for instance, 1-fold overexpression, 2-fold overexpression, 3-fold overexpression or more in comparison to a normal cell. Further, a marker can be a molecule that is inappropriately synthesized in the cancer cell, for instance, a molecule that contains deletions, additions or mutations in comparison to the molecule expressed on a normal cell. Alternatively, such biomarkers are molecules that are underexpressed in a cancer cell in comparison to a non-cancer cell, for instance, 1-fold underexpression, 2-fold underexpression, 3-fold underexpression, or more. Further, a marker can be a molecule that is inappropriately synthesized in cancer, for instance, a molecule that contains deletions, additions or mutations in comparison to the molecule expressed on a normal cell.

It will be understood by the skilled artisan that markers may be used in combination with other markers or tests for any of the uses, e.g., prediction, diagnosis, or prognosis of cancer, disclosed herein.

“Biological sample” includes sections of tissues such as biopsy and autopsy samples, and frozen sections taken for histologic purposes. Such samples include blood and blood fractions or products (e.g., serum, platelets, red blood cells, and the like), sputum, bronchoalveolar lavage, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., lung etc.), the size and type of the tumor, among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue from within the tumor. A diagnosis or prognosis made by endoscopy or radiographic guidance can require a “core-needle biopsy”, or a “fine-needle aspiration biopsy” which generally obtains a suspension of cells from within a target tissue. Biopsy techniques are discussed, for example, in Harrison's Principles of Internal Medicine, Kasper, et al., eds., 16th ed., 2005, Chapter 70, and throughout Part V.

The terms “overexpress,” “overexpression,” or “overexpressed” interchangeably refer to a protein or nucleic acid (RNA) that is translated or transcribed at a detectably greater level, usually in a cancer cell, in comparison to a normal cell. The term includes overexpression due to transcription, post transcriptional processing, translation, post-translational processing, cellular localization (e.g., organelle, cytoplasm, nucleus, cell surface), and RNA and protein stability, as compared to a normal cell. Overexpression can be detected using conventional techniques for detecting mRNA (i.e., RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a normal cell.

The terms “underexpress,” “underexpression,” or “underexpressed” or “downregulated” interchangeably refer to a protein or nucleic acid that is translated or transcribed at a detectably lower level in a cancer cell, in comparison to a normal cell. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In certain instances, underexpression is 1-fold, 2-fold, 3-fold, 4-fold or more lower levels of transcription or translation in comparison to a control.

The term “differentially expressed” or “differentially regulated” refers generally to a protein or nucleic acid that is overexpressed (upregulated) or underexpressed (downregulated) in one sample compared to at least one other sample, generally in a cancer patient compared to a sample of non-cancerous tissue in the context of the present invention.

“Therapeutic treatment” and “cancer therapies” refers to chemotherapy, hormonal therapy, radiotherapy, immunotherapy, and biologic and small molecule targeted therapy.

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that arc later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serino (S), Threonine (T); and 8) Cysteine (C), Methionine (M). See, e.g., Creighton, Proteins (1984).

The phrase “specifically (or selectively) binds” when referring to a protein, nucleic acid, antibody, or small molecule compound refers to a binding reaction that is determinative of the presence of the protein or nucleic acid, such as the differentially expressed genes of the present invention, often in a heterogeneous population of proteins or nucleic acids and other biologics. In the case of antibodies, under designated immunoassay conditions, a specified antibody may bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

The phrase “functional effects” in the context of assays for testing compounds that modulate a marker protein includes the determination of a parameter that is indirectly or directly under the influence of a biomarker of the invention, e.g., a chemical or phenotypic. A functional effect therefore includes ligand binding activity, transcriptional activation or repression, the ability of cells to proliferate, the ability to migrate, among others. “Functional effects” include in vitro, in vivo, and ex vivo activities.

By “determining the functional effect” is meant assaying for a compound that increases or decreases a parameter that is indirectly or directly under the influence of a biomarker of the invention, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers. The functional effects can be evaluated by many means known to those skilled in the art, e.g., microscopy for quantitative or qualitative measures of alterations in morphological features, measurement of changes in RNA or protein levels for other genes expressed in placental tissue, measurement of RNA stability, identification of downstream or reporter gene expression (CAT, luciferase, R-gal, GFP and the like), e.g., via chemiluminescence, fluorescence, colorimetric reactions, antibody binding, inducible markers, etc.

“Inhibitors,” “activators,” and “modulators” of the markers are used to refer to activating, inhibitory, or modulating molecules identified using in vitro and in vivo assays of cancer biomarkers. Inhibitors are compounds that, e.g., bind to, partially or totally block activity, decrease, prevent, delay activation, inactivate, desensitize, or down regulate the activity or expression of cancer biomarkers. “Activators” are compounds that increase, open, activate, facilitate, enhance activation, sensitize, agonize, or up regulate activity of cancer biomarkers, e.g., agonists. Inhibitors, activators, or modulators also include genetically modified versions of cancer biomarkers, e.g., versions with altered activity, as well as naturally occurring and synthetic ligands, antagonists, agonists, antibodies, peptides, cyclic peptides, nucleic acids, antisense molecules, ribozymes, RNAi and siRNA molecules, small organic molecules and the like. Such assays for inhibitors and activators include, e.g., expressing cancer biomarkers in vitro, in cells, or cell extracts, applying putative modulator compounds, and then determining the functional effects on activity, as described above.

Samples or assays comprising cancer biomarkers that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of cancer biomarkers is achieved when the activity value relative to the control is about 80%, preferably 50%, more preferably 25-0%. Activation of cancer biomarkers is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, peptide, circular peptide, lipid, fatty acid, siRNA, polynucleotide, oligonucleotide, etc., to be tested for the capacity to directly or indirectly modulate cancer biomarkers. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In some embodiments are provided a kit that includes a set of probes. A “probe” or “probes” refers to a polynucleotide that is at least eight (8) nucleotides in length and which forms a hybrid structure with a target sequence, due to complementarity of at least one sequence in the probe with a sequence in the target region. The polynucleotide can be composed of DNA and/or RNA. Probes in certain embodiments, are detectably labeled, as discussed in more detail herein. Probes can vary significantly in size. Generally, probes are, for example, at least 8 to 15 nucleotides in length. Other probes are, for example, at least 20, 30 or 40 nucleotides long. Still other probes are somewhat longer, being at least, for example, 50, 60, 70, 80, 90 nucleotides long. Yet other probes are longer still, and are at least, for example, 100, 150, 200 or more nucleotides long. Probes can be of any specific length that falls within the foregoing ranges as well. Preferably, the probe does not contain a sequence complementary to the sequence(s) used to prime for a target sequence during the polymerase chain reaction.

The terms “complementary” or “complementarity” are used in reference to polynucleotides (that is, a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Alternatively, there may be “complete” or “total” complementarity between the nucleic acids. The degree of complementarity between nucleic acid strands has significant effects on the efficiency and strength of hybridization between nucleic acid strands.

“Oligonucleotide” or “polynucleotide” refers to a polymer of a single-stranded or double-stranded deoxyribonucleotide or ribonucleotide, which may be unmodified RNA or DNA or modified RNA or DNA.

“Amplification detection assay” refers to a primer pair and matched probe wherein the primer pair flanks a region of a target nucleic acid, typically a target gene, which defines an amplicon, and wherein the probe binds to the amplicon.

A set of probes typically refers to a set of primers, usually primer pairs, and/or detectably-labeled probes that are used to detect the target genetic variations used in the actionable treatment recommendations of the disclosure. As a non-limiting example, a set of primers that are used to detect variants of EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, HRAS, KIT/PDGFRA, and/or the genes or variants in thereof in Tables 13-17, include at least one primer and typically a pair of amplification primers for each of the aforementioned genes, that are used to amplify a nucleic acid region that spans a particular genetic variant region in the aforementioned genes. As another non-limiting example, a set of amplification detection assays for EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, HRAS, KIT/PDGFRA, and/or the genes in Tables 13-17 and 19, includes a set of primer pairs and matched probes for each of the aforementioned genes. The primer pairs are used in an amplification reaction to define an amplicon that spans a region for a target genetic variation for each of the aforementioned genes. The set of amplicons are detected by a set of matched probes. In an exemplary embodiment, the invention is a set of TaqMan™ (Roche Molecular Systems, Pleasanton, Calif.) assays that are used to detect a set of target genetic variations used in the methods of the invention. For example, in one embodiment, the invention is a set of Taqman assays that detect the detect EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, HRAS, and/or KIT/PDGFRA genes.

In one embodiment, the set of probes are a set of primers used to generate amplicons that are detected by a nucleic acid sequencing reaction, such as a next generation sequencing reaction. In these embodiments, for example, AmpliSEQ™ (Life Technologies/Ion Torrent, Carlsbad, Calif.) or TruSEQ™ (Illumina, San Diego, Calif.) technology can be employed.

A modified ribonucleotide or deoxyribonucleotide refer to molecules that can be used in place of naturally occurring bases in nucleic acid and includes, but is not limited to, modified purines and pyrimidines, minor bases, convertible nucleosides, structural analogs of purines and pyrimidines, labeled, derivatized and modified nucleosides and nucleotides, conjugated nucleosides and nucleotides, sequence modifiers, terminus modifiers, spacer modifiers, and nucleotides with backbone modifications, including, but not limited to, ribose-modified nucleotides, phosphoramidates, phosphorothioates, phosphonamidites, methyl phosphonates, methyl phosphoramidites, methyl phosphonamidites, 5′-β-cyanoethyl phosphoramidites, methylenephosphonates, phosphorodithioates, peptide nucleic acids, achiral and neutral internucleotidic linkages.

In some embodiments are provided a kit that includes a set of probes provided wherein the set of probes specifically hybridize with polynucleotides encoding EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS or muteins thereof. In some embodiments are provided a kit that includes a set of probes provided wherein the set of probes specifically hybridize with polynucleotides encoding AKT1, ALK, BRAF, ERBB2, EGFR, FGFR1, HRAS, KIT, KRAS, MET, PIK3CA, RET and ROS or muteins thereof. In other embodiments, the kit includes a set of probes that specifically hybridize with polynucleotides encoding the genes, or muteins thereof, in Tables 13-17 and 19.

As used herein, “cleavage step” and its derivatives, generally refers to any process by which a cleavable group is cleaved or otherwise removed from a target-specific primer, an amplified sequence, an adapter or a nucleic acid molecule of the sample. In some embodiments, the cleavage step can involves a chemical, thermal, photo-oxidative or digestive process.

“Hybridize” or “hybridization” refers to the binding between nucleic acids. The conditions for hybridization can be varied according to the sequence homology of the nucleic acids to be bound. Thus, if the sequence homology between the subject nucleic acids is high, stringent conditions are used. If the sequence homology is low, mild conditions are used. When the hybridization conditions are stringent, the hybridization specificity increases, and this increase of the hybridization specificity leads to a decrease in the yield of non-specific hybridization products. However, under mild hybridization conditions, the hybridization specificity decreases, and this decrease in the hybridization specificity leads to an increase in the yield of non-specific hybridization products.

“Stringent conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed.

Hybridization between nucleic acids can occur between a DNA molecule and a DNA molecule, hybridization between a DNA molecule and a RNA molecule, and hybridization between a RNA molecule and a RNA molecule.

“AKT1” or “AKT” refers to human v-akt murine thymoma viral oncogene homolog 1, transcript variant 1; a polynucleotide encoding a RAC-alpha serine/threonine-protein kinase and appears as GenBank accession NM_005163.2, as updated on 30 Apr. 2011.

“ALK” refers to anaplastic lymphoma receptor tyrosine kinase, also known as anaplastic lymphoma kinase, is a gene that encodes a receptor tyrosine kinase, which belongs to the insulin receptor superfamily. This gene has been found to be rearranged, mutated, or amplified in a series of tumors including anaplastic large cell lymphomas, neuroblastoma, and non-small cell lung cancer. The chromosomal rearrangements are the most common genetic alterations in this gene, which result in creation of multiple fusion genes in tumorigenesis, including ALK (chromosome 2)/EML4 (chromosome 2), ALK/RANBP2 (chromosome 2), ALK/ATIC (chromosome 2), ALK/TFG (chromosome 3), ALK/NPM1 (chromosome 5), ALK/SQSTM1 (chromosome 5), ALK/KIF5B (chromosome 10), ALK/CLTC (chromosome 17), ALK/TPM4 (chromosome 19), and ALK/MSN (chromosome X). The translocation of ALK and EML4 results in a fusion protein. One polynucleotide encoding the fusion protein appears as GenBank accession AB274722.1, as updated on 11 Jan. 2008. Soda et al. “Identification of the transforming EML4-ALK fusion gene in non-small-cell lung cancer” (2007) Nature 448(7153):561-566. “EML” refers to “echinoderm microtubule associated protein like 4.”

“BRAF” refers to the proto-oncogene B-Raf and v-Raf, also referred to as serine/threonine-protein kinase B-Raf; a polynucleotide encoding a serine/threonine protein kinase and appears as GenBank accession NM_004333.4, as updated on 24 Apr. 2011. Variants of BRAF include polynucleotides encoding amino acid substitutions at amino acid positions 594 and 600. By “amino acid substitution” or “amino acid substitutions” is meant the replacement of an amino acid at a particular position in a parent polypeptide sequence with another amino acid. For example, the substitution D594H refers to a variant polypeptide, in which the aspartic acid at position 594 is replaced with histidine. Other variant polypeptides of BRAF include D594N and V600E.

“EGFR” or “Epidermal growth factor receptor” or “EGFR” refers to a tyrosine kinase cell surface receptor and is encoded by one of four alternative transcripts appearing as GenBank accession NM_005228.3, NM 201282.1, NM_201283.1 and NM_201284.1. Variants of EGFR include a deletion in exon 19, an insertion in exon 20, and amino acid substitutions T790M and L858R.

“ERBB2” also referred to as v-erb-b2 erythroblastic leukemia viral oncogene homolog 2, is a member of the EGFR/ErbB family and appears as GenBank accession NM_004448.2, as updated on 1 May 2011. Variants of ERBB2 include an insertion in Exon 20.

“FGFR1” or “fibroblast growth factor receptor 1” is also referred to as fms-related tyrosine kinase-2 and CD331. The nine alternative transcripts encoding FGFR1 protein appear as GenBank accession NM_023110.2, NM_001174063.1, NM_001174064.1, NM_001174065.1, NM_001174066.1, NM_001174067.1, NM_015850.3, NM_023105.2 and NM_023106.2 all as updated as on 30 Apr. 2011.

“HRAS” or “Harvey rat sarcoma viral oncogene homolog” is encoded by a polynucleotide appearing as GenBank accession NM_005343.2, as updated 17 Apr. 2011. Variants of HRAS include the amino acid substitutions Q61L and Q61R.

“KRAS” or “Kirsten rat sarcoma viral oncogene homolog” is encoded by two alternative transcripts appearing as GenBank accession NM_004985.3 and NM_033360.2. Variants of KRAS include the amino acid substitutions G12A/C/D/F/R/V.

“MET” or “MNNG HOS transforming gene” encodes a protein referred to as hepatocyte growth factor receptor and is encoded by a polynucleotide appearing as GenBank accession NM_000245.2 and NM_001127500.1.

“PIK3CA” or “phosphatidylinositol-4,5-bisphosphate 3-kinase, catalytic subunit alpha” is encoded by a polynucleotide appearing as NM_006218.2, as updated on 1 May 2011. Variants of PIK3CA include the amino acid substitutions E545A/G/K and H1047L/R.

“RET” or “rearranged during transfection” encodes a receptor tyrosine kinase. The chromosomal rearrangements are the most common genetic alterations in this gene, which result in creation of multiple fusion genes in tumorigenesis, including kinesin family member 5B (“KIF5B”)/RET, coiled-coil domain containing 6 (“CCDC6”)/RET and nuclear receptor coactivator 4 (“NCOA4”)/RET. A representative of the polynucleotide encoded by RET appears as NM_020630.4.

“ROS1” or “c-Ros receptor tyrosine kinase” belongs to the sevenless subfamily of tyrosine kinase insulin receptor genes. A representative of the polynucleotide encoded by ROS1 appears as NM_002944.2, as last updated on 28 Jan. 2013.

“KIT/PDGFRA” refers to two genes. “KIT,” also referred to as “proto-oncogene c-Kit” or “tyrosine-protein kinase Kit” encodes a cytokine receptor. A representative of the polynucleotide encoded by PDGFA appears as NM_000222.2. “PDGFA” is the gene encoding “alpha-type platelet-derived growth factor receptor.” A representative of the polynucleotide encoded by PDGFA appears as NM_006206.4.

A “mutein” or “variant” refers to a polynucleotide or polypeptide that differs relative to a wild-type or the most prevalent form in a population of individuals by the exchange, deletion, or insertion of one or more nucleotides or amino acids, respectively. The number of nucleotides or amino acids exchanged, deleted, or inserted can be 1, 2, 3, 4, 5, 6, 7, 8, 9, 10, 11, 12, 13, 14, 15, 16, 17, 18, 19, 20 or more such as 25, 30, 35, 40, 45 or 50. The term mutein can also encompass a translocation, for example the fusion of genes encoding the polypeptides EML4 and ALK. In some embodiments there is provided a kit encompassing a set of probes provided wherein the set of probes specifically hybridize with polynucleotides encoding AKT1, ALK, BRAF, ERBB2, EGFR, FGFR1, HRAS, KIT, KRAS, MET, PIK3CA, RET and ROS or muteins thereof, wherein the set of probes distinguish between the muteins and the muteins include one or more of the polynucleotides encoding AKT1 (E17K), BRAF (L597R, D594H/N, V600E), EGFR (L858R, G719X, T790M), HRAS (Q61L/K/R, G12C/D), KRAS G12A/C/D/F/R/V) and PIK3CA (E545A/G/K, H1047L/R).

“Driver event” or “driver alteration” refers to a mutation or genetic variation that confers a growth and/or survival advantage on the cells carrying them.

“Copy number” or “copy number variation” refers to alterations of the DNA of a genome that result in a cell having an abnormal number of copies of one or more sections of DNA. Copy number variations correspond to relatively large regions of the genome that have been deleted (copy number loss) or duplicated (copy number gain) on certain chromosomes.

“Single nucleotide polymorphism” or “SNP” refers to a DNA sequence variation that occurs when a single nucleotide (A, T, G, or C) in the genome differs between members of a biological species or paired chromosomes in a human.

In other embodiments, the two or more probes are primer pairs.

A “primer” or “primer sequence” refers to an oligonucleotide that hybridizes to a target nucleic acid sequence (for example, a DNA template to be amplified) to prime a nucleic acid synthesis reaction. The primer may be a DNA oligonucleotide, a RNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acid under such hybridization conditions. The upper limit is often determined by the possibility of having a duplex formation in a region other than the pre-determined nucleic acid sequence in the target nucleic acid. Generally, suitable primer lengths are in the range of about 10 to about 40 nucleotides long. In certain embodiments, for example, a primer can be 10-40, 15-30, or 10-20 nucleotides long. A primer is capable of acting as a point of initiation of synthesis on a polynucleotide sequence when placed under appropriate conditions.

The primer will be completely or substantially complementary to a region of the target polynucleotide sequence to be copied. Therefore, under conditions conducive to hybridization, the primer will anneal to the complementary region of the target sequence. Upon addition of suitable reactants, including, but not limited to, a polymerase, nucleotide triphosphates, etc., the primer is extended by the polymerizing agent to form a copy of the target sequence. The primer may be single-stranded or alternatively may be partially double-stranded.

In some embodiments there is provided a kit encompassing at least 4 primer pairs and 4 detectably labeled probes, wherein the at least 4 primer pairs and the at least 4 detectably labeled probes are not any one of the four primer pairs. In these non-limiting embodiments, the 4 primer pairs and 4 detectably labeled probes form 4 amplification detection assays.

“Detection,” “detectable” and grammatical equivalents thereof refers to ways of determining the presence and/or quantity and/or identity of a target nucleic acid sequence. In some embodiments, detection occurs amplifying the target nucleic acid sequence. In other embodiments, sequencing of the target nucleic acid can be characterized as “detecting” the target nucleic acid. A label attached to the probe can include any of a variety of different labels known in the art that can be detected by, for example, chemical or physical means. Labels that can be attached to probes may include, for example, fluorescent and luminescence materials.

“Amplifying,” “amplification,” and grammatical equivalents thereof refers to any method by which at least a part of a target nucleic acid sequence is reproduced in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridg, UK), and self-sustained sequence replication (3SR), including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other places, Sambrook et al. Molecular Cloning, 3^{rd} Edition; Ausbel et al.; PCR Primer: A Laboratory Manual, Diffenbach, Ed., Cold Spring Harbor Press (1995); The Electronic Protocol Book, Chang Bioscience (2002), Msuih et al., J Clin. Micro. 34:501-07 (1996); The Nucleic Acid Protocols Handbook, R. Rapley, ed., Humana Press, Totowa, N.J. (2002).

In some embodiments, one or more of the compositions, methods, kits and systems disclosed herein can include at least one target-specific primer and/or at least one adapter (see U.S. 2012/0295819, incorporated herein in its entirety by reference). In some embodiments, the compositions include a plurality of target-specific primers or adapters that are about 15 to about 40 nucleotides in length. In some embodiments, the compositions include one or more target-specific primers or adapters that include one or more cleavable groups. In some embodiments, one or more types of cleavable groups can be incorporated into a target-specific primer or adapter. In some embodiments, a cleavable group can be located at, or near, the 3′ end of a target-specific primer or adapter. In some embodiments, a cleavable group can be located at a terminal nucleotide, a penultimate nucleotide, or any location that corresponds to less than 50% of the nucleotide length of the target-specific primer or adapter. In some embodiments, a cleavable group can be incorporated at, or near, the nucleotide that is central to the target-specific primer or the adapter. For example, a target specific primer of 40 bases can include a cleavage group at nucleotide positions 15-25. Accordingly, a target-specific primer or an adapter can include a plurality of cleavable groups within its 3′ end, its 5′ end or at a central location. In some embodiments, the 5′ end of a target-specific primer includes only non-cleavable nucleotides. In some embodiments, the cleavable group can include a modified nucleobase or modified nucleotide. In some embodiments, the cleavable group can include a nucleotide or nucleobase that is not naturally occurring in the corresponding nucleic acid. For example, a DNA nucleic acid can include a RNA nucleotide or nucleobase. In one example, a DNA based nucleic acid can include uracil or uridine. In another example, a DNA based nucleic acid can include inosine. In some embodiments, the cleavable group can include a moiety that can be cleaved from the target-specific primer or adapter by enzymatic, chemical or thermal means. In some embodiments, a uracil or uridine moiety can be cleaved from a target-specific primer or adapter using a uracil DNA glycosylase. In some embodiments, a inosine moiety can be cleaved from a target-specific primer or adapter using hAAG or EndoV.

In some embodiments, a target-specific primer, adapter, amplified target sequence or nucleic acid molecule can include one or more cleavable moieties, also referred to herein as cleavable groups. Optionally, the methods can further include cleaving at least one cleavable group of the target-specific primer, adapter, amplified target sequence or nucleic acid molecule. The cleaving can be performed before or after any of the other steps of the disclosed methods. In some embodiments, the cleavage step occurs after the amplifying and prior to the ligating. In one embodiment, the cleaving includes cleaving at least one amplified target sequence prior to the ligating. The cleavable moiety can be present in a modified nucleotide, nucleoside or nucleobase. In some embodiments, the cleavable moiety can include a nucleobase not naturally occurring in the target sequence of interest. In some embodiments, uracil or uridine can be incorporated into a DNA-based nucleic acid as a cleavable group. In one exemplary embodiment, a uracil DNA glycosylase can be used to cleave the cleavable group from the nucleic acid. In another embodiment, inosine can be incorporated into a DNA-based nucleic acid as a cleavable group. In one exemplary embodiment, EndoV can be used to cleave near the inosine residue and a further enzyme such as Klenow can be used to create blunt-ended fragments capable of blunt-ended ligation. In another exemplary embodiment, the enzyme hAAG can be used to cleave inosine residues from a nucleic acid creating abasic sites that can be further processed by one or more enzymes such as Klenow to create blunt-ended fragments capable of blunt-ended ligation.

In some embodiments, one or more cleavable groups can be present in a target-specific primer or adapter. In some embodiments, cleavage of one or more cleavable groups in a target-specific primer or an adapter can generate a plurality of nucleic acid fragments with differing melting temperatures. In one embodiment, the placement of one or more cleavable groups in a target-specific primer or adapter can be regulated or manipulated by determining a comparable maximal minimum melting temperature for each nucleic acid fragment, after cleavage of the cleavable group. In some embodiments the cleavable group can be a uracil or uridine moiety. In some embodiments the cleavable group can be an inosine moiety. In some embodiments, at least 50% of the target-specific primers can include at least one cleavable group. In some embodiments, each target-specific primer includes at least one cleavable group.

In one embodiment, a multiplex nucleic acid amplification is performed that includes a) amplifying one or more target sequences using one or more target-specific primers in the presence of polymerase to produce an amplified target sequence, and b) ligating an adapter to the amplified target sequence to form an adapter-ligated amplified target sequence. In some embodiments, amplifying can be performed in solution such that an amplified target sequence or a target-specific primer is not linked to a solid support or surface. In some embodiments, ligating can be performed in solution such that an amplified target sequence or an adapter is not linked to a solid support or surface. In another embodiment, amplifying and ligating can be performed in solution such that an amplified target sequence, a target-specific primer or an adapter is not linked to a solid support or surface.

In some embodiments, the target-specific primer pairs do not contain a common extension (tail) at the 3′ or 5′ end of the primer. In another embodiment, the target-specific primers do not contain a Tag or universal sequence. In some embodiments, the target-specific primer pairs are designed to eliminate or reduce interactions that promote the formation of non-specific amplification.

In one embodiment, the target-specific primer pairs comprise at least one cleavable group per forward and reverse target-specific primer. In one embodiment, the cleavable group can be a uracil nucleotide. In one embodiment, the target-specific primer pairs are partially or substantially removed after generation of the amplified target sequence. In one embodiment, the removal can include enzymatic, heat or alkali treatment of the target-specific primer pairs as part of the amplified target sequence. In some embodiments, the amplified target sequences are further treated to form blunt-ended amplification products, referred to herein as, blunt-ended amplified target sequences.

According to various embodiments, there are provided methods for designing primers using a design pipeline that allows design of oligonucleotide primers across genomic areas of interest while incorporating various design criteria and considerations including amplicon size, primer composition, potential off-target hybridization, and SNP overlap of the primers. In an embodiment, the design pipeline includes several functional modules that may be sequentially executed as discussed next.

First, in an embodiment, a sequence retrieval module may be configured to retrieve sequences based on instructions of an operator regarding a final product desired by a customer. The operator may request a design of primer pairs for genomic regions which may be specified by chromosome and genome coordinates or by a gene symbol designator. In the latter case, the sequence retrieval module may retrieve the sequence based on the exon coordinates. The operator may also specify whether to include a 5′ UTR sequence (untranslated sequence).

Second, in an embodiment, an assay design module may be configured to design primer pairs using a design engine, which may be a public tool such as Primer3 or another primer design software that can generate primer pairs across the entire sequence regions retrieved by the sequence retrieval module, for example. The primers pairs may be selected to tile densely across the nucleotide sequence. The primer design may be based on various parameters, including: (1) the melting temperature of the primer (which may be calculated using the nearest neighbor algorithm set forth in John SantaLucia, Jr., “A unified view of polymer, dumbbell, and oligonucleotide DNA nearest-neighbor thermodynamics,” Proc. Natl. Acad. Sci. USA, vol. 95, 1460-1465 (1998), the contents of which is incorporated by reference herein in its entirety), (2) the primer composition (e.g., nucleotide composition such as GC content may be determined and filtered and penalized by the software, as may be primer hairpin formation, composition of the GC content in the 3′ end of primer, and specific parameters that may be evaluated are stretches of homopolymeric nucleotides, hairpin formation, GC content, and amplicon size), (3) scores of forward primer, reverse primer and amplicon (the scores may be added up to obtain a probe set score, and the score may reflect how close the amplicon confirms with the intended parameters), and (4) conversion of some of the T's to U's (T's may be placed such that the predicted Tm of the T delimited fragments of a primer have a minimum average Tm.)

Third, in an embodiment, a primer mapping module may be configured to use a mapping software (e.g., e-PCR (NCBI), see Rotmistrovsky et al., “A web server for performing electronic PCR,” Nucleic Acids Research, vol. 32, W108-W112 (2004), and Schuler, “Sequence Mapping by Electronic PCR,” Genome Research, vol. 7, 541-550 (1997), which are both incorporated by reference herein in their entirety, or other similar software) to map primers to a genome. The primers mapping may be scored using a mismatch matrix. In an embodiment, a perfect match may receive a score of 0, and mismatched primers may receive a score of greater than 0. The mismatch matrix takes the position of the mismatch and the nature of the mismatch into account. For example, the mismatch matrix may assign a mismatch score to every combination of a particular motif (e.g., AA, AC, AG, CA, CC, CT, GA, GG, GT, TC, TG, TT, A-, C-, G-, T-, -A, -C, -G, and -T, where ‘-’ denotes an ambiguous base or gap) with a particular position (e.g., base at 3′ end, second base from 3′ end, third base from 3′ end, third base from 5′ end, second base from 5′ end, base at 5′ end, and positions therebetween), which may be derived empirically and may be selected to reflect that mismatches closer to the 3′end tend to weaker PCR reactions more than mismatches closer to the 5′ end and may therefore be generally larger. The mismatch scores for motifs with an ambiguous base or gap may be assigned an average of scores of other motifs consistent therewith (e.g., A- may be assigned an average of the scores of AA, AC, and AG). Based on the number of hits with a certain score threshold, an amplicon cost may be calculated.

Fourth, in an embodiment, a SNP module may be configured to determine underlying SNPs and repeat regions: SNPs may be mapped to the primers and based on the distance of a SNP from the 3′ end, primers may be filtered as potential candidates. Similarly, if a primer overlaps to a certain percentage with a repeat region, the primer might be filtered.

Fifth, in an embodiment, a tiler module may be configured to use a function based on the amplicon cost (see primer mapping) and the number of primers necessary to select a set of primers covering the target while ensuring that selection of tiling primers for a target is independent of other targets that may be in a customer's request so that the same set of primers for a target will be selected whether the customer requested only that target or additional targets and whether amplicons are to help cover on that target or additional targets.

Sixth, in an embodiment, a pooler module may be configured to use a pooling algorithm that prevents amplicon overlaps, and ensures that the average number of primers in a pool does not deviate by more than a preset value.

According to an exemplary embodiment, there is provided a method, comprising: (1) receiving one or more genomic regions or sequences of interest; (2) determining one or more target sequences for the received one or more genomic regions or sequences of interest; (3) providing one or more primer pairs for each of the determined one or more target sequences; (4) scoring the one or more primer pairs, wherein the scoring comprises a penalty based on the performance of in silico PCR for the one or more primer pairs, and wherein the scoring further comprises an analysis of SNP overlap for the one or more primer pairs; and (5) filtering the one or more primer pairs based on a plurality of factors, including at least the penalty and the analysis of SNP overlap, to identify a filtered set of primer pairs corresponding to one or more candidate amplicon sequences for the one or more genomic regions or sequences of interest.

The amount of nucleic acid material required for successful multiplex amplification can be about 1 ng. In some embodiments, the amount of nucleic acid material can be about 10 ng to about 50 ng, about 10 ng to about 100 ng, or about 1 ng to about 200 ng of nucleic acid material. Higher amounts of input material can be used, however one aspect of the disclosure is to selectively amplify a plurality of target sequence from a low (ng) about of starting material.

Analysis of nucleic acid markers can be performed using techniques known in the art including, without limitation, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

“Detectably labeled probe” refers to a molecule used in an amplification reaction, typically for quantitative or real-time PCR analysis, as well as end-point analysis. Such detector probes can be used to monitor the amplification of the target nucleic acid sequence. In some embodiments, detector probes present in an amplification reaction are suitable for monitoring the amount of amplicon(s) produced as a function of time. Such detector probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

Detector probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detector probes can also include two probes, wherein for example a fluor is on one probe, and a quencher is on the other probe, wherein hybridization of the two probes together on a target quenches the signal, or wherein hybridization on the target alters the signal signature via a change in fluorescence. Detector probes can also comprise sulfonate derivatives of fluorescenin dyes with SO₃ instead of the carboxylate group, phosphoramidite forms of fluorescein, phosphoramidite forms of CY 5 (commercially available for example from Amersham). In some embodiments, interchelating labels are used such as ethidium bromide, SYBR® Green I (Molecular Probes), and PicoGreen® (Molecular Probes), thereby allowing visualization in real-time, or end point, of an amplification product in the absence of a detector probe. In some embodiments, real-time visualization can comprise both an intercalating detector probe and a sequence-based detector probe can be employed. In some embodiments, the detector probe is at least partially quenched when not hybridized to a complementary sequence in the amplification reaction, and is at least partially unquenched when hybridized to a complementary sequence in the amplification reaction. In some embodiments, the detector probes of the present teachings have a Tm of 63-69° C., though it will be appreciated that guided by the present teachings routine experimentation can result in detector probes with other Tins. In some embodiments, probes can further comprise various modifications such as a minor groove binder (see for example U.S. Pat. No. 6,486,308) to further provide desirable thermodynamic characteristics.

In some embodiments, detection can occur through any of a variety of mobility dependent analytical techniques based on differential rates of migration between different analyte species. Exemplary mobility-dependent analysis techniques include electrophoresis, chromatography, mass spectroscopy, sedimentation, for example, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described for example in Published P.C.T. Application WO04/46344 to Rosenblum et al., and WO01/92579 to Wenz et al. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, IIlumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003). It will also be appreciated that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. Detection of unlabeled reaction products, for example using mass spectrometry, is also within the scope of the current teachings.

The kits of the present invention may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

In some embodiments is provided a composition comprising a set of probes and a sample, wherein the set of probes specifically recognize the genes AKT1, ALK, BRAF, ERBB2, EGFR, FGFR1, HRAS, KIT, KRAS, MET, PIK3CA, RET and ROS, and wherein the set of probes can recognize and distinguish one or more allelic variants of the genes AKT1, ALK, BRAF, ERBB2, EGFR, HRAS, KRAS, MET, PIK3CA, RET and ROS.

In yet other embodiments, compositions, kits, methods and workflows disclosed herein comprise a set of probes that specifically recognize one or more genes and/or variants thereof, in Tables 13-17 and 19.

Any combination of the disclosed genes and variants can be included in the kits and compositions. For instance, the genes and variants can be selected from a combination of actionability index (AI) categories and variant prevalence, as described in more detail herein. In this regard, in varying embodiments of the disclosed compositions and kits, the gene variants can be selected from an actionability index A, A2, A3, A4, or A5. In other embodiments, gene variants can be selected from an actionability index and percentage prevalence selected from AI1+Prevalence>1%, AI2+Prevalence>1%, AI3+Prevalence>1%, AI1+Prevalence 0.1%-1%, AI2+Prevalence 0.1%-1%, AI3+Prevalence 0.1%-1%, and combinations thereof.

In certain embodiments, methods to determine an actionable treatment recommendation for a subject diagnosed with cancer are provided. Other embodiments include methods to determine the likelihood of a response to a treatment in a subject afflicted with cancer and methods for treating a patient with cancer (e.g., lung cancer).

In one embodiment of the methods, the cancer is lung cancer and the sub type is lung adenocarcinoma. In certain embodiments, the lung cancer subtype is squamous cell lung carcinoma.

The methods comprise the steps of obtaining a sample from a patient, detecting at least one variant in a gene of interest, and determining an A1 or treatment for the patient based on the gene variant detected.

The patient sample can be any bodily tissue or fluid that includes nucleic acids from the lung cancer in the subject. In certain embodiments, the sample will be a blood sample comprising circulating tumor cells or cell free DNA. In other embodiments, the sample can be a tissue, such as a lung tissue. The lung tissue can be from a tumor tissue and may be fresh frozen or formalin-fixed, paraffin-embedded (FFPE). In certain embodiments, a lung tumor FFPE sample is obtained.

Five categories of AIs are provided herein. AI1 represents a category for which there is clinical consensus on a treatment recommendation based on the genetic variant status. The data source for AI1 is the National Comprehensive Cancer Network Practice Guidelines in Oncology (NCCN Guidelines) for non-small cell lung cancer (NSCLC) (Version 2.2013). This index is assigned if the NCCN Guidelines specifically recommends a therapy based on gene and variant type.

AI2 represents a category for which there exists a clinical trial or clinical case report evidence for treatment response in patients based on genetic variant status.

AI3 is a category in which one or more clinical trials are in progress in which genetic variant status is used as an enrollment criteria, that is particular genes and variants are required as part of the clinical trial enrollment criteria (for inclusion or exclusion).

AI4 is a category for which there is preclinical evidence for treatment response based on genetic variant status. The index contains genes and events reported to show an association with preclinical treatment response.

AI5 is a category in which a targeted therapy is available for the gene that is aberrant. This index is based on the requirement for a gene and associated variant in order for the therapy to be considered actionable.

In certain embodiments, lung cancer variants are prioritized based on prevalence of greater than 0.1%. Prevalence was determined from references datasets of lung cancer by counting all of the clinical specimens tested that were found to contain one of the gene variants described in this invention, and expressing that value as a percentage relative to all of the clinical specimens tested. For example, the prevalence of 0.1% to 1% and prevalence of greater than 1% of gene variants in adenocarcinoma and squamous cell carcinoma are shown herein (see Tables 1 and 3), however any subset of the percentage range, or below or above the percentage range, can be used to represent additional genetic variants associated with an A1. The variants include but are not limited to SNPs, insertions, deletions, translocations, and copy number variation (e.g., gain or loss).

TABLE 1
Lung Adenocarcinoma

| Actionabil- | | |
|---|---|---|
| ity Index | Prevalence >1% | Prevalence 0.1%-1% |
| AI1 | EGFR (L858R, Exon 19 | EGFR (G719X) |
| | del, T790M, exon 20 ins) | KRAS (G12S, G13C, |
| | ALK translocation/fusion | G13D, G12R, G12F) |
| | (EML4-ALK) | |
| | ROS1 (EZR-ROS1, | |
| | SLC34A2-ROS1, CD74- | |
| | ROS1, SDC4-ROS1) | |
| | KRAS (G12C, G12V, G12D, | |
| | G12A) | |
| AI2 | BRAF (V600E) | PIK3CA (E545K, E545G, |
| | ERBB2 (Exon 20 ins) | E545A, H1047R, H1047L) |
| | MET CN gain | |
| AI3 | RET translocation | AKT1 (E17K) |
| | EGFR CN gain | BRAF (L597R, D594H/N) |
| | ERBB2 CN gain | HRAS (Q61L/K/R, |
| | FGFR1 CN gain | G12C/D, G13C/S/R/V) |
| | KIT/PDGFRA amplification | PIK3CA (E542K) |

As shown in Table 1, the genetic variants disclosed herein and associated AIs, provide treatment options for over 50% of all primary lung adenocarcinomas. This type of comprehensive screening of lung cancer gene variants and treatment recommendations for over 50% of the lung adenocarcinoma patient population has been heretofore unavailable. The disclosure provides a method of gene variant determination that can be performed in a single assay or panel, which allows greater variant detection using the precious little sample obtained from a typical lung tumor biopsy or surgical resection. It should be understood that the genes and variants identified herein are non-limiting examples and genes and variants can be readily added or removed identify valuable patient variants and treatment options. Further, any combination of A1 and prevalence can be detected in the methods provided herein. For example, in one embodiment, all A1 categories and variants can be determined. In another embodiment, AI1+Prevalence>1%, AI2+Prevalence>1%, AI3+Prevalence>1%, AI1+Prevalence 0.1%-1%, AI2+Prevalence 0.1%-1%, AI3+Prevalence 0.1%-1% and any combination thereof can be determined in the methods disclosed herein.

The disclosure provides treatment options for numerous subsets of the adenocarcinoma and squamous cell carcinoma population depending on the combination of the percentage prevalence of the markers chosen and the A1 categories. As shown in Tables 4-8, by choosing different combinations of AI+% prevalence, treatment options can be provided for varying percentages of the afflicted population (See Example II).

The disclosure further provides actionable treatment recommendations for a subject with lung cancer based on the subject's tumor's genetic variant status. The actionable treatment recommendations can include pharmaceutical therapeutics, surgery, photodynamic therapy (PTD), laser therapy, radiation, dietary guidance, clinical trial suggestions, etc. The actionable treatment recommendations provided herein (see Tables 2 and 3) are exemplary. Additional actionable treatment recommendations can be added or removed as additional data, publications, clinical reports, treatments, and clinical trials become available. Further, additional information can be used to provide actionable treatment recommendations, including, but not limited to, age, gender, family history, lifestyle, dietary, as well as other relevant factors.

In certain embodiments, the method comprises performing the actionable treatment recommendation. Accordingly, performing the actionable treatment recommendation can include, without limitation, administering a therapeutically effective amount of one or more therapeutic agents (chemotherapeutics, targeted therapeutics, antiangiogenics, etc), implementing a dietary regimen, administering radiation and/or enrolling in one or more clinical trials.

Examples of chemotherapeutics to treat lung cancer include: Cisplatin or carboplatin, gemcitabine, paclitaxel, docetaxel, etoposide, and/or vinorelbine. Targeted therapeutics (drugs that specifically block the growth and spread of cancer) include monoclonal antibodies such as, but not limited to, bevacizumab (AVASTIN™) and cetuximab; and tyrosine kinase inhibitors (TKIs) such as, but not limited to, gefitinib (IRESSA™), erlotinib (TARCEVA™) crizotinib and/or vemurafenib.

Additional chemotherapeutics to treat lung cancer include, but are not limited to, TKIs: vandetanib, tofacitinib, sunitinib malate, sorafenib, ruxolitinib, regorafenib, ponatinib, pazopanib, nilotinib, leflunomide, lapatinib ditosylate, imatinib mesilate, gefitinib, erlotinib, dasatinib, crizotinib, cabozantinib, bosutinib, axitinib, radotinib, tivozanib, masitinib, afatinib, XL-647, trebananib, tivantinib, SAR-302503, rilotumumab, ramucirumab, plitidepsin, pacritinib, orantinib, nintedanib, neratinib, nelipepimut-S, motesanib diphosphate, midostaurin, linifanib, lenvatinib, ibrutinib, fostamatinib disodium, elpamotide, dovitinib lactate, dacomitinib, cediranib, baricitinib, apatinib, Angiozyme, X-82, WBI-1001, VX-509, varlitinib, TSR-011, tovetumab, telatinib, RG-7853, RAF-265, R-343, R-333, quizartinib dihydrochloride, PR-610, poziotinib, PLX-3397, PF-04554878, Pablocan, NS-018, momelotinib, MK-1775, milciclib maleate, MGCD-265, linsitinib, LDK-378, KX2-391, KD-020, JNJ-40346527, JI-101, INCB-028060, icrucumab, golvatinib, GLPG-0634, gandotinib, foretinib, famitinib, ENMD-2076, danusertib, CT-327, crenolanib, BMS-911543, BMS-777607, BMS-754807, BMS-690514, bafetinib, AZD-8931, AZD-4547, AVX-901, AVL-301, AT-9283, ASP-015K, AP-26113, AL-39324, AKN-028, AE-37, AC-480, 2586184, X-396, volitinib, VM-206, U3-1565, theliatinib, TAS-115, sulfatinib, SB-1317, SAR-125844, S-49076, rebastinib, R84 antibody, Peregrine, R-548, R-348, PRT-062607, P-2745, ONO-4059, NRC-AN-019, LY-2801653, KB-004, JTE-052, JTE-051, IMC-3C5, ilorasertib, IDN-6439, HM-71224, HM-61713, henatinib, GSK-2256098, epitinib, EMD-1214063, E-3810, EOS, CUDC-101, CT-1578, cipatinib, CDX-301, CC-292, BI-853520, BGJ-398, ASP-3026, ARRY-614, ARRY-382, AMG-780, AMG-337, AMG-208, AL-3818, AC-430, 4SC-203, Z-650, X-379, WEE-1/CSN5, Tekmira Pharmaceuticals, Wee-1 kinase inhibitors, Tekmira Pharmaceuticals, VS-4718, VEGFR2 inhibitor, AB Science, VEGF/rGel, Clayton Biotechnologies, VEGF inhibitors, Interprotein, UR-67767, tyrosine kinase inhibitors, Bristol-Myers Squibb, tyrosine kinase inhibitor, Aurigene Discovery Technologies, tyrosine kinase 2 inhibitors, Sareum, TrkA ZFP TF, TrkA inhibitor, Proximagen, TP-0903, TP-0413, TKI, Allergan, Sym-013, syk kinase inhibitors, Almirall, Syk kinase inhibitors, AbbVie, SYK inhibitor programme, Ziarco, SUN-K706, SN-34003, SN-29966, SIM-930, SIM-6802, SIM-010603, SGI-7079, SEL-24-1, SCIB-2, SAR-397769, RET kinase inhibitor, Bionomics, R-256, PRT-062070, PRT-060318, PRS-110, PLX-7486, ORS-1006, ORB-0006, ORB-0004, ORB-0003, ONO-WG-307, ON-044580, NVP-BSK805, NNI-351, NMS-P948, NMS-E628, NMS-173, MT-062, MRLB-11055, MG-516, KX2-361, KIT816 inhibitor, AB Science, janus kinase inhibitor, Celgene, JAK3-inhibitor, Principia BioPharma, Jak1 inhibitor, Genentech, JAK inhibitors, Almirall, INCB-16562, hRl-derivatives, Immunomedics, HMPL-281, HM-018, GTX-186, GSK-143, GS-9973, GFB-204, gastrointestinal stromal tumour therapy, Clovis Oncology, G-801, FX-007, FLT4 kinase inhibitors, Sareum, FLT3/cKit inhibitor, Johnson & Johnson, flt-4 kinase inhibitors, Sareum, flt-3 kinase inhibitors, Sareum, FAK inhibitors, Takeda, FAK inhibitor, Verastem, EN-3351, DNX-04040, DNX-02079, DLX-521, deuterated tofacitinib, Auspex Pharmaceuticals, DCC-2721, DCC-2701, DCC-2618, CTX-0294945, CTx-0294886, CT-340, CT-053, CST-102, CS-510, CPL-407-22, CH-5451098, CG-206481, CG-026828, CFAK-C4, CCT-137690, CC-509, c-Met kinase inhibitors, Rhizen, BXL-1H5, BTK inhibitors, Mannkind, Btk inhibitor, Pharmacyclics-3, Btk inhibitor, Aurigene Discovery Technologies, BGB-324, BGB-001, Bcr-Abl/Lyn inhibitor, AB Science, aurora kinase+FLT3 kinase inhibitor, Sareum, aurora kinase+ALK inhibitor, Sareum, aurora kinase+ALK inhibitor, AstraZeneca, ASP-502D, ASP-08112, ARYY-111, AR-523, anticancer, leukemia, Critical, anticancer therapy, Agios-1, ANG-3070, ALK inhibitors, AstraZeneca, Alk inhibitor, Cephalon-3, ALK inhibitor, Aurigene Discovery Technologies, AL-2846, TrkB modulators, Hermo Pharma, TLK-60596, TLK-60404, CYC-116, ARRY-380, ZD-4190, Yissum Project No. B-1146, XL-999, XL-820, XL-228, VX-667, vatalanib, tyrosine protein kinase inhibs, tyrosine kinase inhibs, Yissum, tyrosine kinase inhibs, CSL, tyrosine kinase antags, ICRT, tozasertib lactate, TG-100-13, tandutinib, TAK-593, TAK-285, Symadex, Syk kinase inhibitor, SGX, SU-5271, SU-14813, SGX-523, semaxanib, saracatinib, RP 53801, RG-14620, RG-13291, RG-13022, R-112, PLX-647, PKI-166, Pharmaprojects No. 6085, Pharmaprojects No. 4960, Pharmaprojects No. 4923, Pharmaprojects No. 4863, Pharmaprojects No. 3624, Pharmaprojects No. 3292, Pharmaprojects No. 3054, PF-562271, PF-4217903, NVP-TAE226, mubritinib, MEDI-547, lestaurtinib, KW-2449, KSB-102, KRN-633, IMC-EB10, GW-282974, Flt3-kinase inhibitor, Lilly, FCE-26806, EphA2 vaccine, MedImmune, EMD-55900, EMD-1204831, desmal, degrasyns, CNF-201 series, CGP-57148, CEP-7055, CEP-5214, CEP-075, CE-245677, CDP-860, canertinib dihydrochloride, cancer vaccine, Ajinomoto, bscEphA2xCD3, MedImmune, brivanib alaninate, breast cancer therapy, Galapago, BIBX-1382, AZD-9935, AZD-6918, AZD-4769, AZD-1480, AVE-0950, Argos, AP-23464, AP-23451, AP-22408, anti-HER2/neu mimetic, Cyclacel, anti-HER-2/neu antisense, Tekm, amuvatinib, AG-490, AG-18, AG-13958, AEG-41174, ZM-254530, ZK-CDK, ZK-261991, ZD-1838, ZAP70 kinase inhibitors, Kinex, ZAP-70 inhibitors, Cellzome, ZAP inhibitors, Ariad, ZAP 70 inhibitors, Galapagos, ZAP 70 inhibitors, Celgene, YW327.6S2, YM-359445, YM-231146, YM-193306, XV-615, XL-019, XC-441, XB-387, Wee-1 kinase inhibitor, Banyu, VX-322, VRT-124894, VEGFR2 kinase inhibitors, Takeda, VEGFR/EGFR inhib, Amphora, VEGFR-2 kinase inhibitors, Hanmi, VEGFR-2 antagonist, Affymax, VEGF/rGel, Targa, VEGF-TK inhibitors, AstraZeneca, VEGF-R inhibitors, Novartis, VEGF modulators, 3-D, VEGF inhibitors, Onconova, VEGF inhibitor, Chugai, V-930, U3-1800, U3-1784, tyrphostins, Yissum, tyrosine kinase inhibs, Novar-2, tyrosine kinase inhibs, Sanofi, tyrosine kinase inhib, Abbott-2, tyrosine kinase inhib, Pfizer, tyrosine kinase inhib, IQB, tyrosine kinase inhib, Abbott, tyrosine kinase inhi, Abbott-3, trkB inhibitors, Amphora, TrkA inhibitors, Telik, TrkA blocker, Pfizer, TLN-232, TKM-0150, Tie-2 kinase inhibitors, GSK, TIE-2 inhibitors, Ontogen, Tie-2 inhibitors, AstraZeneca, Tie-2 inhibitors, Amgen-3, Tie-2 inhibitors, Amgen-2, Tie-2 inhibitors, Amgen, Tie-2 antagonists, Semaia, Tie-1R IFP, Receptor BioLogix, TG-101-223, TG-101-209, TG-100948, TG-100435, TG-100-96, TG-100-801, TG-100-598, TAE-684, T3-106, T-cell kinase inhibitors, Cell, syk kinase inhibitor, Bayer, Syk inhibitors, CrystalGenomics, Syk inhibitors, Astellas-2, Syk inhibitors, Amphora, SU-11657, SU-0879, SSR-106462, SRN-004, Src/Abl inhibitors, Ariad, Src non-RTK antagonists, SUGEN, Src inhibitors, Amphora, spiroindolines, Pfizer, SP-5.2, sorafenib bead, Biocompatibles, SMi-11958, SH2 inhibitors, NIH, SH-268, SGX-393, SGX-126, SGI-1252, SC-102380, SC-101080, SB-238039, SAR-131675, RWJ-64777, RWJ-540973, RPR-127963E, RP-1776, Ro-4383596, RNAi cancer therapy, Benitec Biopharma, RM-6427, rheumatoid arthritis therapy, SRI International, RET inhibitors, Cell T, RB-200h, R545, Rigel, R3Mab, R-723, R-507, R-499, R-1530, QPM5-986, QPAB-1556, PX-104.1, PS-608504, prostate cancer ther, Sequenom, prodigiosin, PRI-105, PP1, Scripps, PN-355, phenylalanine derivatives, NIH, Pharmaprojects No. 6492, Pharmaprojects No. 6291, Pharmaprojects No. 6271, Pharmaprojects No. 6267, Pharmaprojects No. 6140, Pharmaprojects No. 6138, Pharmaprojects No. 6083, Pharmaprojects No. 6059, Pharmaprojects No. 6013, Pharmaprojects No. 5330, Pharmaprojects No. 4855, Pharmaprojects No. 4597, Pharmaprojects No. 4368, Pharmaprojects No. 4164, Pharmaprojects No. 3985, Pharmaprojects No. 3495, Pharmaprojects No. 3135, PF-371989, PF-337210, PF-00120130, pelitinib, pegdinetanib, PDGFR-alpha inhibitors, Deciphera, PDGFR inhibitor, Pulmokine, PDGFR inhibitor, Array, PDGF receptor inhibitor, Kyowa, PDGF receptor inhibitor, Array, PDGF kinase inhibitors, Kinex, PD-180970, PD-173956, PD-171026, PD-169540, PD-166285, PD-154233, PD-153035, PD-0166285, PCI-31523, pazopanib hydrochloride (ophthalmic), pan-HER kinase inhib, Ambit-2, pan-HER inhibitor, SUGEN, pan-HER ACL, p56lck inhibitors, BI, OSI-930, OSI-817, OSI-632, OSI-296, ONC-101, ON-88210, ON-045270, NVP-AEW541, NVP-AAK980-NX, NV-50, NSC-242557, NNC-47-0011, NMS-P626, NL-0031, nilotinib, once-daily, nicotinamide derivatives, Bristol-Myers Squibb, neuT MAb, Philadelphia, multi-kinase inhibitors, Amphor, mullerian inhibiting subst, Ma, MS therapy, Critical Outcome Technologies, MP-371, MLN-608, MK-8033, MK-2461, Met/Ron kinase inhibs, SGX, Met/Gabl antagonist, Semaia, Met RTK antagonists, SUGEN, Met receptor inhibs, Ontogen, Met kinase inhibitor, BMS, Met inhibitors, Amphora, MEDI-548, MED-A300, ME-103, MC-2002, Lyn kinase inhibitor, CRT, Lyn B inhibitors, Onconova, lymphostin, LP-590, leflunomide, SUGEN, lck/Btk kinase inhibitors, AEgera, lck kinase inhibitors, Kinex, lck kinase inhibitors, Celgene, Lck inhibitors, Green Cross, lck inhibitors, Amphora, lck inhibitors, Amgen, lck inhibitors, Abbott, lavendustin A analogues, NIH, LAT inhibitors, NIH, L-000021649, KX-2-377, KST-638, KRX-211, KRX-123, KRN-383, KM-2550, kit inhibitor, Amphora, kinase inhibitors, SGX-2, kinase inhibitors, SGX-1, kinase inhibitors, MethylGene, kinase inhibitors, Amgen, kinase inhibitor, Cephalon, KIN-4104, Ki-8751, Ki-20227, Ki-11502, KF-250706, KDR kinase inhibs, Celltech, KDR kinase inhibitors, Merck & Co-2, KDR kinase inhibitors, Merck & Co-1, Kdr kinase inhibitors, Amgen, KDR inhibitors, Abbott, KDR inhibitor, LGLS, K252a, JNJ-38877605, JNJ-26483327, JNJ-17029259, JNJ-141, Janex-1, JAK3 inhibitors, Pharmacopeia-2, Jak3 inhibitors, Portola, JAK2 inhibitors, Merck & Co, JAK2 inhibitors, Deciphera, JAK2 inhibitors, Amgen, JAK2 inhibitors, Abbott, JAK2 inhibitor, CV, Cytopia, JAK2 inhibitor, cancer, Cytopia, JAK2 inhibitor, Astex, JAK-3 inhibitors, Cellzome, JAK inhibitors, Genentech, JAK inhibitors, BioCryst, JAK inhibitor, Pulmokine, JAK 1/3 inhibitor, Rigel, ITK inhibitors, GlaxoSmithKline, ISU-101, interleukin-2 inducible T-cell kinase inhibitors, Vertex, INSM-18, inherbins, Enkam, IMC-1C11, imatinib, sublingual, Kedem Pharmaceuticals, IGF-1R inhibitor, Allostera, IGF-1 inhibitors, Ontogen, HMPL-010, HM-95091, HM-60781, HM-30XXX series, Her2/neu & EGFR Ab, Fulcrum, HER2 vaccine, ImmunoFrontier, HER-2 binder, Borean, Her-1/Her-2 dual inhibitor, Hanmi, Her inhibitors, Deciphera, HEM-80322, HDAC multi-target inhibitors, Curis, GW-771806, GW-654652, GSK-1838705A, GNE-A, glioblastoma gene therapy, Biogen Idec, genistein, gene therapy, UCSD, focal adhesion kinase inhibitor, Kinex, FMS kinase inhibitors, Cytopia, FLT-3 MAb, ImClone, Flt-3 inhibitor, Elan, Flt 3/4 anticancer, Sentinel, FAK/JAK2 inhibitors, Cephalon, FAK inhibitors, Ontogen, FAK inhibitors, Novartis, FAK inhibitors, GlaxoSmithKline, FAK inhibitors, Cytopia, EXEL-6309, Etk/BMX kinase inhibitors, SuperGen, erbstatin, erbB-2 PNV, UAB, erbB-2 inhibitors, Cengent, ER-068224, ephrin-B4 sol receptor, VasGene, ephrin-B4 RTK inhib, VasGene, EphA2 receptor tyrosine kinase inhibitor, Pfizer, ENMD-981693, EHT-102, EHT-0101, EGFR/Her-2 kinase inhibitors, Shionogi, EGFR-CA, EGFR kinase inhibitors, Kinex, EGF-genistein, Wayne, EGF-593A, EG-3306, DX-2240, DP-4577, DP-4157, DP-2629, DP-2514, doramapimod, DNX-5000 series, DN-30 Fab, dianilinophthalimide, deuterated erlotinib, CoNCERT, dendritic cell modulators, Antisoma, DD-2, Jak inhibitors, DD-2, dual Jak3/Syk, DCC-2909, DCC-2157, D-69491, CYT-977, CYT-645, CX-4715, curcumin analogues, Onconova, CUDC-107, CT-100, CT-052923, CS-230, CP-724714, CP-673451, CP-564959, CP-292597, CP-127374, Cmpd-1, CL-387785, CKD-712, CHIR-200131, CH-330331, CGP-53716, CGP-52411, CGI-1746, CGEN-B2, CGEN-241, CFAK-Y15, CEP-37440, CEP-33779, CEP-28122, CEP-2563 dihydrochloride, CEP-18050, CEP-17940, celastrol, CDP-791, CB-173, cancer vaccine, bcr-abl, Mologen, cancer therapeutics, Cephalon, CAB-051, c-Src kinase inhibs, AstraZene, c-Met/Her inhibitors, Decipher, c-Met kinase inhibitor, Cephalon, c-Met inhibitors, Roche, c-Met inhibitor, Merck, c-kit inhibitors, Deciphera, c-kit inhibitors, Cell, c-Abl inhibitors, Plexxikon, c-Abl inhibitors, Onconova, BVB-808, Btk inhibitors, Bristol-Myers Squibb, Btk inhibitor, Pharmacyclics-2, BSF-466895, Brk/PTK6 inhibitors, Merck & Co, BreMel/rGel, BPI-703010, BPI-702001, BP-100-2.01, BMX kinase inhibitors, Amphora, BMS-817378, BMS-754807 back-up, BMS-743816, BMS-577098, BLZ-945, BIW-8556, BIO-106, Behcet's disease therapy, Cr, BAY-85-3474, AZM-475271, AZD-0424, AZ-Takl, AZ-23, Axl kinase inhibitors, SuperGen, Axl inhibitors, Deciphera, Axl inhibitors, CRT, AVL-101, AV-412, aurora/FLT3 kinase inhibs, Im, AST-6, AST-487, ARRY-872, ARRY-768, ARRY-470, ARRY-333786, apricoxib+EGFR-TKI, Tragara, AP-23994, AP-23485, anticancers, CoNCERT, anticancers, Bracco, anticancers, Avila-4, anticancers, Avila-3, anticancers, Avila-2, anticancer ZFPs, ToolGen, anticancer therapy, Ariad, anticancer MAbs, Xencor-2, anticancer MAbs, Kolltan, antiangiogenic ther, Deciphera, anti-Tie-1 MAb, Dyax, anti-PDGF-B MAbs, Mill, anti-inflammatory, Kinex, anti-inflammatory, Avila, anti-inflammatory ther, Vitae, anti-HER2neu scFv, Micromet, anti-HER2/Flt3 ligand, Symbi, anti-HER2 MAb, Abiogen, anti-Flt-1 MAbs, ImClone, anti-fak oligonucleotides, anti-ErbB-2 MAbs, Enzon, anti-EphA4 MAb, MedImmune, anti-EGFRvIII MAbs, Amgen, anti-EGFR MAb, Xencor, anti-EGFR immunotoxin, IVAX, anti-CD20/Flt3 ligand, Symbi, Anti-Cancer Ligands, Enchira, anti-ALK MAb, MedImmune, angiopoietins, Regeneron, AMG-Jak2-01, AMG-458, AMG-191, ALK inhibitors, PharmaDesign, ALK inhibitors, Lilly, ALK inhibitors, Cephalon-2, AI-1008, AHNP, Fulcrum, AGN-211745, AGN-199659, AG-957, AG-1295, AEE-788, and ADL-681.

ErbB tyrosine kinase inhibitor (ERbB) include but are not limited to; vandetanib, lapatinib ditosylate, gefitinib, erlotinib, afatinib, XL-647, neratinib, nelipepimut-S, dovitinib lactate, dacomitinib, varlitinib, RAF-265, PR-610, poziotinib, KD-020, BMS-690514, AZD-8931, AVX-901, AVL-301, AE-37, AC-480, VM-206, theliatinib, IDN-6439, HM-61713, epitinib, CUDC-101, cipatinib, Z-650, SN-34003, SN-29966, MT-062, CST-102, ARRY-380, XL-999, vatalanib, TAK-285, SU-5271, PKI-166, Pharmaprojects No. 4960, Pharmaprojects No. 3624, mubritinib, KSB-102, GW-282974, EMD-55900, CNF-201 series, canertinib dihydrochloride, cancer vaccine, Ajinomoto, breast cancer therapy, Galapago, BIBX-1382, AZD-4769, Argos, AP-23464, anti-HER2/neu mimetic, Cyclacel, anti-HER-2/neu antisense, Tekm, AG-18, ZM-254530, ZD-1838, VEGFR/EGFR inhib, Amphora, VEGF-TK inhibitors, AstraZeneca, V-930, RNAi cancer therapy, Benitec Biopharma, RM-6427, RB-200h, PX-104.1, Pharmaprojects No. 6291, Pharmaprojects No. 6271, Pharmaprojects No. 4164, Pharmaprojects No. 3985, Pharmaprojects No. 3495, pelitinib, PD-169540, PD-166285, PD-154233, PD-153035, pan-HER kinase inhib, Ambit-2, pan-HER inhibitor, SUGEN, pan-HER ACL, ON-045270, NSC-242557, NL-0031, mullerian inhibiting subst, Ma, ME-103, kinase inhibitors, Amgen, JNJ-26483327, ISU-101, INSM-18, inherbins, Enkam, HM-60781, HM-30XXX series, Her2/neu & EGFR Ab, Fulcrum, HER2 vaccine, ImmunoFrontier, HER-2 binder, Borean, Her-1/Her-2 dual inhibitor, Hanmi, Her inhibitors, Deciphera, HEM-80322, gene therapy, UCSD, erbB-2 PNV, UAB, erbB-2 inhibitors, Cengent, EHT-102, EGFR/Her-2 kinase inhibitors, Shionogi, EGFR-CA, EGFR kinase inhibitors, Kinex, EGF-593A, dianilinophthalimide, deuterated erlotinib, CoNCERT, D-69491, curcumin analogues, Onconova, CUDC-107, CP-724714, CP-292597, CL-387785, CGEN-B2, CAB-051, c-Met/Her inhibitors, Decipher, BreMel/rGel, BIO-106, AV-412, AST-6, ARRY-333786, apricoxib+EGFR-TKI, Tragara, anticancers, CoNCERT, anticancer MAbs, Xencor-2, anti-HER2neu scFv, Micromet, anti-HER2 MAb, Abiogen, anti-ErbB-2 MAbs, Enzon, anti-EGFRvIII MAbs, Amgen, anti-EGFR MAb, Xencor, anti-EGFR immunotoxin, IVAX, Anti-Cancer Ligands, Enchira, AHNP, Fulcrum, AEE-788, and ADL-681.

MEK1 or MEK2 (MEK) include, but are not limited to: Trametinib, ARRY-438162, WX-554, Selumetinib, Pimasertib, E-6201, BAY-86-9766, TAK-733, PD-0325901, GDC-0623, BI-847325, AS-703988, ARRY-704, Antroquinonol, CI-1040, SMK-17, RO-5068760, PD-98059, and ER-803064.

PIK3CA related treatments include, but are not limited to: perifosine, BKM-120, ZSTK-474, XL-765, XL-147, PX-866, PKI-587, pictilisib, PF-04691502, BYL-719, BEZ-235, BAY-80-6946, PWT-33597, PI3 kinase/mTOR inhibitor, Lilly, INK-1117, GSK-2126458, GDC-0084, GDC-0032, DS-7423, CUDC-907, BAY-1082439, WX-037, SB-2343, PI3/mTOR kinase inhibitors, Amgen, mTOR inhibitor/PI3 kinase inhibitor, Lilly-1, LOR-220, HMPL-518, HM-032, GNE-317, CUDC908, CLR-1401, anticancers, Progenics, anticancer therapy, Sphaera Pharma-1, AMG-511, AEZS-136, AEZS-132, AEZS-131, AEZS-129, pictilisib, companion diagnostic, GDC-0980, companion diagnostic, GDC-0032, companion diagnostic, AZD-8055, VEL-015, SF-2523, SF-2506, SF-1126, PX-2000, PKI-179, PI3K p110alpha inhibitors, Ast, PI3K inhibitors, Semafore-2, PI3K inhibitors, Invitrogen, PI3K inhibitor conjugate, Semaf, PI3K conjugates, Semafore, PI3-irreversible alpha inhibitors, Pathway, PI3-alpha/delta inhibitors, Pathway Therapeutics, PI3-alpha inhibitors, Pathway Therapeutics, PI3 kinase inhibitors, Wyeth, PI3 kinase inhibitors, Telik, PI3 kinase alpha selective inhibitors, Xcovery, PI-620, PF-4989216, PF-04979064, PF-00271897, PDK1 inhibitors, GlaxoSmithKline, ONC-201, KN-309, isoform-selective PI3a/B kinase inhibitors, Sanofi, inositol kinase inhibs, ICRT, HM-5016699, hepatocellular carcinoma therapy, Sonitu, GSK-1059615, glioblastoma therapy, Hoffmann-La Roche, EZN-4150, CU-906, CU-903, CNX-1351, antithrombotic, Cerylid, 4-methylpteridinones.

Treatments directed to ALK include, but are not limited to: crizotinib, companion diagnostic, AbbVie, crizotinib, TSR-011, RG-7853, LDK-378, AP-26113, X-396, ASP-3026, NMS-E628, DLX-521, aurora kinase+ALK inhibitor, Sareum, aurora kinase+ALK inhibitor, AstraZeneca, ALK inhibitors, AstraZeneca, Alk inhibitor, Cephalon-3, ALK inhibitor, Aurigene Discovery Technologies, LDK-378, companion diagnostic, crizotinib, companion diagnostic, Roche, TAE-684, kinase inhibitor, Cephalon, GSK-1838705A, EXEL-6309, Cmpd-1, CEP-37440, CEP-28122, CEP-18050, cancer therapeutics, Cephalon, anti-ALK MAb, MedImmune, ALK inhibitors, PharmaDesign, ALK inhibitors, Lilly, ALK inhibitors, and Cephalon-2.

Treatments directed to RET include, but are not limited to: vandetanib, sunitinib malate, sorafenib, regorafenib, cabozantinib, SAR-302503, motesanib diphosphate, apatinib, RET kinase inhibitor, Bionomics, NMS-173, MG-516, sorafenib bead, Biocompatibles, RET inhibitors, Cell T, MP-371, kinase inhibitors, MethylGene, JNJ-26483327, DCC-2157, and AST-487.

Accordingly, these and other agents can be used alone or in combination to treat NSCLC and can be included as an actionable treatment recommendation as disclosed herein.

Methods directed to determining a likelihood of a positive or negative response to a treatment and/or treating a subject based on the gene variant detected in the subject's sample are also provided herein. Referring to Tables 2 and 3, in certain embodiments, an actionable treatment recommendation refers to a particular treatment. For example, an EML4-ALK fusion present in a tumor sample leads to a recommendation of treatment with crizotinib. In contrast, the presence of an EGFR T790M mutation indicates that an EGFR tyrosine kinase inhibitor (TKI) would not be an appropriate treatment as this variant renders the tumor cell resistant to TKIs. The actionable treatment recommendation can be used to administer a treatment or withhold a treatment, depending on the variant status of a subject's tumor.

TABLE 2
Lung Adenocarcinoma

| AI Cat- | | | Actionable treatment |
|---|---|---|---|
| egory | Genetic | Variant | recommendation |
| AI1 | ALK | EML4-ALK, KIF5B- | Crizotinib |
| | | ALK, KLC1-ALK, | |
| | | TGF-ALK fusions | |
| AI1 | EGFR | L858R, Exon | EGFR TKIs |
| | | 19 deletion | |
| AI1 | EGFR | Exon 20 insertion | Resistant to EGFR |
| | | (in frame, 3-18 | TKIs |
| | | base pairs) | |
| AI1 | EGFR | T790M | Resistant to EGFR |
| | | | TKIs |
| AI1/AI2 | KRAS | G12C, G12V, G12D, | Resistant to EGFR |
| | | G12A, G12S, G13C, | TKI (AI1) |
| | | G13D, G12R, G12F | Sensitive to MEK |
| | | | inhibitors (AI2) |
| AI1 | ROS1 | EZR-ROS1, SLC34A2- | Crizotinib |
| | | ROS1, CD74-ROS1, | |
| | | SDC4-ROS1 | |
| AI2 | BRAF | V600E | Vemurafenib |
| AI2 | ERBB2 | Exon 20 insertion | Irreversible pan-erb |
| | | | inhibitors (e.g., |
| | | | afatinib, neratinib) |
| AI2 | MET | CN gain | Resistant to EGFR |
| | | | TKIs |
| | | | Sensitive to Crizotinib |
| AI2 | PIK3CA | E545K, E545G, E545A, | PIK3CA inhibitors |
| | | H1047R, H1047L | (e.g., BKM120) |
| AD | AKT1 | E17K | 1 Open Phase II Trial |
| | | | (Lung cancer, AKT |
| | | | mutation) |
| AI3 | BRAF | L597R | 3 Open Phase I trials |
| | | | (solid cancer), 1 Open |
| | | | Phase II trial (lung |
| | | | cancer, BRAF mutation) |
| AI3 | BRAF | G469R, D594H/N | 3 Open Phase I trials |
| | | | (solid cancer), 1 Open |
| | | | Phase II trial (lung |
| | | | cancer, BRAF mutation) |
| AD | EGFR | G719X | 1 Open Phase I |
| | | | (NSCLC), 1 Open |
| | | | Phase 1 (solid |
| | | | cancer), 1 open Phase |
| | | | II (NSCLC) |
| AD | HRAS | Q61L/K/R, G12C/D, | 1 Open Phase II (lung |
| | | G13C/S/R/V | cancer, HRAS mutations) |
| AD | PIK3CA | E542K | 2 Open Phase I (solid |
| | | | cancer), 1 Open Phase |
| | | | II trial (NSCLC, |
| | | | PIK3CA mutation) |

TABLE 3
Squamous Cell Lung Carcinoma

| AI Cat- | | | Actionable treatment |
|---|---|---|---|
| egory | Prevalence >1% | Prevalence 0.1%-1% | recommendation |
| AI1 | EGFR (L858R, | EGFR (G719X) | EGFR TKIs |
| | Exon 19 del) | | |
| AI1/AI2 | KRAS (G12C, | KRAS (G12A, | Resistant to TKIs |
| | G12D) | G12V) | (AI1); Sensitive to |
| | | | MEK Inhibitors (AI2) |
| AI2 | MET CN gain | | Resistant to TKIs; |
| | | | Sensitive to Crizotinib |
| AI2 | PIK3CA (E545K, | | PIK3CA Inhibitors |
| | E542K, H1047R) | | (e.g., BKM120) |
| AI3 | AKT1 (E17K) | | 1 Open Phase II Trial |
| | | | (Lung cancer, AKT mutation) |
| AI3 | | HRAS (Q61, /K/R, | 1 Open Phase II (Lung |
| | | G12C/D) | cancer; HRAS mutation) |
| AI3 | EGFR CN gain | | 1 Open Phase II (lung |
| | | | cancer; EGFR amplification) |
| AI3 | ERBB2 CN gain | | 2 Open Phase II |
| | | | (Lung cancer; ERBB2 |
| | | | amplification) |
| AI3 | FGFR1 CN gain | | 2 Open Phase I; Phase |
| | | | II (Solid cancer; |
| | | | FGFR1 amplification) |
| AI3 | KIT/PDGFRA | | 1 Open Phase II |
| | CN gain | | (Lung cancer; |
| | | | PDGFRA amplification) |
| AI3 | PTEN Del | | 4 Open Phase I/II |
| | | | (NSCLC, PTEN alterations) |

TABLE 4
Adenocarcinoma

| AI1-AI2-AI3-Gene-Event | No. | Percentage |
|---|---|---|
| ALK- Fusion | 2 | 1% |
| BRAF-Mutation | 3 | 2% |
| BRAF-Mutation; PIK3CA- mutation* | 1 | 1% |
| EGFR-CN Amp | 3 | 2% |
| EGFR-Mutation | 13 | 8% |
| EGFR-Mutation; EGFR-CN Amp* | 3 | 2% |
| ERBB2-CN Amp | 3 | 2% |
| ERBB2-mutation | 3 | 2% |
| FGFR1-CN Amp | 2 | 1% |
| HRAS-Mutation | 1 | 1% |
| KIT- CN Amp | 1 | 1% |
| KRAS-Mutation; PIK3CA- Mutation* | 2 | 1% |
| KRAS-Mutation | 39 | 24% |
| KRAS-Mutation; EGFR-CN Amp* | 1 | 1% |
| MET-CN Amp | 3 | 2% |
| PIK3CA-mutation | 3 | 2% |
| RET- Fusion | 1 | 1% |
| ROS1- Fusion | 2 | 1% |
| WT | 79 | 48% |

TABLE 5
Adenocarcinoma

| AI1-AI2-AI3-Gene-Variant | No | Percentage |
|---|---|---|
| BRAF-D594H; PIK3CA-E542K* | 1 | 1% |
| BRAF-D594N | 1 | 1% |
| BRAF-V600E | 2 | 1% |
| CCDC6-RET Fusion | 1 | 1% |
| CD74-ROS1 Fusion | 1 | 1% |
| EGFR-CN Amp | 3 | 2% |
| EGFR-E19Del | 4 | 2% |
| EGFR-E19Del; EGFR-CN Amp* | 3 | 2% |
| EGFR-G719A | 1 | 1% |
| EGFR-L858R | 7 | 4% |
| EGFR-L858R; EGFR-T790M* | 1 | 1% |
| EML4-ALK Fusion | 2 | 1% |
| ERBB2-CN Amp | 3 | 2% |
| ERBB2-E20Ins | 3 | 2% |
| FGFR1-CN Amp | 2 | 1% |
| HRAS-Q61L | 1 | 1% |
| KIT- CN Amp | 1 | 1% |
| KRAS-G12A | 4 | 2% |
| KRAS-G12C | 21 | 13% |
| KRAS-G12C; EGFR-CN Amp* | 1 | 1% |
| KRAS-G12C; PIK3CA-E545K* | 2 | 1% |
| KRAS-G12D | 2 | 1% |
| KRAS-G12V | 11 | 7% |
| KRAS-G13D | 1 | 1% |
| MET-CN Amp | 3 | 2% |
| PIK3CA-E545K | 2 | 1% |
| PIK3CA-H1047R | 1 | 1% |
| SLC34A2-ROS1 Fusion | 1 | 1% |
| WT | 79 | 48% |

*Double mutant genotypes

TABLE 6
Adenocarcinoma

| AI1, AI2 Gene event | No. | Percentage |
|---|---|---|
| MET-CN Gain | 1 | 1% |
| PIK3CA-Mutation | 14 | 8% |
| PIK3CA-Mutation; MET-CN Gain* | 1 | 1% |
| WT | 161 | 91% |

*Double mutant genotypes

TABLE 7
Squamous Cell Carcinoma

| AI1, AI2, AI3-Gene event | No. | Percentage |
|---|---|---|
| EGFR- CN Gain | 12 | 7% |
| ERBB2-CN Gain | 1 | 1% |
| FGFR1- CN Gain | 23 | 13% |
| KIT-CN Gain | 1 | 1% |
| MET-CN Gain | 1 | 1% |
| PIK3CA-Mutation | 11 | 6% |
| PIK3CA-Mutation; EGFR- CN Gain* | 1 | 1% |
| PIK3CA-Mutation; FGFR1- CN Gain* | 2 | 1% |
| PIK3CA-Mutation; MET-CN Gain* | 1 | 1% |
| PTEN- CN Loss | 2 | 1% |
| WT | 122 | 69% |

*Double mutant genotypes

TABLE 8
Squamous Cell Carcinoma

| AI1, AI2 Gene Events | No. | Percentage |
|---|---|---|
| AI2 | 16 | 9% |
| WT | 161 | 91% |

TABLE 9
Highly Actionable Molecular Targets in NSCLC

| Source | Type | Gene Target |
|---|---|---|
| DNA | Oncogenes | EGFR, ERBB2, ERBB4, MET, FGFR1, |
| | | FGFR2, FGFR3, DDR2, ALK |
| | EGFR Pathway | KRAS, NRAS, PIK3CA, BRAF, |
| | | MAP2K1, AKT1 |
| | Tumor Suppressor | PTEN, TP53, CTNNB1, NOTCH1, |
| | Genes | STK11, SMED4, FBXW7 |
| RNA | Fusion Genes | ALK, RET, ROS |

TABLE 11

| | | | Approved | | Limita- |
|---|---|---|---|---|---|
| Gene | Variant | Level of | targeted | Indications and | tions of |
| Symbol | Type | evidence | agent | uses | usage |
| ALK | Fusion | 1 | crizotinib | Xalkori- kinase | |
| | | | | inhibitor indicated | |
| | | | | for treatment of | |
| | | | | patients with | |
| | | | | metastatic NSCLC | |
| | | | | whose tumors are | |
| | | | | ALK-positive as | |
| | | | | detected by an | |
| | | | | FDA-approved test | |
| ALK | Fusion | 1 | | | |
| RET | Fusion | 2 | None | None | None |
| RET | Fusion | 2 | | | |
| ROS1 | Fusion | 1 | None | None | None |

TABLE 13
Biomarkers

| | | | | |
|---|---|---|---|---|
| ABL1 | CD274 | GATA3 | MLL4 | RAF1 |
| ACVRL1 | CD44 | GNA11 | MPL | RARA |
| AKT1 | CDH1 | GNAQ | MYC | RB1 |
| AKT3 | CDK4 | GNAS | MYCL1 | RET |
| ALK | CDK6 | HRAS | MYCN | RHEB |
| APC | CDKN2A | IDH1 | MYD88 | RHOA |
| APEX1 | CSNK2A1 | IDH2 | NCOR1 | ROS1 |
| AR | CTCF | IFITM1 | NF1 | RPS6KB1 |
| ARHGAP35 | CTNNB1 | IFITM3 | NFE2L2 | SETD2 |
| ARID1A | DNMT3A | IGF1R | NKX2-1 | SF3B1 |
| ARID1B | EGFR | IL6 | NOTCH1 | SMO |
| ARID2 | ERBB2 | JAK1 | NRAS | SOX2 |
| ATM | ERBB3 | JAK2 | NSD1 | SPEN |
| ATRX | ERG | JAK3 | PAX5 | SPOP |
| BCL2L1 | ETV1 | KIT | PBRM1 | STAT3 |
| BCL9 | ETV4 | KRAS | PDGFRA | STK11 |
| BIRC2 | ETV5 | MAGOH | PDGFRB | TERT |
| BIRC3 | EZH2 | MAP2K1 | PIK3C2A | TIAF1 |
| BRAF | FAT1 | MAP3K1 | PIK3CA | TP53 |
| BRCA1 | FBXW7 | MAPK1 | PIK3R1 | U2AF1 |
| BRCA2 | FGFR1 | MAX | PNP | VHL |
| C15orf23 | FGFR2 | MCL1 | PPARG | WT1 |
| CBL | FGFR3 | MDM2 | PPP2R1A | XPO1 |
| CCND1 | FLT3 | MDM4 | PTEN | ZC3H13 |
| CCND2 | FOXL2 | MED12 | PTPN11 | ZNF217 |
| CCND3 | GAS6 | MET | RAC1 | |
| CCNE1 | GATA2 | MGA | | |

TABLE 14
Hot Spots

| | | | | | |
|---|---|---|---|---|---|
| ABL1 | GNAQ | MYD88 | EGFR | KIT | RHEB |
| AKT1 | GNAS | NFE2L2 | ERBB2 | KRAS | RHOA |
| ALK | HRAS | NRAS | ERBB3 | MAGOH | SF3B1 |
| AR | IDH1 | PAX5 | EZH2 | MAP2K1 | SMO |
| BRAF | IDH2 | PDGFRA | FGFR2 | MAPK1 | SPOP |
| C15orf23 | IFITM1 | PIK3CA | FGFR3 | MAX | SRC |
| CBL | IFITM3 | PPP2R1A | FLT3 | MED12 | STAT3 |
| CDK4 | JAK1 | PTPN11 | FOXL2 | MET | U2AF1 |
| CTNNB1 | JAK2 | RAC1 | GATA2 | MPL | XPO1 |
| DNMT3A | JAK3 | RET | GNA11 | | |

TABLE 15
Copy Number Amplifications

| | | | | | | | |
|---|---|---|---|---|---|---|---|
| ACVRL1 | BIRC2 | CD44 | FGFR1 | IGF1R | MDM4 | NKX2-1 | RPS6KB1 |
| AKT1 | BIRC3 | CDK4 | FGFR2 | IL6 | MET | PDGFRA | SOX2 |
| AR | CCND1 | CDK6 | FGFR3 | KIT | MYC | PIK3CA | TERT |
| APEX1 | CCNE1 | CSNK2A1 | FLT3 | KRAS | MYCL1 | PNP | TIAF1 |
| BCL2L1 | CD274 | EGFR | GAS6 | MCL1 | MYCN | PPARG | ZNF217 |
| BCL9 | | ERBB2 | | MDM2 | | | |

TABLE 16
Gene Fusions

| | | | | | | |
|---|---|---|---|---|---|---|
| AKT3 | ALK | BRAF | CDK4 | ERG | ETV1 | ETV4 |
| ETV5 | FGFR3 | HER2 | NTRK3 | RAFI | RET | ROS1 |

TABLE 17
Tumor Suppressor Genes

| | | | | | |
|---|---|---|---|---|---|
| APC | ATRX | FAT1 | NCOR1 | PTEN | VHL |
| ARHGAP35 | BRCA1 | FBXW7 | NF1 | RB1 | WT1 |
| ARID1A | BRCA2 | GATA3 | NOTCH1 | SETD2 | ZC3H13 |
| ARID1B | CDH1 | MAP3K1 | NSD1 | SPEN | |
| ARID2 | CDKN2A | MGA | PBRM1 | STK11 | |
| ATM | CTCF | MLL4 | PIK3R1 | TP53 | |

TABLE 18
Types of Cancers

| | | |
|---|---|---|
| Adrenocortical Carcinoma | Germ Cell Tumor, Extragonadal | Osteosarcoma |
| Anal Cancer | Gestational Trophoblastic | Ovarian Epithelial Cancer |
| | Tumor | |
| Aplastic Anemia | Laryngeal Cancer and | Ovarian Germ Cell Tumor |
| | Hypopharyngeal Cancer | |
| Bile Duct Cancer | Leukemia | Pancreatic Cancer, Exocrine |
| Bladder Cancer | Leukemia in Children | Pancreatic Cancer, Islet Cell |
| | | Carcinoma |
| Blood Cancers Treatment | Leukemia, Acute | Parathyroid Cancer |
| | Lymphoblastic, Adult | |
| Bone Cancer | Leukemia, Acute | Penile Cancer |
| | Lymphoblastic, Childhood | |
| Brain/CNS Tumor, Adult | Leukemia, Acute Myeloid, | Pituitary Cancer |
| | Adult | |
| Brain/CNS Tumor, Brain Stem | Leukemia, Acute Myeloid, | Plasma Cell Neoplasm |
| Glioma, Childhood | Childhood | |
| Brain Tumor, Cerebellar | Leukemia, Chronic | Prostate Cancer |
| Astrocytoma, Childhood | Lymphocytic (CLL) | |
| Brain Tumor, Cerebral | Leukemia, Chronic | Rhabdomyosarcoma, |
| Astrocytoma, Childhood | Myelogenous (CML) | Childhood |
| Brain Tumor, Ependymoma, | Lip and Oral Cavity Cancer | Rectal Cancer |
| Childhood | | |
| Brain Tumor, Childhood | Liver Cancer, Adult (Primary) | Renal Cell Cancer (cancer of |
| (Other) | | the kidney) |
| Breast Cancer | Liver Cancer, Childhood | Renal Pelvis and Ureter, |
| | (Primary) | Transitional Cell |
| Breast Cancer, Male | Lung Cancer, Non-Small Cell | Rhabdomyosarcoma |
| Cancer in Children/Cancer of | Lung Cancer, Small Cell | Salivary Gland Cancer |
| Unknown Primary | | |
| Carcinoid Tumor, | Lung Carcinoid Tumor | Sarcoma - Adult Soft Tissue |
| Gastrointestinal | | Cancer |
| Carcinoma of Unknown | Lymphoma, AIDS-Related | Sezary Syndrome |
| Primary | | |
| Castleman Disease | Lymphoma of the skin | Skin Cancer |
| Cervical Cancer | Lymphoma, Central Nervous | Skin Cancer - Basal and |
| | System (Primary) | Squamous Cell |
| Colon Cancer | Lymphoma, Cutaneous T-Cell | Skin Cancer, Cutaneous T-Cell |
| | | Lymphoma |
| Endometrial Cancer | Lymphoma, Hodgkin's Disease, | Skin Cancer, Kaposi's Sarcoma |
| | Adult | |
| Esophageal Cancer | Lymphoma, Hodgkin's Disease, | Skin Cancer, Melanoma |
| | Childhood | |
| Extrahepatic Bile Duct Cancer | Lymphoma, Non-Hodgkin's | Small Intestine Cancer |
| | Disease, Adult | |
| Ewings Family of Tumors | Lymphoma, Non-Hodgkin's | Soft Tissue Sarcoma, Adult |
| (PNET) | Disease, Childhood | |
| Extracranial Germ Cell Tumor, | Malignant Mesothelioma | Soft Tissue Sarcoma, Child |
| Childhood | | |
| Eye Cancer, Intraocular | Melanoma | Stomach Cancer |
| Melanoma | | |
| Gallbladder Cancer | Merkel Cell Carcinoma | Testicular Cancer |
| Gastrointestinal Stromal Tumor | Metasatic Squamous Neck | Thymoma, Malignant |
| (GIST) | Cancer with Occult Primary | |
| Gastric Cancer (Stomach) | Multiple Myeloma and Other | Thyroid Cancer |
| | Plasma Cell Neoplasms | |
| Germ Cell Tumor, | Mycosis Fungoides | Urethral Cancer |
| Extragonadal | | |
| Gestational Trophoblastic | Myelodysplastic Syndrome | Uterine Cancer, Sarcoma |
| Tumor | | |
| Head and Neck Cancer | Myeloproliferative Disorders | Unusual Cancer of Childhood |
| Hypopharyngeal Cancer | Nasal Cavity and Paranasal | Vaginal Cancer |
| | Sinus Cancer | |
| Islet Cell Carcinoma | Nasopharyngeal Cancer | Vulvar Cancer |
| Kaposi Sarcoma | Neuroblastoma | Waldenstrom |
| | | Macroglobulinemia |
| Kidney Cancer (renal cell | Oral Cancer | Wilms' Tumor |
| cancer) | | |
| Gallbladder Cancer | Oral Cavity Cancer | |
| Gastric Cancer (Stomach) | Oropharyngeal Cancer | |

### SEE TABLE 19

In certain embodiments compositions, kits and methods are disclosed for detection of driver alterations for cancer. The cancer can be any type of cancer (see, for example, Table 18). In certain embodiments, the compositions, kits and methods comprise detecting driver alterations associated with a large number of cancer types. In certain embodiments, the compositions, kits and methods comprise detecting all driver mutations associated with all known cancer types.

Comprehensive screening can be performed in a single panel and therefore can be performed utilizing a single biological sample, thus preserving valuable sample. Sample input can be as low as 100 ng, 90 ng, 80 ng, 70 ng, 60 ng, 50 ng, 40 ng, 30 ng, 20 ng, 10 ng, or less. In certain embodiments, 50 ng is required. In yet other embodiments, less than 50 ng, such as 10 ng, 5 ng, 1 ng, is required.

In one embodiment, compositions and kits are provided that comprise a plurality (i.e, greater than 1) of sets of probes that specifically recognize the nucleic acids of the genes in Tables 13-17 and 19. The compositions and kits can comprise a set of probes that specifically recognize any number and combination of the genes in Tables 13-17 and 19. In certain embodiments the number of genes is greater than 5, 10, 15, 20, 50, 70, 100, 110, 120, 130, 150, 200, 250, and greater than 250, such as 300, 400, 500, 1000 or more (and each integer in between). In certain embodiments, the compositions and kits can comprise a set of probes that specifically recognize each of the genes in Tables 13-17 and 19.

Driver alterations can be any form of genetic variance that confers a growth and/or survival advantage on the cells carrying them, specifically, a cancer cell. In certain embodiments, the driver alteration provides an actionable target. That is, the driver alteration is associated with a drug response or a clinical decision support. An exemplary list of driver alterations is provided in Tables 13-17 and 19, which include cancer hotspot mutations, copy number variation, tumor suppressor genes, and gene fusions.

Table 19 provides an exemplary list of gene fusions. Referring to item 11, in which the driver gene is ALK. The 5′gene is EML4 and the 3′gene is ALK. The 5′ and 3′ Entrez Id's are provided and the source of the fusion with this particular break point is the OncoNetwork. Other sources can include NGS, Cosmic, ARUP, alone or in combination. The 5′ Exon number, in item 11, indicates that Exon 17 coding sequence (cds) of EML4 is involved in this fusion and the 3′ Exon number indicates that Exon 20 coding sequence of ALK is involved in this fusion. Additional information found in Table 19 includes: Cosmid Ids and remarks, observed or inferred, are provided (where relevant) and 5′ and 3′ breakpoint sites.

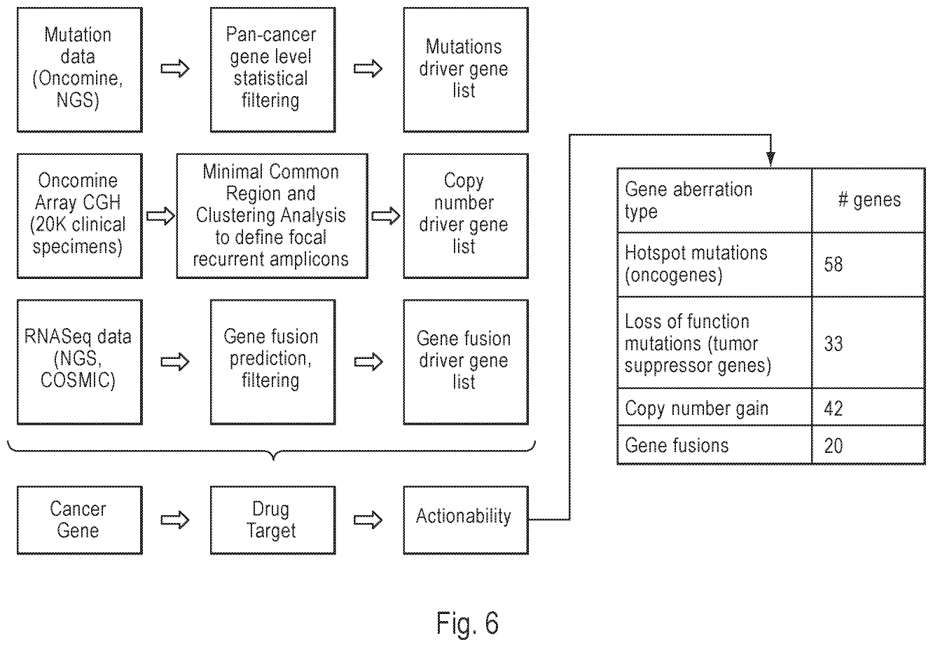

FIG. 6 provides an exemplary work flow of how gene content can be defined by cancer driver analysis. In this workflow, a cancer gene can be associated with a drug target and an actionability index determined and recommended action can be identified.

In certain embodiments, one or more driver mutations can be detected or identified by various sequencing methods. Non-limiting examples of sequence analysis include Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry, and sequencing by hybridization. Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

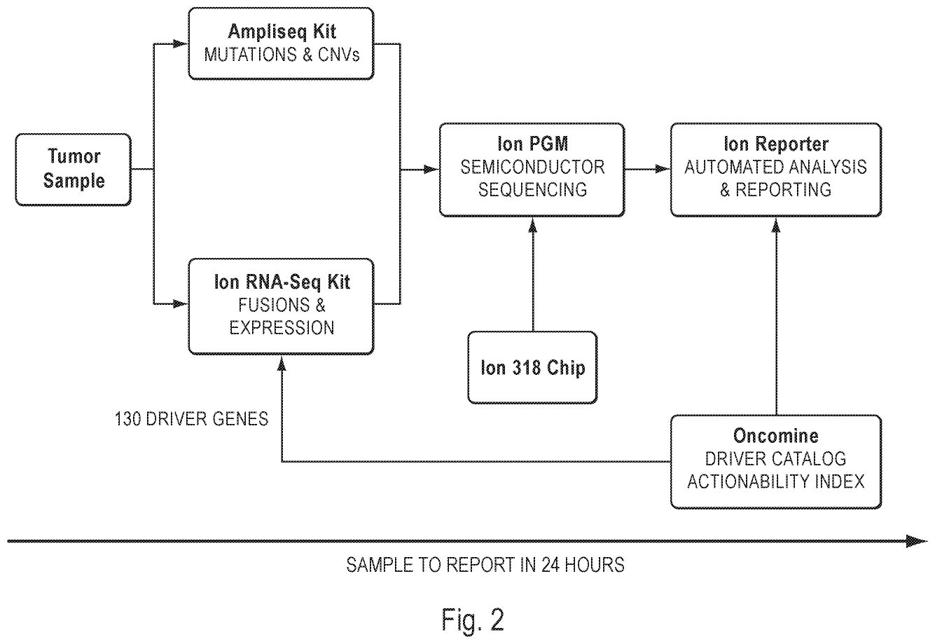

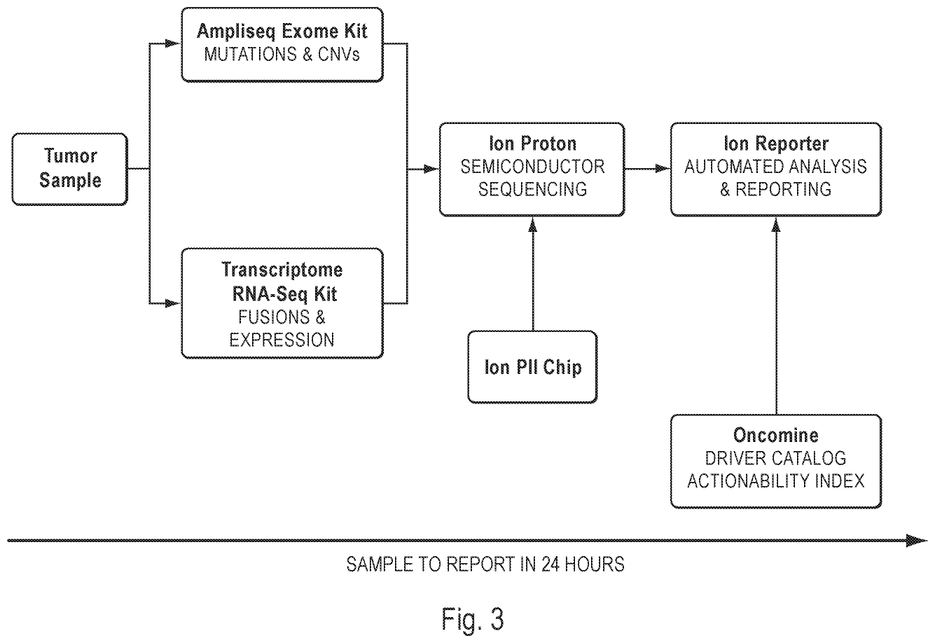

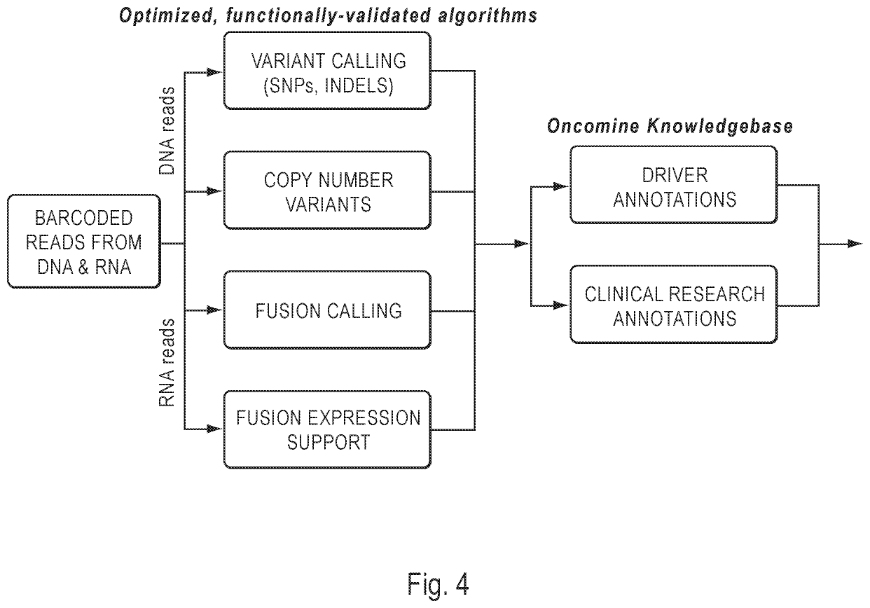

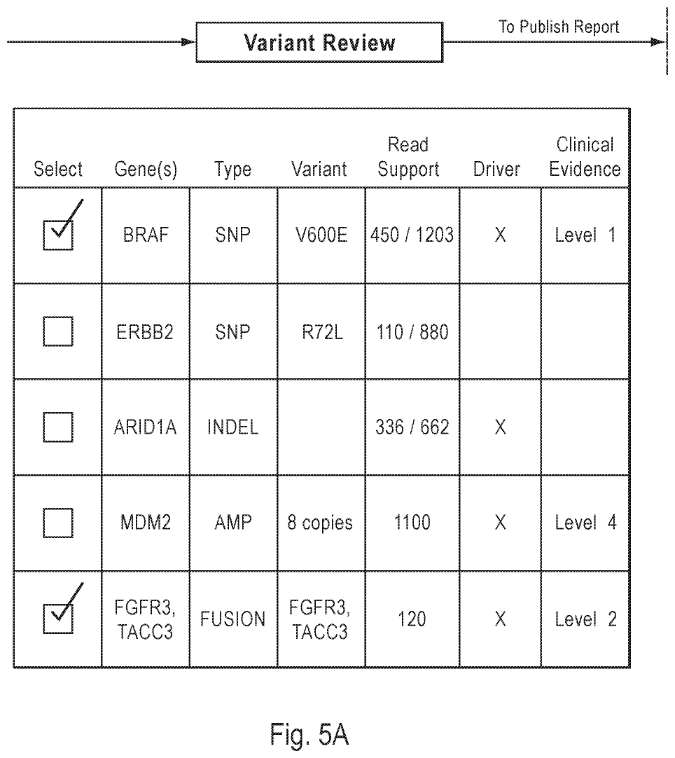

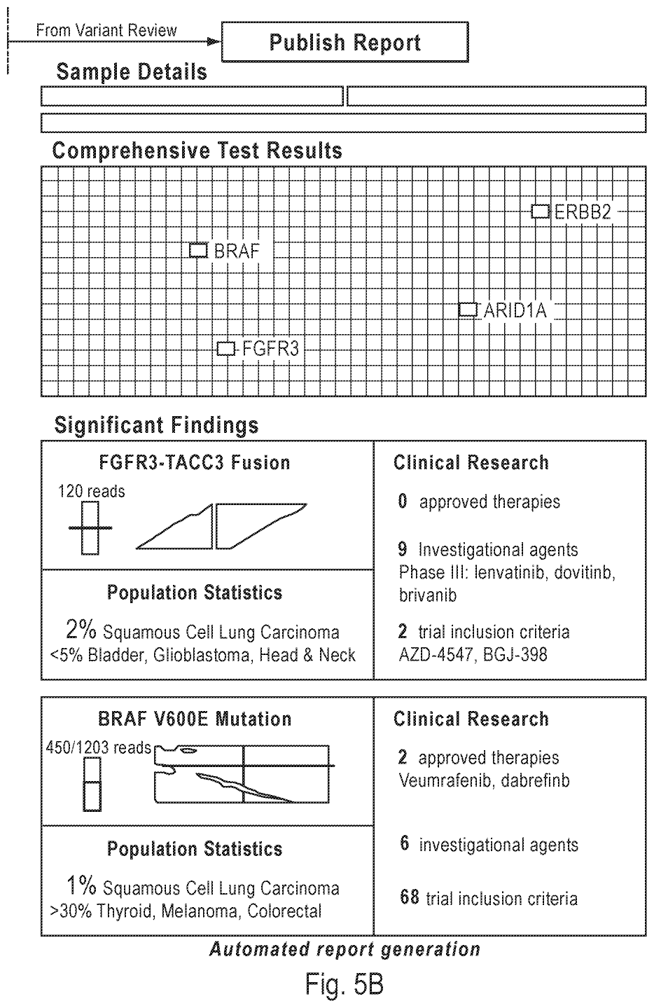

In one embodiment a tumor sample is sequenced for at least one variant, e.g. a mutation, copy number variation, fusion, altered expression, and a combination thereof. The sample is sequenced, for example, with NGS, such as semiconductor sequencing technology. The sample is automatically analyzed for driver mutation status and a report is generated. See FIGS. 2 and 3.

In another embodiment, one or more driver mutations are detected by next generation sequencing and subsequently by confirmed by one or other additional methods disclosed above. These confirmatory methods are referred to as Reflex Tests. The Reflex Test. In certain embodiment, sequencing with NGS is followed by a non-NGS reflex test. For example, sequencing with NGS can be followed by a Reflext Test with sequence analysis methods including include Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry, and sequencing by hybridization. In certain embodiments, NGS is followed by a Reflex Test with Sanger sequencing or thermocycler sequencing, such as qPCR.

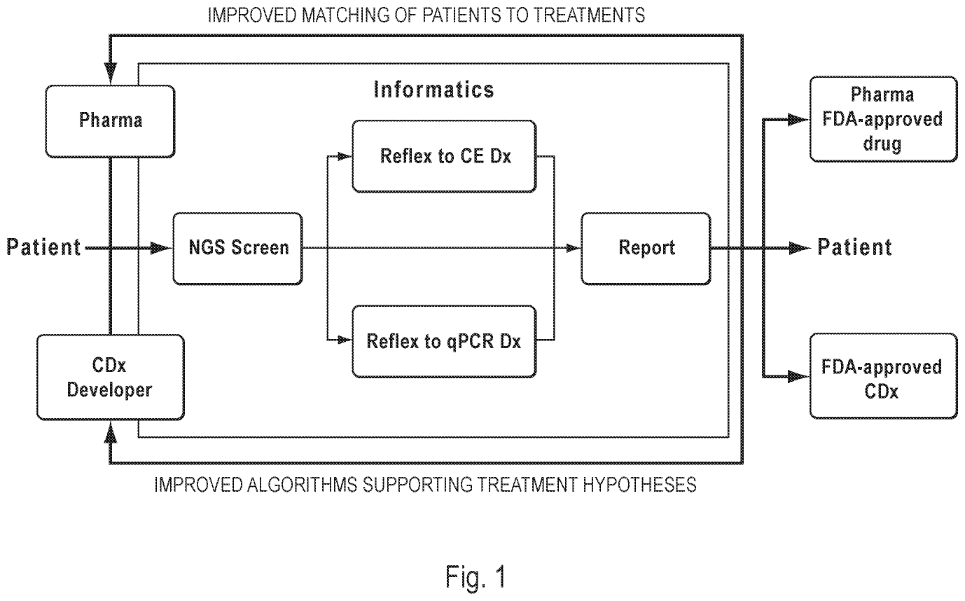

In certain embodiments, a treatment is determined for a patient with cancer. Multiple workflows are disclosed herein that can be used to determine the treatment. For example, a sample can be obtained from a subject with can be obtained and screened for genetic variants utilizing next generation sequencing. Depending on the variant detected with NGS, a confirmatory test can be performed using either CE or aPCR. When the genetic variant identified is confirmed, a report is generated. The report can comprise suggestions or recommendations for an FDA approved drug, a companion diagnostic assay, a clinical trial, etc. These recommendations can be based on the A1 associated with the patient's results. The recommendation is communicated in a report to an oncologist and/or the patient. The oncologist can then utilize the recommendations in the report to inform his clinical treatment plan for the patient. See FIG. 1.

In certain embodiments, the workflow from sample prep to report is complete in less than 1 week, less than 6, 5, or 4 days, less than 3 or 2 days, etc. In certain embodiments, the workflow form sample prep to report time is approximately 24 hours.

In embodiments where certain next generation sequencing methodologies are employed,

### Reports

In another aspect, the invention features a report indicating a prognosis or treatment response prediction of a subject with cancer. The report can, for example, be in electronic or paper form. The report can include basic patient information, including a subject identifier (e.g., the subject's name, a social security number, a medical insurance number, or a randomly generated number), physical characteristics of the subject (e.g., age, weight, or sex), the requesting physician's name, the date the prognosis was generated, and the date of sample collection. The reported prognosis can relate to likelihood of survival for a certain period of time, likelihood of response to certain treatments within a certain period of time (e.g., chemotherapeutic or surgical treatments), and/or likelihood of recurrence of cancer. The reported prognosis can be in the form of a percentage chance of survival for a certain period of time, percentage chance of favorable response to treatment (favorable response can be defined, e.g., tumor shrinkage or slowing of tumor growth), or recurrence over a defined period of time (e.g., 20% chance of survival over a five year period). In another embodiment, the reported prognosis can be a general description of the likelihood of survival, treatment recommendations (ie, FDA approved pharmaceutical, further classification via companion diagnostic test, clinical trials, etc), response to treatment, or recurrence over a period of time. In another embodiment, the reported prognosis can be in the form of a graph. In addition to the gene expression levels and gene variants/mutations, the reported prognosis may also take into account additional characteristics of the subject (e.g., age, stage of cancer, gender, previous treatment, fitness, cardiovascular health, and mental health).

In addition to a prognosis, the report can optionally include raw data concerning the expression level or mutation status of genes of interest.

### EXAMPLES

### Example I

Genomic and gene variant data was obtained from Life Technologies and Compendia Bioscience's ONCOMINE™ Concepts Edition and ONCOMINE™ Power Tools, a suite of web applications and web browsers that integrates and unifies high-throughput cancer profiling data by systematic collection, curation, ontologization and analysis. In addition, mutation gene variant data was also obtained from Life Technologies and Compendia Bioscience's curation and analysis of next generation sequencing data available from The Cancer Genome Atlas (TCGA) Portal.

Data obtained from the TCGA contains mutation results from datasets processed and annotated by different genome sequencing centers. All of the mutation data characterized in TCGA was somatic mutation data containing mutation variants specific to the tumor specimen and not observed in the normal tissue specimen obtained from the same individual. To obtain consistent variant annotation, the mutations obtained from TCGA were re-annotated based on a single set of transcripts and variant classification rules. A standard annotation pipeline ensured that mutations were evaluated consistently and were subject to common interpretation during the identification of lung cancer gene variants. In the Mutation Annotation step, the mutations obtained from TCGA were re-annotated against a standard transcript set. This transcript set included RefGene transcripts from hg18 and hg19 genome builds, obtained from UCSC on Feb. 19, 2012.

Mutation data incorporated into ONCOMINE Power Tools was derived from multiple sources including the Sanger Institute's Catalogue of Somatic Mutations in Cancer (COSMIC). Mutation data sourced from COSMIC retained its original annotation.

Recurrent gene mutations in multiple clinical samples were identified based on the position of the variant in the gene coding sequence. Missense mutation variants were inferred if the mutation was a single nucleotide polymorphism (SNP) in a coding exon that changed the encoded amino acid. Such missense mutation gene variants were recurrent if the same gene contained the same SNP in multiple samples. Hotspot in frame insertion/deletion mutation variants were inferred if the nucleotide mutation was an insertion or deletion divisible by 3 nucleotides.

The frequency of recurrent hotspot missense mutation and/or hotspot in frame insertion/deletion mutation in different genes in lung cancer was characterized by counting all of the clinical specimens tested that were found to contain the gene variants and expressing that value as a percentage relative to all of the clinical specimens tested. A list of all the genes with prevalent hotspot missense mutations in lung cancer was derived.

Gene copy number data for lung cancer was obtained from the ONCOMINE DNA Copy PowerTool. A minimal common region analysis was performed to identify chromosomal regions of focal amplification in lung cancer. Contiguous chromosomal regions (common regions) containing copy gain (>0.9 log 2 copy number) in 2 or more samples were identified. Within each common region, the genes that were aberrant in the highest number of samples (n) and also those that were aberrant in one less the highest number (n−1) were identified. Alternatively, genes aberrant in 95% of the highest number of samples (n) were identified. The frequency of these peak regions was determined by calculating the number of samples with copy gain relative to the total number of samples analyzed and expressing this value as a percentage. The most prevalent peak regions in lung cancer typically contained known cancer genes such as MET, FGFR1, EGFR, ERBB2, KIT/PDGFRA.

Gene variants with prevalent hotspot missense mutations, focal amplification, or gene fusion were investigated further to determine whether they had actionability evidence associated with actionability index levels 1-3.

Gene variants associated with AI1 were identified in the National Comprehensive Cancer Network Practice Guidelines in Oncology (NCCN Guidelines) for non-small cell lung cancer (NSCLC) (Version 2.2013). Such gene variants were those that the Guidelines provided specific treatment recommendations. For example, patients with lung adenocarcinoma whose tumor specimen was found to contain EGFR L858R variants were recommended to consider treatment with an EGFR inhibitor such as erlotinib or gefitnib.

Gene variants associated with AI2 were identified in public literature sources such as the National Center for Biotechnology Information (NCBI) PubMed, a web browser containing citations for biomedical literature.

Gene variants associated with AI3 were identified by searching databases of clinical trial information such as ClinicalTrials.Gov and Citeline© TrialTrove for matching gene and variant type annotation in the enrollment criteria of ongoing clinical trials.

Referring to Tables 4-5, the methods disclosed herein provide an actionable treatment recommendation for 50% of adenocarcinoma subjects. A cohort of 165 patients with primary lung adenocarcinoma was characterized by next generation sequencing methods. The gene variants were mapped onto this population. Most patients were observed to have only a single aberration out of the entire panel. Collectively, approximately 52% of subjects were positive for at least one genetic variance. The prevalence of gene variants in combinations of the AI1, AI2, and AI3 categories are shown in Tables 4-6.

### Example II

A 177 cohort of patients with lung squamous cell carcinoma were characterized by next generation sequencing methods and gene variants were mapped onto this population, according to the methods of Example I. The prevalence of gene variants in AI1, AI2, and AI3 categories in the TCGA squamous cell carcinoma 177 patient cohort are shown in Tables 7-8.

Additional genes and their levels of evidence and corresponding actionabilities are shown in Tables 9-12

### 13. Example III

A patient presents with late stage NSCLC. A test is conducted to determine the mutation status of highly actionable NSCLC biomarkers in Table 9 in one panel to preserve limited tumor biopsy sample. A report is generated outlining the mutation status of the sample and corresponding actionability indices. A course of treatment is determined based on the mutation status of the patient's tumor.

### Example IV

Actionability content is generated based on a subject's gene variant status. An FFPE sample comprising a NSCLC tumor cell is obtained from a subject. The sample is prepared for mutation, copy number, gene fusion, and expression analysis. The sample is sequenced using NGS, in particular using semiconductor sequencing. Based on results obtained from NGS, a Reflex Test is performed to confirm variant status. A report is generated comprising an Actionability Index and recommended action associated with the variant status. In this regard, the tumor cell comprises an ALK translocation. Prescribing information includes treatment with a kinase inhibitor for locally advanced or metastatic NSCLC. The treatment is in accordance with NCCN Clinical guidelines for NSCLC, which is supported by early clinical evidence. Enrolling and pending clinical trial information is further provided in the report (See Example V).

### Example V

An exemplary report. A report is generated related with content related to an ALK translocation. The report contains actionability content as follows:

ALK Translocation: Prescribing information: XALKORI (crizotinib) is a kinase inhibitor indicated for the treatment of patients with locally advanced or metastatic non-small cell lung cancer (NSCLC) that is anaplastic lymphoma kinase (ALK)-positive as detected by an FDA approved test.¹

NCCN Clinical Guidelines (NSCLC): Anaplastic lymphoma kinase (ALK) gene rearrangements represent the fusion between ALK and various partner genes, including echinoderm microtubule-associated protein like 4 (EML4). ALK fusions have been identified in a subset of patients with NSCLC and represent a unique subset of NSCLC patients for whom ALK inhibitors may represent an effective therapeutic strategy. XALKORI (crizotinib) is an oral ALK inhibitor that is approved by the FDA for patients with locally advanced or metastatic NSCLC who have the ALK gene rearrangement (i.e. ALK positive).²

Early clinical evidence: In a Phase I trial, a second-generation ALK inhibitor, LDK378, showed a marked clinical response in 78 patients with ALK positive metastatic non-small cell lung cancer (NSCLC) who had progressed during or after crizotinib therapy or had not been previously treated with crizotinib. Currently, LDK378 is in Phase II clinical trials and Phase III trials are planned.³

Clinical trials: As of 9 Jul. 2013, 10 clinical trials for ALK positive NSCLC patients were recruiting participants.⁴

As of 9 Jul. 2013, 3 Phase I, 2 Phase I/II, 3 Phase II and 2 Phase III clinical trials were recruiting ALK positive NSCLC patients.⁴

In addition, several clinical trials for investigational ALK tyrosine kinase inhibitors were recruiting patients with NSCLC and advanced cancers.⁴

The report further comprises references related to the actionability content reported: (1) http://www.accessdata.fda.gov/drugsatfda_docs/label/2012/202570s002lbl.pdf; (2) NCCN Guidelines Version 2.2013 Non-Small Cell Lung Cancer; (3) Shaw A, et al. J Clin Oncol 31, 2013 (suppl; abstr TPS8119); (4) http://clinicaltrials.gov/; http://www.mycancergenome.org.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

2TABLE 10

| | | | | | Trial | | |
|---|---|---|---|---|---|---|---|
| | | | | | Phase | | |
| Row Id | Gene | Variant (if specified) | Trial Title | Disease Type(s) | (s) | Patient Segment(s) | Primary Drugs |
| 2 | ALK | Mutations | A Phase I, Non-randomized, Open- | Breast | I | Second line or | ASP-3026 |
| | | | label, Repeat Oral Administration | Colorectal | | greater/Refractory/Relapsed | |
| | | | Study of ASP3026 in Patients With | Liver | | Stage III | |
| | | | Solid Tumors | Lung, Non-Small Cell | | Stage IV | |
| | | | | Soft Tissue Sarcoma | | | |
| | | | | Unspecified Solid Tumor | | | |
| 3 | ALK | Positive | A Phase I/IIa Open-Label, Dose | Lung, Non-Small Cell | I/II | Aggressive | TSR-011 |
| | | | Escalation and Cohort Expansion Trial | Lymphoma, Hodgkin's | | Classical | |
| | | | of Oral TSR-011 in Patients With | Lymphoma, Non-Hodgkin's | | First line | |
| | | | Advanced Solid Tumors and | Pancreas | | Indolent | |
| | | | Lymphomas | Thyroid | | Nodular lymphocyte-predominant | |
| | | | | | | Second line or | |
| | | | | | | greater/Refractory/Relapsed | |
| | | | | | | Stage II | |
| | | | | | | Stage III | |
| | | | | | | Stage IV | |
| 1 | AKT1 | Unspecified | A Phase I, First-in-Human, Dose | Breast | I | Aggressive | MSC-2363318A |
| | | | Escalation Trial of MSC2363318A, a | Lung, Non-Small Cell | | Classical | |
| | | | Dual p70S6K/Akt Inhibitor, in | Lymphoma, Hodgkin's | | HER2 positive | |
| | | | Subjects With Advanced Malignancies | Lymphoma, Non-Hodgkin's | | Indolent | |
| | | | | Unspecified Solid Tumor | | Nodular lymphocyte-predominant | |
| | | | | | | Second line or | |
| | | | | | | greater/Refractory/Relapsed | |
| | | | | | | Stage III | |
| | | | | | | Stage IV | |
| 1 | BRAF | Unspecified | An Open-Label, Phase lb Dose | Breast | I | HER2 negative | pimasertib |
| | | | Escalation Trial of Oral Combination | Colorectal | | Locally advanced | XL-765 |
| | | | Therapy With MSC1936369B and | Endometrial | | Metastatic | |
| | | | SAR245409 in Subjects With Locally | Lung, Non-Small Cell | | Recurrent | |
| | | | Advanced or Metastatic Solid Tumors. | Melanoma | | Second line or | |
| | | | | Ovarian | | greater/Refractory/Relapsed | |
| | | | | Pancreas | | Stage II | |
| | | | | Renal | | Stage III | |
| | | | | Thyroid | | Stage IV | |
| | | | | Unspecified Solid Tumor | | Triple receptor negative | |
| 2 | BRAF | Unspecified | A Phase Ib Open-label, Multi-center, | Breast | I | HER2 negative | ARRY-438162 |
| | | | Dose Escalation and Expansion Study | Colorectal | | Second line or | BYL-719 |
| | | | of Orally Administered MEK162 Plus | Esophageal | | greater/Refractory/Relapsed | |
| | | | BYL719 in Adult Patients With | Lung, Non-Small Cell | | Stage II | |
| | | | Selected Advanced Solid Tumors | Melanoma | | Stage III | |
| | | | | Pancreas | | Stage IV | |
| | | | | Unspecified Solid Tumor | | Triple receptor negative | |
| 3 | BRAF | V600E | A Phase II Study of the Selective BRAF | Lung, Non-Small Cell | II | Second line or | dabrafenib |
| | | | Kinase Inhibitor GSK2118436 in | | | greater/Refractory/Relapsed | |
| | | | Subjects With Advanced Non-small | | | Stage IV | |
| | | | Cell Lung Cancer and BRAF Mutations | | | | |
| 4 | BRAF | inactivating | Phase II Trial of Dasatinib in Subjects | Lung, Non-Small Cell | II | First line | dasatinib (tablet) |
| | | mutations or | With Advanced Cancers Harboring | Melanoma | | Second line or | |
| | | uncharacterized | DDR2 Mutation or Inactivating B-RAF | | | greater/Refractory/Relapsed | |
| | | mutations | Mutation | | | Stage III | |
| | | | | | | Stage IV | |
| 5 | BRAF | V600 mutant | A Phase Ib, Open-Label Study | Colorectal | I | First line | cobimetinib |
| | | | Evaluating the Safety, Tolerability, | Lung, Non-Small Cell | | Second line or | onartuzumab |
| | | | and Pharmacokinetics of | Melanoma | | greater/Refractory/Relapsed | vemurafenib |
| | | | Onartuzumab in Combination With | Unspecified Solid Tumor | | Stage III | |
| | | | Vemurafenib and/or Cobimetinib in | | | Stage IV | |
| | | | Patients with Advanced Solid | | | | |
| | | | Malignancies | | | | |
| 1 | DDR2 | Unspecified | Phase II Trial of Dasatinib in Subjects | Lung, Non-Small Cell | II | First line | dasatinib (tablet) |
| | | | With Advanced Cancers Harboring | Melanoma | | Second line or | |
| | | | DDR2Mutation or Inactivating B-RAF | | | greater/Refractory/Relapsed | |
| | | | Mutation | | | Stage III | |
| | | | | | | Stage IV | |
| 32 | EGFR | Unspecified | Ipilimumab Plus Targeted Inhibitor | Lung, Non-Small Cell | I | First line | crizotinib |
| | | | (Erlotinib or Crizotinib) for EGFR or | | | Second line or | erlotinib |
| | | | ALK Mutated Stage IV Non-small Cell | | | greater/Refractory/Relapsed | ipilimumab |
| | | | Lung Cancer: Phase lb with Expansion | | | Stage IV | |
| | | | Cohorts | | | | |
| 33 | EGFR | Unspecified | Phase I Trial Evaluating Safety and | Lung, Non-Small Cell | I | First line | afatinib |
| | | | Tolerability of the Irreversible | | | Second line or | dasatinib |
| | | | Epidermal Growth Factor Receptor | | | greater/Refractory/Relapsed | |
| | | | Inhibitor Afatinib (BIBW 2992) in | | | Stage III | |
| | | | Combination With the SRC Kinase | | | Stage IV | |
| | | | Inhibitor Dasatinib for Patients With | | | | |
| | | | Non-small Cell Lung Cancer (NSCLC) | | | | |
| 34 | EGFR | T790M | Phase I Trial Evaluating Safety and | Lung, Non-Small Cell | I | First line | afatinib |
| | | | Tolerability of the Irreversible | | | Second line or | dasatinib |
| | | | Epidermal Growth Factor Receptor | | | greater/Refractory/Relapsed | |
| | | | Inhibitor Afatinib (BIBW 2992) in | | | Stage III | |
| | | | Combination With the SRC Kinase | | | Stage IV | |
| | | | Inhibitor Dasatinib for Patients With | | | | |
| | | | Non-small Cell Lung Cancer (NSCLC) | | | | |
| 35 | EGFR | G719X, exon 19 | Phase I Study of INC280 Plus Erlotinib | Lung, Non-Small Cell | I | (N/A) | INCB-028060 |
| | | deletion, L858R, | in Patients With C-Met Expressing | | | Second line or | |
| | | L861Q | Non-Small Cell Lung Cancer | | | greater/Refractory/Relapsed | |
| 36 | EGFR | activating | A Phase Ib Open-label Study to | Lung, Non-Small Cell | I | Line of therapy N/A | MEDI-4736 |
| | | mutation | Evaluate the Safety and Tolerability of | | | Stage III Stage IV | |
| | | | MEDI4736 in Combination with | | | | |
| | | | Tremelimumab in Subjects with | | | | |
| | | | Advanced Non-small Cell Lung Cancer | | | | |
| 37 | EGFR | exon 19 deletion, | A Randomized Phase II Study of | Lung, Non-Small Cell | II | First line | crizotinib |
| | | L858 | Individualized Combined Modality | | | Maintenance/Consolidation | erlotinib |
| | | | Therapy for Stage III Non-Small Cell | | | Stage III | |
| | | | Lung Cancer (NSCLC) | | | | |
| 38 | EGFR | activating mutation | An Open-Label, Single-Center, Dose- | Lung, Non-Small Cell | I | Second line or | RTA-408 |
| | | | Escalation, Phase 1 Study of the | | | greater/Refractory/Relapsed | |
| | | | Safety, Tolerability, | | | Stage III | |
| | | | Pharmacodynamics, and | | | Stage IV | |
| | | | Pharmacokinetics of RTA 408 in the | | | | |
| | | | Treatment of Patients With | | | | |
| | | | Metastatic Non-Small Cell Lung | | | | |
| | | | Cancer | | | | |
| 39 | EGFR | activating mutation | EValuation of Erlotinib as a | Lung, Non-Small Cell | II | Neoadjuvant | erlotinib |
| | | | Neoadjuvant Therapy in Stage III Non- | | | Stage III | |
| | | | small Cell Lung Cancer Patients With | | | | |
| | | | EGFR Mutations (EVENT Trial) | | | | |
| 1 | ERBB2 | Unspecified | An Open-Label, Phase Ib Dose | Breast | I | HER2 negative | pimasertib |
| | | | Escalation Trial of Oral Combination | Colorectal | | Locally advanced | XL-765 |
| | | | Therapy With MSC1936369B and | Endometrial | | Metastatic | |
| | | | SAR245409 in Subjects With Locally | Lung, Non-Small Cell | | Recurrent | |
| | | | Advanced or Metastatic Solid Tumors. | Melanoma | | Second line or | |
| | | | | Ovarian | | greater/Refractory/Relapsed | |
| | | | | Pancreas | | Stage II | |
| | | | | Renal | | Stage III | |
| | | | | Thyroid | | Stage IV | |
| | | | | Unspecified Solid Tumor | | Triple receptor negative | |
| 2 | ERBB2 | Activating mutation | A Phase II Study of Neratinib and | Lung, Non-Small Cell | II | Second line or | neratinib |
| | | | Neratinib Plus Temsirolimus in | | | greater/Refractory/Relapsed | |
| | | | Patients With Non-Small Cell Lung | | | Stage III | |
| | | | Cancer Carrying Known HER2 | | | Stage IV | |
| | | | ActivatingMutations. | | | | |
| 3 | ERBB2 | Unspecified | A Phase I, First-in-Human, Dose | Breast | I | Aggressive | MSC-2363318A |
| | | | Escalation Trial of MSC2363318A, a | Lung, Non-Small Cell | | Classical | |
| | | | Dual p70S6K/Akt Inhibitor, in Subjects | Lymphoma, Hodgkin's | | HER2 positive | |
| | | | With Advanced Malignancies | Lymphoma, Non-Hodgkin's | | Indolent | |
| | | | | Unspecified Solid Tumor | | Nodular lymphocyte-predominant | |
| | | | | | | Second line or | |
| | | | | | | greater/Refractory/Relapsed | |
| | | | | | | Stage III | |
| | | | | | | Stage IV | |
| 1 | FGFR3 | Unspecified | A Phase I, Open-label, Multi-center, | Bladder | I | Second line or | BGJ-398 |
| | | | Dose Escalation Study of Oral | Breast | | greater/Refractory/Relapsed | |
| | | | BGJ398, a Pan FGF-R Kinase Inhibitor, | Gastric | | Stage III | |
| | | | in Adult Patients With Advanced Solid | Lung, Non-Small Cell | | Stage IV | |
| | | | Malignancies | Lung, Small Cell | | | |
| | | | | Unspecified Solid Tumor | | | |
| 1 | KRAS | Unspecified | A Phase I Dose Escalation Open-Label | Breast | I | HER2 negative | GSK-2141795 |
| | | | Safety and Pharmacokinetic Study to | Colorectal | | Recurrent | trametinib |
| | | | Determine the Recommended Phase | Endometrial | | Second line or | |
| | | | II Dose of GSK1120212 Dosed in | Head/Neck | | greater/Refractory/Relapsed | |
| | | | Combination With GSK2141795 in | Lung, Non-Small Cell | | Stage III | |
| | | | Subjects With Solid Tumors (Part 1) | Melanoma | | Stage IV | |
| | | | and in Subjects With Pancreatic | Ovarian | | Triple receptor negative | |
| | | | Cancer, Endometrial Cancer or | Pancreas | | | |
| | | | Colorectal Cancer (Part 2) | Thyroid | | | |
| | | | | Unspecified Solid Tumor | | | |
| 2 | KRAS | Unspecified | An Open-Label, Phase Ib Dose | Breast | I | HER2 negative | pimasertib |
| | | | Escalation Trial of Oral Combination | Colorectal | | Locally advanced | XL-765 |
| | | | Therapy With MSC1936369B and | Endometrial | | Metastatic | |
| | | | SAR245409 in Subjects With Locally | Lung, Non-Small Cell | | Recurrent | |
| | | | Advanced or Metastatic Solid Tumors. | Melanoma | | Second line or | |
| | | | | Ovarian | | greater/Refractory/Relapsed | |
| | | | | Pancreas | | Stage II | |
| | | | | Renal | | Stage III | |
| | | | | Thyroid | | Stage IV | |
| | | | | Unspecified Solid Tumor | | Triple receptor negative | |
| 3 | KRAS | Unspecified | A Phase Ib Open-label, Multi-center, | Breast | I | HER2 negative | ARRY-438162 |
| | | | Dose Escalation and Expansion Study | Colorectal | | Second line or | BYL-719 |
| | | | of Orally Administered MEK162 Plus | Esophageal | | greater/Refractory/Relapsed | |
| | | | BYL719 in Adult Patients With | Lung, Non-Small Cell | | Stage II | |
| | | | Selected Advanced Solid Tumors | Melanoma | | Stage III | |
| | | | | Pancreas | | Stage IV | |
| | | | | Unspecified Solid Tumor | | Triple receptor negative | |
| 4 | KRAS | Unspecified | A Phase Ib/II Study of Retaspimycin | Lung, Non-Small Cell | I/II | Second line or | retaspimycin |
| | | | HCI (IPI-504) in Combination With | | | greater/Refractory/Relapsed | |
| | | | Everolimus in Patients With KRAS | | | Stage III | |
| | | | Mutant NSCLC | | | Stage IV | |
| 5 | KRAS | Unspecified | A Phase II Randomized Open-label | Lung, Non-Small Cell | II | Second line or | tivantinib |
| | | | Study of Erlotinib Plus ARQ 197 | | | greater/Refractory/Relapsed | |
| | | | Versus Single Agent Chemotherapy in | | | Stage III | |
| | | | Previously Treated KRAS Mutation | | | Stage IV | |
| | | | Positive Subjects With Locally | | | | |
| | | | Advanced or Metastatic Non-Small | | | | |
| | | | Cell Lung Cancer | | | | |
| 6 | KRAS | G12D | A Phase II Trial of Bortezomib in KRAS- | Lung, Non-Small Cell | II | Second line or | bortezomib (SC) |
| | | | Mutant Non-Small Cell Lung Cancer in | | | greater/Refractory/Relapsed | |
| | | | Never Smokers or Those With KRAS | | | Stage III | |
| | | | G12D | | | Stage IV | |
| 7 | KRAS | Unspecified | A Phase I/1B Trial of MEK162 in | Lung, Non-Small Cell | I | First line | binimetinib |
| | | | Combination With Erlotinib in Non- | | | Second line or | |
| | | | Small Cell Lung Cancer (NSCLC) | | | greater/Refractory/Relapsed | |
| | | | Harboring KRAS or EGFRMutation | | | Stage IV | |
| 8 | KRAS | G12/G13/Q61 | A Phase I Study of Trametinib in | Lung, Non-Small Cell | I | Maintenance/Consolidation | 3D-CRT |
| | | | Combination With Chemoradiation | | | Stage II | carboplatin (iv) |
| | | | for KRAS Mutant Non-Small Cell Lung | | | Stage III | intensity- |
| | | | Cancer | | | | modulated |
| | | | | | | | radiation |
| | | | | | | | therapy |
| | | | | | | | paclitaxel |
| 9 | KRAS | Unspecified | A Phase Ib, Open-Label Study | Colorectal | I | First line | cobimetinib |
| | | | Evaluating the Safety, Tolerability, | Lung, Non-Small Cell | | Second line or | onartuzumab |
| | | | and Pharmacokinetics of | Melanoma | | greater/Refractory/Relapsed | vemurafenib |
| | | | Onartuzumab in Combination With | Unspecified Solid Tumor | | Stage III | |
| | | | Vemurafenib and/or Cobimetinib in | | | Stage IV | |
| | | | Patients with Advanced Solid | | | | |
| | | | Malignancies | | | | |
| 10 | KRAS | Unspecified | A Phase Ib, Open-Label, Dose- | Colorectal | I | Second line or | cobimetinib |
| | | | Escalation Study of The Safety, | Lung, Non-Small Cell | | greater/Refractory/Relapsed | MEHD-7945A |
| | | | Tolerability, and Pharmacokinetics Of | Unspecified Solid Tumor | | Stage III | |
| | | | MEHD7945A and GDC-0973 In | | | Stage IV | |
| | | | Patients with Locally Advanced or | | | | |
| | | | Metastatic Solid Tumors with Mutant | | | | |
| | | | Kras | | | | |
| 11 | KRAS | Unspecified | A Phase Ib Study of the Safety and | Colorectal | I | Second line or | cobimetinib |
| | | | Pharmacology of MPDL3280A | Lung, Non-Small Cell | | greater/Refractory/Relapsed | RG-7446 |
| | | | Administered with Cobimetinib in | Melanoma | | Stage III | |
| | | | Patients with Locally Advanced or | Unspecified Solid Tumor | | Stage IV | |
| | | | Metastatic Solid Tumors | | | | |
| 12 | KRAS | Unspecified | Phase II Study of VS-6063, A Focal | Lung, Non-Small Cell | II | Second line or | defactinib |
| | | | Adhesion Kinase (FAK) Inhibitor, in | | | greater/Refractory/Relapsed | |
| | | | Patients With KRAS Mutant Non- | | | Stage IV | |
| | | | Small Cell Lung Cancer | | | | |
| 13 | KRAS | Unspecified | A Phase III, Double-Blind, | Lung, Non-Small Cell | III | Second line or | selumetinib |
| | | | Randomised, Placebo-Controlled | | | greater/Refractory/Relapsed | (capsule) |
| | | | Study to Assess the Efficacy and | | | Stage III | |
| | | | Safety of Selumetinib (AZD6244; | | | Stage IV | |
| | | | ARRY-142886) (Hyd-Sulfate) in | | | | |
| | | | Combination With Docetaxel, in | | | | |
| | | | Patients Receiving Second Line | | | | |
| | | | Treatment for KRASMutation- | | | | |
| | | | Positive Locally Advanced or | | | | |
| | | | Metastatic Non Small Cell Lung | | | | |
| | | | Cancer (Stage IIIB-IV) (SELECT 1) | | | | |
| | | | SELumetinib Evaluation as | | | | |
| | | | Combination Therapy-1 (SELECT-1) | | | | |
| 14 | KRAS | G12R, G12C, | A Phase Ib Dose-Escalation Study of | Breast | I | HER2 positive | lapatinib ditosylate |
| | | G12V, G12D, | the AKT Inhibitor MK-2206 (NSC# | Lung, Non-Small Cell | | Second line or | MK-2206 |
| | | G13 | 749607) Plus Lapatinib(NSC# 727989) | Lung, Small Cell | | greater/Refractory/Relapsed | |
| | | | Administered in Patients With HER2 | Unspecified Solid Tumor | | Stage III | |
| | | | Positive Metastatic Breast Cancer | | | Stage IV | |
| 15 | KRAS | Unspecified | Phase I/II Study of the CDK4/6 | Lung, Non-Small Cell | I/II | Second line or | palbociclib |
| | | | Inhibitor Palbociclib (PD-0332991) in | Unspecified Solid Tumor | | greater/Refractory/Relapsed | PD-0325901 |
| | | | Combination With the MEK Inhibitor | | | Stage III | |
| | | | PD-0325901 for Patients With KRAS | | | Stage IV | |
| | | | Mutant Non-Small Cell Lung Cancer | | | | |
| | | | and Other Solid Tumors | | | | |
| 2 | MET | Unspecified | Phase I/II Safety, Pharmacokinetic | (N/A) | I/II | Aggressive | crizotinib |
| | | | And Pharmacodynamic Study Of PF- | Bladder | | First line | |
| | | | 02341066, A c-Met/HGFR Selective | CNS, Glioblastoma | | Locally advanced | |
| | | | Tyrosine Kinase Inhibitor, | Colorectal | | Metastatic | |
| | | | Administered Orally To Patients With | Gastric | | Peripheral T-cell lymphoma (PTCL) | |
| | | | Advanced Cancer | Head/Neck | | Second line or | |
| | | | | Lung, Non-Small Cell | | greater/Refractory/Relapsed | |
| | | | | Lymphoma, Non-Hodgkin's | | Stage III | |
| | | | | Ovarian | | Stage IV | |
| | | | | Pancreas | | | |
| | | | | Renal | | | |
| | | | | Soft Tissue Sarcoma | | | |
| 3 | MET | Unspecified | A Randomized, Phase III, Multicenter, | Lung, Non-Small Cell | III | First line | onartuzumab |
| | | | Double-blind, Placebo-controlled | | | Stage III | |
| | | | Study Evaluating The Efficacy And | | | Stage IV | |
| | | | Safety of Onartuzumab in | | | | |
| | | | Combination with Erlotinib as First- | | | | |
| | | | line treatment for patients with MET- | | | | |
| | | | Positive unresectable stage IIIb or IV | | | | |
| | | | non-small cell lung cancer (NSCLC) | | | | |
| | | | carrying an activating EGFRMutation | | | | |
| 4 | MET | Unspecified | A Phase I Open-label, Non- | Bladder | I | Second line or | EMD-1214063 |
| | | | randomized, Dose-escalation | Lung, Non-Small Cell | | greater/Refractory/Relapsed | |
| | | | First-in-man Trial to Investigate | Prostate | | Stage III | |
| | | | the c-Met Kinase Inhibitor EMD | Unspecified Solid Tumor | | Stage IV | |
| | | | 1214063 Under Two Different | | | | |
| | | | Regimens in Subjects With | | | | |
| | | | Advanced Solid Tumors. | | | | |
| 5 | MET | Unspecified | Phase I Study of INC280 Plus | Lung, Non-Small Cell | I | (N/A) | INCB-028060 |
| | | | Erlotinib in Patients With C-Met | | | Second line or | |
| | | | Expressing Non-Small Cell Lung | | | greater/Refractory/Relapsed | |
| | | | Cancer | | | | |
| 1 | NRAS | Unspecified | An Open-Label, Phase Ib Dose | Breast | I | HER2 negative | pimasertib |
| | | | Escalation Trial of Oral Combination | Colorectal | | Locally advanced | XL-765 |
| | | | Therapy With MSC1936369B and | Endometrial | | Metastatic | |
| | | | SAR245409 in Subjects With Locally | Lung, Non-Small Cell | | Recurrent | |
| | | | Advanced or Metastatic Solid Tumors. | Melanoma | | Second line or | |
| | | | | Ovarian | | greater/Refractory/Relapsed | |
| | | | | Pancreas | | Stage II | |
| | | | | Renal | | Stage III | |
| | | | | Thyroid | | Stage IV | |
| | | | | Unspecified Solid Tumor | | Triple receptor negative | |
| 2 | NRAS | Unspecified | A Phase Ib Open-label, Multi-center, | Breast | I | HER2 negative | ARRY-438162 |
| | | | Dose Escalation and Expansion Study | Colorectal | | Second line or | BYL-719 |
| | | | of Orally Administered MEK162 Plus | Esophageal | | greater/Refractory/Relapsed | |
| | | | BYL719 in Adult Patients With | Lung, Non-Small Cell | | Stage II | |
| | | | Selected Advanced Solid Tumors | Melanoma | | Stage III | |
| | | | | Pancreas | | Stage IV | |
| | | | | Unspecified Solid Tumor | | Triple receptor negative | |
| 3 | NRAS | Unspecified | A Phase Ib, Open-label, Multi-center, | Breast | I/II | HER2 negative | ARRY-438162 |
| | | | Dose-escalation and Expansion Study | Colorectal | | Line of therapy N/A | buparlisib |
| | | | of an Orally Administered | Lung, Non-Small Cell | | Stage II | |
| | | | Combination of BKM120 Plus | Melanoma | | Stage III | |
| | | | MEK162 in Adult Patients With | Pancreas | | Stage IV | |
| | | | Selected Advanced Solid Tumors | Unspecified Solid Tumor | | Triple receptor negative | |
| 1 | PIK3CA | Unspecified | An Open-Label, Phase Ib Dose | Breast | I | HER2 negative | pimasertib |
| | | | Escalation Trial of Oral Combination | Colorectal | | Locally advanced | XL-765 |
| | | | Therapy With MSC1936369B and | Endometrial | | Metastatic | |
| | | | SAR245409 in Subjects With Locally | Lung, Non-Small Cell | | Recurrent | |
| | | | Advanced or Metastatic Solid Tumors. | Melanoma | | Second line or | |
| | | | | Ovarian | | greater/Refractory/Relapsed | |
| | | | | Pancreas | | Stage II | |
| | | | | Renal | | Stage III | |
| | | | | Thyroid | | Stage IV | |
| | | | | Unspecified Solid Tumor | | Triple receptor negative | |
| 2 | PIK3CA | Unspecified | A Phase Ib Open-label, Multi-center, | Breast | I | HER2 negative | ARRY-438162 |
| | | | Dose Escalation and Expansion Study | Colorectal | | Second line or | BYL-719 |
| | | | of Orally Administered MEK162 Plus | Esophageal | | greater/Refractory/Relapsed | |
| | | | BYL719 in Adult Patients With | Lung, Non-Small Cell | | Stage II | |
| | | | Selected Advanced Solid Tumors | Melanoma | | Stage III | |
| | | | | Pancreas | | Stage IV | |
| | | | | Unspecified Solid Tumor | | Triple receptor negative | |
| 3 | PIK3CA | Unspecified | An Open Label Two-stage Study of | Lung, Non-Small Cell | II | Second line or | buparlisib |
| | | | Orally Administered BKM120 in | | | greater/Refractory/Relapsed | |
| | | | Patients With Metastatic Non-small | | | Stage IV | |
| | | | Cell Lung Cancer With Activated PI3K | | | | |
| | | | Pathway | | | | |
| 4 | PIK3CA | E542K, E545K, | A Phase I b Dose-Escalation Study | Breast | I | HER2 positive | lapatinib |
| | | H1047R, H1047L | of the AKT Inhibitor MK-2206 | Lung, Non-Small Cell | | Second line or | MK-2206 |
| | | | (NSC# 749607) Plus | Lung, Small Cell | | greater/Refractory/Relapsed | |
| | | | Lapatinib(NSC# 727989) | Unspecified Solid Tumor | | Stage III | |
| | | | Administered in Patients With | | | Stage IV | |
| | | | HER2 Positive Metastatic Breast | | | | |
| | | | Cancer | | | | |
| 5 | PIK3CA | Unspecified | Phase I/II Study of the | CNS, Glioblastoma | I/II | Second line or | buparlisib |
| | | | Combination of BKM120 and | Colorectal | | greater/Refractory/Relapsed | |
| | | | Bevacizumab in Patients With | Lung, Non-Small Cell | | Stage III | |
| | | | Refractory Solid Tumors (Phase | Unspecified Solid Tumor | | Stage IV | |
| | | | I) and Relapsed/Refractory | | | | |
| | | | Glioblastoma Multiforme | | | | |
| | | | (Phase II) | | | | |
| 6 | PIK3CA | Unspecified | A Phase I, First-in-Human, Dose | Breast | I | Aggressive | MSC-2363318A |
| | | | Escalation Trial of | Lung, Non-Small Cell | | Classical | |
| | | | MSC2363318A, a Dual | Lymphoma, Hodgkin's | | HER2 positive | |
| | | | p70S6K/Akt Inhibitor, in Subjects | Lymphoma, Non-Hodgkin's | | Indolent | |
| | | | With Advanced Malignancies | Unspecified Solid Tumor | | Nodular lymphocyte-predominant | |
| | | | | | | Second line or | |
| | | | | | | greater/Refractory/Relapsed | |
| | | | | | | Stage III | |
| | | | | | | Stage IV | |
| 2 | PTEN | Unspecified | An Open Label Two-stage Study of | Lung, Non-Small Cell | II | Second line or | buparlisib |
| | | | Orally Administered BKM120 in | | | greater/Refractory/Relapsed | |
| | | | Patients With Metastatic Non-small | | | Stage IV | |
| | | | Cell Lung Cancer With Activated PI3K | | | | |
| | | | Pathway | | | | |
| 3 | PTEN | Unspecified | A Phase I, First-in-Human, Dose | Breast | I | Aggressive | MSC-2363318A |
| | | | Escalation Trial of | Lung, Non-Small Cell | | Classical | |
| | | | MSC2363318A, a Dual | Lymphoma, Hodgkin's | | HER2 positive | |
| | | | p70S6K/Akt Inhibitor, in Subjects | Lymphoma, Non-Hodgkin's | | Indolent | |
| | | | With Advanced Malignancies | Unspecified Solid Tumor | | Nodular lymphocyte-predominant | |
| | | | | | | Second line or | |
| | | | | | | greater/Refractory/Relapsed | |
| | | | | | | Stage III | |
| | | | | | | Stage IV | |
| 1 | STK11 | Unspecified | A Phase I, First-in-Human, Dose | Breast | I | Aggressive | MSC-2363318A |
| | (LKB1) | | Escalation Trial of | Lung, Non-Small Cell | | Classical | |
| | | | MSC2363318A, a Dual | Lymphoma, Hodgkin's | | HER2 positive | |
| | | | p70S6K/Akt Inhibitor, in Subjects | Lymphoma, Non-Hodgkin's | | Indolent | |
| | | | With Advanced Malignancies | Unspecified Solid Tumor | | Nodular lymphocyte-predominant | |
| | | | | | | Second line or | |
| | | | | | | greater/Refractory/Relapsed | |
| | | | | | | Stage III | |
| | | | | | | Stage IV | |
| 1 | ALK | Fusion | A Phase I, Multi-center, Open Label | Breast | I | First line | LDK-378 |
| | | | Dose Escalation Study of LDK378, | Colorectal | | Locally advanced | |
| | | | Administered Orally in Adult Patients | Lung, Non-Small Cell | | Metastatic | |
| | | | With Tumors Characterized by | Soft Tissue Sarcoma | | Second line or | |
| | | | Genetic Abnormalities in Anaplastic | Unspecified Solid Tumor | | greater/Refractory/Relapsed | |
| | | | Lymphoma Kinase (ALK) | | | Stage III | |
| | | | | | | Stage IV | |
| | | | | | | Untreated | |
| 2 | ALK | Fusion | A Phase I, Open-Label, Multiple- | (N/A) | I | Hormone refractory | DS-2248 |
| | | | Ascending-Dose Study of DS-2248, an | Lung, Non-Small Cell | | Second line or | |
| | | | Orally Bioavailable Heat Shock | Prostate | | greater/Refractory/Relapsed | |
| | | | Unspecified Solid Tumor | | | Stage III | |
| | | | Protein 90 Inhibitor, in Subjects With | | | | |
| | | | Advanced Solid Tumors | Stage IV | | | |
| 3 | ALK | Fusion | Phase I/II Safety, Pharmacokinetic | (N/A) | I/II | Aggressive | crizotinib |
| | | | And Pharmacodynamic Study Of PF- | Bladder | | First line | |
| | | | 02341066, A c-Met/HGFR Selective | CNS, Glioblastoma | | Locally advanced | |
| | | | Tyrosine Kinase Inhibitor, | Colorectal | | Metastatic | |
| | | | Administered Orally To Patients With | Gastric | | Peripheral T-cell lymphoma (PTCL) | |
| | | | Advanced Cancer | Head/Neck | | Second line or | |
| | | | | Lung, Non-Small Cell | | greater/Refractory/Relapsed | |
| | | | | Lymphoma, Non-Hodgkin's | | Stage III | |
| | | | | Ovarian | | Stage IV | |
| | | | | Pancreas | | | |
| | | | | Renal | | | |
| | | | | Soft Tissue Sarcoma | | | |
| 4 | ALK | Fusion | A Phase I/II Study of the Safety, | (N/A) | I/II | Aggressive | AP-26113 |
| | | | Tolerability, Pharmacokinetics and | Colorectal | | Classical | |
| | | | Preliminary Anti-Tumor Activity of | Liver | | Diffuse large B-cell lymphoma (DLBCL) | |
| | | | the Oral ALK/EG FR Inhibitor AP26113 | Lung, Non-Small Cell | | Nodular lymphocyte-predominant | |
| | | | | Lymphoma, Hodgkin's | | Peripheral T-cell lymphoma (PTCL) | |
| | | | | Lymphoma, Non-Hodgkin's | | Second line or | |
| | | | | Pancreas | | greater/Refractory/Relapsed | |
| | | | | Unspecified Solid Tumor | | Stage II | |
| | | | | | | Stage III | |
| | | | | | | Stage IV | |
| 5 | ALK | Fusion | A Phase I/II Study of Crizotinib and | Lung, Non-Small Cell | I/II | First line | ganetespib |
| | | | STA-9090 in ALK Positive Lung Cancers | | | Second line or | |
| | | | | | | greater/Refractory/Relapsed | |
| | | | | | | Stage III | |
| | | | | | | Stage IV | |
| 6 | ALK | Fusion | An open-label, non-randomized, | Lung, Non-Small Cell | I/II | First line | alectinib |
| | | | multicenter phase I/II trial of | | | Second line or | |
| | | | RO5424802 given orally to non - | | | greater/Refractory/Relapsed | |
| | | | small cell lung cancer patients who | | | Stage III | |
| | | | have ALK mutation and failed | | | Stage IV | |
| | | | crizotinib treatment | | | | |
| 7 | ALK | Fusion | A Single Arm, Phase II Study of | Lung, Non-Small Cell | II | Second line or | ganetespib |
| | | | Ganetespib in Subjects with | | | greater/Refractory/Relapsed | |
| | | | Advanced Non-Small-Cell Lung | | | Stage III | |
| | | | Cancer With Anaplastic Lymphoma | | | Stage IV | |
| | | | Kinase Gene Rearrangement (ALK- | | | | |
| | | | Positive NSCLC) Evaluating | | | | |
| | | | CHaperone Inhibition in Alk | | | | |
| | | | Rearranged lung cAncer-CHIARA | | | | |
| 8 | ALK | Fusion | A Phase II, Multicenter, Single-arm | Lung, Non-Small Cell | II | Second line or | LDK-378 |
| | | | Study of Oral LDK378 in Crizotinib | | | greater/Refractory/Relapsed | |
| | | | na [symbol] Adult Patients With ALK- | | | Stage III | |
| | | | activated Non-small Cell Lung Cancer | | | Stage IV | |
| 9 | ALK | Fusion | A Randomized Phase II Trial of | Lung, Non-Small Cell | II | Second line or | azacitidine (oral) |
| | | | Cytotoxic Chemotherapy With or | | | greater/Refractory/Relapsed | azacitidine (sc) |
| | | | Without Epigenetic Priming in | | | Stage III | entinostat |
| | | | Patients With Advanced Non-Small | | | Stage IV | |
| | | | Cell Lung Cancer. | | | | |
| 10 | ALK | Fusion | An Open-label, Multi-center, | Lung, Non-Small Cell | II | Second line or | LDK-378 |
| | | | Expanded Treatment Protocol (ETP) | | | greater/Refractory/Relapsed | |
| | | | of Oral LDK378 in Adult Patients With | | | Stage III | |
| | | | Non-small Cell Lung Cancer (NSCLC) | | | Stage IV | |
| | | | Characterized by ALK(+) | | | | |
| | | | Rearrangements in Patients | | | | |
| | | | Previously Treated With an ALK | | | | |
| | | | Inhibitor | | | | |
| 11 | ALK | Fusion | Molecular Determinants of Acquired | Lung, Non-Small Cell | Other | Line of therapy N/A | crizotinib |
| | | | Clinical Resistance to Crizotinib in | | | | |
| | | | Non-small Cell Lung Cancer Harboring | | | | |
| | | | a Translocation or Inversion Event | | | | |
| | | | Involving the ALK Gene Locus | | | | |
| 12 | ALK | Fusion | A Phase Ib, Open-label, Dose | Lung, Non-Small Cell | I | Second line or | AUY-922 |
| | | | Escalation Study of LDK378 and | | | greater/Refractory/Relapsed | LDK-378 |
| | | | AUY922 in Patients With ALK- | | | Stage III | |
| | | | rearranged Non-small Cell Lung | | | Stage IV | |
| | | | Cancer | | | | |
| 13 | ALK | Fusion | A Phase I/II Study of the ALK Inhibitor | Lung, Non-Small Cell | I/II | Second line or | alectinib |
| | | | CH5424802/RO5424802 in Patients | | | greater/Refractory/Relapsed | hydrochloride |
| | | | With ALK-Rearranged Non-Small Cell | | | Stage III | |
| | | | Lung Cancer Previously Treated With | | | Stage IV | |
| | | | Chemotherapy and Crizotinib | | | | |
| 14 | ALK | Fusion | A Phase II, Multicenter, Single-Arm | Lung, Non-Small Cell | II | First line | RG-7446 |
| | | | Study of MPDL3280A in Patients with | | | Second line or | |
| | | | PD-L1-Positive Locally Advanced or | | | greater/Refractory/Relapsed | |
| | | | Metastatic Non-small Cell Lung | | | Stage III | |
| | | | Cancer | | | Stage IV | |
| 15 | ALK | Fusion | Phase IB Study of Single Agent MK- | (N/A) | I | First line | lambrolizumab |
| | | | 3475 in Patients With Progressive | Colorectal | | Locally advanced | |
| | | | Locally Advanced or Metastatic | Lung, Non-Small Cell | | Metastatic | |
| | | | Carcinoma, Melanoma, and Non- | Melanoma | | Second line or | |
| | | | Small Cell Lung Carcinoma | Osteosarcoma | | greater/Refractory/Relapsed | |
| | | | Soft Tissue Sarcoma | | | Stage III | |
| 16 | ALK | Fusion | A Phase I, Non-randomized, Open- | Breast | I | Second line or | ASP-3026 |
| | | | label, Repeat Oral Administration | Colorectal | | greater/Refractory/Relapsed | |
| | | | Study of A5P3026 in Patients With | Liver | | Stage III | |
| | | | Solid Tumors | Lung, Non-Small Cell | | Stage IV | |
| | | | | Soft Tissue Sarcoma | | | |
| | | | | Unspecified Solid Tumor | | | |
| 17 | ALK | Fusion | Phase II, Open-Label Single Arm | Lung, Non-Small Cell | II | Second line or | crizotinib (tablet) |
| | | | Study Of The Efficacy And Safety Of | | | greater/Refractory/Relapsed | |
| | | | PF-02341066 In Patients With Non- | | | Stage III | |
| | | | Small Cell Lung Cancer Harboring A | | | Stage IV | |
| | | | Translocation Or Inversion Event | | | | |
| | | | Involving The Anaplastic Lymphoma | | | | |
| | | | Kinase (ALK) Gene | | | | |
| 18 | ALK | Fusion | A Randomized Phase II Study of | Lung, Non-Small Cell | II | First line | crizotinib |
| | | | Individualized Combined Modality | | | Maintenance/Consolidation | erlotinib |
| | | | Therapy for Stage III Non-Small Cell | | | Stage III | |
| | | | Lung Cancer (NSCLC) | | | | |
| 19 | ALK | Fusion | A Phase I/II Study of PF- | (N/A) | I/II | (N/A) | crizotinib |
| | | | 02341066, an Oral Small | CNS, Glioblastoma | | Indolent | |
| | | | Molecule Inhibitor of Anaplastic | CNS, Medulloblastoma | | Pediatric or Adolescent | |
| | | | Lymphoma Kinase (ALK) and c- | CNS, Other | | Peripheral T-cell lymphoma (PTCL) | |
| | | | Met, in Children With | Lung, Non-Small Cell | | Second line or | |
| | | | Relapsed/Refractory Solid | Lymphoma, Non-Hodgkin's | | greater/Refractory/Relapsed | |
| | | | Tumors, Primary CNS Tumors, | Unspecified Solid Tumor | | | |
| | | | and Anaplastic Large Cell | | | | |
| | | | Lymphoma | | | | |

TABLE 12

| Row ID | Gene Symbol | Accession_number | COSMIC_id | CDS_mut_syntax | AA_mut_syntax | Oncomine Gene Classification | Oncomine Variant Classification |
|---|---|---|---|---|---|---|---|
| 1 | AKT1 | ENST00000349310 | 33765 | c.49G>A | p.E17K | Gain of function | Missense_Mutation |
| 2 | ALK | NM_004304 | 28059 | c.3521T>G | p.F1174C | Gain of function | Missense_Mutation |
| 3 | ALK | NM_004304 | 28491 | c.3520T>A | p.F1174I | Gain of function | Missense_Mutation |
| 4 | ALK | NM_004304 | 28055 | c.3522C>A | p.F1174L | Gain of function | Missense_Mutation |
| 5 | ALK | NM_004304 | 28057 | c.3520T>C | p.F1174L | Gain of function | Missense_Mutation |
| 6 | ALK | NM_004304 | 28061 | c.3522C>G | p.F1174L | Gain of function | Missense_Mutation |
| 7 | ALK | NM_004304 | 28054 | c.3520T>G | p.F1174V | Gain of function | Missense_Mutation |
| 8 | ALK | NM_004304 | 99137 | c.3586C>A | p.L1196M | Gain of function | Missense_Mutation |
| 9 | ALK | NM_004304 | 98478 | c.3452C>T | p.T1151M | Gain of function | Missense_Mutation |
| 10 | BRAF | NM_004333 | 26625 | c.1794_1795insGTT | p.A598_T599insV | Gain of function | In_Frame_Ins |
| 11 | BRAF | NM_004333 | 21549 | c.1793C>T | p.A598V | Gain of function | Missense_Mutation |
| 12 | BRAF | NM_004333 | 467 | c.1781A>G | p.D594G | Gain of function | Missense_Mutation |
| 13 | BRAF | NM_004333 | 27639 | c.1780G>A | p.D594N | Gain of function | Missense_Mutation |
| 14 | BRAF | NM_004333 | 466 | c.1781A>T | p.D594V | Gain of function | Missense_Mutation |
| 15 | BRAF | NM_004333 | 1118 | c.1758A>G | p.E586E | Gain of function | Synonymous_Mutation |
| 16 | BRAF | NM_004333 | 463 | c.1756G>A | p.E586K | Gain of function | Missense_Mutation |
| 17 | BRAF | NM_004333 | 1116 | c.1749T>C | p.F583F | Gain of function | Synonymous_Mutation |
| 18 | BRAF | NM_004333 | 468 | c.1785T>G | p.F595L | Gain of function | Missense_Mutation |
| 19 | BRAF | NM_004333 | 1123 | c.1784T>C | p.F595S | Gain of function | Missense_Mutation |
| 20 | BRAF | NM_004333 | 449 | c.1391G>A | p.G464E | Gain of function | Missense_Mutation |
| 21 | BRAF | NM_004333 | 450 | c.1391G>T | p.G464V | Gain of function | Missense_Mutation |
| 22 | BRAF | NM_004333 | 26506 | c.1787G>A | p.G596D | Gain of function | Missense_Mutation |
| 23 | BRAF | NM_004333 | 469 | c.1786G>C | p.G596R | Gain of function | Missense_Mutation |
| 24 | BRAF | NM_004333 | 1137 | c.1817G>A | p.G606E | Gain of function | Missense_Mutation |
| 25 | BRAF | NM_004333 | 1138 | c.1823A>G | p.H608R | Gain of function | Missense_Mutation |
| 26 | BRAF | NM_004333 | 1115 | c.1746A>G | p.I582M | Gain of function | Missense_Mutation |
| 27 | BRAF | NM_004333 | 1119 | c.1776A>G | p.I592M | Gain of function | Missense_Mutation |
| 28 | BRAF | NM_004333 | 1120 | c.1774A>G | p.I592V | Gain of function | Missense_Mutation |
| 29 | BRAF | NM_004333 | 478 | c.1801A>G | p.K601E | Gain of function | Missense_Mutation |
| 30 | BRAF | NM_004333 | 1132 | c.1803A>C | p.K601N | Gain of function | Missense_Mutation |
| 31 | BRAF | NM_004333 | 6265 | c.1803A>T | p.K601N | Gain of function | Missense_Mutation |
| 32 | BRAF | NM_004333 | 28010 | c.1750C>T | p.L584F | Gain of function | Missense_Mutation |
| 33 | BRAF | NM_004333 | 1125 | c.1790T>A | p.L597Q | Gain of function | Missense_Mutation |
| 34 | BRAF | NM_004333 | 471 | c.1790T>G | p.L597R | Gain of function | Missense_Mutation |
| 35 | BRAF | NM_004333 | 1126 | c.1789_1790CT>TC | p.L5975 | Gain of function | Missense_Mutation |
| 36 | BRAF | NM_004333 | 470 | c.1789C>G | p.L597V | Gain of function | Missense_Mutation |
| 37 | BRAF | NM_004333 | 462 | c.1742A>G | p.N581S | Gain of function | Splice_Site |
| 38 | BRAF | NM_004333 | 21492 | c.1357C>A | p.P453T | Gain of function | Missense_Mutation |
| 39 | BRAF | NM_004333 | 6262 | c.1330C>T | p.R444W | Gain of function | Missense_Mutation |
| 40 | BRAF | NM_004333 | 447 | c.1385G>T | p.R462I | Gain of function | Missense_Mutation |
| 41 | BRAF | NM_004333 | 1117 | c.1752T>C | p.L584L | Gain of function | Synonymous_Mutation |
| 42 | BRAF | NM_004333 | 1124 | c.1791A>G | p.L597L | Gain of function | Synonymous_Mutation |
| 43 | BRAF | NM_004333 | 33729 | c.1807C>T | p.R603* | Gain of function | NonsenseMutation |
| 44 | BRAF | NM_004333 | 1135 | c.1813_1814AG>TT | p.S605F | Gain of function | Missense_Mutation |
| 45 | BRAF | NM_004333 | 21542 | c.1813A>G | p.S605G | Gain of function | Missense_Mutation |
| 46 | BRAF | NM_004333 | 1136 | c.1814G>A | p.S605N | Gain of function | Missense_Mutation |
| 47 | BRAF | NM_004333 | 144982 | c.1797_1798insACA | p.T599_V600insT | Gain of function | In_Frame_Ins |
| 48 | BRAF | NM_004333 | 30730 | c.1796_1797insTAC | p.T599_V600insT | Gain of function | In_Frame_Ins |
| 49 | BRAF | NM_004333 | 1128 | c.1797_1797A>TACTACG | p.T599_V600insTT | Gain of function | In_Frame_Ins |
| 50 | BRAF | NM_004333 | 472 | c.1796C>T | p.T599I | Gain of function | Missense_Mutation |
| 51 | BRAF | NM_004333 | 1133 | c.1799_1801delTGA | p.V600_K601>E | Gain of function | In_Frame_Del |
| 52 | BRAF | NM_004333 | 18443 | c.1799T>C | p.V600A | Gain of function | Missense_Mutation |
| 53 | BRAF | NM_004333 | 477 | c.1799_1800TG>AT | p.V600D | Gain of function | Missense_Mutation |
| 54 | BRAF | NM_004333 | 6137 | c.1799T>G | p.V600G | Gain of function | Missense_Mutation |
| 55 | BRAF | NM_004333 | 473 | c.1798_1799GT>AA | p.V600K | Gain of function | Missense_Mutation |
| 56 | BRAF | NM_004333 | 219798 | c.1798G>C | p.V600L | Gain of function | Missense_Mutation |
| 57 | BRAF | NM_004333 | 33808 | c.1798G>T | p.V600L | Gain of function | Missense_Mutation |
| 58 | BRAF | NM_004333 | 1130 | c.1798G>A | p.V600M | Gain of function | Missense_Mutation |
| 59 | BRAF | NM_004333 | 249889 | c.1798_1799GT>CA | p.V600Q | Gain of function | Missense_Mutation |
| 60 | BRAF | NM_004333 | 474 | c.1798_1799GT>AG | p.V600R | Gain of function | Missense_Mutation |
| 61 | BRAF | NM_004333 | 6267 | c.1808_1810delGAT | p.W604del | Gain of function | In_Frame_Del |
| 62 | BRAF | NM_004333 | 1134 | c.1810T>G | p.W604G | Gain of function | Missense_Mutation |
| 63 | BRAF | NM_004333 | 453 | c.1397G>A | p.G466E | Gain of function | Missense_Mutation |
| 64 | BRAF | NM_004333 | 1112 | c.1396G>C | p.G466R | Gain of function | Missense_Mutation |
| 65 | BRAF | NM_004333 | 451 | c.1397G>T | p.G466V | Gain of function | Missense_Mutation |
| 66 | BRAF | NM_004333 | 460 | c.1406G>C | p.G469L | Gain of function | Missense_Mutation |
| 67 | BRAF | NM_004333 | 460 | c.1406G>C | p.G469A | Gain of function | Missense_Mutation |
| 68 | BRAF | NM_004333 | 461 | c.1406G>A | p.G469E | Gain of function | Missense_Mutation |
| 69 | BRAF | NM_004333 | 457 | c.1405G>A | p.G469R | Gain of function | Missense_Mutation |
| 70 | BRAF | NM_004333 | 458 | c.1405_1406GG>TC | p.G469S | Gain of function | Missense_Mutation |
| 71 | BRAF | NM_004333 | 459 | c.1406G>T | p.G469V | Gain of function | Missense_Mutation |
| 72 | BRAF | NM_004333 | 475 | c.1799_1800TG>AA | p.V600E | Gain of function | Missense_Mutation |
| 73 | BRAF | NM_004333 | 475 | c.1799_1800TG>AA | p.V600E | Gain of function | Missense_Mutation |
| 74 | BRAF | NM_004333 | 475 | c.1799_1800TG>AA | p.V600E | Gain of function | Missense_Mutation |
| 75 | BRAF | NM_004333 | 476 | c.1799T>A | p.V600E | Gain of function | Missense_Mutation |
| 76 | BRAF | NM_004333 | 476 | c.1799T>A | p.V600E | Gain of function | Missense_Mutation |
| 77 | BRAF | NM_004333 | 476 | c.1799T>A | p.V600E | Gain of function | Missense_Mutation |
| 78 | CTNNB1 | NM_001904 | 5747 | c.37G>A | p.A13T | Gain of function | Missense_Mutation |
| 79 | CTNNB1 | NM_001904 | 5702 | c.59C>T | p.A20V | Gain of function | Missense_Mutation |
| 80 | CTNNB1 | NM_001904 | 5738 | c.61G>A | p.A21T | Gain of function | Missense_Mutation |
| 81 | CTNNB1 | NM_001904 | 5753 | c.116C>G | p.A39G | Gain of function | Missense_Mutation |
| 82 | CTNNB1 | NM_001904 | 5762 | c.115G>A | p.A39T | Gain of function | Missense_Mutation |
| 83 | CTNNB1 | NM_001904 | 5744 | c.127G>C | p.A43P | Gain of function | Missense_Mutation |
| 84 | CTNNB1 | NM_001904 | 5758 | c.127G>A | p.A43T | Gain of function | Missense_Mutation |
| 85 | CTNNB1 | NM_001904 | 5699 | c.128C>T | p.A43V | Gain of function | Missense_Mutation |
| 86 | CTNNB1 | NM_001904 | 5690 | c.95A>C | p.D32A | Gain of function | Missense_Mutation |
| 87 | CTNNB1 | NM_001904 | 5681 | c.95A>G | p.D32G | Gain of function | Missense_Mutation |
| 88 | CTNNB1 | NM_001904 | 5668 | c.94G>C | p.D32H | Gain of function | Missense_Mutation |
| 89 | CTNNB1 | NM_001904 | 5672 | c.94G>A | p.D32N | Gain of function | Missense_Mutation |
| 90 | CTNNB1 | NM_001904 | 5691 | c.95A>T | p.D32V | Gain of function | Missense_Mutation |
| 91 | CTNNB1 | NM_001904 | 5661 | c.94G>T | p.D32Y | Gain of function | Missense_Mutation |
| 92 | CTNNB1 | NM_001904 | 49161 | c.43G>A | p.E15K | Gain of function | Missense_Mutation |
| 93 | CTNNB1 | NM_001904 | 5671 | c.101G>A | p.G34E | Gain of function | Missense_Mutation |
| 94 | CTNNB1 | NM_001904 | 5684 | c.100G>C | p.G34R | Gain of function | Missense_Mutation |
| 95 | CTNNB1 | NM_001904 | 5686 | c.100G>A | p.G34R | Gain of function | Missense_Mutation |
| 96 | CTNNB1 | NM_001904 | 5670 | c.101G>T | p.G34V | Gain of function | Missense_Mutation |
| 97 | CTNNB1 | NM_001904 | 5713 | c.113G>A | p.G38D | Gain of function | Missense_Mutation |
| 98 | CTNNB1 | NM_001904 | 5678 | c.107A>C | p.H36P | Gain of function | Missense_Mutation |
| 99 | CTNNB1 | NM_001904 | 27378 | c.107A>G | p.H36R | Gain of function | Missense_Mutation |
| 100 | CTNNB1 | NM_001904 | 5703 | c.106C>T | p.H36Y | Gain of function | Missense_Mutation |
| 101 | CTNNB1 | NM_001904 | 5674 | c.104T>G | p.135S | Gain of function | Missense_Mutation |
| 102 | CTNNB1 | NM_001904 | 13168 | c.104T>C | p.I35T | Gain of function | Missense_Mutation |
| 103 | CTNNB1 | NM_001904 | 5721 | c.91C>T | p.L31L | Gain of function | Synonymous_Mutation |
| 104 | CTNNB1 | NM_001904 | 13175 | c.138G>A | p.L46L | Gain of function | Synonymous_Mutation |
| 105 | CTNNB1 | NM_001904 | 17661 | c.130C>G | p.P44A | Gain of function | Missense_Mutation |
| 106 | CTNNB1 | NM_001904 | 5761 | c.131C>T | p.P44L | Gain of function | Missense_Mutation |
| 107 | CTNNB1 | NM_001904 | 5704 | c.130C>T | p.P44S | Gain of function | Missense_Mutation |
| 108 | CTNNB1 | NM_001904 | 6057 | c.67_99del33 | p.S23_S33del | Gain of function | In_Frame_Del |
| 109 | CTNNB1 | NM_001904 | 17941 | c.67A>G | p.S23G | Gain of function | Missense_Mutation |
| 110 | CTNNB1 | NM_001904 | 5714 | c.67A>C | p.S23R | Gain of function | Missense_Mutation |
| 111 | CTNNB1 | NM_001904 | 5694 | c.86C>T | p.S29F | Gain of function | Missense_Mutation |
| 112 | CTNNB1 | NM_001904 | 5683 | c.97T>G | p.S33A | Gain of function | Missense_Mutation |
| 113 | CTNNB1 | NM_001904 | 5677 | c.98C>G | p.S33C | Gain of function | Missense_Mutation |
| 114 | CTNNB1 | NM_001904 | 5669 | c.98C>T | p.S33F | Gain of function | Missense_Mutation |
| 115 | CTNNB1 | NM_001904 | 6098 | c.97_98TC>CT | p.S33L | Gain of function | Missense_Mutation |
| 116 | CTNNB1 | NM_001904 | 6099 | c.97_98TC>AA | p.S33N | Gain of function | Missense_Mutation |
| 117 | CTNNB1 | NM_001904 | 5682 | c.97T>C | p.S33P | Gain of function | Missense_Mutation |
| 118 | CTNNB1 | NM_001904 | 5673 | c.98C>A | p.S33Y | Gain of function | Missense_Mutation |
| 119 | CTNNB1 | NM_001904 | 5675 | c.109T>G | p.S37A | Gain of function | Missense_Mutation |
| 120 | CTNNB1 | NM_001904 | 5679 | c.110C>G | p.S37C | Gain of function | Missense_Mutation |
| 121 | CTNNB1 | NM_001904 | 5662 | c.110C>T | p.S37F | Gain of function | Missense_Mutation |
| 122 | CTNNB1 | NM_001904 | 5687 | c.109T>C | p.S37P | Gain of function | Missense_Mutation |
| 123 | CTNNB1 | NM_001904 | 5666 | c.110C>A | p.S37Y | Gain of function | Missense_Mutation |
| 124 | CTNNB1 | NM_001904 | 5685 | c.133T>G | p.S45A | Gain of function | Missense_Mutation |
| 125 | CTNNB1 | NM_001904 | 5689 | c.134C>G | p.S45C | Gain of function | Missense_Mutation |
| 126 | CTNNB1 | NM_001904 | 5667 | c.134C>T | p.S45F | Gain of function | Missense_Mutation |
| 127 | CTNNB1 | NM_001904 | 5663 | c.133T>C | p.S45P | Gain of function | Missense_Mutation |
| 128 | CTNNB1 | NM_001904 | 5692 | c.134C>A | p.S45Y | Gain of function | Missense_Mutation |
| 129 | CTNNB1 | NM_001904 | 5708 | c.119C>T | p.T40I | Gain of function | Missense_Mutation |
| 130 | CTNNB1 | NM_001904 | 6140 | c.120T>C | p.T40T | Gain of function | Synonymous_Mutation |
| 131 | CTNNB1 | NM_001904 | 5664 | c.121A>G | p.T41A | Gain of function | Missense_Mutation |
| 132 | CTNNB1 | NM_001904 | 5676 | c.122C>T | p.T41I | Gain of function | Missense_Mutation |
| 133 | CTNNB1 | NM_001904 | 5730 | c.122C>A | p.T41N | Gain of function | Missense_Mutation |
| 134 | CTNNB1 | NM_001904 | 5688 | c.121A>C | p.T41P | Gain of function | Missense_Mutation |
| 135 | CTNNB1 | NM_001904 | 5701 | c.122C>G | p.T41S | Gain of function | Missense_Mutation |
| 136 | CTNNB1 | NM_001904 | 5716 | c.121A>T | p.T41S | Gain of function | Missense_Mutation |
| 137 | CTNNB1 | NM_001904 | 5717 | c.123C>T | p.T41T | Gain of function | Synonymous_Mutation |
| 138 | CTNNB1 | NM_001904 | 29289 | c.125_126delCA | p.T42fs*7 | Gain of function | Frame_Shift_Del |
| 139 | CTNNB1 | NM_001904 | 5696 | c.125C>T | p.T42I | Gain of function | Missense_Mutation |
| 140 | CTNNB1 | NM_001904 | 5732 | c.125C>G | p.T42R | Gain of function | Missense_Mutation |
| 141 | CTNNB1 | NM_001904 | 6050 | c.64_114del51 | p.V22_G38del | Gain of function | In_Frame_Del |
| 142 | CTNNB1 | NM_001904 | 6052 | c.64_99del36 | p.V22_S33del | Gain of function | In_Frame_Del |
| 143 | CTNNB1 | NM_001904 | 5706 | c.65T>C | p.V22A | Gain of function | Missense_Mutation |
| 144 | CTNNB1 | NM_001904 | 22566 | c.64G>A | p.V22I | Gain of function | Missense_Mutation |
| 145 | CTNNB1 | NM_001904 | 6064 | c.74_97del24 | p.W25_D32del | Gain of function | In_Frame_Del |
| 146 | CTNNB1 | NM_001904 | 5749 | c.74G>T | p.W25L | Gain of function | Missense_Mutation |
| 147 | CTNNB1 | NM_001904 | 14256 | c.73_96del24 | p.WQQQSYLD25? | Gain of function | N/A |
| 148 | CTNNB1 | NM_001904 | 34125 | c.90C>G | p.Y30* | Gain of function | NonsenseMutation |
| 149 | CTNNB1 | NM_001904 | 6076 | c.88_99del12 | p.Y30_S33del | Gain of function | In_Frame_Del |
| 150 | DDR2_ENS | ENST00000367922 | 173712 | c.390C>T | p.I130I | Unclassified | Synonymous_Mutation |
| 151 | DDR2_ENS | ENST00000367922 | 94126 | c.1783C>G | p.L595V | Unclassified | Missense_Mutation |
| 152 | DDR2 | NM_006182 | 48314 | c.1367A>G | p.N456S | Unclassified | Missense_Mutation |
| 153 | DDR2_ENS | ENST00000367922 | 140388 | c.691C>A | p.Q231K | Unclassified | Missense_Mutation |
| 154 | DDR2 | NM_006182 | 12821 | c.313C>A | p.R105S | Unclassified | Missense_Mutation |
| 155 | DDR2 | NM_006182 | 140390 | c.1598C>A | p.T533K | Unclassified | Missense_Mutation |
| 156 | DDR2_ENS | ENST00000367922 | 140389 | c.1598C>A | p.T533K | Unclassified | Missense_Mutation |
| 157 | EGFR | NM_005228 | 236670 | c.1476C>A | p.S492R | Gain of function | Missense_Mutation |
| 158 | EGFR | NM_005228 | 41905 | c.2092G>A | p.A698T | Gain of function | Missense_Mutation |
| 159 | EGFR | NM_005228 | 28508 | c.2104G>T | p.A702S | Gain of function | Missense_Mutation |
| 160 | EGFR | NM_005228 | 13427 | c.2126A>C | p.E709A | Gain of function | Missense_Mutation |
| 161 | EGFR | NM_005228 | 13009 | c.2126A>G | p.E709G | Gain of function | Missense_Mutation |
| 162 | EGFR | NM_005228 | 12988 | c.2125G>A | p.E709K | Gain of function | Missense_Mutation |
| 163 | EGFR | NM_005228 | 12371 | c.2126A>T | p.E709V | Gain of function | Missense_Mutation |
| 164 | EGFR | NM_005228 | 41603 | c.2134T>C | p.F712L | Gain of function | Missense_Mutation |
| 165 | EGFR | NM_005228 | 28601 | c.2135T>C | p.F712S | Gain of function | Missense_Mutation |
| 166 | EGFR | NM_005228 | 6239 | c.2156G>C | p.G719A | Gain of function | Missense_Mutation |
| 167 | EGFR | NM_005228 | 6239 | c.2156G>C | p.G719A | Gain of function | Missense_Mutation |
| 168 | EGFR | NM_005228 | 6239 | c.2156G>C | p.G719A | Gain of function | Missense_Mutation |
| 169 | EGFR | NM_005228 | 18441 | c.2154_2155GG>TT | p.G719C | Gain of function | Missense_Mutation |
| 170 | EGFR | NM_005228 | 18441 | c.2154_2155GG>TT | p.G719C | Gain of function | Missense_Mutation |
| 171 | EGFR | NM_005228 | 18441 | c.2154_2155GG>TT | p.G719C | Gain of function | Missense_Mutation |
| 172 | EGFR | NM_005228 | 6253 | c.2155G>T | p.G719C | Gain of function | Missense_Mutation |
| 173 | EGFR | NM_005228 | 6253 | c.2155G>T | p.G719C | Gain of function | Missense_Mutation |
| 174 | EGFR | NM_005228 | 6253 | c.2155G>T | p.G719C | Gain of function | Missense_Mutation |
| 175 | EGFR | NM_005228 | 18425 | c.2156G>A | p.G719D | Gain of function | Missense_Mutation |
| 176 | EGFR | NM_005228 | 18425 | c.2156G>A | p.G719D | Gain of function | Missense_Mutation |
| 177 | EGFR | NM_005228 | 18425 | c.2156G>A | p.G719D | Gain of function | Missense_Mutation |
| 178 | EGFR | NM_005228 | 6252 | c.2155G>A | p.G719S | Gain of function | Missense_Mutation |
| 179 | EGFR | NM_005228 | 6252 | c.2155G>A | p.G719S | Gain of function | Missense_Mutation |
| 180 | EGFR | NM_005228 | 6252 | c.2155G>A | p.G719S | Gain of function | Missense_Mutation |
| 181 | EGFR | NM_005228 | 28510 | c.2162G>C | p.G721A | Gain of function | Missense_Mutation |
| 182 | EGFR | NM_005228 | 22992 | c.2161G>A | p.G721S | Gain of function | Missense_Mutation |
| 183 | EGFR | NM_005228 | 13979 | c.2170G>A | p.G724S | Gain of function | Missense_Mutation |
| 184 | EGFR | NM_005228 | 28511 | c.2108T>C | p.L703P | Gain of function | Missense_Mutation |
| 185 | EGFR | NM_005228 | 12373 | c.2159C>T | p.S720F | Gain of function | Missense_Mutation |
| 186 | EGFR | NM_005228 | 26509 | c.2227G>A | p.A743T | Gain of function | Missense_Mutation |
| 187 | EGFR | NM_005228 | 27042 | c.2282A>G | p.D761G | Gain of function | Missense_Mutation |
| 188 | EGFR | NM_005228 | 21984 | c.2281G>T | p.D761Y | Gain of function | Missense_Mutation |
| 189 | EGFR | NM_005228 | 13184 | c.2236G>A | p.E746K | Gain of function | Missense_Mutation |
| 190 | EGFR | NM_005228 | 13182 | c.2203G>A | p.G735S | Gain of function | Missense_Mutation |
| 191 | EGFR | NM_005228 | 85993 | c.2260A>G | p.K754E | Gain of function | Missense_Mutation |
| 192 | EGFR | NM_005228 | 24267 | c.2239_2240TT>CC | p.L747P | Gain of function | Missense_Mutation |
| 193 | EGFR | NM_005228 | 26704 | c.2240T>C | p.L747S | Gain of function | Missense_Mutation |
| 194 | EGFR | NM_005228 | 13181 | c.2198C>T | p.P733L | Gain of function | Missense_Mutation |
| 195 | EGFR | NM_005228 | 53194 | c.2197C>T | p.P733S | Gain of function | Missense_Mutation |
| 196 | EGFR | NM_005228 | 17570 | c.2222C>T | p.P741L | Gain of function | Missense_Mutation |
| 197 | EGFR | NM_005228 | 6268 | c.2257C>T | p.P7535 | Gain of function | Missense_Mutation |
| 198 | EGFR | NM_005228 | 29274 | c.2254T>C | p.S752P | Gain of function | Missense_Mutation |
| 199 | EGFR | NM_005228 | 13185 | c.2252C>T | p.T751I | Gain of function | Missense_Mutation |
| 200 | EGFR | NM_005228 | 27041 | c.2213T>G | p.V738G | Gain of function | Missense_Mutation |
| 201 | EGFR | NM_005228 | 13432 | c.2193G>A | p.W731* | Gain of function | Nonsense_Mutation |
| 202 | EGFR | NM_005228 | 6223 | c.2235_2249del15 | p.E746_A750del | Gain of function | In_Frame_Del |
| 203 | EGFR | NM_005228 | 6223 | c.2235_2249del15 | p.E746_A750del | Gain of function | In_Frame_Del |
| 204 | EGFR | NM_005228 | 6223 | c.2235_2249del15 | p.E746_A750del | Gain of function | In_Frame_Del |
| 205 | EGFR | NM_005228 | 6225 | c.2236_2250del15 | p.E746_A750del | Gain of function | In_Frame_Del |
| 206 | EGFR | NM_005228 | 6225 | c.2236_2250del15 | p.E746_A750del | Gain of function | In_Frame_Del |
| 207 | EGFR | NM_005228 | 6225 | c.2236_2250del15 | p.E746_A750del | Gain of function | In_Frame_Del |
| 208 | EGFR | NM_005228 | 28517 | c.2235_2246del12 | p.E746_E749del | Gain of function | In_Frame_Del |
| 209 | EGFR | NM_005228 | 28517 | c.2235_2246del12 | p.E746_E749del | Gain of function | In_Frame_Del |
| 210 | EGFR | NM_005228 | 28517 | c.2235_2246del12 | p.E746_E749del | Gain of function | In_Frame_Del |
| 211 | EGFR | NM_005228 | 12367 | c.2237_2254del18 | p.E746_S752>A | Gain of function | In_Frame_Del |
| 212 | EGFR | NM_005228 | 12367 | c.2237_2254del18 | p.E746_S752>A | Gain of function | In_Frame_Del |
| 213 | EGFR | NM_005228 | 12367 | c.2237_2254del18 | p.E746_S752>A | Gain of function | In_Frame_Del |
| 214 | EGFR | NM_005228 | 6220 | c.2238_2255del18 | p.E746_S752>D | Gain of function | In_Frame_Del |
| 215 | EGFR | NM_005228 | 6220 | c.2238_2255del18 | p.E746_S752>D | Gain of function | In_Frame_Del |
| 216 | EGFR | NM_005228 | 6220 | c.2238_2255del18 | p.E746_S752>D | Gain of function | In_Frame_Del |
| 217 | EGFR | NM_005228 | 12384 | c.2237_2255>T | p.E746_S752>V | Gain of function | In_Frame_Del |
| 218 | EGFR | NM_005228 | 12384 | c.2237_2255>T | p.E746_S752>V | Gain of function | In_Frame_Del |
| 219 | EGFR | NM_005228 | 12384 | c.2237_2255>T | p.E746_S752>V | Gain of function | In_Frame_Del |
| 220 | EGFR | NM_005228 | 133189 | c.2236_2256del21 | p.E746_S752del | Gain of function | In_Frame_Del |
| 221 | EGFR | NM_005228 | 133189 | c.2236_2256del21 | p.E746_S752del | Gain of function | In_Frame_Del |
| 222 | EGFR | NM_005228 | 133189 | c.2236_2256del21 | p.E746_S752del | Gain of function | In_Frame_Del |
| 223 | EGFR | NM_005228 | 12678 | c.2237_2251del15 | p.E746_T751>A | Gain of function | In_Frame_Del |
| 224 | EGFR | NM_005228 | 12678 | c.2237_2251del15 | p.E746_T751>A | Gain of function | In_Frame_Del |
| 225 | EGFR | NM_005228 | 12678 | c.2237_2251del15 | p.E746_T751>A | Gain of function | In_Frame_Del |
| 226 | EGFR | NM_005228 | 12386 | c.2237_2252>T | p.E746_T751>V | Gain of function | In_Frame_Del |
| 227 | EGFR | NM_005228 | 12386 | c.2237_2252>T | p.E746_T751>V | Gain of function | In_Frame_Del |
| 228 | EGFR | NM_005228 | 12386 | c.2237_2252>T | p.E746_T751>V | Gain of function | In_Frame_Del |
| 229 | EGFR | NM_005228 | 12728 | c.2236_2253del18 | p.E746_T751del | Gain of function | In_Frame_Del |
| 230 | EGFR | NM_005228 | 12728 | c.2236_2253del18 | p.E746_T751del | Gain of function | In_Frame_Del |
| 231 | EGFR | NM_005228 | 12728 | c.2236_2253del18 | p.E746_T751del | Gain of function | In_Frame_Del |
| 232 | EGFR | NM_005228 | 26038 | c.2233_2247del15 | p.K745_E749del | Gain of function | In_Frame_Del |
| 233 | EGFR | NM_005228 | 26038 | c.2233_2247del15 | p.K745_E749del | Gain of function | In_Frame_Del |
| 234 | EGFR | NM_005228 | 26038 | c.2233_2247del15 | p.K745_E749del | Gain of function | In_Frame_Del |
| 235 | EGFR | NM_005228 | 12382 | c.2239_2248TTAAGAGAAG>C | p.L747_A750>P | Gain of function | In_Frame_Del |
| 236 | EGFR | NM_005228 | 12382 | c.2239_2248TTAAGAGAAG>C | p.L747_A750>P | Gain of function | In_Frame_Del |
| 237 | EGFR | NM_005228 | 12382 | c.2239_2248TTAAGAGAAG>C | p.L747_A750>P | Gain of function | In_Frame_Del |
| 238 | EGFR | NM_005228 | 6218 | c.2239_2247del9 | p.L747_E749del | Gain of function | In_Frame_Del |
| 239 | EGFR | NM_005228 | 6218 | c.2239_2247del9 | p.L747_E749del | Gain of function | In_Frame_Del |
| 240 | EGFR | NM_005228 | 6218 | c.2239_2247del9 | p.L747_E749del | Gain of function | In_Frame_Del |
| 241 | EGFR | NM_005228 | 12370 | c.2240_2257del18 | p.L747_P753>S | Gain of function | In_Frame_Del |
| 242 | EGFR | NM_005228 | 12370 | c.2240_2257del18 | p.L747_P753>S | Gain of function | In_Frame_Del |
| 243 | EGFR | NM_005228 | 12370 | c.2240_2257del18 | p.L747_P753>S | Gain of function | In_Frame_Del |
| 244 | EGFR | NM_005228 | 133197 | c.2239_2257>T | p.L747_P753>S | Gain of function | In_Frame_Del |
| 245 | EGFR | NM_005228 | 133197 | c.2239_2257>T | p.L747_P753>S | Gain of function | In_Frame_Del |
| 246 | EGFR | NM_005228 | 133197 | c.2239_2257>T | p.L747_P753>S | Gain of function | In_Frame_Del |
| 247 | EGFR | NM_005228 | 6255 | c.2239_2256del18 | p.L747_S752del | Gain of function | In_Frame_Del |
| 248 | EGFR | NM_005228 | 6255 | c.2239_2256del18 | p.L747_S752del | Gain of function | In_Frame_Del |
| 249 | EGFR | NM_005228 | 6255 | c.2239_2256del18 | p.L747_S752del | Gain of function | In_Frame_Del |
| 250 | EGFR | NM_005228 | 12383 | c.2239_2251>C | p.L747_T751>P | Gain of function | In_Frame_Del |
| 251 | EGFR | NM_005228 | 12383 | c.2239_2251>C | p.L747_T751>P | Gain of function | In_Frame_Del |
| 252 | EGFR | NM_005228 | 12383 | c.2239_2251>C | p.L747_T751>P | Gain of function | In_Frame_Del |
| 253 | EGFR | NM_005228 | 6210 | c.2240_2251del12 | p.L747_T751>S | Gain of function | In_Frame_Del |
| 254 | EGFR | NM_005228 | 6210 | c.2240_2251del12 | p.L747_T751>S | Gain of function | In_Frame_Del |
| 255 | EGFR | NM_005228 | 6210 | c.2240_2251del12 | p.L747_T751>S | Gain of function | In_Frame_Del |
| 256 | EGFR | NM_005228 | 12369 | c.2240_2254del15 | p.L747_T751del | Gain of function | In_Frame_Del |
| 257 | EGFR | NM_005228 | 12369 | c.2240_2254del15 | p.L747_T751del | Gain of function | In_Frame_Del |
| 258 | EGFR | NM_005228 | 12369 | c.2240_2254del15 | p.L747_T751del | Gain of function | In_Frame_Del |
| 259 | EGFR | NM_005228 | 23571 | c.2238_2252del15 | p.L747_T751del | Gain of function | In_Frame_Del |
| 260 | EGFR | NM_005228 | 23571 | c.2238_2252del15 | p.L747_T751del | Gain of function | In_Frame_Del |
| 261 | EGFR | NM_005228 | 23571 | c.2238_2252del15 | p.L747_T751del | Gain of function | In_Frame_Del |
| 262 | EGFR | NM_005228 | 6254 | c.2239_2253del15 | p.L747_T751del | Gain of function | In_Frame_Del |
| 263 | EGFR | NM_005228 | 6254 | c.2239_2253del15 | p.L747_T751del | Gain of function | In_Frame_Del |
| 264 | EGFR | NM_005228 | 6254 | c.2239_2253del15 | p.L747_T751del | Gain of function | In_Frame_Del |
| 265 | EGFR | NM_005228 | 13556 | c.2253_2276del24 | p.S752_I759del | Gain of function | In_Frame_Del |
| 266 | EGFR | NM_005228 | 13556 | c.2253_2276del24 | p.S752_I759del | Gain of function | In_Frame_Del |
| 267 | EGFR | NM_005228 | 13556 | c.2253_2276del24 | p.S752_I759del | Gain of function | In_Frame_Del |
| 268 | EGFR | NM_005228 | 6256 | c.2254_2277del24 | p.S752_I759del | Gain of function | In_Frame_Del |
| 269 | EGFR | NM_005228 | 6256 | c.2254_2277del24 | p.S752_I759del | Gain of function | In_Frame_Del |
| 270 | EGFR | NM_005228 | 6256 | c.2254_2277del24 | p.S752_I759del | Gain of function | In_Frame_Del |
| 271 | EGFR | NM_005228 | 96856 | c.2252_2276>A | p.T751_I759>N | Gain of function | In_Frame_Del |
| 272 | EGFR | NM_005228 | 96856 | c.2252_2276>A | p.T751_I759>N | Gain of function | In_Frame_Del |
| 273 | EGFR | NM_005228 | 96856 | c.2252_2276>A | p.T751_I759>N | Gain of function | In_Frame_Del |
| 274 | EGFR | NM_005228 | 133207 | c.2252_2275del24 | p.T751_I759del | Gain of function | In_Frame_Del |
| 275 | EGFR | NM_005228 | 133207 | c.2252_2275del24 | p.T751_I759del | Gain of function | In_Frame_Del |
| 276 | EGFR | NM_005228 | 133207 | c.2252_2275del24 | p.T751_I759del | Gain of function | In_Frame_Del |
| 277 | EGFR | NM_005228 | 26445 | c.2300C>T | p.A767V | Gain of function | Missense_Mutation |
| 278 | EGFR | NM_005228 | 22954 | c.2324G>A | p.C775Y | Gain of function | Missense_Mutation |
| 279 | EGFR | NM_005228 | 14068 | c.2308G>A | p.D770N | Gain of function | Missense_Mutation |
| 280 | EGFR | NM_005228 | 22951 | c.2384T>C | p.F795S | Gain of function | Missense_Mutation |
| 281 | EGFR | NM_005228 | 13007 | c.2335_2336GG>TT | p.G779F | Gain of function | Missense_Mutation |
| 282 | EGFR | NM_005228 | 27568 | c.2387G>C | p.G796A | Gain of function | Missense_Mutation |
| 283 | EGFR | NM_005228 | 133565 | c.2387G>A | p.G796D | Gain of function | Missense_Mutation |
| 284 | EGFR | NM_005228 | 20891 | c.2386G>A | p.G796S | Gain of function | Missense_Mutation |
| 285 | EGFR | NM_005228 | 13005 | c.2318A>T | p.H773L | Gain of function | Missense_Mutation |
| 286 | EGFR | NM_005228 | 13433 | c.2318A>G | p.H773R | Gain of function | Missense_Mutation |
| 287 | EGFR | NM_005228 | 13190 | c.2375T>C | p.L792P | Gain of function | Missense_Mutation |
| 288 | EGFR | NM_005228 | 6226 | c.2326C>T | p.R776C | Gain of function | Missense_Mutation |
| 289 | EGFR | NM_005228 | 22940 | c.2327G>A | p.R776H | Gain of function | Missense_Mutation |
| 290 | EGFR | NM_005228 | 6241 | c.2303G>T | p.S768I | Gain of function | Missense_Mutation |
| 291 | EGFR | NM_005228 | 13189 | c.2351C>T | p.S784F | Gain of function | Missense_Mutation |
| 292 | EGFR | NM_005228 | 28513 | c.2350T>C | p.S784P | Gain of function | Missense_Mutation |
| 293 | EGFR | NM_005228 | 6240 | c.2369C>T | p.T790M | Gain of function | Missense_Mutation |
| 294 | EGFR | NM_005228 | 6240 | c.2369C>T | p.T790M | Gain of function | Missense_Mutation |
| 295 | EGFR | NM_005228 | 6240 | c.2369C>T | p.T790M | Gain of function | Missense_Mutation |
| 296 | EGFR | NM_005228 | 6240 | c.2369C>T | p.T790M | Gain of function | Missense_Mutation |
| 297 | EGFR | NM_005228 | 6240 | c.2369C>T | p.T790M | Gain of function | Missense_Mutation |
| 298 | EGFR | NM_005228 | 6240 | c.2369C>T | p.T790M | Gain of function | Missense_Mutation |
| 299 | EGFR | NM_005228 | 28603 | c.2293G>A | p.V765M | Gain of function | Missense_Mutation |
| 300 | EGFR | NM_005228 | 6242 | c.2305G>T | p.V769L | Gain of function | Missense_Mutation |
| 301 | EGFR | NM_005228 | 13006 | c.2320G>A | p.V774M | Gain of function | Missense_Mutation |
| 302 | EGFR | NM_005228 | 27110 | c.2356G>A | p.V786M | Gain of function | Missense_Mutation |
| 303 | EGFR | NM_005228 | 12378 | c.2310_2311insGGT | p.D770_N771insG | Gain of function | In_Frame_Ins |
| 304 | EGFR | NM_005228 | 12378 | c.2310_2311insGGT | p.D770_N771insG | Gain of function | In_Frame_Ins |
| 305 | EGFR | NM_005228 | 12378 | c.2310_2311insGGT | p.D770_N771insG | Gain of function | In_Frame_Ins |
| 306 | EGFR | NM_005228 | 48922 | c.2311_2312insGCGTGGACA | p.D770_N771insSVD | Gain of function | In_Frame_Ins |
| 307 | EGFR | NM_005228 | 48922 | c.2311_2312insGCGTGGACA | p.D770_N771insSVD | Gain of function | In_Frame_Ins |
| 308 | EGFR | NM_005228 | 48922 | c.2311_2312insGCGTGGACA | p.D770_N771insSVD | Gain of function | In_Frame_Ins |
| 309 | EGFR | NM_005228 | 12427 | c.2308_2309insGTT | p.D770>GY | Gain of function | In_Frame_Ins |
| 310 | EGFR | NM_005228 | 12427 | c.2308_2309insGTT | p.D770>GY | Gain of function | In_Frame_Ins |
| 311 | EGFR | NM_005228 | 12427 | c.2308_2309insGTT | p.D770>GY | Gain of function | In_Frame_Ins |
| 312 | EGFR | NM_005228 | 12377 | c.2319_2320insCAC | p.H773_V774insH | Gain of function | In_Frame_Ins |
| 313 | EGFR | NM_005228 | 12377 | c.2319_2320insCAC | p.H773_V774insH | Gain of function | In_Frame_Ins |
| 314 | EGFR | NM_005228 | 12377 | c.2319_2320insCAC | p.H773_V774insH | Gain of function | In_Frame_Ins |
| 315 | EGFR | NM_005228 | 12675 | c.2575G>A | p.A859T | Gain of function | Missense_Mutation |
| 316 | EGFR | NM_005228 | 13197 | c.2590G>A | p.A864T | Gain of function | Missense_Mutation |
| 317 | EGFR | NM_005228 | 13008 | c.2612C>G | p.A871G | Gain of function | Missense_Mutation |
| 318 | EGFR | NM_005228 | 28605 | c.2611G>A | p.A871T | Gain of function | Missense_Mutation |
| 319 | EGFR | NM_005228 | 28607 | c.2603A>G | p.E868G | Gain of function | Missense_Mutation |
| 320 | EGFR | NM_005228 | 14070 | c.2588G>A | p.G863D | Gain of function | Missense_Mutation |
| 321 | EGFR | NM_005228 | 13199 | c.2618G>A | p.G873E | Gain of function | Missense_Mutation |
| 322 | EGFR | NM_005228 | 26438 | c.2620G>A | p.G874S | Gain of function | Missense_Mutation |
| 323 | EGFR | NM_005228 | 33725 | c.2609A>G | p.H870R | Gain of function | Missense_Mutation |
| 324 | EGFR | NM_005228 | 53292 | c.2608C>T | p.H870Y | Gain of function | Missense_Mutation |
| 325 | EGFR | NM_005228 | 26129 | c.2572C>T | p.L858L | Gain of function | Synonymous_Mutation |
| 326 | EGFR | NM_005228 | 12366 | c.2572C>A | p.L858M | Gain of function | Missense_Mutation |
| 327 | EGFR | NM_005228 | 12429 | c.2573_2574TG>GT | p.L858R | Gain of function | Missense_Mutation |
| 328 | EGFR | NM_005228 | 12429 | c.2573_2574TG>GT | p.L858R | Gain of function | Missense_Mutation |
| 329 | EGFR | NM_005228 | 12429 | c.2573_2574TG>GT | p.L858R | Gain of function | Missense_Mutation |
| 330 | EGFR | NM_005228 | 6224 | c.2573T>G | p.L858R | Gain of function | Missense_Mutation |
| 331 | EGFR | NM_005228 | 6224 | c.2573T>G | p.L858R | Gain of function | Missense_Mutation |
| 332 | EGFR | NM_005228 | 6224 | c.2573T>G | p.L858R | Gain of function | Missense_Mutation |
| 333 | EGFR | NM_005228 | 6213 | c.2582T>A | p.L861Q | Gain of function | Missense_Mutation |
| 334 | EGFR | NM_005228 | 6213 | c.2582T>A | p.L861Q | Gain of function | Missense_Mutation |
| 335 | EGFR | NM_005228 | 12374 | c.2582T>G | p.L861R | Gain of function | Missense_Mutation |
| 336 | EGFR | NM_005228 | 12374 | c.2582T>G | p.L861R | Gain of function | Missense_Mutation |
| 337 | ERBB2 | NM_004448 | 13170 | c.2305G>C | p.D769H | Gain of function | Missense_Mutation |
| 338 | ERBB2 | NM_004448 | 51317 | c.2301C>G | p.I767M | Gain of function | Missense_Mutation |
| 339 | ERBB2 | NM_004448 | 683 | c.2263_2264TT>CC | p.L755P | Gain of function | Missense_Mutation |
| 340 | ERBB2 | NM_004448 | 14060 | c.2264T>C | p.L755S | Gain of function | Missense_Mutation |
| 341 | ERBB2 | NM_004448 | 18609 | c.2327G>T | p.G776V | Gain of function | Missense_Mutation |
| 342 | ERBB2 | NM_004448 | 35496 | c.2330T>C | p.V777A | Gain of function | Missense_Mutation |
| 343 | ERBB2 | NM_004448 | 14062 | c.2329G>T | p.V777L | Gain of function | Missense_Mutation |
| 344 | ERBB2 | NM_004448 | 12552 | c.2326_2327insTTT | p.G776>VC | Gain of function | In_Frame_Ins |
| 345 | ERBB2 | NM_004448 | 12552 | c.2326_2327insTTT | p.G776>VC | Gain of function | In_Frame_Ins |
| 346 | ERBB2 | NM_004448 | 12553 | c.2326_2327insTGT | p.G776>VC | Gain of function | In_Frame_Ins |
| 347 | ERBB2 | NM_004448 | 12553 | c.2326_2327insTGT | p.G776>VC | Gain of function | In_Frame_Ins |
| 348 | ERBB2 | NM_004448 | 26681 | c.2333_2334insGGG | p.G778_S779insG | Gain of function | In_Frame_Ins |
| 349 | ERBB2 | NM_004448 | 26681 | c.2333_2334insGGG | p.G778_S779insG | Gain of function | In_Frame_Ins |
| 350 | ERBB2 | NM_004448 | 21985 | c.2632C>T | p.H878Y | Gain of function | Missense_Mutation |
| 351 | ERBB2 | NM_004448 | 14065 | c.2524G>A | p.V842I | Gain of function | Missense_Mutation |
| 352 | ERBB4 | NM_005235 | 95705 | c.421+1G>T | p.? | Gain of | N/A |
| 353 | ERBB4 | NM_005235 | 48364 | c.1784A>T | p.D595V | Gain of | Missense_Mutation |
| 354 | ERBB4 | NM_005235 | 131772 | c.1825G>A | p.D609N | Gain of | Missense_Mutation |
| 355 | ERBB4 | NM_005235 | 131765 | c.949G>A | p.E317K | Gain of | Missense_Mutation |
| 356 | ERBB4 | NM_005235 | 170797 | c.1748T>A | p.F583Y | Gain of | Missense_Mutation |
| 357 | ERBB4 | NM_005235 | 108015 | c.2806G>A | p.G936R | Gain of | Missense_Mutation |
| 358 | ERBB4 | NM_005235 | 160825 | c.885T>G | p.H295Q | Gain of | Missense_Mutation |
| 359 | ERBB4 | NM_005235 | 48363 | c.1853A>C | p.H618P | Gain of | Missense_Mutation |
| 360 | ERBB4 | NM_005235 | 96313 | c.1852C>T | p.H618Y | Gain of | Missense_Mutation |
| 361 | ERBB4 | NM_005235 | 131764 | c.939G>A | p.M313I | Gain of | Missense_Mutation |
| 362 | ERBB4 | NM_005235 | 48369 | c.542A>G | p.N181S | Gain of | Missense_Mutation |
| 363 | ERBB4 | NM_005235 | 138342 | c.1856C>T | p.P619L | Gain of | Missense_Mutation |
| 364 | ERBB4 | NM_005235 | 48366 | c.916C>A | p.R3065 | Gain of | Missense_Mutation |
| 365 | ERBB4 | NM_005235 | 232263 | c.1835G>A | p.R612Q | Gain of | Missense_Mutation |
| 366 | ERBB4 | NM_005235 | 12833 | c.908C>A | p.S303Y | Gain of | Missense_Mutation |
| 367 | ERBB4 | NM_005235 | 110095 | c.1022C>T | p.S341L | Gain of | Missense_Mutation |
| 368 | ERBB4 | NM_005235 | 20392 | c.419C>T | p.T140I | Gain of | Missense_Mutation |
| 369 | ERBB4 | NM_005235 | 48368 | c.731C>G | p.T244R | Gain of | Missense_Mutation |
| 370 | ERBB4 | NM_005235 | 209862 | c.803A>G | p.Y268C | Gain of | Missense_Mutation |
| 371 | ERBB4 | NM_005235 | 48367 | c.854A>G | p.Y285C | Gain of | Missense_Mutation |
| 372 | FBXW7 | NM_033632.1 | 22971 | c.832C>T | p.R278* | Loss of Function | Nonsense_Mutation |
| 373 | FBXW7 | NM_033632.1 | 22973 | c.1177C>T | p.R393* | Loss of Function | Nonsense_Mutation |
| 374 | FBXW7 | NM_033632.1 | 22932 | c.1393C>T | p.R465C | Loss of Function | Missense_Mutation |
| 375 | FBXW7 | NM_033632.1 | 22965 | c.1394G>A | p.R465H | Loss of Function | Missense_Mutation |
| 376 | FBXW7 | NM_033632.1 | 33762 | c.1394G>T | p.R465L | Loss of Function | Missense_Mutation |
| 377 | FBXW7 | NM_033632.1 | 133115 | c.1393_1394CG>TA | p.R465Y | Loss of Function | Missense_Mutation |
| 378 | FBXW7 | NM_033632.1 | 22975 | c.1513C>T | p.R505C | Loss of Function | Missense_Mutation |
| 379 | FBXW7 | NM_033632.1 | 99604 | c.1513C>G | p.R505G | Loss of Function | Missense_Mutation |
| 380 | FBXW7 | NM_033632.1 | 25812 | c.1514G>A | p.R505H | Loss of Function | Missense_Mutation |
| 381 | FBXW7 | NM_033632.1 | 23000 | c.1514G>T | p.R505L | Loss of Function | Missense_Mutation |
| 382 | FBXW7 | NM_033632.1 | 22979 | c.1745C>T | p.S582L | Loss of Function | Missense_Mutation |
| 383 | FBXW7 | NM_033632.1 | 27055 | c.1510G>A | p.V504I | Loss of Function | Missense_Mutation |
| 384 | FBXW7_EN | ENST00000281708 | 170727 | c.1393C>T | p.R465C | Loss of Function | Missense_Mutation |
| 385 | FBXW7_EN | ENST00000281708 | 117310 | c.1394G>A | p.R465H | Loss of Function | Missense_Mutation |
| 386 | FBXW7_EN | ENST00000281708 | 108572 | c.1513C>T | p.R505C | Loss of Function | Missense_Mutation |
| 387 | FBXW7_EN | ENST00000281708 | 99606 | c.1513C>G | p.R505G | Loss of Function | Missense_Mutation |
| 388 | FBXW7_EN | ENST00000534231 | 170726 | c.676C>T | p.R226C | Loss of Function | Missense_Mutation |
| 389 | FBXW7_EN | ENST00000534231 | 117309 | c.677G>A | p.R226H | Loss of Function | Missense_Mutation |
| 390 | FBXW7_EN | ENST00000534231 | 108571 | c.796C>T | p.R266C | Loss of Function | Missense_Mutation |
| 391 | FBXW7_EN | ENST00000534231 | 99605 | c.796C>G | p.R266G | Loss of Function | Missense_Mutation |
| 392 | FBXW7_N | NM_018315.2 | 170725 | c.1153C>T | p.R385C | Loss of Function | Missense_Mutation |
| 393 | FBXW7_N | NM_018315.2 | 117308 | c.1154G>A | p.R385H | Loss of Function | Missense_Mutation |
| 394 | FBXW7_N | NM_018315.2 | 74637 | c.1273C>T | p.R425C | Loss of Function | Missense_Mutation |
| 395 | FBXW7_N | NM_018315.2 | 99603 | c.1273C>G | p.R425G | Loss of Function | Missense_Mutation |
| 396 | FGFR1 | NM_000604 | 98903 | c.397G>A | p.D133N | Unclassified | Missense_Mutation |
| 397 | FGFR1 | NM_000604 | 187237 | c.448C>T | p.P150S | Unclassified | Missense_Mutation |
| 398 | FGFR1 | NM_000604 | 12834 | c.754C>A | p.P252T | Unclassified | Missense_Mutation |
| 399 | FGFR1 | NM_000604 | 601 | c.374C>T | p.S125L | Unclassified | Missense_Mutation |
| 400 | FGFR1 | NM_000604 | 48380 | c.422C>G | p.T141R | Unclassified | Missense_Mutation |
| 401 | FGFR1_EN | ENST00000425967 | 98901 | c.130G>A | p.D44N | Unclassified | Missense_Mutation |
| 402 | FGFR1_EN | ENST00000425967 | 187239 | c.181C>T | p.P61S | Unclassified | Splice_Site |
| 403 | FGFR1_EN | ENST00000447712 | 98902 | c.397G>A | p.D133N | Unclassified | Missense_Mutation |
| 404 | FGFR2 | NM_000141.2 | 36906 | c.1144T>C | p.C382R | Gain of function | Missense_Mutation |
| 405 | FGFR2 | NM_000141.2 | 36901 | c.929A>G | p.K310R | Gain of function | Missense_Mutation |
| 406 | FGFR2 | NM_000141.2 | 36902 | c.1647T>G | p.N549K | Gain of function | Missense_Mutation |
| 407 | FGFR2 | NM_000141.2 | 36912 | c.1647T>A | p.N549K | Gain of function | Missense_Mutation |
| 408 | FGFR2 | NM_000141.2 | 49170 | c.758C>G | p.P253R | Gain of function | Missense_Mutation |
| 409 | FGFR2 | NM_000141.2 | 36903 | c.755C>G | p.S252W | Gain of function | Missense_Mutation |
| 410 | FGFR2 | NM_000141.2 | 36904 | c.1124A>G | p.Y375C | Gain of function | Missense_Mutation |
| 411 | FGFR3 | NM_000142 | 722 | c.1107G>T | p.A369A | Gain of function | Synonymous_Mutation |
| 412 | FGFR3 | NM_000142 | 721 | c.1172C>A | p.A391E | Gain of function | Missense_Mutation |
| 413 | FGFR3 | NM_000142 | 29438 | c.1921G>A | p.D641N | Gain of function | Missense_Mutation |
| 414 | FGFR3 | NM_000142 | 27139 | c.2349_2350delAG | p.D785fs*23 | Gain of function | Frame_Shift_Ins |
| 415 | FGFR3 | NM_000142 | 724 | c.1150T>C | p.F384L | Gain of function | Missense_Mutation |
| 416 | FGFR3 | NM_000142 | 716 | c.1108G>T | p.G370C | Gain of function | Missense_Mutation |
| 417 | FGFR3 | NM_000142 | 24842 | c.1138G>A | p.G380R | Gain of function | Missense_Mutation |
| 418 | FGFR3 | NM_000142 | 24802 | c.2089G>T | p.G697C | Gain of function | Missense_Mutation |
| 419 | FGFR3 | NM_000142 | 29446 | c.753C>T | p.H251H | Gain of function | Synonymous_Mutation |
| 420 | FGFR3 | NM_000142 | 719 | c.1948A>G | p.K650E | Gain of function | Missense_Mutation |
| 421 | FGFR3 | NM_000142 | 720 | c.1949A>T | p.K650M | Gain of function | Missense_Mutation |
| 422 | FGFR3 | NM_000142 | 726 | c.1948A>C | p.K650Q | Gain of function | Missense_Mutation |
| 423 | FGFR3 | NM_000142 | 731 | c.1949A>C | p.K650T | Gain of function | Missense_Mutation |
| 424 | FGFR3 | NM_000142 | 729 | c.2381_2381T>GA | p.L794fs*23 | Gain of function | Frame_Shift_Ins |
| 425 | FGFR3 | NM_000142 | 725 | c.2382_2421+2del42 | p.P795fs*139 | Gain of function | Frame_Shift_Ins |
| 426 | FGFR3 | NM_000142 | 714 | c.742C>T | p.R248C | Gain of function | Missense_Mutation |
| 427 | FGFR3 | NM_000142 | 715 | c.746C>G | p.S249C | Gain of function | Missense_Mutation |
| 428 | FGFR3 | NM_000142 | 17461 | c.1111A>T | p.S371C | Gain of function | Missense_Mutation |
| 429 | FGFR3 | NM_000142 | 718 | c.1118A>G | p.Y373C | Gain of function | Missense_Mutation |
| 430 | KRAS | NM_004985 | 87301 | c.33_34insGGAGCT | p.A11_612insGA | Gain of function | In_Frame_Ins |
| 431 | KRAS | NM_004985 | 510 | c.31G>C | p.A11P | Gain of function | Missense_Mutation |
| 432 | KRAS | NM_004985 | 511 | c.32C>T | p.A11V | Gain of function | Missense_Mutation |
| 433 | KRAS | NM_004985 | 19905 | c.436G>C | p.A146P | Gain of function | Missense_Mutation |
| 434 | KRAS | NM_004985 | 19404 | c.436G>A | p.A146T | Gain of function | Missense_Mutation |
| 435 | KRAS | NM_004985 | 19900 | c.437C>T | p.A146V | Gain of function | Missense_Mutation |
| 436 | KRAS | NM_004985 | 542 | c.53C>A | p.A18D | Gain of function | Missense_Mutation |
| 437 | KRAS | NM_004985 | 547 | c.176C>A | p.A59E | Gain of function | Missense_Mutation |
| 438 | KRAS | NM_004985 | 28518 | c.176C>G | p.A59G | Gain of function | Missense_Mutation |
| 439 | KRAS | NM_004985 | 546 | c.175G>A | p.A59T | Gain of function | Missense_Mutation |
| 440 | KRAS | NM_004985 | 12654 | c.30_31insGGA | p.G10_A11insG | Gain of function | In_Frame_Ins |
| 441 | KRAS | NM_004985 | 12655 | c.36_37insGGT | p.G12_613insG | Gain of function | In_Frame_Ins |
| 442 | KRAS | NM_004985 | 522 | c.35G>C | p.G12A | Gain of function | Missense_Mutation |
| 443 | KRAS | NM_004985 | 522 | c.35G>C | p.G12A | Gain of function | Missense_Mutation |
| 444 | KRAS | NM_004985 | 522 | c.35G>C | p.G12A | Gain of function | Missense_Mutation |
| 445 | KRAS | NM_004985 | 516 | c.34G>T | p.G12C | Gain of function | Missense_Mutation |
| 446 | KRAS | NM_004985 | 516 | c.34G>T | p.G12C | Gain of function | Missense_Mutation |
| 447 | KRAS | NM_004985 | 516 | c.34G>T | p.G12C | Gain of function | Missense_Mutation |
| 448 | KRAS | NM_004985 | 14209 | c.35_36GT>AC | p.G12D | Gain of function | Missense_Mutation |
| 449 | KRAS | NM_004985 | 14209 | c.35_36GT>AC | p.G12D | Gain of function | Missense_Mutation |
| 450 | KRAS | NM_004985 | 14209 | c.35_36GT>AC | p.G12D | Gain of function | Missense_Mutation |
| 451 | KRAS | NM_004985 | 521 | c.35G>A | p.G12D | Gain of function | Missense_Mutation |
| 452 | KRAS | NM_004985 | 521 | c.35G>A | p.G12D | Gain of function | Missense_Mutation |
| 453 | KRAS | NM_004985 | 521 | c.35G>A | p.G12D | Gain of function | Missense_Mutation |
| 454 | KRAS | NM_004985 | 519 | c.35_36GT>AA | p.G12E | Gain of function | Missense_Mutation |
| 455 | KRAS | NM_004985 | 519 | c.35_36GT>AA | p.G12E | Gain of function | Missense_Mutation |
| 456 | KRAS | NM_004985 | 519 | c.35_36GT>AA | p.G12E | Gain of function | Missense_Mutation |
| 457 | KRAS | NM_004985 | 512 | c.34_35GG>TT | p.G12F | Gain of function | Missense_Mutation |
| 458 | KRAS | NM_004985 | 512 | c.34_35GG>TT | p.G12F | Gain of function | Missense_Mutation |
| 459 | KRAS | NM_004985 | 512 | c.34_35GG>TT | p.G12F | Gain of function | Missense_Mutation |
| 460 | KRAS | NM_004985 | 523 | c.36T>C | p.G12G | Gain of function | Synonymous_Mutation |
| 461 | KRAS | NM_004985 | 523 | c.36T>C | p.G12G | Gain of function | Synonymous_Mutation |
| 462 | KRAS | NM_004985 | 523 | c.36T>C | p.G12G | Gain of function | Synonymous_Mutation |
| 463 | KRAS | NM_004985 | 524 | c.36T>A | p.G12G | Gain of function | Synonymous_Mutation |
| 464 | KRAS | NM_004985 | 524 | c.36T>A | p.G12G | Gain of function | Synonymous_Mutation |
| 465 | KRAS | NM_004985 | 524 | c.36T>A | p.G12G | Gain of function | Synonymous_Mutation |
| 466 | KRAS | NM_004985 | 34144 | c.34_35GG>AT | p.G12I | Gain of function | Missense_Mutation |
| 467 | KRAS | NM_004985 | 34144 | c.34_35GG>AT | p.G12I | Gain of function | Missense_Mutation |
| 468 | KRAS | NM_004985 | 34144 | c.34_35GG>AT | p.G12I | Gain of function | Missense_Mutation |
| 469 | KRAS | NM_004985 | 514 | c.34_35GG>CT | p.G12L | Gain of function | Missense_Mutation |
| 470 | KRAS | NM_004985 | 514 | c.34_35GG>CT | p.G12L | Gain of function | Missense_Mutation |
| 471 | KRAS | NM_004985 | 514 | c.34_35GG>CT | p.G12L | Gain of function | Missense_Mutation |
| 472 | KRAS | NM_004985 | 518 | c.34G>C | p.G12R | Gain of function | Missense_Mutation |
| 473 | KRAS | NM_004985 | 518 | c.34G>C | p.G12R | Gain of function | Missense_Mutation |
| 474 | KRAS | NM_004985 | 518 | c.34G>C | p.G12R | Gain of function | Missense_Mutation |
| 475 | KRAS | NM_004985 | 517 | c.34G>A | p.G12S | Gain of function | Missense_Mutation |
| 476 | KRAS | NM_004985 | 517 | c.34G>A | p.G12S | Gain of function | Missense_Mutation |
| 477 | KRAS | NM_004985 | 517 | c.34G>A | p.G12S | Gain of function | Missense_Mutation |
| 478 | KRAS | NM_004985 | 515 | c.35_36GT>TC | p.G12V | Gain of function | Missense_Mutation |
| 479 | KRAS | NM_004985 | 515 | c.35_36GT>TC | p.G12V | Gain of function | Missense_Mutation |
| 480 | KRAS | NM_004985 | 515 | c.35_36GT>TC | p.G12V | Gain of function | Missense_Mutation |
| 481 | KRAS | NM_004985 | 520 | c.35G>T | p.G12V | Gain of function | Missense_Mutation |
| 482 | KRAS | NM_004985 | 520 | c.35G>T | p.G12V | Gain of function | Missense_Mutation |
| 483 | KRAS | NM_004985 | 520 | c.35G>T | p.G12V | Gain of function | Missense_Mutation |
| 484 | KRAS | NM_004985 | 25081 | c.34_35GG>TA | p.G12Y | Gain of function | Missense_Mutation |
| 485 | KRAS | NM_004985 | 25081 | c.34_35GG>TA | p.G12Y | Gain of function | Missense_Mutation |
| 486 | KRAS | NM_004985 | 25081 | c.34_35GG>TA | p.G12Y | Gain of function | Missense_Mutation |
| 487 | KRAS | NM_004985 | 219781 | c.39_40insGGC | p.G13_V14insG | Gain of function | In_Frame_Ins |
| 488 | KRAS | NM_004985 | 533 | c.38G>C | p.G13A | Gain of function | Missense_Mutation |
| 489 | KRAS | NM_004985 | 533 | c.38G>C | p.G13A | Gain of function | Missense_Mutation |
| 490 | KRAS | NM_004985 | 533 | c.38G>C | p.G13A | Gain of function | Missense_Mutation |
| 491 | KRAS | NM_004985 | 527 | c.37G>T | p.G13C | Gain of function | Missense_Mutation |
| 492 | KRAS | NM_004985 | 527 | c.37G>T | p.G13C | Gain of function | Missense_Mutation |
| 493 | KRAS | NM_004985 | 527 | c.37G>T | p.G13C | Gain of function | Missense_Mutation |
| 494 | KRAS | NM_004985 | 531 | c.38_39GC>AT | p.G13D | Gain of function | Missense_Mutation |
| 495 | KRAS | NM_004985 | 531 | c.38_39GC>AT | p.G13D | Gain of function | Missense_Mutation |
| 496 | KRAS | NM_004985 | 531 | c.38_39GC>AT | p.G13D | Gain of function | Missense_Mutation |
| 497 | KRAS | NM_004985 | 532 | c.38G>A | p.G13D | Gain of function | Missense_Mutation |
| 498 | KRAS | NM_004985 | 532 | c.38G>A | p.G13D | Gain of function | Missense_Mutation |
| 499 | KRAS | NM_004985 | 532 | c.38G>A | p.G13D | Gain of function | Missense_Mutation |
| 500 | KRAS | NM_004985 | 87280 | c.38_39GC>AA | p.G13E | Gain of function | Missense_Mutation |
| 501 | KRAS | NM_004985 | 87280 | c.38_39GC>AA | p.G13E | Gain of function | Missense_Mutation |
| 502 | KRAS | NM_004985 | 87280 | c.38_39GC>AA | p.G13E | Gain of function | Missense_Mutation |
| 503 | KRAS | NM_004985 | 535 | c.39C>G | p.G13G | Gain of function | Synonymous_Mutation |
| 504 | KRAS | NM_004985 | 536 | c.39C>T | p.G13G | Gain of function | Synonymous_Mutation |
| 505 | KRAS | NM_004985 | 537 | c.39C>A | p.G13G | Gain of function | Synonymous_Mutation |
| 506 | KRAS | NM_004985 | 529 | c.37G>C | p.G13R | Gain of function | Missense_Mutation |
| 507 | KRAS | NM_004985 | 529 | c.37G>C | p.G13R | Gain of function | Missense_Mutation |
| 508 | KRAS | NM_004985 | 529 | c.37G>C | p.G13R | Gain of function | Missense_Mutation |
| 509 | KRAS | NM_004985 | 528 | c.37G>A | p.G13S | Gain of function | Missense_Mutation |
| 510 | KRAS | NM_004985 | 528 | c.37G>A | p.G13S | Gain of function | Missense_Mutation |
| 511 | KRAS | NM_004985 | 528 | c.37G>A | p.G13S | Gain of function | Missense_Mutation |
| 512 | KRAS | NM_004985 | 12721 | c.38_39GC>TT | p.G13V | Gain of function | Missense_Mutation |
| 513 | KRAS | NM_004985 | 12721 | c.38_39GC>TT | p.G13V | Gain of function | Missense_Mutation |
| 514 | KRAS | NM_004985 | 12721 | c.38_39GC>TT | p.G13V | Gain of function | Missense_Mutation |
| 515 | KRAS | NM_004985 | 534 | c.38G>T | p.G13V | Gain of function | Missense_Mutation |
| 516 | KRAS | NM_004985 | 534 | c.38G>T | p.G13V | Gain of function | Missense_Mutation |
| 517 | KRAS | NM_004985 | 534 | c.38G>T | p.G13V | Gain of function | Missense_Mutation |
| 518 | KRAS | NM_004985 | 538 | c.43G>A | p.G15S | Gain of function | Missense_Mutation |
| 519 | KRAS | NM_004985 | 19940 | c.351A>C | p.K117N | Gain of function | Missense_Mutation |
| 520 | KRAS | NM_004985 | 28519 | c.351A>T | p.K117N | Gain of function | Missense_Mutation |
| 521 | KRAS | NM_004985 | 12703 | c.57G>C | p.L19F | Gain of function | Missense_Mutation |
| 522 | KRAS | NM_004985 | 20818 | c.57G>T | p.L19F | Gain of function | Missense_Mutation |
| 523 | KRAS | NM_004985 | 543 | c.64C>A | p.Q22K | Gain of function | Missense_Mutation |
| 524 | KRAS | NM_004985 | 550 | c.181C>G | p.Q61E | Gain of function | Missense_Mutation |
| 525 | KRAS | NM_004985 | 550 | c.181C>G | p.Q61E | Gain of function | Missense_Mutation |
| 526 | KRAS | NM_004985 | 550 | c.181C>G | p.Q61E | Gain of function | Missense_Mutation |
| 527 | KRAS | NM_004985 | 554 | c.183A>C | p.Q61H | Gain of function | Missense_Mutation |
| 528 | KRAS | NM_004985 | 554 | c.183A>C | p.Q61H | Gain of function | Missense_Mutation |
| 529 | KRAS | NM_004985 | 554 | c.183A>C | p.Q61H | Gain of function | Missense_Mutation |
| 530 | KRAS | NM_004985 | 555 | c.183A>T | p.Q61H | Gain of function | Missense_Mutation |
| 531 | KRAS | NM_004985 | 555 | c.183A>T | p.Q61H | Gain of function | Missense_Mutation |
| 532 | KRAS | NM_004985 | 555 | c.183A>T | p.Q61H | Gain of function | Missense_Mutation |
| 533 | KRAS | NM_004985 | 549 | c.181C>A | p.Q61K | Gain of function | Missense_Mutation |
| 534 | KRAS | NM_004985 | 549 | c.181C>A | p.Q61K | Gain of function | Missense_Mutation |
| 535 | KRAS | NM_004985 | 549 | c.181C>A | p.Q61K | Gain of function | Missense_Mutation |
| 536 | KRAS | NM_004985 | 87298 | c.180_181TC>AA | p.Q61K | Gain of function | Missense_Mutation |
| 537 | KRAS | NM_004985 | 87298 | c.180_181TC>AA | p.Q61K | Gain of function | Missense_Mutation |
| 538 | KRAS | NM_004985 | 87298 | c.180_181TC>AA | p.Q61K | Gain of function | Missense_Mutation |
| 539 | KRAS | NM_004985 | 553 | c.182A>T | p.Q61L | Gain of function | Missense_Mutation |
| 540 | KRAS | NM_004985 | 553 | c.182A>T | p.Q61L | Gain of function | Missense_Mutation |
| 541 | KRAS | NM_004985 | 553 | c.182A>T | p.Q61L | Gain of function | Missense_Mutation |
| 542 | KRAS | NM_004985 | 551 | c.182A>C | p.Q61P | Gain of function | Missense_Mutation |
| 543 | KRAS | NM_004985 | 551 | c.182A>C | p.Q61P | Gain of function | Missense_Mutation |
| 544 | KRAS | NM_004985 | 551 | c.182A>C | p.Q61P | Gain of function | Missense_Mutation |
| 545 | KRAS | NM_004985 | 552 | c.182A>G | p.Q61R | Gain of function | Missense_Mutation |
| 546 | KRAS | NM_004985 | 552 | c.182A>G | p.Q61R | Gain of function | Missense_Mutation |
| 547 | KRAS | NM_004985 | 552 | c.182A>G | p.Q61R | Gain of function | Missense_Mutation |
| 548 | KRAS | NM_004985 | 87288 | c.173C>T | p.T58I | Gain of function | Missense_Mutation |
| 549 | KRAS | NM_004985 | 12722 | c.40G>A | p.V14I | Gain of function | Missense_Mutation |
| 550 | KRAS | NM_004985 | 507 | c.24A>G | p.V8V | Gain of function | Synonymous_Mutation |
| 551 | MAP2K1E | ENST00000307102 | 236154 | c.171G>T | p.K57N | Gain of function | Missense_Mutation |
| 552 | MET | NM_000245 | 201908 | c.3350A>G | p.D1117G | Gain of function | Missense_Mutation |
| 553 | MET | NM_000245 | 706 | c.504G>T | p.E168D | Gain of function | Missense_Mutation |
| 554 | MET | NM_000245 | 698 | c.3335A>T | p.H1112L | Gain of function | Missense_Mutation |
| 555 | MET | NM_000245 | 703 | c.3335A>G | p.H1112R | Gain of function | Missense_Mutation |
| 556 | MET | NM_000245 | 696 | c.3334C>T | p.H1112Y | Gain of function | Missense_Mutation |
| 557 | MET | NM_000245 | 697 | c.3370C>G | p.H1124D | Gain of function | Missense_Mutation |
| 558 | MET | NM_000245 | 701 | c.3390G>A | p.L1130L | Gain of function | Synonymous_Mutation |
| 559 | MET | NM_000245 | 691 | c.3803T>C | p.M1268T | Gain of function | Missense_Mutation |
| 560 | MET | NM_000245 | 702 | c.3352A>T | p.N1118Y | Gain of function | Missense_Mutation |
| 561 | MET | NM_000245 | 710 | c.1124A>G | p.N375S | Gain of function | Missense_Mutation |
| 562 | MET | NM_000245 | 707 | c.3029C>T | p.T1010I | Gain of function | Missense_Mutation |
| 563 | MET | NM_000245 | 699 | c.3743A>G | p.Y1248C | Gain of function | Missense_Mutation |
| 564 | MET | NM_000245 | 700 | c.3757T>G | p.Y1253D | Gain of function | Missense_Mutation |
| 565 | NOTCH1 | NM_017617.2 | 13050 | c.4778T>C | p.F1593S | Loss of Function | Missense_Mutation |
| 566 | NOTCH1 | NM_017617.2 | 12772 | c.4724T>C | p.L1575P | Loss of Function | Missense_Mutation |
| 567 | NOTCH1 | NM_017617.2 | 24673 | c.4724T>C | p.L1575P | Loss of Function | Missense_Mutation |
| 568 | NOTCH1 | NM_017617.2 | 13046 | c.4757T>C | p.L1586P | Loss of Function | Missense_Mutation |
| 569 | NOTCH1 | NM_017617.2 | 25839 | c.4757T>G | p.L1586R | Loss of Function | Missense_Mutation |
| 570 | NOTCH1 | NM_017617.2 | 13042 | c.4781T>C | p.L1594P | Loss of Function | Missense_Mutation |
| 571 | NOTCH1 | NM_017617.2 | 24888 | c.4790T>A | p.L1597H | Loss of Function | Missense_Mutation |
| 572 | NOTCH1 | NM_017617.2 | 12771 | c.4802T>C | p.L1601P | Loss of Function | Missense_Mutation |
| 573 | NOTCH1 | NM_017617.2 | 28524 | c.4802T>A | p.L1601Q | Loss of Function | Missense_Mutation |
| 574 | NOTCH1 | NM_017617.2 | 13053 | c.4796G>C | p.R1599P | Loss of Function | Missense_Mutation |
| 575 | NOTCH1 | NM_017617.2 | 25836 | c.4730T>A | p.V1577E | Loss of Function | Missense_Mutation |
| 576 | NOTCH1 | NM_017617.2 | 13047 | c.4735_4737delGTG | p.V1579del | Loss of Function | In_Frame_Del |
| 577 | NOTCH1 | NM_017617.2 | 13040 | c.5030T>A | p.V1677D | Loss of Function | Missense_Mutation |
| 578 | NRAS | NM_002524 | 558 | c.31G>A | p.A11T | Gain of function | Missense_Mutation |
| 579 | NRAS | NM_002524 | 577 | c.52G>A | p.A18T | Gain of function | Missense_Mutation |
| 580 | NRAS | NM_002524 | 565 | c.35G>C | p.G12A | Gain of function | Missense_Mutation |
| 581 | NRAS | NM_002524 | 562 | c.34G>T | p.G12C | Gain of function | Missense_Mutation |
| 582 | NRAS | NM_002524 | 564 | c.35G>A | p.G12D | Gain of function | Missense_Mutation |
| 583 | NRAS | NM_002524 | 567 | c.36T>C | p.G12G | Gain of function | Synonymous_Mutation |
| 584 | NRAS | NM_002524 | 12723 | c.34_35GG>AA | p.G12N | Gain of function | Missense_Mutation |
| 585 | NRAS | NM_002524 | 561 | c.34G>C | p.G12R | Gain of function | Missense_Mutation |
| 586 | NRAS | NM_002524 | 563 | c.34G>A | p.G12S | Gain of function | Missense_Mutation |
| 587 | NRAS | NM_002524 | 566 | c.35G>T | p.G12V | Gain of function | Missense_Mutation |
| 588 | NRAS | NM_002524 | 575 | c.38G>C | p.G13A | Gain of function | Missense_Mutation |
| 589 | NRAS | NM_002524 | 570 | c.37G>T | p.G13C | Gain of function | Missense_Mutation |
| 590 | NRAS | NM_002524 | 573 | c.38G>A | p.G13D | Gain of function | Missense_Mutation |
| 591 | NRAS | NM_002524 | 576 | c.39T>C | p.G13G | Gain of function | Synonymous_Mutation |
| 592 | NRAS | NM_002524 | 569 | c.37G>C | p.G13R | Gain of function | Missense_Mutation |
| 593 | NRAS | NM_002524 | 571 | c.37G>A | p.G13S | Gain of function | Missense_Mutation |
| 594 | NRAS | NM_002524 | 574 | c.38G>T | p.G13V | Gain of function | Missense_Mutation |
| 595 | NRAS | NM_002524 | 28673 | c.179G>A | p.G60E | Gain of function | Missense_Mutation |
| 596 | NRAS | NM_002524 | 581 | c.181C>G | p.Q61E | Gain of function | Missense_Mutation |
| 597 | NRAS | NM_002524 | 585 | c.183A>T | p.Q61H | Gain of function | Missense_Mutation |
| 598 | NRAS | NM_002524 | 586 | c.183A>C | p.Q61H | Gain of function | Missense_Mutation |
| 599 | NRAS | NM_002524 | 12730 | c.180_181AC>TA | p.Q61K | Gain of function | Missense_Mutation |
| 600 | NRAS | NM_002524 | 580 | c.181C>A | p.Q61K | Gain of function | Missense_Mutation |
| 601 | NRAS | NM_002524 | 12725 | c.181_182CA>TT | p.Q61L | Gain of function | Missense_Mutation |
| 602 | NRAS | NM_002524 | 30646 | c.182_183AA>TG | p.Q61L | Gain of function | Missense_Mutation |
| 603 | NRAS | NM_002524 | 583 | c.182A>T | p.Q61L | Gain of function | Missense_Mutation |
| 604 | NRAS | NM_002524 | 582 | c.182A>C | p.Q61P | Gain of function | Missense_Mutation |
| 605 | NRAS | NM_002524 | 587 | c.183A>G | p.Q61Q | Gain of function | Synonymous_Mutation |
| 606 | NRAS | NM_002524 | 33693 | c.182_183AA>GG | p.Q61R | Gain of function | Missense_Mutation |
| 607 | NRAS | NM_002524 | 579 | c.181_182CA>AG | p.Q61R | Gain of function | Missense_Mutation |
| 608 | NRAS | NM_002524 | 584 | c.182A>G | p.Q61R | Gain of function | Missense_Mutation |
| 609 | NRAS | NM_002524 | 589 | c.193A>T | p.S65C | Gain of function | Missense_Mutation |
| 610 | PIK3CA | NM_006218.1 | 17449 | c.3207A>G | p.*1069_*1069insW | Gain of function | N/A |
| 611 | PIK3CA | NM_006218.1 | 249908 | c.3207+29T>C | p.? | Gain of function | N/A |
| 612 | PIK3CA | NM_006218.1 | 28938 | c.3059C>T | p.A1020V | Gain of function | Missense_Mutation |
| 613 | PIK3CA | NM_006218.1 | 17445 | c.3104C>T | p.A1035V | Gain of function | Missense_Mutation |
| 614 | PIK3CA | NM_006218.1 | 27156 | c.3137C>A | p.A1046E | Gain of function | Missense_Mutation |
| 615 | PIK3CA | NM_006218.1 | 27273 | c.3136G>A | p.A1046T | Gain of function | Missense_Mutation |
| 616 | PIK3CA | NM_006218.1 | 36286 | c.3085G>C | p.D1029H | Gain of function | Missense_Mutation |
| 617 | PIK3CA | NM_006218.1 | 25086 | c.3133G>A | p.D1045N | Gain of function | Missense_Mutation |
| 618 | PIK3CA | NM_006218.1 | 760 | c.1624G>A | p.E542K | Gain of function | Missense_Mutation |
| 619 | PIK3CA | NM_006218.1 | 17442 | c.1624G>C | p.E542Q | Gain of function | Missense_Mutation |
| 620 | PIK3CA | NM_006218.1 | 762 | c.1625A>T | p.E542V | Gain of function | Missense_Mutation |
| 621 | PIK3CA | NM_006218.1 | 12458 | c.1634A>C | p.E545A | Gain of function | Missense_Mutation |
| 622 | PIK3CA | NM_006218.1 | 27374 | c.1635G>C | p.E545D | Gain of function | Missense_Mutation |
| 623 | PIK3CA | NM_006218.1 | 765 | c.1635G>T | p.E545D | Gain of function | Missense_Mutation |
| 624 | PIK3CA | NM_006218.1 | 764 | c.1634A>G | p.E545G | Gain of function | Missense_Mutation |
| 625 | PIK3CA | NM_006218.1 | 763 | c.1633G>A | p.E545K | Gain of function | Missense_Mutation |
| 626 | PIK3CA | NM_006218.1 | 27133 | c.1633G>C | p.E545Q | Gain of function | Missense_Mutation |
| 627 | PIK3CA | NM_006218.1 | 27155 | c.1634A>T | p.E545V | Gain of function | Missense_Mutation |
| 628 | PIK3CA | NM_006218.1 | 29110 | c.3115T>C | p.F1039L | Gain of function | Missense_Mutation |
| 629 | PIK3CA | NM_006218.1 | 27158 | c.3146G>C | p.G1049A | Gain of function | Missense_Mutation |
| 630 | PIK3CA | NM_006218.1 | 12597 | c.3145G>C | p.G1049R | Gain of function | Missense_Mutation |
| 631 | PIK3CA | NM_006218.1 | 777 | c.3145G>A | p.G1049S | Gain of function | Missense_Mutation |
| 632 | PIK3CA | NM_006218.1 | 776 | c.3140A>T | p.H1047L | Gain of function | Missense_Mutation |
| 633 | PIK3CA | NM_006218.1 | 24714 | c.3141T>G | p.H1047Q | Gain of function | Missense_Mutation |
| 634 | PIK3CA | NM_006218.1 | 775 | c.3140A>G | p.H1047R | Gain of function | Missense_Mutation |
| 635 | PIK3CA | NM_006218.1 | 774 | c.3139C>T | p.H1047Y | Gain of function | Missense_Mutation |
| 636 | PIK3CA | NM_006218.1 | 36289 | c.3143A>G | p.H1048R | Gain of function | Missense_Mutation |
| 637 | PIK3CA | NM_006218.1 | 778 | c.2102A>C | p.H701P | Gain of function | Missense_Mutation |
| 638 | PIK3CA | NM_006218.1 | 25085 | c.3120G>A | p.M1040I | Gain of function | Missense_Mutation |
| 639 | PIK3CA | NM_006218.1 | 29313 | c.3129G>A | p.M1043I | Gain of function | Missense_Mutation |
| 640 | PIK3CA | NM_006218.1 | 773 | c.3129G>T | p.M1043I | Gain of function | Missense_Mutation |
| 641 | PIK3CA | NM_006218.1 | 94984 | c.3129G>C | p.M1043I | Gain of function | Missense_Mutation |
| 642 | PIK3CA | NM_006218.1 | 12463 | c.3128T>C | p.M1043T | Gain of function | Missense_Mutation |
| 643 | PIK3CA | NM_006218.1 | 12591 | c.3127A>G | p.M1043V | Gain of function | Missense_Mutation |
| 644 | PIK3CA | NM_006218.1 | 27134 | c.3130A>G | p.N1044D | Gain of function | Missense_Mutation |
| 645 | PIK3CA | NM_006218.1 | 12592 | c.3132T>A | p.N1044K | Gain of function | Missense_Mutation |
| 646 | PIK3CA | NM_006218.1 | 759 | c.1616C>G | p.P539R | Gain of function | Missense_Mutation |
| 647 | PIK3CA | NM_006218.1 | 6147 | c.1636C>G | p.Q546E | Gain of function | Missense_Mutation |
| 648 | PIK3CA | NM_006218.1 | 24712 | c.1638G>T | p.Q546H | Gain of function | Missense_Mutation |
| 649 | PIK3CA | NM_006218.1 | 766 | c.1636C>A | p.Q546K | Gain of function | Missense_Mutation |
| 650 | PIK3CA | NM_006218.1 | 25041 | c.1637A>T | p.Q546L | Gain of function | Missense_Mutation |
| 651 | PIK3CA | NM_006218.1 | 767 | c.1637A>C | p.Q546P | Gain of function | Missense_Mutation |
| 652 | PIK3CA | NM_006218.1 | 12459 | c.1637A>G | p.Q546R | Gain of function | Missense_Mutation |
| 653 | PIK3CA | NM_006218.1 | 13594 | c.3068G>A | p.R1023Q | Gain of function | Missense_Mutation |
| 654 | PIK3CA | NM_006218.1 | 771 | c.3073A>G | p.T1025A | Gain of function | Missense_Mutation |
| 655 | PIK3CA | NM_006218.1 | 36285 | c.3074C>T | p.T1025I | Gain of function | Missense_Mutation |
| 656 | PIK3CA | NM_006218.1 | 772 | c.3074C>A | p.T1025N | Gain of function | Missense_Mutation |
| 657 | PIK3CA | NM_006218.1 | 12590 | c.3073A>T | p.T1025S | Gain of function | Missense_Mutation |
| 658 | PIK3CA | NM_006218.1 | 21451 | c.3075C>T | p.T1025T | Gain of function | Synonymous_Mutation |
| 659 | PIK3CA | NM_006218.1 | 249872 | c.1631C>A | p.T544N | Gain of function | Missense_Mutation |
| 660 | PIK3CA | NM_006218.1 | 12461 | c.3062A>G | p.Y1021C | Gain of function | Missense_Mutation |
| 661 | PIK3CA | NM_006218.1 | 17444 | c.3061T>C | p.Y1021H | Gain of function | Missense_Mutation |
| 662 | PIK3CA_EN | ENST00000263967 | 125370 | c.1633G>A | p.E545K | Gain of function | Missense_Mutation |
| 663 | PIK3CA_EN | ENST00000263967 | 94987 | c.3140A>T | p.H1047L | Gain of function | Missense_Mutation |
| 664 | PIK3CA_EN | ENST00000263967 | 94986 | c.3140A>G | p.H1047R | Gain of function | Missense_Mutation |
| 665 | PIK3CA_EN | ENST00000263967 | 94985 | c.3129G>C | p.M1043I | Gain of function | Missense_Mutation |
| 666 | PTEN | NM_000314.4 | 14087 | c.165_209del45 | p.? | Loss of Function | N/A |
| 667 | PTEN | NM_000314.4 | 19564 | c.1026+1G>T | p.? | Loss of Function | N/A |
| 668 | PTEN | NM_000314.4 | 249834 | c.635-2A>G | p.? | Loss of Function | N/A |
| 669 | PTEN | NM_000314.4 | 27365 | c.635-91G>C | p.? | Loss of Function | N/A |
| 670 | PTEN | NM_000314.4 | 28920 | c.635-1G>A | p.? | Loss of Function | N/A |
| 671 | PTEN | NM_000314.4 | 5907 | c.493-12delT | p.? | Loss of Function | N/A |
| 672 | PTEN | NM_000314.4 | 5915 | c.1-9C>G | p.? | Loss of Function | N/A |
| 673 | PTEN | NM_000314.4 | 5916 | c.209+5G>A | p.? | Loss of Function | N/A |
| 674 | PTEN | NM_000314.4 | 5932 | c.635-9A>G | p.? | Loss of Function | N/A |
| 675 | PTEN | NM_000314.4 | 5950 | c.635-12T>G | p.? | Loss of Function | N/A |
| 676 | PTEN | NM_000314.4 | 5951 | c.635-17T>G | p.? | Loss of Function | N/A |
| 677 | PTEN | NM_000314.4 | 5957 | c.1026+1G>T | | Loss of Function | N/A |
| 678 | PTEN | NM_000314.4 | 5958 | c.165-1G>T | p.? | Loss of Function | N/A |
| 679 | PTEN | NM_000314.4 | 5959 | c.165-2A>C | p.? | Loss of Function | N/A |
| 680 | PTEN | NM_000314.4 | 5960 | c.165-1G>A | p.? | Loss of Function | N/A |
| 681 | PTEN | NM_000314.4 | 5961 | c.493-1G>A | p.? | Loss of Function | N/A |
| 682 | PTEN | NM_000314.4 | 5971 | c.635-1G>T | p.? | Loss of Function | N/A |
| 683 | PTEN | NM_000314.4 | 5974 | c.209+1G>C | p.? | Loss of Function | N/A |
| 684 | PTEN | NM_000314.4 | 5975 | c.209+1delGT | p.? | Loss of Function | N/A |
| 685 | PTEN | NM_000314.4 | 5976 | c.209+1G>T | p.? | Loss of Function | N/A |
| 686 | PTEN | NM_000314.4 | 5979 | c.209+1delGTAA | p.? | Loss of Function | N/A |
| 687 | PTEN | NM_000314.4 | 4912 | c.750_751delTG | p.C250fs*2 | Loss of Function | Frame_Shift_Del |
| 688 | PTEN | NM_000314.4 | 5125 | c.755A>G | p.D252G | Loss of Function | Missense_Mutation |
| 689 | PTEN | NM_000314.4 | 5246 | c.754G>T | p.D252Y | Loss of Function | Missense_Mutation |
| 690 | PTEN | NM_000314.4 | 5814 | c.993delC | p.D331fs*13 | Loss of Function | Frame_Shift_Del |
| 691 | PTEN | NM_000314.4 | 5093 | c.992A>G | p.D331G | Loss of Function | Missense_Mutation |
| 692 | PTEN | NM_000314.4 | 5292 | c.703G>T | p.E235* | Loss of Function | Nonsense_Mutation |
| 693 | PTEN | NM_000314.4 | 88109 | c.724G>T | p.E242* | Loss of Function | Nonsense_Mutation |
| 694 | PTEN | NM_000314.4 | 26404 | c.723_724insT | p.E242fs*1 | Loss of Function | Frame_Shift_Ins |
| 695 | PTEN | NM_000314.4 | 5888 | c.723_724insTT | p.E242fs*15 | Loss of Function | Frame_Shift_Ins |
| 696 | PTEN | NM_000314.4 | 17564 | c.766G>A | p.E256K | Loss of Function | Missense_Mutation |
| 697 | PTEN | NM_000314.4 | 5314 | c.862G>T | p.E288* | Loss of Function | Nonsense_Mutation |
| 698 | PTEN | NM_000314.4 | 13452 | c.863delA | p.E288fs*3 | Loss of Function | Frame_Shift_Del |
| 699 | PTEN | NM_000314.4 | 28906 | c.871G>T | p.E291* | Loss of Function | Nonsense_Mutation |
| 700 | PTEN | NM_000314.4 | 5298 | c.19G>T | p.E7* | Loss of Function | Nonsense_Mutation |
| 701 | PTEN | NM_000314.4 | 4885 | c.1011_1014delTTCT | p.F337fs*6 | Loss of Function | Frame_Shift_Del |
| 702 | PTEN | NM_000314.4 | 5869 | c.1009delT | p.F337fs*7 | Loss of Function | Frame_Shift_Del |
| 703 | PTEN | NM_000314.4 | 5255 | c.1021T>G | p.F341V | Loss of Function | Missense_Mutation |
| 704 | PTEN | NM_000314.4 | 5257 | c.166T>G | p.F56V | Loss of Function | Missense_Mutation |
| 705 | PTEN | NM_000314.4 | 5114 | c.494G>A | p.G165E | Loss of Function | Splice_Site |
| 706 | PTEN | NM_000314.4 | 5091 | c.493G>A | p.G165R | Loss of Function | Splice_Site |
| 707 | PTEN | NM_000314.4 | 5220 | c.751G>T | p.G251C | Loss of Function | Missense_Mutation |
| 708 | PTEN | NM_000314.4 | 13981 | c.752G>A | p.G251D | Loss of Function | Missense_Mutation |
| 709 | PTEN | NM_000314.4 | 28914 | c.878delG | p.G293fs*14 | Loss of Function | Frame_Shift_Del |
| 710 | PTEN | NM_000314.4 | 5042 | c.182A>G | p.H61R | Loss of Function | Missense_Mutation |
| 711 | PTEN | NM_000314.4 | 5230 | c.758T>A | p.I253N | Loss of Function | Missense_Mutation |
| 712 | PTEN | NM_000314.4 | 5037 | c.37A>G | p.K13E | Loss of Function | Missense_Mutation |
| 713 | PTEN | NM_000314.4 | 5887 | c.711_712insAA | p.K237fs*19 | Loss of Function | Frame_Shift_Ins |
| 714 | PTEN | NM_000314.4 | 4908 | c.760_764delAAAGT | p.K254fs*42 | Loss of Function | Frame_Shift_Del |
| 715 | PTEN | NM_000314.4 | 133713 | c.780_780delA | p.K260fs*6 | Loss of Function | Frame_Shift_Del |
| 716 | PTEN | NM_000314.4 | 43075 | c.787A>T | p.K263* | Loss of Function | Nonsense_Mutation |
| 717 | PTEN | NM_000314.4 | 41768 | c.179_179delA | p.K60fs*39 | Loss of Function | Frame_Shift_Del |
| 718 | PTEN | NM_000314.4 | 5048 | c.196A>G | p.K66E | Loss of Function | Missense_Mutation |
| 719 | PTEN | NM_000314.4 | 5191 | c.198G>T | p.K66N | Loss of Function | Missense_Mutation |
| 720 | PTEN | NM_000314.4 | 4929 | c.17_18delAA | p.K6fs*4 | Loss of Function | Frame_Shift_Del |
| 721 | PTEN | NM_000314.4 | 4937 | c.16_17delAA | p.K6fs*4 | Loss of Function | Frame_Shift_Del |
| 722 | PTEN | NM_000314.4 | 39615 | c.950_953delTACT | p.L318fs*2 | Loss of Function | Frame_Shift_Del |
| 723 | PTEN | NM_000314.4 | 4894 | c.952_955delCTTA | p.L318fs*2 | Loss of Function | Frame_Shift_Del |
| 724 | PTEN | NM_000314.4 | 4903 | c.954_957delTACT | p.L318fs*2 | Loss of Function | Frame_Shift_Del |
| 725 | PTEN | NM_000314.4 | 4916 | c.953_956delTTAC | p.L318fs*2 | Loss of Function | Frame_Shift_Del |
| 726 | PTEN | NM_000314.4 | 5000 | c.170_171insT | p.L57fs*6 | Loss of Function | Frame_Shift_Ins |
| 727 | PTEN | NM_000314.4 | 5127 | c.170T>C | p.L57S | Loss of Function | Missense_Mutation |
| 728 | PTEN | NM_000314.4 | 5253 | c.170T>G | p.L57W | Loss of Function | Missense_Mutation |
| 729 | PTEN | NM_000314.4 | 23626 | c.962_963insA | p.N323fs*2 | Loss of Function | Frame_Shift_Ins |
| 730 | PTEN | NM_000314.4 | 4990 | c.968_969insA | p.N323fs*2 | Loss of Function | Frame_Shift_Ins |
| 731 | PTEN | NM_000314.4 | 5801 | c.968delA | p.N323fs*21 | Loss of Function | Frame_Shift_Del |
| 732 | PTEN | NM_000314.4 | 4932 | c.987_990delTAAA | p.N329fs*14 | Loss of Function | Frame_Shift_Del |
| 733 | PTEN | NM_000314.4 | 4942 | c.187_188delAA | p.N63fs*10 | Loss of Function | Frame_Shift_Del |
| 734 | PTEN | NM_000314.4 | 5811 | c.188delA | p.N63fs*36 | Loss of Function | Frame_Shift_Del |
| 735 | PTEN | NM_000314.4 | 17561 | c.638C>A | p.P213H | Loss of Function | Missense_Mutation |
| 736 | PTEN | NM_000314.4 | 241294 | c.638C>G | p.P213R | Loss of Function | Missense_Mutation |
| 737 | PTEN | NM_000314.4 | 30729 | c.637C>T | p.P213S | Loss of Function | Missense_Mutation |
| 738 | PTEN | NM_000314.4 | 5822 | c.738delG | p.P246fs*10 | Loss of Function | Frame_Shift_Del |
| 739 | PTEN | NM_000314.4 | 5111 | c.737C>T | p.P246L | Loss of Function | Missense_Mutation |
| 740 | PTEN | NM_000314.4 | 23644 | c.743C>G | p.P248? | Loss of Function | N/A |
| 741 | PTEN | NM_000314.4 | 4986 | c.741_742insA | p.P248fs*5 | Loss of Function | Frame_Shift_Ins |
| 742 | PTEN | NM_000314.4 | 23657 | c.1015C>T | p.P339S | Loss of Function | Missense_Mutation |
| 743 | PTEN | NM_000314.4 | 5153 | c.49C>T | p.Q17* | Loss of Function | Nonsense_Mutation |
| 744 | PTEN | NM_000314.4 | 5149 | c.511C>T | p.Q171* | Loss of Function | Nonsense_Mutation |
| 745 | PTEN | NM_000314.4 | 5200 | c.511C>G | p.Q171E | Loss of Function | Missense_Mutation |
| 746 | PTEN | NM_000314.4 | 5244 | c.512A>C | p.Q171P | Loss of Function | Missense_Mutation |
| 747 | PTEN | NM_000314.4 | 4976 | c.49_51delCAA | p.Q17del | Loss of Function | In_Frame_Del |
| 748 | PTEN | NM_000314.4 | 5159 | c.733C>T | p.Q245* | Loss of Function | Nonsense_Mutation |
| 749 | PTEN | NM_000314.4 | 5160 | c.781C>T | p.Q261* | Loss of Function | Nonsense_Mutation |
| 750 | PTEN | NM_000314.4 | 5156 | c.892C>T | p.Q298* | Loss of Function | Nonsense_Mutation |
| 751 | PTEN | NM_000314.4 | 5101 | c.40A>G | p.R14G | Loss of Function | Missense_Mutation |
| 752 | PTEN | NM_000314.4 | 5232 | c.44G>T | p.R15I | Loss of Function | Missense_Mutation |
| 753 | PTEN | NM_000314.4 | 5270 | c.45A>T | p.R155 | Loss of Function | Missense_Mutation |
| 754 | PTEN | NM_000314.4 | 5089 | c.517C>T | p.R173C | Loss of Function | Missense_Mutation |
| 755 | PTEN | NM_000314.4 | 5825 | c.517delC | p.R173fs*10 | Loss of Function | Frame_Shift_Del |
| 756 | PTEN | NM_000314.4 | 5039 | c.518G>A | p.R173H | Loss of Function | Missense_Mutation |
| 757 | PTEN | NM_000314.4 | 5151 | c.1003C>T | p.R335* | Loss of Function | Nonsense_Mutation |
| 758 | PTEN | NM_000314.4 | 5775 | c.1002_1003CC>TT | p.R335* | Loss of Function | Nonsense_Mutation |
| 759 | PTEN | NM_000314.4 | 5049 | c.29G>A | p.S10N | Loss of Function | Missense_Mutation |
| 760 | PTEN | NM_000314.4 | 5218 | c.509G>T | p.S170I | Loss of Function | Missense_Mutation |
| 761 | PTEN | NM_000314.4 | 5045 | c.509G>A | p.S170N | Loss of Function | Missense_Mutation |
| 762 | PTEN | NM_000314.4 | 4931 | c.881_885delGTCTA | p.S294f5*2 | Loss of Function | Frame_Shift_Del |
| 763 | PTEN | NM_000314.4 | 5313 | c.176C>A | p.S59* | Loss of Function | Nonsense_Mutation |
| 764 | PTEN | NM_000314.4 | 5052 | c.499A>G | p.T167A | Loss of Function | Missense_Mutation |
| 765 | PTEN | NM_000314.4 | 4982 | c.955_957delACT | p.T319del | Loss of Function | Frame_Shift_Del |
| 766 | PTEN | NM_000314.4 | 4958 | c.955_958delACTT | p.T319fs*1 | Loss of Function | Frame_Shift_Del |
| 767 | PTEN | NM_000314.4 | 4896 | c.956_959delCTTT | p.T319fs*24 | Loss of Function | Frame_Shift_Del |
| 768 | PTEN | NM_000314.4 | 5008 | c.955_956insA | p.T319fs*6 | Loss of Function | Frame_Shift_Ins |
| 769 | PTEN | NM_000314.4 | 5823 | c.963delA | p.T321fs*23 | Loss of Function | Frame_Shift_Del |
| 770 | PTEN | NM_000314.4 | 4994 | c.963_964insA | p.T321fs*3 | Loss of Function | Frame_Shift_Ins |
| 771 | PTEN | NM_000314.4 | 5816 | c.867delA | p.V290fs*1 | Loss of Function | Frame_Shift_Del |
| 772 | PTEN | NM_000314.4 | 4898 | c.950_953delTACT | p.V317fs*3 | Loss of Function | Frame_Shift_Del |
| 773 | PTEN | NM_000314.4 | 4899 | c.951_954delACTT | p.V317fs*3 | Loss of Function | Frame_Shift_Del |
| 774 | PTEN | NM_000314.4 | 4943 | c.950_954delTACTT | p.V317fs*6 | Loss of Function | Frame_Shift_Del |
| 775 | PTEN | NM_000314.4 | 5133 | c.47A>G | p.Y16C | Loss of Function | Missense_Mutation |
| 776 | PTEN | NM_000314.4 | 5878 | c.46_47insT | p.Y16fs*28 | Loss of Function | Frame_Shift_Ins |
| 777 | PTEN | NM_000314.4 | 28897 | c.520T>G | p.Y174D | Loss of Function | Missense_Mutation |
| 778 | PTEN | NM_000314.4 | 4969 | c.526_528delTAT | p.Y176del | Loss of Function | In_Frame_Del |
| 779 | PTEN | NM_000314.4 | 33702 | c.530A>G | p.Y177C | Loss of Function | Missense_Mutation |
| 780 | PTEN | NM_000314.4 | 5290 | c.1008C>G | p.Y336* | Loss of Function | Nonsense_Mutation |
| 781 | PTEN | NM_000314.4 | 5296 | c.195C>A | p.Y65* | Loss of Function | Nonsense_Mutation |
| 782 | PTEN | NM_000314.4 | 5317 | c.195C>G | p.Y65* | Loss of Function | Nonsense_Mutation |
| 783 | PTEN | NM_000314.4 | 43077 | c.203A>G | p.Y68C | Loss of Function | Missense_Mutation |
| 784 | PTEN | NM_000314.4 | 4889 | c.202_203delTA | p.Y68fs*5 | Loss of Function | Frame_Shift_Del |
| 785 | PTEN | NM_000314.4 | 5036 | c.202T>C | p.Y68H | Loss of Function | Missense_Mutation |
| 786 | Q7Z252_H | ENST00000341462 | 98900 | c.397G>A | p.D133N | Unclassified | Missense_Mutation |
| 787 | Q7Z252_H | ENST00000341462 | 187238 | c.448C>T | p.P150S | Unclassified | Missense_Mutation |
| 788 | SMAD4 | NM_005359.3 | 14167 | c.955+5G>C | p.? | Loss of Function | N/A |
| 789 | SMAD4 | NM_005359.3 | 14215 | c.353C>T | p.A118V | Loss of Function | Missense_Mutation |
| 790 | SMAD4 | NM_005359.3 | 14105 | c.1394_1395insT | p.A466fs*28 | Loss of Function | Frame_Shift_Ins |
| 791 | SMAD4 | NM_005359.3 | 14216 | c.363_364insA | p.C123fs*2 | Loss of Function | Frame_Shift_Ins |
| 792 | SMAD4 | NM_005359.3 | 25274 | c.366_367insA | p.C123fs*2 | Loss of Function | Frame_Shift_Ins |
| 793 | SMAD4 | NM_005359.3 | 14221 | c.1496G>A | p.C499Y | Loss of Function | Missense_Mutation |
| 794 | SMAD4 | NM_005359.3 | 14115 | c.1569C>G | p.C523W | Loss of Function | Missense_Mutation |
| 795 | SMAD4 | NM_005359.3 | 14135 | c.1051G>C | p.D351H | Loss of Function | Missense_Mutation |
| 796 | SMAD4 | NM_005359.3 | 14232 | c.1064A>G | p.D355G | Loss of Function | Missense_Mutation |
| 797 | SMAD4 | NM_005359.3 | 14110 | c.989A>C | p.E330A | Loss of Function | Missense_Mutation |
| 798 | SMAD4 | NM_005359.3 | 14134 | c.1576G>T | p.E526* | Loss of Function | Nonsense_Mutation |
| 799 | SMAD4 | NM_005359.3 | 14121 | c.1015_1029del15 | p.F339_5343del | Loss of Function | In_Frame_Del |
| 800 | SMAD4 | NM_005359.3 | 14118 | c.502G>T | p.G168* | Loss of Function | Nonsense_Mutation |
| 801 | SMAD4 | NM_005359.3 | 14174 | c.1072G>T | p.G358* | Loss of Function | Nonsense_Mutation |
| 802 | SMAD4 | NM_005359.3 | 14126 | c.1519A>C | p.K507Q | Loss of Function | Missense_Mutation |
| 803 | SMAD4 | NM_005359.3 | 14057 | c.733C>T | p.Q245* | Loss of Function | Nonsense_Mutation |
| 804 | SMAD4 | NM_005359.3 | 14163 | c.931C>T | p.Q311* | Loss of Function | Nonsense_Mutation |
| 805 | SMAD4 | NM_005359.3 | 14223 | c.1229_1230insCA | p.Q410fs*6 | Loss of Function | Frame_Shift_Ins |
| 806 | SMAD4 | NM_005359.3 | 14124 | c.1341_1365del25 | p.Q448f5*20 | Loss of Function | Frame_Shift_Del |
| 807 | SMAD4 | NM_005359.3 | 14140 | c.1081C>T | p.R361C | Loss of Function | Missense_Mutation |
| 808 | SMAD4 | NM_005359.3 | 14122 | c.1082G>A | p.R361H | Loss of Function | Missense_Mutation |
| 809 | SMAD4 | NM_005359.3 | 14113 | c.1490G>A | p.R497H | Loss of Function | Missense_Mutation |
| 810 | SMAD4 | NM_005359.3 | 14129 | c.1543A>T | p.R515* | Loss of Function | Nonsense_Mutation |
| 811 | SMAD4 | NM_005359.3 | 14111 | c.1028C>G | p.S343* | Loss of Function | Nonsense_Mutation |
| 812 | SMAD4 | NM_005359.3 | 14177 | c.1546_1553delCAGAGCAT | p.S517fs*7 | Loss of Function | Frame_Shift_Del |
| 813 | SMAD4 | NM_005359.3 | 14217 | c.776_777delCT | p.T259fs*4 | Loss of Function | Frame_Shift_Del |
| 814 | SMAD4 | NM_005359.3 | 14220 | c.1058A>G | p.Y353C | Loss of Function | Missense_Mutation |
| 815 | SMAD4 | NM_005359.3 | 14175 | c.1236C>G | p.Y412* | Loss of Function | Nonsense_Mutation |
| 816 | STK11 | NM_000455 | 25847 | c.580G>A | p.D194N | Loss of Function | Missense_Mutation |
| 817 | STK11 | NM_000455 | 20944 | c.580G>T | p.D194Y | Loss of Function | Missense_Mutation |
| 818 | STK11 | NM_000455 | 25229 | c.595G>T | p.E199* | Loss of Function | Nonsense_Mutation |
| 819 | STK11 | NM_000455 | 21212 | c.169delG | p.E57fs*7 | Loss of Function | In_Frame_Del |
| 820 | STK11 | NM_000455 | 20857 | c.787_790delTTGT | p.F264fs*22 | Loss of Function | In_Frame_Del |
| 821 | STK11 | NM_000455 | 21360 | c.1062C>G | p.F354L | Loss of Function | Missense_Mutation |
| 822 | STK11 | NM_000455 | 25851 | c.842_843insC | p.L282fs*3 | Loss of Function | In_Frame_Ins |
| 823 | STK11 | NM_000455 | 12924 | c.842delC | p.P281fs*6 | Loss of Function | In_Frame_Del |
| 824 | STK11 | NM_000455 | 20871 | c.837delC | p.P281fs*6 | Loss of Function | In_Frame_Del |
| 825 | STK11 | NM_000455 | 21355 | c.842C>T | p.P281L | Loss of Function | Missense_Mutation |
| 826 | STK11 | NM_000455 | 12925 | c.109C>T | p.Q37* | Loss of Function | Nonsense_Mutation |
| 827 | STK11 | NM_000455 | 21378 | c.96C>G | p.T32T | Loss of Function | Synonymous_Mutation |
| 828 | STK11 | NM_000455 | 18652 | c.996G>A | p.W332* | Loss of Function | Nonsense_Mutation |
| 829 | STK11 | NM_000455 | 29005 | c.816C>T | p.Y272Y | Loss of Function | Synonymous_Mutation |
| 830 | STK11 | NM_000455 | 27322 | c.180delC | p.Y60fs*1 | Loss of Function | In_Frame_Del |
| 831 | TP53 | NM_000546 | 18657 | c.560-2A>G | p.? | Loss of Function | N/A |
| 832 | TP53 | NM_000546 | 21572 | c.376-1G>A | p.? | Loss of Function | N/A |
| 833 | TP53 | NM_000546 | 22908 | c.376-1G>T | p.? | Loss of Function | N/A |
| 834 | TP53 | NM_000546 | 43541 | c.559+3G>C | p.? | Loss of Function | N/A |
| 835 | TP53 | NM_000546 | 43753 | c.560-1G>A | p.? | Loss of Function | N/A |
| 836 | TP53 | NM_000546 | 43841 | c.560-1G>T | p.? | Loss of Function | N/A |
| 837 | TP53 | NM_000546 | 43872 | c.560-1G>C | p.? | Loss of Function | N/A |
| 838 | TP53 | NM_000546 | 43927 | c.559+9C>T | p.? | Loss of Function | N/A |
| 839 | TP53 | NM_000546 | 44268 | c.559+1G>T | p.? | Loss of Function | N/A |
| 840 | TP53 | NM_000546 | 44297 | c.376-3C>T | p.? | Loss of Function | N/A |
| 841 | TP53 | NM_000546 | 44495 | c.559+2T>A | p.? | Loss of Function | N/A |
| 842 | TP53 | NM_000546 | 44933 | c.376-4A>G | p.? | Loss of Function | N/A |
| 843 | TP53 | NM_000546 | 45026 | c.560-2A>T | p.? | Loss of Function | N/A |
| 844 | TP53 | NM_000546 | 45364 | c.376-1delG | p.? | Loss of Function | N/A |
| 845 | TP53 | NM_000546 | 45672 | c.376-2A>G | p.? | Loss of Function | N/A |
| 846 | TP53 | NM_000546 | 45711 | c.559+2T>G | p.? | Loss of Function | N/A |
| 847 | TP53 | NM_000546 | 45809 | c.376-1G>C | p.? | Loss of Function | N/A |
| 848 | TP53 | NM_000546 | 46049 | c.376-2A>C | p.? | Loss of Function | N/A |
| 849 | TP53 | NM_000546 | 46059 | c.560-3T>G | p.? | Loss of Function | N/A |
| 850 | TP53 | NM_000546 | 6900 | c.376-1G>A | p.? | Loss of Function | N/A |
| 851 | TP53 | NM_000546 | 6901 | c.559+1G>A | p.? | Loss of Function | N/A |
| 852 | TP53 | NM_000546 | 44966 | c.385G>A | p.A129T | Loss of Function | Missense_Mutation |
| 853 | TP53 | NM_000546 | 44550 | c.386C>T | p.A129V | Loss of Function | Missense_Mutation |
| 854 | TP53 | NM_000546 | 44130 | c.477C>T | p.A159A | Loss of Function | Synonymous_Mutation |
| 855 | TP53 | NM_000546 | 11496 | c.476C>A | p.A159D | Loss of Function | Missense_Mutation |
| 856 | TP53 | NM_000546 | 45057 | c.475delG | p.A159fs*11 | Loss of Function | Frame_Shift_Del |
| 857 | TP53 | NM_000546 | 43836 | c.475G>C | p.A159P | Loss of Function | Missense_Mutation |
| 858 | TP53 | NM_000546 | 45286 | c.475G>T | p.A159S | Loss of Function | Missense_Mutation |
| 859 | TP53 | NM_000546 | 43626 | c.475G>A | p.A159T | Loss of Function | Missense_Mutation |
| 860 | TP53 | NM_000546 | 11148 | c.476C>T | p.A159V | Loss of Function | Missense_Mutation |
| 861 | TP53 | NM_000546 | 44119 | c.483C>T | p.A161A | Loss of Function | Synonymous_Mutation |
| 862 | TP53 | NM_000546 | 11323 | c.482C>A | p.A161D | Loss of Function | Missense_Mutation |
| 863 | TP53 | NM_000546 | 44230 | c.481delG | p.A161fs*9 | Loss of Function | Frame_Shift_Del |
| 864 | TP53 | NM_000546 | 10739 | c.481G>A | p.A161T | Loss of Function | Missense_Mutation |
| 865 | TP53 | NM_000546 | 43689 | c.482C>T | p.A161V | Loss of Function | Missense_Mutation |
| 866 | TP53 | NM_000546 | 45029 | c.565_591del27 | p.A189_V197delAPP | Loss of Function | In_Frame_Del |
| 867 | TP53 | NM_000546 | 45440 | c.567C>T | p.A189A | Loss of Function | Synonymous_Mutation |
| 868 | TP53 | NM_000546 | 43698 | c.566C>G | p.A189G | Loss of Function | Missense_Mutation |
| 869 | TP53 | NM_000546 | 44923 | c.565G>C | p.A189P | Loss of Function | Missense_Mutation |
| 870 | TP53 | NM_000546 | 43537 | c.565G>A | p.A189T | Loss of Function | Missense_Mutation |
| 871 | TP53 | NM_000546 | 44349 | c.566C>T | p.A189V | Loss of Function | Missense_Mutation |
| 872 | TP53 | NM_000546 | 45268 | c.827C>A | p.A276D | Loss of Function | Missense_Mutation |
| 873 | TP53 | NM_000546 | 45695 | c.827C>G | p.A276G | Loss of Function | Missense_Mutation |
| 874 | TP53 | NM_000546 | 43663 | c.826G>C | p.A276P | Loss of Function | Missense_Mutation |
| 875 | TP53 | NM_000546 | 45467 | c.826G>T | p.A276S | Loss of Function | Missense_Mutation |
| 876 | TP53 | NM_000546 | 44114 | c.826G>A | p.A276T | Loss of Function | Missense_Mutation |
| 877 | TP53 | NM_000546 | 10756 | c.827C>T | p.A276V | Loss of Function | Missense_Mutation |
| 878 | TP53 | NM_000546 | 44019 | c.226_270del45 | p.A76_590del15 | Loss of Function | In_Frame_Del |
| 879 | TP53 | NM_000546 | 45200 | c.233C>T | p.A78V | Loss of Function | Missense_Mutation |
| 880 | TP53 | NM_000546 | 44075 | c.251C>G | p.A84G | Loss of Function | Missense_Mutation |
| 881 | TP53 | NM_000546 | 44194 | c.251C>T | p.A84V | Loss of Function | Missense_Mutation |
| 882 | TP53 | NM_000546 | 44231 | c.262delG | p.A88fs*35 | Loss of Function | Frame_Shift_Del |
| 883 | TP53 | NM_000546 | 44319 | c.405C>A | p.C135* | Loss of Function | Nonsense_Mutation |
| 884 | TP53 | NM_000546 | 43704 | c.405C>T | p.C135C | Loss of Function | Synonymous_Mutation |
| 885 | TP53 | NM_000546 | 10647 | c.404G>T | p.C135F | Loss of Function | Missense_Mutation |
| 886 | TP53 | NM_000546 | 44670 | c.400delT | p.C135fs*35 | Loss of Function | Frame_Shift_Del |
| 887 | TP53 | NM_000546 | 44829 | c.403T>G | p.C135G | Loss of Function | Missense_Mutation |
| 888 | TP53 | NM_000546 | 10684 | c.403T>C | p.C135R | Loss of Function | Missense_Mutation |
| 889 | TP53 | NM_000546 | 44643 | c.404G>C | p.C1355 | Loss of Function | Missense_Mutation |
| 890 | TP53 | NM_000546 | 44910 | c.403T>A | p.C1355 | Loss of Function | Missense_Mutation |
| 891 | TP53 | NM_000546 | 44219 | c.405C>G | p.C135W | Loss of Function | Missense_Mutation |
| 892 | TP53 | NM_000546 | 10801 | c.404G>A | p.C135Y | Loss of Function | Missense_Mutation |
| 893 | TP53 | NM_000546 | 43734 | c.528C>A | p.C176* | Loss of Function | Nonsense_Mutation |
| 894 | TP53 | NM_000546 | 45399 | c.526_543del18 | p.C176_R181delCPH | Loss of Function | In_Frame_Del |
| 895 | TP53 | NM_000546 | 10645 | c.527G>T | p.C176F | Loss of Function | Missense_Mutation |
| 896 | TP53 | NM_000546 | 44759 | c.526delT | p.C176fs*71 | Loss of Function | Frame_Shift_Del |
| 897 | TP53_ENST | ENST00000269305 | 99601 | c.404G>A | p.C135Y | Loss of Function | Missense_Mutation |
| 898 | TP53 | NM_000546 | 44948 | c.526T>C | p.C176R | Loss of Function | Missense_Mutation |
| 899 | TP53 | NM_000546 | 44146 | c.526T>A | p.C176S | Loss of Function | Missense_Mutation |
| 900 | TP53_ENST | ENST00000413465 | 99598 | c.404G>A | p.C135Y | Loss of Function | Missense_Mutation |
| 901 | TP53_ENST | ENST00000545858 | 99625 | c.434G>T | p.C145F | Loss of Function | Missense_Mutation |
| 902 | TP53 | NM_000546 | 10687 | c.527G>A | p.C176Y | Loss of Function | Missense_Mutation |
| 903 | TP53 | NM_000546 | 45562 | c.546C>A | p.C182* | Loss of Function | Nonsense_Mutation |
| 904 | TP53_ENST | ENST00000269305 | 117398 | c.527G>T | p.C176F | Loss of Function | Missense_Mutation |
| 905 | TP53_ENST | ENST00000413465 | 117395 | c.527G>T | p.C176F | Loss of Function | Missense_Mutation |
| 906 | TP53 | NM_000546 | 44692 | c.526T>G | p.C176G | Loss of Function | Missense_Mutation |
| 907 | TP53 | NM_000546 | 44645 | c.527G>C | p.C176S | Loss of Function | Missense_Mutation |
| 908 | TP53 | NM_000546 | 45394 | c.687T>A | p.C229* | Loss of Function | Nonsense_Mutation |
| 909 | TP53 | NM_000546 | 45654 | c.685_699del15 | p.C229_H233delCTT | Loss of Function | In_Frame_Del |
| 910 | TP53 | NM_000546 | 43648 | c.685_686delTG | p.C229fs*10 | Loss of Function | Frame_Shift_Del |
| 911 | TP53 | NM_000546 | 44360 | c.686_687delGT | p.C229fs*10 | Loss of Function | Frame_Shift_Del |
| 912 | TP53 | NM_000546 | 11114 | c.528C>G | p.C176W | Loss of Function | Missense_Mutation |
| 913 | TP53 | NM_000546 | 46288 | c.546C>T | p.C182C | Loss of Function | Synonymous_Mutation |
| 914 | TP53 | NM_000546 | 45677 | c.714T>A | p.C238* | Loss of Function | Nonsense_Mutation |
| 915 | TP53 | NM_000546 | 43778 | c.713G>T | p.C238F | Loss of Function | Missense_Mutation |
| 916 | TP53 | NM_000546 | 44563 | c.544T>C | p.C182R | Loss of Function | Missense_Mutation |
| 917 | TP53 | NM_000546 | 43828 | c.544T>A | p.C182S | Loss of Function | Missense_Mutation |
| 918 | TP53 | NM_000546 | 43700 | c.712T>A | p.C238S | Loss of Function | Missense_Mutation |
| 919 | TP53 | NM_000546 | 44653 | c.713G>C | p.C238S | Loss of Function | Missense_Mutation |
| 920 | TP53 | NM_000546 | 44676 | c.714T>G | p.C238W | Loss of Function | Missense_Mutation |
| 921 | TP53 | NM_000546 | 11059 | c.713G>A | p.C238Y | Loss of Function | Missense_Mutation |
| 922 | TP53 | NM_000546 | 44378 | c.726C>A | p.C242* | Loss of Function | Nonsense_Mutation |
| 923 | TP53 | NM_000546 | 45691 | c.726C>T | p.C242C | Loss of Function | Synonymous_Mutation |
| 924 | TP53 | NM_000546 | 10810 | c.725G>T | p.C242F | Loss of Function | Missense_Mutation |
| 925 | TP53 | NM_000546 | 44657 | c.722delC | p.C242fs*5 | Loss of Function | Frame_Shift_Del |
| 926 | TP53 | NM_000546 | 6530 | c.723delC | p.C242fs*5 | Loss of Function | Frame_Shift_Del |
| 927 | TP53 | NM_000546 | 44546 | c.545G>A | p.C182Y | Loss of Function | Missense_Mutation |
| 928 | TP53 | NM_000546 | 45612 | c.685T>C | p.C229R | Loss of Function | Missense_Mutation |
| 929 | TP53 | NM_000546 | 11133 | c.725G>C | p.C242S | Loss of Function | Missense_Mutation |
| 930 | TP53 | NM_000546 | 44935 | c.724T>A | p.C242S | Loss of Function | Missense_Mutation |
| 931 | TP53 | NM_000546 | 44313 | c.686G>A | p.C229Y | Loss of Function | Missense_Mutation |
| 932 | TP53 | NM_000546 | 10646 | c.725G>A | p.C242Y | Loss of Function | Missense_Mutation |
| 933 | TP53 | NM_000546 | 44735 | c.825T>C | p.C275C | Loss of Function | Synonymous_Mutation |
| 934 | TP53 | NM_000546 | 10701 | c.824G>T | p.C275F | Loss of Function | Missense_Mutation |
| 935 | TP53_ENST | ENST00000269305 | 99626 | c.713G>T | p.C238F | Loss of Function | Missense_Mutation |
| 936 | TP53_ENST | ENST00000413465 | 99624 | c.713G>T | p.C238F | Loss of Function | Missense_Mutation |
| 937 | TP53 | NM_000546 | 45413 | c.824G>C | p.C275S | Loss of Function | Missense_Mutation |
| 938 | TP53 | NM_000546 | 46336 | c.712T>G | p.C238G | Loss of Function | Missense_Mutation |
| 939 | TP53 | NM_000546 | 10893 | c.824G>A | p.C275Y | Loss of Function | Missense_Mutation |
| 940 | TP53 | NM_000546 | 44972 | c.831T>A | p.C277* | Loss of Function | Nonsense_Mutation |
| 941 | TP53 | NM_000546 | 45109 | c.831T>C | p.C277C | Loss of Function | Synonymous_Mutation |
| 942 | TP53 | NM_000546 | 10749 | c.830G>T | p.C277F | Loss of Function | Missense_Mutation |
| 943 | TP53 | NM_000546 | 44321 | c.712T>C | p.C238R | Loss of Function | Missense_Mutation |
| 944 | TP53 | NM_000546 | 44135 | c.724T>G | p.C242G | Loss of Function | Missense_Mutation |
| 945 | TP53 | NM_000546 | 11738 | c.724T>C | p.C242R | Loss of Function | Missense_Mutation |
| 946 | TP53 | NM_000546 | 45338 | c.552T>C | p.D184D | Loss of Function | Synonymous_Mutation |
| 947 | TP53 | NM_000546 | 11356 | c.726C>G | p.C242W | Loss of Function | Missense_Mutation |
| 948 | TP53_ENST | ENST00000269305 | 99932 | c.824G>T | p.C275F | Loss of Function | Missense_Mutation |
| 949 | TP53 | NM_000546 | 11501 | c.823T>G | p.C275G | Loss of Function | Missense_Mutation |
| 950 | TP53 | NM_000546 | 45823 | c.558T>C | p.D186D | Loss of Function | Synonymous_Mutation |
| 951 | TP53 | NM_000546 | 45838 | c.556delG | p.D186fs*61 | Loss of Function | Frame_Shift_Del |
| 952 | TP53 | NM_000546 | 43902 | c.823T>C | p.C275R | Loss of Function | Missense_Mutation |
| 953 | TP53 | NM_000546 | 43823 | c.825T>G | p.C275W | Loss of Function | Missense_Mutation |
| 954 | TP53_ENST | ENST00000269305 | 165084 | c.824G>A | p.C275Y | Loss of Function | Missense_Mutation |
| 955 | TP53 | NM_000546 | 45074 | c.829T>G | p.C277G | Loss of Function | Missense_Mutation |
| 956 | TP53 | NM_000546 | 45299 | c.831T>G | p.C277W | Loss of Function | Missense_Mutation |
| 957 | TP53 | NM_000546 | 45796 | c.623A>G | p.D208G | Loss of Function | Missense_Mutation |
| 958 | TP53 | NM_000546 | 43737 | c.830G>A | p.C277Y | Loss of Function | Missense_Mutation |
| 959 | TP53 | NM_000546 | 44249 | c.623A>T | p.D208V | Loss of Function | Missense_Mutation |
| 960 | TP53_ENST | ENST00000414315 | 99599 | c.86>A | p.C3Y | Loss of Function | Missense_Mutation |
| 961 | TP53 | NM_000546 | 45028 | c.684C>T | p.D228D | Loss of Function | Synonymous_Mutation |
| 962 | TP53_ENST | ENST00000545858 | 99600 | c.125G>A | p.C42Y | Loss of Function | Missense_Mutation |
| 963 | TP53_ENST | ENST00000545858 | 117397 | c.248G>T | p.C83F | Loss of Function | Missense_Mutation |
| 964 | TP53_ENST | ENST00000414315 | 117396 | c.131G>T | p.C44F | Loss of Function | Missense_Mutation |
| 965 | TP53 | NM_000546 | 43797 | c.550G>C | p.D184H | Loss of Function | Missense_Mutation |
| 966 | TP53 | NM_000546 | 44029 | c.550G>A | p.D184N | Loss of Function | Missense_Mutation |
| 967 | TP53 | NM_000546 | 11665 | c.842A>C | p.D281A | Loss of Function | Missense_Mutation |
| 968 | TP53 | NM_000546 | 43958 | c.843C>T | p.D281D | Loss of Function | Synonymous_Mutation |
| 969 | TP53 | NM_000546 | 43837 | c.843C>G | p.D281E | Loss of Function | Missense_Mutation |
| 970 | TP53 | NM_000546 | 43906 | c.843C>A | p.D281E | Loss of Function | Missense_Mutation |
| 971 | TP53 | NM_000546 | 44202 | c.550G>T | p.D184Y | Loss of Function | Missense_Mutation |
| 972 | TP53 | NM_000546 | 10943 | c.841G>C | p.D281H | Loss of Function | Missense_Mutation |
| 973 | TP53 | NM_000546 | 43596 | c.841G>A | p.D281N | Loss of Function | Missense_Mutation |
| 974 | TP53 | NM_000546 | 45729 | c.842A>T | p.D281V | Loss of Function | Missense_Mutation |
| 975 | TP53 | NM_0005 | 11516 | c.841G>T | p.D281Y | Loss of Function | Missense_Mutation |
| 976 | TP53 | NM_000546 | 44837 | c.556G>C | p.D186H | Loss of Function | Missense_Mutation |
| 977 | TP53 | NM_000546 | 10996 | c.511G>T | p.E171* | Loss of Function | Nonsense_Mutation |
| 978 | TP53 | NM_000546 | 46095 | c.511delG | p.E171fs*3 | Loss of Function | Frame_Shift_Del |
| 979 | TP53 | NM_000546 | 44700 | c.556G>A | p.D186N | Loss of Function | Missense_Mutation |
| 980 | TP53 | NM_000546 | 44312 | c.511G>A | p.E171K | Loss of Function | Missense_Mutation |
| 981 | TP53 | NM_000546 | 45519 | c.620A>G | p.D207G | Loss of Function | Missense_Mutation |
| 982 | TP53 | NM_000546 | 43597 | c.538G>T | p.E180* | Loss of Function | Nonsense_Mutation |
| 983 | TP53 | NM_000546 | 45372 | c.540G>T | p.E180D | Loss of Function | Missense_Mutation |
| 984 | TP53 | NM_000546 | 45707 | c.624C>A | p.D208E | Loss of Function | Missense_Mutation |
| 985 | TP53 | NM_000546 | 44241 | c.592G>T | p.E198* | Loss of Function | Nonsense_Mutation |
| 986 | TP53 | NM_000546 | 45851 | c.624C>G | p.D208E | Loss of Function | Missense_Mutation |
| 987 | TP53 | NM_000546 | 43987 | c.622G>A | p.D208N | Loss of Function | Missense_Mutation |
| 988 | TP53 | NM_000546 | 10804 | c.610G>T | p.E204* | Loss of Function | Nonsense_Mutation |
| 989 | TP53 | NM_000546 | 43761 | c.612G>A | p.E204E | Loss of Function | Synonymous_Mutation |
| 990 | TP53 | NM_000546 | 44011 | c.610delG | p.E204fs*43 | Loss of Function | Frame_Shift_Del |
| 991 | TP53 | NM_000546 | 43862 | c.683A>C | p.D228A | Loss of Function | Missense_Mutation |
| 992 | TP53 | NM_000546 | 43853 | c.684C>G | p.D228E | Loss of Function | Missense_Mutation |
| 993 | TP53 | NM_000546 | 44817 | c.661G>T | p.E221* | Loss of Function | Nonsense_Mutation |
| 994 | TP53 | NM_000546 | 43960 | c.683A>G | p.D228G | Loss of Function | Missense_Mutation |
| 995 | TP53 | NM_000546 | 44398 | c.682G>A | p.D228N | Loss of Function | Missense_Mutation |
| 996 | TP53 | NM_000546 | 43750 | c.811G>T | p.E271* | Loss of Function | Nonsense_Mutation |
| 997 | TP53 | NM_000546 | 45529 | c.683A>T | p.D228V | Loss of Function | Missense_Mutation |
| 998 | TP53 | NM_000546 | 45786 | c.682G>T | p.D228Y | Loss of Function | Missense_Mutation |
| 999 | TP53 | NM_000546 | 11232 | c.842A>G | p.D281G | Loss of Function | Missense_Mutation |
| 1000 | TP53 | NM_000546 | 10719 | c.811G>A | p.E271K | Loss of Function | Missense_Mutation |
| 1001 | TP53 | NM_000546 | 11606 | c.31G>C | p.E11Q | Loss of Function | Missense_Mutation |
| 1002 | TP53 | NM_000546 | 44469 | c.812A>T | p.E271V | Loss of Function | Missense_Mutation |
| 1003 | TP53 | NM_000546 | 44388 | c.853G>T | p.E285* | Loss of Function | Nonsense_Mutation |
| 1004 | TP53 | NM_000546 | 44732 | c.512A>G | p.E171G | Loss of Function | Missense_Mutation |
| 1005 | TP53 | NM_000546 | 44709 | c.855G>A | p.E285E | Loss of Function | Synonymous_Mutation |
| 1006 | TP53 | NM_000546 | 45751 | c.511G>C | p.E171Q | Loss of Function | Missense_Mutation |
| 1007 | TP53 | NM_000546 | 10722 | c.853G>A | p.E285K | Loss of Function | Missense_Mutation |
| 1008 | TP53 | NM_000546 | 43772 | c.538G>A | p.E180K | Loss of Function | Missense_Mutation |
| 1009 | TP53 | NM_000546 | 43690 | c.592G>A | p.E198K | Loss of Function | Missense_Mutation |
| 1010 | TP53 | NM_000546 | 43919 | c.856G>T | p.E286* | Loss of Function | Nonsense_Mutation |
| 1011 | TP53 | NM_000546 | 44292 | c.858A>G | p.E286E | Loss of Function | Synonymous_Mutation |
| 1012 | TP53 | NM_000546 | 44651 | c.856_863delGAAGAGAA | p.E286fs*17 | Loss of Function | Frame_Shift_Del |
| 1013 | TP53 | NM_000546 | 45277 | c.856delG | p.E286fs*59 | Loss of Function | Frame_Shift_Del |
| 1014 | TP53 | NM_000546 | 43565 | c.857A>G | p.E286G | Loss of Function | Missense_Mutation |
| 1015 | TP53 | NM_000546 | 10726 | c.856G>A | p.E286K | Loss of Function | Missense_Mutation |
| 1016 | TP53 | NM_000546 | 44250 | c.856G>C | p.E286Q | Loss of Function | Missense_Mutation |
| 1017 | TP53 | NM_000546 | 43936 | c.857A>T | p.E286V | Loss of Function | Missense_Mutation |
| 1018 | TP53 | NM_000546 | 44133 | c.859G>T | p.E287* | Loss of Function | Nonsense_Mutation |
| 1019 | TP53 | NM_000546 | 43776 | c.861G>A | p.E287E | Loss of Function | Synonymous_Mutation |
| 1020 | TP53 | NM_000546 | 45449 | c.592G>C | p.E198Q | Loss of Function | Missense_Mutation |
| 1021 | TP53 | NM_000546 | 45253 | c.611A>G | p.E204G | Loss of Function | Missense_Mutation |
| 1022 | TP53 | NM_000546 | 10856 | c.880G>T | p.E294* | Loss of Function | Nonsense_Mutation |
| 1023 | TP53 | NM_000546 | 43990 | c.610G>A | p.E204K | Loss of Function | Missense_Mutation |
| 1024 | TP53 | NM_000546 | 45516 | c.662A>G | p.E221G | Loss of Function | Missense_Mutation |
| 1025 | TP53 | NM_000546 | 44207 | c.874delA | p.E294fs*51 | Loss of Function | Frame_Shift_Del |
| 1026 | TP53 | NM_000546 | 45670 | c.877delG | p.E294fs*51 | Loss of Function | Frame_Shift_Del |
| 1027 | TP53 | NM_000546 | 6621 | c.880delG | p.E294fs*51 | Loss of Function | Frame_Shift_Del |
| 1028 | TP53 | NM_000546 | 44853 | c.661G>A | p.E221K | Loss of Function | Missense_Mutation |
| 1029 | TP53 | NM_000546 | 44441 | c.813G>C | p.E271D | Loss of Function | Missense_Mutation |
| 1030 | TP53 | NM_000546 | 45284 | c.813G>A | p.E271E | Loss of Function | Synonymous_Mutation |
| 1031 | TP53 | NM_000546 | 10710 | c.892G>T | p.E298* | Loss of Function | Nonsense_Mutation |
| 1032 | TP53 | NM_000546 | 43879 | c.812A>G | p.E271G | Loss of Function | Missense_Mutation |
| 1033 | TP53 | NM_000546 | 11291 | c.1006G>T | p.E336* | Loss of Function | Nonsense_Mutation |
| 1034 | TP53 | NM_000546 | 11286 | c.1015G>T | p.E339* | Loss of Function | Nonsense_Mutation |
| 1035 | TP53 | NM_000546 | 11078 | c.1027G>T | p.E343* | Loss of Function | Nonsense_Mutation |
| 1036 | TP53 | NM_000546 | 10770 | c.1045G>T | p.E349* | Loss of Function | Nonsense_Mutation |
| 1037 | TP53 | NM_000546 | 43706 | c.811G>C | p.E271Q | Loss of Function | Missense_Mutation |
| 1038 | TP53 | NM_000546 | 43614 | c.854A>C | p.E285A | Loss of Function | Missense_Mutation |
| 1039 | TP53 | NM_000546 | 45649 | c.854A>G | p.E285G | Loss of Function | Missense_Mutation |
| 1040 | TP53 | NM_000546 | 44654 | c.400T>C | p.F134L | Loss of Function | Missense_Mutation |
| 1041 | TP53_ENST | ENST00000269305 | 137087 | c.853G>A | p.E285K | Loss of Function | Missense_Mutation |
| 1042 | TP53 | NM_000546 | 43941 | c.400T>G | p.F134V | Loss of Function | Missense_Mutation |
| 1043 | TP53 | NM_000546 | 44162 | c.635_636delTT | p.F212fs*3 | Loss of Function | Frame_Shift_Del |
| 1044 | TP53 | NM_000546 | 44695 | c.634_635delTT | p.F212fs*3 | Loss of Function | Frame_Shift_Del |
| 1045 | TP53 | NM_000546 | 45138 | c.853G>C | p.E285Q | Loss of Function | Missense_Mutation |
| 1046 | TP53 | NM_000546 | 44227 | c.854A>T | p.E285V | Loss of Function | Missense_Mutation |
| 1047 | TP53_ENST | ENST00000269305 | 99924 | c.856G>A | p.E286K | Loss of Function | Missense_Mutation |
| 1048 | TP53 | NM_000546 | 43621 | c.809T>G | p.F270C | Loss of Function | Missense_Mutation |
| 1049 | TP53 | NM_000546 | 43809 | c.808T>A | p.F270I | Loss of Function | Missense_Mutation |
| 1050 | TP53 | NM_000546 | 44156 | c.810T>A | p.F270L | Loss of Function | Missense_Mutation |
| 1051 | TP53 | NM_000546 | 44262 | c.808T>C | p.F270L | Loss of Function | Missense_Mutation |
| 1052 | TP53 | NM_000546 | 45297 | c.810T>G | p.F270L | Loss of Function | Missense_Mutation |
| 1053 | TP53 | NM_000546 | 11305 | c.809T>C | p.F270S | Loss of Function | Missense_Mutation |
| 1054 | TP53 | NM_000546 | 44737 | c.860A>G | p.E287G | Loss of Function | Missense_Mutation |
| 1055 | TP53 | NM_000546 | 44225 | c.859G>A | p.E287K | Loss of Function | Missense_Mutation |
| 1056 | TP53 | NM_000546 | 42813 | c.313G>T | p.G105C | Loss of Function | Missense_Mutation |
| 1057 | TP53 | NM_000546 | 44481 | c.313G>T | p.G105C | Loss of Function | Missense_Mutation |
| 1058 | TP53 | NM_000546 | 45801 | c.312delG | p.G105fs*18 | Loss of Function | Frame_Shift_Del |
| 1059 | TP53 | NM_000546 | 45179 | c.313G>C | p.G105R | Loss of Function | Missense_Mutation |
| 1060 | TP53 | NM_000546 | 45534 | c.882G>T | p.E294D | Loss of Function | Missense_Mutation |
| 1061 | TP53 | NM_000546 | 44715 | c.460G>T | p.G154C | Loss of Function | Missense_Mutation |
| 1062 | TP53 | NM_000546 | 44412 | c.882G>A | p.E294E | Loss of Function | Synonymous_Mutation |
| 1063 | TP53 | NM_000546 | 44726 | c.460_466delGGCACCC | p.G154fs*14 | Loss of Function | Frame_Shift_Del |
| 1064 | TP53 | NM_000546 | 43666 | c.462C>T | p.G154G | Loss of Function | Synonymous_Mutation |
| 1065 | TP53 | NM_000546 | 44298 | c.462C>A | p.G154G | Loss of Function | Synonymous_Mutation |
| 1066 | TP53 | NM_000546 | 43746 | c.881A>G | p.E294G | Loss of Function | Missense_Mutation |
| 1067 | TP53 | NM_000546 | 44127 | c.880G>A | p.E294K | Loss of Function | Missense_Mutation |
| 1068 | TP53 | NM_000546 | 6815 | c.461G>T | p.G154V | Loss of Function | Missense_Mutation |
| 1069 | TP53 | NM_000546 | 45824 | c.880G>C | p.E294Q | Loss of Function | Missense_Mutation |
| 1070 | TP53 | NM_000546 | 45820 | c.893A>T | p.E298V | Loss of Function | Missense_Mutation |
| 1071 | TP53 | NM_000546 | 44026 | c.559G>A | p.G187S | Loss of Function | Splice_Site |
| 1072 | TP53 | NM_000546 | 45169 | c.326T>C | p.F109S | Loss of Function | Missense_Mutation |
| 1073 | TP53 | NM_000546 | 44537 | c.595G>T | p.G199* | Loss of Function | NonsenseMutation |
| 1074 | TP53 | NM_000546 | 43949 | c.401T>G | p.F134C | Loss of Function | Missense_Mutation |
| 1075 | TP53 | NM_000546 | 45051 | c.597A>G | p.G199G | Loss of Function | Synonymous_Mutation |
| 1076 | TP53 | NM_000546 | 11319 | c.402T>G | p.F134L | Loss of Function | Missense_Mutation |
| 1077 | TP53 | NM_000546 | 44140 | c.596G>T | p.G199V | Loss of Function | Missense_Mutation |
| 1078 | TP53 | NM_000546 | 44506 | c.401T>C | p.F134S | Loss of Function | Missense_Mutation |
| 1079 | TP53 | NM_000546 | 45703 | c.634T>A | p.F212I | Loss of Function | Missense_Mutation |
| 1080 | TP53 | NM_000546 | 45868 | c.678C>T | p.G226G | Loss of Function | Synonymous_Mutation |
| 1081 | TP53 | NM_000546 | 44846 | c.636T>A | p.F212L | Loss of Function | Missense_Mutation |
| 1082 | TP53 | NM_000546 | 46214 | c.635T>C | p.F212S | Loss of Function | Missense_Mutation |
| 1083 | TP53 | NM_000546 | 44956 | c.808T>G | p.F270V | Loss of Function | Missense_Mutation |
| 1084 | TP53 | NM_000546 | 11524 | c.730G>T | p.G244C | Loss of Function | Missense_Mutation |
| 1085 | TP53 | NM_000546 | 10883 | c.731G>A | p.G244D | Loss of Function | Missense_Mutation |
| 1086 | TP53 | NM_000546 | 44940 | c.730delG | p.G244fs*3 | Loss of Function | Frame_Shift_Del |
| 1087 | TP53 | NM_000546 | 43656 | c.732C>G | p.G244G | Loss of Function | Synonymous_Mutation |
| 1088 | TP53 | NM_000546 | 44513 | c.732C>A | p.G244G | Loss of Function | Synonymous_Mutation |
| 1089 | TP53 | NM_000546 | 44787 | c.732C>T | p.G244G | Loss of Function | Synonymous_Mutation |
| 1090 | TP53 | NM_000546 | 44221 | c.730G>C | p.G244R | Loss of Function | Missense_Mutation |
| 1091 | TP53 | NM_000546 | 10941 | c.730G>A | p.G244S | Loss of Function | Missense_Mutation |
| 1092 | TP53 | NM_000546 | 43918 | c.809T>A | p.F270Y | Loss of Function | Missense_Mutation |
| 1093 | TP53 | NM_000546 | 13119 | c.322_324delGGT | p.G108del | Loss of Function | In_Frame_Del |
| 1094 | TP53 | NM_000546 | 11081 | c.733G>T | p.G245C | Loss of Function | Missense_Mutation |
| 1095 | TP53 | NM_000546 | 39293 | c.734G>A | p.G245D | Loss of Function | Missense_Mutation |
| 1096 | TP53 | NM_000546 | 43606 | c.734G>A | p.G245D | Loss of Function | Missense_Mutation |
| 1097 | TP53 | NM_000546 | 44642 | c.733delG | p.G245fs*2 | Loss of Function | Frame_Shift_Del |
| 1098 | TP53 | NM_000546 | 44900 | c.735C>T | p.G245G | Loss of Function | Synonymous_Mutation |
| 1099 | TP53_ENST | ENST00000545858 | 179807 | c.455G>A | p.G152D | Loss of Function | Missense_Mutation |
| 1100 | TP53 | NM_000546 | 10957 | c.733G>C | p.G245R | Loss of Function | Missense_Mutation |
| 1101 | TP53 | NM_000546 | 6932 | c.733G>A | p.G245S | Loss of Function | Missense_Mutation |
| 1102 | TP53 | NM_000546 | 11196 | c.734G>T | p.G245V | Loss of Function | Missense_Mutation |
| 1103 | TP53 | NM_000546 | 44891 | c.796G>T | p.G266* | Loss of Function | NonsenseMutation |
| 1104 | TP53_ENST | ENST00000545858 | 121037 | c.454G>A | p.G152S | Loss of Function | Missense_Mutation |
| 1105 | TP53 | NM_000546 | 10867 | c.797G>A | p.G266E | Loss of Function | Missense_Mutation |
| 1106 | TP53 | NM_000546 | 10794 | c.796G>A | p.G266R | Loss of Function | Missense_Mutation |
| 1107 | TP53 | NM_000546 | 11205 | c.796G>C | p.G266R | Loss of Function | Missense_Mutation |
| 1108 | TP53 | NM_000546 | 10958 | c.797G>T | p.G266V | Loss of Function | Missense_Mutation |
| 1109 | TP53 | NM_000546 | 43714 | c.836G>A | p.G279E | Loss of Function | Missense_Mutation |
| 1110 | TP53 | NM_000546 | 44896 | c.835_838delGGGA | p.G279fs*65 | Loss of Function | Frame_Shift_Del |
| 1111 | TP53 | NM_000546 | 46284 | c.837G>A | p.G279G | Loss of Function | Synonymous_Mutation |
| 1112 | TP53 | NM_000546 | 44603 | c.835G>A | p.G279R | Loss of Function | Missense_Mutation |
| 1113 | TP53 | NM_000546 | 45622 | c.461G>A | p.G154D | Loss of Function | Missense_Mutation |
| 1114 | TP53 | NM_000546 | 45506 | c.460_461GG>AT | p.G154I | Loss of Function | Missense_Mutation |
| 1115 | TP53 | NM_000546 | 44128 | c.879G>A | p.G293G | Loss of Function | Synonymous_Mutation |
| 1116 | TP53 | NM_000546 | 44131 | c.879G>C | p.G293G | Loss of Function | Synonymous_Mutation |
| 1117 | TP53 | NM_000546 | 43692 | c.460G>A | p.G154S | Loss of Function | Missense_Mutation |
| 1118 | TP53 | NM_000546 | 45275 | c.559G>T | p.G187C | Loss of Function | Missense_Mutation |
| 1119 | TP53 | NM_000546 | 87513 | c.902_903insC | p.G302fs*4 | Loss of Function | Frame_Shift_Ins |
| 1120 | TP53 | NM_000546 | 45998 | c.906G>C | p.G302G | Loss of Function | Synonymous_Mutation |
| 1121 | TP53 | NM_000546 | 11514 | c.1001G>T | p.G334V | Loss of Function | Missense_Mutation |
| 1122 | TP53 | NM_000546 | 44023 | c.560G>A | p.G187D | Loss of Function | Missense_Mutation |
| 1123 | TP53 | NM_000546 | 45479 | c.504C>T | p.H168H | Loss of Function | Synonymous_Mutation |
| 1124 | TP53 | NM_000546 | 44801 | c.503A>T | p.H168L | Loss of Function | Missense_Mutation |
| 1125 | TP53 | NM_000546 | 44808 | c.503A>C | p.H168P | Loss of Function | Missense_Mutation |
| 1126 | TP53 | NM_000546 | 45240 | c.560G>T | p.G187V | Loss of Function | Missense_Mutation |
| 1127 | TP53 | NM_000546 | 43989 | c.596G>A | p.G199E | Loss of Function | Missense_Mutation |
| 1128 | TP53 | NM_000546 | 44901 | c.532C>G | p.H178D | Loss of Function | Missense_Mutation |
| 1129 | TP53 | NM_000546 | 43978 | c.529delC | p.H178fs*69 | Loss of Function | Frame_Shift_Del |
| 1130 | TP53 | NM_000546 | 44134 | c.528delC | p.H178fs*69 | Loss of Function | Frame_Shift_Del |
| 1131 | TP53 | NM_000546 | 44659 | c.532delC | p.H178fs*69 | Loss of Function | Frame_Shift_Del |
| 1132 | TP53 | NM_000546 | 44971 | c.534C>T | p.H178H | Loss of Function | Synonymous_Mutation |
| 1133 | TP53 | NM_000546 | 43749 | c.595G>A | p.G199R | Loss of Function | Missense_Mutation |
| 1134 | TP53 | NM_000546 | 45739 | c.677G>C | p.G226A | Loss of Function | Missense_Mutation |
| 1135 | TP53 | NM_000546 | 11998 | c.534C>A | p.H178Q | Loss of Function | Missense_Mutation |
| 1136 | TP53 | NM_000546 | 46163 | c.534C>G | p.H178Q | Loss of Function | Missense_Mutation |
| 1137 | TP53 | NM_000546 | 44547 | c.677G>A | p.G226D | Loss of Function | Missense_Mutation |
| 1138 | TP53 | NM_000546 | 44776 | c.535C>G | p.H179D | Loss of Function | Missense_Mutation |
| 1139 | TP53 | NM_000546 | 44793 | c.537T>C | p.H179H | Loss of Function | Synonymous_Mutation |
| 1140 | TP53 | NM_000546 | 43635 | c.536A>T | p.H179L | Loss of Function | Missense_Mutation |
| 1141 | TP53 | NM_000546 | 44151 | c.535C>A | p.H179N | Loss of Function | Missense_Mutation |
| 1142 | TP53 | NM_000546 | 44218 | c.536A>C | p.H179P | Loss of Function | Missense_Mutation |
| 1143 | TP53 | NM_000546 | 11249 | c.537T>G | p.H179Q | Loss of Function | Missense_Mutation |
| 1144 | TP53 | NM_000546 | 44214 | c.537T>A | p.H179Q | Loss of Function | Missense_Mutation |
| 1145 | TP53 | NM_000546 | 10889 | c.536A>G | p.H179R | Loss of Function | Missense_Mutation |
| 1146 | TP53 | NM_000546 | 10768 | c.535C>T | p.H179Y | Loss of Function | Missense_Mutation |
| 1147 | TP53 | NM_000546 | 43584 | c.534_535CC>TT | p.H179Y | Loss of Function | Missense_Mutation |
| 1148 | TP53 | NM_000546 | 44002 | c.577C>G | p.H193D | Loss of Function | Missense_Mutation |
| 1149 | TP53 | NM_000546 | 44848 | c.579T>C | p.H193H | Loss of Function | Synonymous_Mutation |
| 1150 | TP53 | NM_000546 | 11066 | c.578A>T | p.H193L | Loss of Function | Missense_Mutation |
| 1151 | TP53 | NM_000546 | 45607 | c.676G>A | p.G226S | Loss of Function | Missense_Mutation |
| 1152 | TP53 | NM_000546 | 43833 | c.578A>C | p.H193P | Loss of Function | Missense_Mutation |
| 1153 | TP53 | NM_000546 | 10742 | c.578A>G | p.H193R | Loss of Function | Missense_Mutation |
| 1154 | TP53 | NM_000546 | 10672 | c.577C>T | p.H193Y | Loss of Function | Missense_Mutation |
| 1155 | TP53 | NM_000546 | 44399 | c.677G>T | p.G226V | Loss of Function | Missense_Mutation |
| 1156 | TP53 | NM_000546 | 44372 | c.640delC | p.H214fs*33 | Loss of Function | Frame_Shift_Del |
| 1157 | TP53 | NM_000546 | 44638 | c.640_647delCATAGTGT | p.H214fs*5 | Loss of Function | Frame_Shift_Del |
| 1158 | TP53 | NM_000546 | 12013 | c.731G>C | p.G244A | Loss of Function | Missense_Mutation |
| 1159 | TP53 | NM_000546 | 42811 | c.641A>G | p.H214R | Loss of Function | Missense_Mutation |
| 1160 | TP53 | NM_000546 | 43687 | c.641A>G | p.H214R | Loss of Function | Missense_Mutation |
| 1161 | TP53 | NM_000546 | 43652 | c.731G>T | p.G244V | Loss of Function | Missense_Mutation |
| 1162 | TP53 | NM_000546 | 43965 | c.734G>C | p.G245A | Loss of Function | Missense_Mutation |
| 1163 | TP53_ENST | ENST00000269305 | 179806 | c.734G>A | p.G245D | Loss of Function | Missense_Mutation |
| 1164 | TP53_ENST | ENST00000413465 | 179805 | c.734G>A | p.G245D | Loss of Function | Missense_Mutation |
| 1165 | TP53 | NM_000546 | 45410 | c.733_734GG>AA | p.G245N | Loss of Function | Missense_Mutation |
| 1166 | TP53 | NM_000546 | 45069 | c.886delC | p.H296fs*49 | Loss of Function | Frame_Shift_Del |
| 1167 | TP53_ENST | ENST00000269305 | 121035 | c.733G>A | p.G245S | Loss of Function | Missense_Mutation |
| 1168 | TP53_ENST | ENST00000413465 | 121036 | c.733G>A | p.G245S | Loss of Function | Missense_Mutation |
| 1169 | TP53 | NM_000546 | 45488 | c.797G>C | p.G266A | Loss of Function | Missense_Mutation |
| 1170 | TP53_ENST | ENST00000269305 | 99952 | c.797G>T | p.G266V | Loss of Function | Missense_Mutation |
| 1171 | TP53 | NM_000546 | 46032 | c.836G>T | p.G279V | Loss of Function | Missense_Mutation |
| 1172 | TP53 | NM_000546 | 43674 | c.835G>T | p.G279W | Loss of Function | Missense_Mutation |
| 1173 | TP53 | NM_000546 | 45417 | c.877G>A | p.G293R | Loss of Function | Missense_Mutation |
| 1174 | TP53 | NM_000546 | 43988 | c.905G>A | p.G302E | Loss of Function | Missense_Mutation |
| 1175 | TP53 | NM_000546 | 44830 | c.1066G>T | p.G356W | Loss of Function | Missense_Mutation |
| 1176 | TP53_ENST | ENST00000545858 | 99918 | c.299A>T | p.H100L | Loss of Function | Missense_Mutation |
| 1177 | TP53 | NM_000546 | 43545 | c.503A>G | p.H168R | Loss of Function | Missense_Mutation |
| 1178 | TP53 | NM_000546 | 43861 | c.502C>T | p.H168Y | Loss of Function | Missense_Mutation |
| 1179 | TP53 | NM_000546 | 44633 | c.583A>T | p.I195F | Loss of Function | Missense_Mutation |
| 1180 | TP53 | NM_000546 | 44877 | c.584T>A | p.I195N | Loss of Function | Missense_Mutation |
| 1181 | TP53 | NM_000546 | 44539 | c.584T>G | p.1195S | Loss of Function | Missense_Mutation |
| 1182 | TP53 | NM_000546 | 11089 | c.584T>C | p.I195T | Loss of Function | Missense_Mutation |
| 1183 | TP53 | NM_000546 | 43550 | c.694A>T | p.I232F | Loss of Function | Missense_Mutation |
| 1184 | TP53 | NM_000546 | 10715 | c.695T>A | p.I232N | Loss of Function | Missense_Mutation |
| 1185 | TP53 | NM_000546 | 45045 | c.695T>G | p.1232S | Loss of Function | Missense_Mutation |
| 1186 | TP53 | NM_000546 | 44601 | c.695T>C | p.I232T | Loss of Function | Missense_Mutation |
| 1187 | TP53 | NM_000546 | 44068 | c.532C>A | p.H178N | Loss of Function | Missense_Mutation |
| 1188 | TP53 | NM_000546 | 44457 | c.751_759delATCCTCACC | p.1251_T253delILT | Loss of Function | In_Frame_Del |
| 1189 | TP53 | NM_000546 | 44215 | c.533A>C | p.H178P | Loss of Function | Missense_Mutation |
| 1190 | TP53 | NM_000546 | 43967 | c.751A>T | p.I251F | Loss of Function | Missense_Mutation |
| 1191 | TP53 | NM_000546 | 44064 | c.748delC | p.I251fs*94 | Loss of Function | Frame_Shift_Del |
| 1192 | TP53 | NM_000546 | 44124 | c.751delA | p.I251fs*94 | Loss of Function | Frame_Shift_Del |
| 1193 | TP53 | NM_000546 | 44511 | c.753C>A | p.I251I | Loss of Function | Synonymous_Mutation |
| 1194 | TP53 | NM_000546 | 44120 | c.532C>T | p.H178Y | Loss of Function | Missense_Mutation |
| 1195 | TP53 | NM_000546 | 11374 | c.752T>A | p.I251N | Loss of Function | Missense_Mutation |
| 1196 | TP53 | NM_000546 | 43829 | c.752T>G | p.1251S | Loss of Function | Missense_Mutation |
| 1197 | TP53_ENST | ENST00000269305 | 129848 | c.535C>T | p.H179Y | Loss of Function | Missense_Mutation |
| 1198 | TP53_ENST | ENST00000413465 | 129849 | c.535C>T | p.H179Y | Loss of Function | Missense_Mutation |
| 1199 | TP53_ENST | ENST00000269305 | 99919 | c.578A>T | p.H193L | Loss of Function | Missense_Mutation |
| 1200 | TP53_ENST | ENST00000413465 | 99916 | c.578A>T | p.H193L | Loss of Function | Missense_Mutation |
| 1201 | TP53 | NM_000546 | 43935 | c.577C>A | p.H193N | Loss of Function | Missense_Mutation |
| 1202 | TP53 | NM_000546 | 45035 | c.761T>G | p.1254S | Loss of Function | Missense_Mutation |
| 1203 | TP53 | NM_000546 | 45115 | c.640C>G | p.H214D | Loss of Function | Missense_Mutation |
| 1204 | TP53 | NM_000546 | 44407 | c.642T>G | p.H214Q | Loss of Function | Missense_Mutation |
| 1205 | TP53 | NM_000546 | 43651 | c.763A>T | p.I255F | Loss of Function | Missense_Mutation |
| 1206 | TP53 | NM_000546 | 11244 | c.764T>A | p.I255N | Loss of Function | Missense_Mutation |
| 1207 | TP53 | NM_000546 | 10788 | c.764T>G | p.1255S | Loss of Function | Missense_Mutation |
| 1208 | TP53 | NM_000546 | 44112 | c.640C>T | p.H214Y | Loss of Function | Missense_Mutation |
| 1209 | TP53 | NM_000546 | 46031 | c.697C>G | p.H233D | Loss of Function | Missense_Mutation |
| 1210 | TP53 | NM_000546 | 45959 | c.698A>T | p.H233L | Loss of Function | Missense_Mutation |
| 1211 | TP53 | NM_000546 | 44641 | c.394A>T | p.K132* | Loss of Function | NonsenseMutation |
| 1212 | TP53 | NM_000546 | 10813 | c.394A>G | p.K132E | Loss of Function | Missense_Mutation |
| 1213 | TP53 | NM_000546 | 43661 | c.394delA | p.K132fs*38 | Loss of Function | Frame_Shift_Del |
| 1214 | TP53 | NM_000546 | 43592 | c.395A>T | p.K132M | Loss of Function | Missense_Mutation |
| 1215 | TP53 | NM_000546 | 10991 | c.396G>T | p.K132N | Loss of Function | Missense_Mutation |
| 1216 | TP53 | NM_000546 | 43963 | c.396G>C | p.K132N | Loss of Function | Missense_Mutation |
| 1217 | TP53 | NM_000546 | 44350 | c.699C>G | p.H233Q | Loss of Function | Missense_Mutation |
| 1218 | TP53 | NM_000546 | 11582 | c.395A>G | p.K132R | Loss of Function | Missense_Mutation |
| 1219 | TP53 | NM_000546 | 43912 | c.395A>C | p.K132T | Loss of Function | Missense_Mutation |
| 1220 | TP53 | NM_000546 | 10750 | c.490A>T | p.K164* | Loss of Function | Nonsense_Mutation |
| 1221 | TP53 | NM_000546 | 10762 | c.490A>G | p.K164E | Loss of Function | Missense_Mutation |
| 1222 | TP53 | NM_000546 | 45187 | c.490_499del10 | p.K164fs*3 | Loss of Function | Frame_Shift_Del |
| 1223 | TP53 | NM_000546 | 44861 | c.490delA | p.K164fs*6 | Loss of Function | Frame_Shift_Del |
| 1224 | TP53 | NM_000546 | 45103 | c.492G>A | p.K164K | Loss of Function | Synonymous_Mutation |
| 1225 | TP53 | NM_000546 | 44705 | c.697C>T | p.H233Y | Loss of Function | Missense_Mutation |
| 1226 | TP53 | NM_000546 | 44522 | c.887A>T | p.H296L | Loss of Function | Missense_Mutation |
| 1227 | TP53 | NM_000546 | 45306 | c.886C>A | p.H296N | Loss of Function | Missense_Mutation |
| 1228 | TP53 | NM_000546 | 43915 | c.886C>T | p.H296Y | Loss of Function | Missense_Mutation |
| 1229 | TP53 | NM_000546 | 44475 | c.871A>T | p.K291* | Loss of Function | Nonsense_Mutation |
| 1230 | TP53 | NM_000546 | 45494 | c.888_889CC>TT | p.H297Y | Loss of Function | Missense_Mutation |
| 1231 | TP53 | NM_000546 | 44897 | c.871_889del19 | p.K291fs*48 | Loss of Function | Frame_Shift_Del |
| 1232 | TP53 | NM_000546 | 46224 | c.873G>A | p.K291K | Loss of Function | Synonymous_Mutation |
| 1233 | TP53 | NM_000546 | 45803 | c.889C>T | p.H297Y | Loss of Function | Missense_Mutation |
| 1234 | TP53_ENST | ENST00000414315 | 129851 | c.139C>T | p.H47Y | Loss of Function | Missense_Mutation |
| 1235 | TP53_ENST | ENST00000414315 | 99917 | c.182A>T | p.H61L | Loss of Function | Missense_Mutation |
| 1236 | TP53_ENST | ENST00000545858 | 129850 | c.256C>T | p.H86Y | Loss of Function | Missense_Mutation |
| 1237 | TP53 | NM_000546 | 44320 | c.484A>T | p.I162F | Loss of Function | Missense_Mutation |
| 1238 | TP53 | NM_000546 | 44694 | c.486C>A | p.1162I | Loss of Function | Missense_Mutation |
| 1239 | TP53 | NM_000546 | 45627 | c.486C>T | p.1162I | Loss of Function | Missense_Mutation |
| 1240 | TP53 | NM_000546 | 43773 | c.913A>T | p.K305* | Loss of Function | Nonsense_Mutation |
| 1241 | TP53 | NM_000546 | 44125 | c.486C>G | p.I162M | Loss of Function | Missense_Mutation |
| 1242 | TP53 | NM_000546 | 11966 | c.485T>A | p.I162N | Loss of Function | Missense_Mutation |
| 1243 | TP53 | NM_000546 | 11449 | c.388C>T | p.L130F | Loss of Function | Missense_Mutation |
| 1244 | TP53 | NM_000546 | 43898 | c.485T>G | p.1162S | Loss of Function | Missense_Mutation |
| 1245 | TP53 | NM_000546 | 45077 | c.390C>T | p.L130L | Loss of Function | Synonymous_Mutation |
| 1246 | TP53 | NM_000546 | 44413 | c.484A>G | p.I162V | Loss of Function | Missense_Mutation |
| 1247 | TP53 | NM_000546 | 11462 | c.388C>G | p.L130V | Loss of Function | Missense_Mutation |
| 1248 | TP53 | NM_000546 | 10995 | c.580C>T | p.L194F | Loss of Function | Missense_Mutation |
| 1249 | TP53 | NM_000546 | 43623 | c.581T>A | p.L194H | Loss of Function | Missense_Mutation |
| 1250 | TP53 | NM_000546 | 43929 | c.582T>C | p.L194L | Loss of Function | Synonymous_Mutation |
| 1251 | TP53 | NM_000546 | 43827 | c.581T>C | p.L194P | Loss of Function | Missense_Mutation |
| 1252 | TP53 | NM_000546 | 44571 | c.581T>G | p.L194R | Loss of Function | Missense_Mutation |
| 1253 | TP53 | NM_000546 | 44622 | c.694A>G | p.I232V | Loss of Function | Missense_Mutation |
| 1254 | TP53 | NM_000546 | 44650 | c.751_753delATC | p.1251del | Loss of Function | In_Frame_Del |
| 1255 | TP53 | NM_000546 | 44157 | c.601delT | p.L201fs*46 | Loss of Function | Frame_Shift_Del |
| 1256 | TP53 | NM_000546 | 43793 | c.617T>A | p.L206* | Loss of Function | Nonsense_Mutation |
| 1257 | TP53 | NM_000546 | 44852 | c.617delT | p.L206fs*41 | Loss of Function | Frame_Shift_Del |
| 1258 | TP53 | NM_000546 | 10931 | c.751A>C | p.I251L | Loss of Function | Missense_Mutation |
| 1259 | TP53 | NM_000546 | 11213 | c.752T>C | p.I251T | Loss of Function | Missense_Mutation |
| 1260 | TP53 | NM_000546 | 44541 | c.754delC | p.L252fs*93 | Loss of Function | Frame_Shift_Del |
| 1261 | TP53 | NM_000546 | 45407 | c.751A>G | p.I251V | Loss of Function | Missense_Mutation |
| 1262 | TP53 | NM_000546 | 44769 | c.755T>C | p.L252P | Loss of Function | Missense_Mutation |
| 1263 | TP53 | NM_000546 | 11929 | c.760_761AT>GA | p.I254D | Loss of Function | Missense_Mutation |
| 1264 | TP53 | NM_000546 | 11011 | c.794T>C | p.L265P | Loss of Function | Missense_Mutation |
| 1265 | TP53 | NM_000546 | 44092 | c.794T>G | p.L265R | Loss of Function | Missense_Mutation |
| 1266 | TP53 | NM_000546 | 45446 | c.865C>T | p.L289F | Loss of Function | Missense_Mutation |
| 1267 | TP53 | NM_000546 | 45688 | c.867C>T | p.L289L | Loss of Function | Synonymous_Mutation |
| 1268 | TP53 | NM_000546 | 45647 | c.760A>T | p.I254F | Loss of Function | Missense_Mutation |
| 1269 | TP53 | NM_000546 | 44535 | c.761T>A | p.I254N | Loss of Function | Missense_Mutation |
| 1270 | TP53 | NM_000546 | 46015 | c.1043T>A | p.L348* | Loss of Function | Nonsense_Mutation |
| 1271 | TP53 | NM_000546 | 46348 | c.1044G>T | p.L348F | Loss of Function | Missense_Mutation |
| 1272 | TP53 | NM_000546 | 45586 | c.397delA | p.M133fs*37 | Loss of Function | Frame_Shift_Del |
| 1273 | TP53 | NM_000546 | 44058 | c.761T>C | p.I254T | Loss of Function | Missense_Mutation |
| 1274 | TP53 | NM_000546 | 11781 | c.398T>A | p.M133K | Loss of Function | Missense_Mutation |
| 1275 | TP53 | NM_000546 | 44030 | c.760A>G | p.I254V | Loss of Function | Missense_Mutation |
| 1276 | TP53 | NM_000546 | 11181 | c.764T>C | p.I255T | Loss of Function | Missense_Mutation |
| 1277 | TP53 | NM_000546 | 44290 | c.763A>G | p.I255V | Loss of Function | Missense_Mutation |
| 1278 | TP53 | NM_000546 | 44986 | c.302A>G | p.K101R | Loss of Function | Missense_Mutation |
| 1279 | TP53 | NM_000546 | 11224 | c.394A>C | p.K132Q | Loss of Function | Missense_Mutation |
| 1280 | TP53 | NM_000546 | 44841 | c.491A>T | p.K164M | Loss of Function | Missense_Mutation |
| 1281 | TP53 | NM_000546 | 11369 | c.492G>T | p.K164N | Loss of Function | Missense_Mutation |
| 1282 | TP53 | NM_000546 | 44126 | c.507G>A | p.M169I | Loss of Function | Missense_Mutation |
| 1283 | TP53 | NM_000546 | 45490 | c.507G>T | p.M169I | Loss of Function | Missense_Mutation |
| 1284 | TP53 | NM_000546 | 44521 | c.490A>C | p.K164Q | Loss of Function | Missense_Mutation |
| 1285 | TP53 | NM_000546 | 44387 | c.491A>C | p.K164T | Loss of Function | Missense_Mutation |
| 1286 | TP53 | NM_000546 | 45162 | c.709delA | p.M237fs*10 | Loss of Function | Frame_Shift_Del |
| 1287 | TP53 | NM_000546 | 45862 | c.710delT | p.M237fs*10 | Loss of Function | Frame_Shift_Del |
| 1288 | TP53 | NM_000546 | 10834 | c.711G>A | p.M237I | Loss of Function | Missense_Mutation |
| 1289 | TP53 | NM_000546 | 11063 | c.711G>T | p.M237I | Loss of Function | Missense_Mutation |
| 1290 | TP53 | NM_000546 | 44415 | c.711G>C | p.M237I | Loss of Function | Missense_Mutation |
| 1291 | TP53 | NM_000546 | 43952 | c.710T>A | p.M237K | Loss of Function | Missense_Mutation |
| 1292 | TP53 | NM_000546 | 45050 | c.871A>G | p.K291E | Loss of Function | Missense_Mutation |
| 1293 | TP53 | NM_000546 | 44446 | c.873G>C | p.K291N | Loss of Function | Missense_Mutation |
| 1294 | TP53 | NM_000546 | 43747 | c.872A>G | p.K291R | Loss of Function | Missense_Mutation |
| 1295 | TP53 | NM_000546 | 44525 | c.709A>G | p.M237V | Loss of Function | Missense_Mutation |
| 1296 | TP53 | NM_000546 | 44433 | c.872A>C | p.K291T | Loss of Function | Missense_Mutation |
| 1297 | TP53 | NM_000546 | 44451 | c.874A>G | p.K292E | Loss of Function | Missense_Mutation |
| 1298 | TP53 | NM_000546 | 45611 | c.876A>C | p.K292N | Loss of Function | Missense_Mutation |
| 1299 | TP53 | NM_000546 | 43624 | c.875A>G | p.K292R | Loss of Function | Missense_Mutation |
| 1300 | TP53 | NM_000546 | 44346 | c.875A>C | p.K292T | Loss of Function | Missense_Mutation |
| 1301 | TP53 | NM_000546 | 44345 | c.915G>T | p.K305N | Loss of Function | Missense_Mutation |
| 1302 | TP53 | NM_000546 | 43743 | c.914A>G | p.K305R | Loss of Function | Missense_Mutation |
| 1303 | TP53 | NM_000546 | 46114 | c.389T>A | p.L130H | Loss of Function | Missense_Mutation |
| 1304 | TP53 | NM_000546 | 44664 | c.736_750del15 | p.M246_P250delMN | Loss of Function | In_Frame_Del |
| 1305 | TP53 | NM_000546 | 44903 | c.736delA | p.M246fs*1 | Loss of Function | Frame_Shift_Del |
| 1306 | TP53 | NM_000546 | 10757 | c.738G>C | p.M246I | Loss of Function | Missense_Mutation |
| 1307 | TP53 | NM_000546 | 44310 | c.738G>A | p.M246I | Loss of Function | Missense_Mutation |
| 1308 | TP53 | NM_000546 | 46136 | c.738G>T | p.M246I | Loss of Function | Missense_Mutation |
| 1309 | TP53 | NM_000546 | 44063 | c.389T>G | p.L130R | Loss of Function | Missense_Mutation |
| 1310 | TP53 | NM_000546 | 43777 | c.603G>C | p.L201F | Loss of Function | Missense_Mutation |
| 1311 | TP53 | NM_000546 | 11376 | c.737T>G | p.M246R | Loss of Function | Missense_Mutation |
| 1312 | TP53 | NM_000546 | 45489 | c.603G>T | p.L201F | Loss of Function | Missense_Mutation |
| 1313 | TP53 | NM_000546 | 43555 | c.736A>G | p.M246V | Loss of Function | Missense_Mutation |
| 1314 | TP53 | NM_000546 | 44247 | c.754_756delCTC | p.L252del | Loss of Function | In_Frame_Del |
| 1315 | TP53 | NM_000546 | 44054 | c.754C>T | p.L252F | Loss of Function | Missense_Mutation |
| 1316 | TP53 | NM_000546 | 45882 | c.391delA | p.N131fs*39 | Loss of Function | Frame_Shift_Del |
| 1317 | TP53 | NM_000546 | 45091 | c.755T>A | p.L252H | Loss of Function | Missense_Mutation |
| 1318 | TP53 | NM_000546 | 45393 | c.793C>A | p.L265M | Loss of Function | Missense_Mutation |
| 1319 | TP53 | NM_000546 | 43968 | c.866T>C | p.L289P | Loss of Function | Missense_Mutation |
| 1320 | TP53 | NM_000546 | 44070 | c.1031T>C | p.L344P | Loss of Function | Missense_Mutation |
| 1321 | TP53 | NM_000546 | 43859 | c.598delA | p.N200fs*47 | Loss of Function | Frame_Shift_Del |
| 1322 | TP53 | NM_000546 | 44206 | c.399G>T | p.M133I | Loss of Function | Missense_Mutation |
| 1323 | TP53 | NM_000546 | 43730 | c.398T>G | p.M133R | Loss of Function | Missense_Mutation |
| 1324 | TP53 | NM_000546 | 43641 | c.628delA | p.N210fs*37 | Loss of Function | Frame_Shift_Del |
| 1325 | TP53 | NM_000546 | 43723 | c.398T>C | p.M133T | Loss of Function | Missense_Mutation |
| 1326 | TP53_ENST | ENST00000545858 | 99647 | c.432G>A | p.M144I | Loss of Function | Missense_Mutation |
| 1327 | TP53 | NM_000546 | 43891 | c.480G>A | p.M160I | Loss of Function | Missense_Mutation |
| 1328 | TP53 | NM_000546 | 45674 | c.480G>T | p.M160I | Loss of Function | Missense_Mutation |
| 1329 | TP53 | NM_000546 | 44305 | c.479T>A | p.M160K | Loss of Function | Missense_Mutation |
| 1330 | TP53 | NM_000546 | 44842 | c.478A>C | p.M160L | Loss of Function | Missense_Mutation |
| 1331 | TP53 | NM_000546 | 44328 | c.478A>G | p.M160V | Loss of Function | Missense_Mutation |
| 1332 | TP53 | NM_000546 | 45134 | c.715_726del12 | p.N239_C242delNSS | Loss of Function | In_Frame_Del |
| 1333 | TP53 | NM_000546 | 43851 | c.506T>C | p.M169T | Loss of Function | Missense_Mutation |
| 1334 | TP53 | NM_000546 | 10777 | c.715A>G | p.N239D | Loss of Function | Missense_Mutation |
| 1335 | TP53 | NM_000546 | 69195 | c.714_715insT | p.N239fs*1 | Loss of Function | Frame_Shift_Ins |
| 1336 | TP53 | NM_000546 | 44183 | c.715delA | p.N239fs*8 | Loss of Function | Frame_Shift_Del |
| 1337 | TP53 | NM_000546 | 44431 | c.505A>G | p.M169V | Loss of Function | Missense_Mutation |
| 1338 | TP53_ENST | ENST00000269305 | 99648 | c.711G>A | p.M237I | Loss of Function | Missense_Mutation |
| 1339 | TP53 | NM_000546 | 44094 | c.716A>G | p.N239S | Loss of Function | Missense_Mutation |
| 1340 | TP53_ENST | ENST00000413465 | 99646 | c.711G>A | p.M237I | Loss of Function | Missense_Mutation |
| 1341 | TP53 | NM_000546 | 44965 | c.709A>T | p.M237L | Loss of Function | Missense_Mutation |
| 1342 | TP53 | NM_000546 | 6546 | c.741_742CC>AT | p.N247_R248>KW | Loss of Function | In_Frame_Del |
| 1343 | TP53 | NM_000546 | 45032 | c.710T>G | p.M237R | Loss of Function | Missense_Mutation |
| 1344 | TP53 | NM_000546 | 43995 | c.740A>T | p.N247I | Loss of Function | Missense_Mutation |
| 1345 | TP53 | NM_000546 | 45329 | c.710T>C | p.M237T | Loss of Function | Missense_Mutation |
| 1346 | TP53 | NM_000546 | 44428 | c.741C>T | p.N247N | Loss of Function | Synonymous_Mutation |
| 1347 | TP53 | NM_000546 | 44129 | c.729G>A | p.M243I | Loss of Function | Missense_Mutation |
| 1348 | TP53 | NM_000546 | 46228 | c.729G>C | p.M243I | Loss of Function | Missense_Mutation |
| 1349 | TP53 | NM_000546 | 44322 | c.728T>A | p.M243K | Loss of Function | Missense_Mutation |
| 1350 | TP53 | NM_000546 | 43726 | c.727A>T | p.M243L | Loss of Function | Missense_Mutation |
| 1351 | TP53 | NM_000546 | 43765 | c.727A>C | p.M243L | Loss of Function | Missense_Mutation |
| 1352 | TP53 | NM_000546 | 46208 | c.859_872del14 | p.N288fs*13 | Loss of Function | Frame_Shift_Del |
| 1353 | TP53 | NM_000546 | 45459 | c.862delA | p.N288fs*57 | Loss of Function | Frame_Shift_Del |
| 1354 | TP53 | NM_000546 | 44514 | c.728T>G | p.M243R | Loss of Function | Missense_Mutation |
| 1355 | TP53 | NM_000546 | 44536 | c.728T>C | p.M243T | Loss of Function | Missense_Mutation |
| 1356 | TP53 | NM_000546 | 44879 | c.1033delA | p.N345fs*25 | Loss of Function | Frame_Shift_Del |
| 1357 | TP53 | NM_000546 | 44396 | c.382delC | p.P128fs*42 | Loss of Function | Frame_Shift_Del |
| 1358 | TP53 | NM_000546 | 46131 | c.380delC | p.P128fs*42 | Loss of Function | Frame_Shift_Del |
| 1359 | TP53 | NM_000546 | 44844 | c.727A>G | p.M243V | Loss of Function | Missense_Mutation |
| 1360 | TP53 | NM_000546 | 44103 | c.737T>A | p.M246K | Loss of Function | Missense_Mutation |
| 1361 | TP53 | NM_000546 | 44749 | c.453C>T | p.P151P | Loss of Function | Synonymous_Mutation |
| 1362 | TP53 | NM_000546 | 45594 | c.453C>G | p.P151P | Loss of Function | Synonymous_Mutation |
| 1363 | TP53 | NM_000546 | 45992 | c.736A>T | p.M246L | Loss of Function | Missense_Mutation |
| 1364 | TP53 | NM_000546 | 10790 | c.455C>T | p.P152L | Loss of Function | Missense_Mutation |
| 1365 | TP53 | NM_000546 | 44061 | c.456G>A | p.P152P | Loss of Function | Synonymous_Mutation |
| 1366 | TP53 | NM_000546 | 44613 | c.455C>A | p.P152Q | Loss of Function | Missense_Mutation |
| 1367 | TP53 | NM_000546 | 45505 | c.455C>G | p.P152R | Loss of Function | Missense_Mutation |
| 1368 | TP53 | NM_000546 | 43582 | c.454C>T | p.P152S | Loss of Function | Missense_Mutation |
| 1369 | TP53 | NM_000546 | 11355 | c.737T>C | p.M246T | Loss of Function | Missense_Mutation |
| 1370 | TP53 | NM_000546 | 44212 | c.391_393delAAC | p.N131del | Loss of Function | In_Frame_Del |
| 1371 | TP53 | NM_000546 | 43964 | c.459C>T | p.P153P | Loss of Function | Synonymous_Mutation |
| 1372 | TP53 | NM_000546 | 44416 | c.459C>A | p.P153P | Loss of Function | Synonymous_Mutation |
| 1373 | TP53 | NM_000546 | 44589 | c.393_395delCAA | p.N131del | Loss of Function | In_Frame_Del |
| 1374 | TP53 | NM_000546 | 43535 | c.391A>C | p.N131H | Loss of Function | Missense_Mutation |
| 1375 | TP53 | NM_000546 | 43570 | c.529_546del18 | p.P177_C182delPHH | Loss of Function | In_Frame_Del |
| 1376 | TP53 | NM_000546 | 44730 | c.529_545del17 | p.P177fs*3 | Loss of Function | Frame_Shift_Del |
| 1377 | TP53 | NM_000546 | 44794 | c.392A>T | p.N131I | Loss of Function | Missense_Mutation |
| 1378 | TP53 | NM_000546 | 44097 | c.530C>T | p.P177L | Loss of Function | Missense_Mutation |
| 1379 | TP53 | NM_000546 | 43679 | c.531C>T | p.P177P | Loss of Function | Synonymous_Mutation |
| 1380 | TP53 | NM_000546 | 44818 | c.531C>G | p.P177P | Loss of Function | Synonymous_Mutation |
| 1381 | TP53 | NM_000546 | 10651 | c.530C>G | p.P177R | Loss of Function | Missense_Mutation |
| 1382 | TP53 | NM_000546 | 44474 | c.392A>G | p.N131S | Loss of Function | Missense_Mutation |
| 1383 | TP53 | NM_000546 | 43533 | c.391A>T | p.N131Y | Loss of Function | Missense_Mutation |
| 1384 | TP53 | NM_000546 | 46107 | c.599A>T | p.N200I | Loss of Function | Missense_Mutation |
| 1385 | TP53 | NM_000546 | 39455 | c.569delC | p.P190fs*57 | Loss of Function | Frame_Shift_Del |
| 1386 | TP53 | NM_000546 | 45320 | c.569delC | p.P190fs*57 | Loss of Function | Frame_Shift_Del |
| 1387 | TP53 | NM_000546 | 43657 | c.569C>T | p.P190L | Loss of Function | Missense_Mutation |
| 1388 | TP53 | NM_000546 | 44502 | c.599A>G | p.N200S | Loss of Function | Missense_Mutation |
| 1389 | TP53 | NM_000546 | 45441 | c.629A>G | p.N210S | Loss of Function | Missense_Mutation |
| 1390 | TP53 | NM_000546 | 11542 | c.703A>G | p.N235D | Loss of Function | Missense_Mutation |
| 1391 | TP53 | NM_000546 | 44784 | c.703_705delAAC | p.N235del | Loss of Function | In_Frame_Del |
| 1392 | TP53 | NM_000546 | 43860 | c.704A>T | p.N235I | Loss of Function | Missense_Mutation |
| 1393 | TP53 | NM_000546 | 45341 | c.571delC | p.P191fs*56 | Loss of Function | Frame_Shift_Del |
| 1394 | TP53 | NM_000546 | 43616 | c.704A>G | p.N235S | Loss of Function | Missense_Mutation |
| 1395 | TP53 | NM_000546 | 45620 | c.704A>C | p.N235T | Loss of Function | Missense_Mutation |
| 1396 | TP53 | NM_000546 | 45172 | c.703A>T | p.N235Y | Loss of Function | Missense_Mutation |
| 1397 | TP53 | NM_000546 | 45055 | c.715_720delAACAGT | p.N239_5240delNS | Loss of Function | In_Frame_Del |
| 1398 | TP53 | NM_000546 | 44689 | c.657C>T | p.P219P | Loss of Function | Synonymous_Mutation |
| 1399 | TP53 | NM_000546 | 44510 | c.717C>G | p.N239K | Loss of Function | Missense_Mutation |
| 1400 | TP53 | NM_000546 | 44647 | c.717C>A | p.N239K | Loss of Function | Missense_Mutation |
| 1401 | TP53 | NM_000546 | 43801 | c.716A>C | p.N239T | Loss of Function | Missense_Mutation |
| 1402 | TP53 | NM_000546 | 45870 | c.715A>T | p.N239Y | Loss of Function | Missense_Mutation |
| 1403 | TP53 | NM_000546 | 45005 | c.739A>G | p.N247D | Loss of Function | Missense_Mutation |
| 1404 | TP53 | NM_000546 | 45632 | c.741C>A | p.N247K | Loss of Function | Missense_Mutation |
| 1405 | TP53 | NM_000546 | 10771 | c.749C>T | p.P250L | Loss of Function | Missense_Mutation |
| 1406 | TP53 | NM_000546 | 44512 | c.740A>G | p.N247S | Loss of Function | Missense_Mutation |
| 1407 | TP53 | NM_000546 | 43588 | c.740A>C | p.N247T | Loss of Function | Missense_Mutation |
| 1408 | TP53 | NM_000546 | 43864 | c.739A>T | p.N247Y | Loss of Function | Missense_Mutation |
| 1409 | TP53 | NM_000546 | 43979 | c.802A>C | p.N268H | Loss of Function | Missense_Mutation |
| 1410 | TP53 | NM_000546 | 10814 | c.832C>G | p.P278A | Loss of Function | Missense_Mutation |
| 1411 | TP53 | NM_000546 | 44868 | c.804C>T | p.N268N | Loss of Function | Synonymous_Mutation |
| 1412 | TP53 | NM_000546 | 44871 | c.833delC | p.P278fs*67 | Loss of Function | Frame_Shift_Del |
| 1413 | TP53 | NM_000546 | 45178 | c.832delC | p.P278fs*67 | Loss of Function | Frame_Shift_Del |
| 1414 | TP53 | NM_000546 | 43755 | c.833C>A | p.P278H | Loss of Function | Missense_Mutation |
| 1415 | TP53 | NM_000546 | 10863 | c.833C>T | p.P278L | Loss of Function | Missense_Mutation |
| 1416 | TP53 | NM_000546 | 10887 | c.833C>G | p.P278R | Loss of Function | Missense_Mutation |
| 1417 | TP53 | NM_000546 | 10939 | c.832C>T | p.P278S | Loss of Function | Missense_Mutation |
| 1418 | TP53 | NM_000546 | 43697 | c.832C>A | p.P278T | Loss of Function | Missense_Mutation |
| 1419 | TP53 | NM_000546 | 44523 | c.863A>G | p.N288S | Loss of Function | Missense_Mutation |
| 1420 | TP53 | NM_000546 | 45332 | c.885T>C | p.P295P | Loss of Function | Synonymous_Mutation |
| 1421 | TP53 | NM_000546 | 43725 | c.862A>T | p.N288Y | Loss of Function | Missense_Mutation |
| 1422 | TP53 | NM_000546 | 45131 | c.383C>T | p.P128L | Loss of Function | Missense_Mutation |
| 1423 | TP53 | NM_000546 | 44397 | c.382C>T | p.P128S | Loss of Function | Missense_Mutation |
| 1424 | TP53 | NM_000546 | 44788 | c.454C>G | p.P152A | Loss of Function | Missense_Mutation |
| 1425 | TP53 | NM_000546 | 45184 | c.902delC | p.P301fs*44 | Loss of Function | Frame_Shift_Del |
| 1426 | TP53 | NM_000546 | 45487 | c.898delC | p.P301fs*44 | Loss of Function | Frame_Shift_Del |
| 1427 | TP53 | NM_000546 | 45546 | c.901delC | p.P301fs*44 | Loss of Function | Frame_Shift_Del |
| 1428 | TP53 | NM_000546 | 44165 | c.903A>G | p.P301P | Loss of Function | Synonymous_Mutation |
| 1429 | TP53_ENST | ENST00000269305 | 129856 | c.455C>T | p.P152L | Loss of Function | Missense_Mutation |
| 1430 | TP53_ENST | ENST00000413465 | 129857 | c.455C>T | p.P152L | Loss of Function | Missense_Mutation |
| 1431 | TP53 | NM_000546 | 44561 | c.454C>A | p.P152T | Loss of Function | Missense_Mutation |
| 1432 | TP53 | NM_000546 | 44367 | c.458C>T | p.P153L | Loss of Function | Missense_Mutation |
| 1433 | TP53 | NM_000546 | 43675 | c.457C>T | p.P153S | Loss of Function | Missense_Mutation |
| 1434 | TP53 | NM_000546 | 45660 | c.457C>A | p.P153T | Loss of Function | Missense_Mutation |
| 1435 | TP53 | NM_000546 | 45326 | c.530C>A | p.P177H | Loss of Function | Missense_Mutation |
| 1436 | TP53 | NM_000546 | 10650 | c.529C>T | p.P1775 | Loss of Function | Missense_Mutation |
| 1437 | TP53 | NM_000546 | 44426 | c.568C>G | p.P190A | Loss of Function | Missense_Mutation |
| 1438 | TP53 | NM_000546 | 44032 | c.298C>T | p.Q100* | Loss of Function | Nonsense_Mutation |
| 1439 | TP53 | NM_000546 | 10886 | c.310C>T | p.Q104* | Loss of Function | Nonsense_Mutation |
| 1440 | TP53 | NM_000546 | 11166 | c.406C>T | p.Q136* | Loss of Function | Nonsense_Mutation |
| 1441 | TP53 | NM_000546 | 43767 | c.406C>G | p.Q136E | Loss of Function | Missense_Mutation |
| 1442 | TP53 | NM_000546 | 45089 | c.407A>C | p.Q136P | Loss of Function | Missense_Mutation |
| 1443 | TP53 | NM_000546 | 44665 | c.568_570delCCT | p.P190del | Loss of Function | In_Frame_Del |
| 1444 | TP53 | NM_000546 | 43632 | c.493C>T | p.Q165* | Loss of Function | Nonsense_Mutation |
| 1445 | TP53 | NM_000546 | 44004 | c.569C>G | p.P190R | Loss of Function | Missense_Mutation |
| 1446 | TP53 | NM_000546 | 44682 | c.568C>T | p.P1905 | Loss of Function | Missense_Mutation |
| 1447 | TP53 | NM_000546 | 44438 | c.568C>A | p.P190T | Loss of Function | Missense_Mutation |
| 1448 | TP53 | NM_000546 | 11333 | c.499C>T | p.Q167* | Loss of Function | Nonsense_Mutation |
| 1449 | TP53 | NM_000546 | 44275 | c.499_500delCA | p.Q167f5*13 | Loss of Function | Frame_Shift_Del |
| 1450 | TP53 | NM_000546 | 51646 | c.498_499insC | p.Q167f5*14 | Loss of Function | Frame_Shift_Ins |
| 1451 | TP53 | NM_000546 | 44336 | c.499delC | p.Q167f5*3 | Loss of Function | Frame_Shift_Del |
| 1452 | TP53 | NM_000546 | 44234 | c.571_573delCCT | p.P191del | Loss of Function | In_Frame_Del |
| 1453 | TP53 | NM_000546 | 45140 | c.572_574delCTC | p.P191del | Loss of Function | In_Frame_Del |
| 1454 | TP53 | NM_000546 | 44299 | c.501G>A | p.Q1670 | Loss of Function | Synonymous_Mutation |
| 1455 | TP53_ENST | ENST00000269305 | 111724 | c.572_574delCTC | p.P191delP | Loss of Function | In_Frame_Del |
| 1456 | TP53 | NM_000546 | 10733 | c.574C>T | p.Q192* | Loss of Function | Nonsense_Mutation |
| 1457 | TP53_ENST | ENST00000413465 | 111721 | c.572_574delCTC | p.P191delP | Loss of Function | In_Frame_Del |
| 1458 | TP53 | NM_000546 | 43782 | c.576G>A | p.Q192Q | Loss of Function | Synonymous_Mutation |
| 1459 | TP53 | NM_000546 | 44351 | c.572C>T | p.P191L | Loss of Function | Missense_Mutation |
| 1460 | TP53 | NM_000546 | 44172 | c.572C>G | p.P191R | Loss of Function | Missense_Mutation |
| 1461 | TP53 | NM_000546 | 43682 | c.328C>T | p.R110C | Loss of Function | Missense_Mutation |
| 1462 | TP53 | NM_000546 | 43702 | c.571C>T | p.P191S | Loss of Function | Missense_Mutation |
| 1463 | TP53 | NM_000546 | 10716 | c.329G>T | p.R110L | Loss of Function | Missense_Mutation |
| 1464 | TP53 | NM_000546 | 11250 | c.329G>C | p.R110P | Loss of Function | Missense_Mutation |
| 1465 | TP53_ENST | ENST00000414315 | 129859 | c.59C>T | p.P20L | Loss of Function | Missense_Mutation |
| 1466 | TP53 | NM_000546 | 46124 | c.466C>T | p.R156C | Loss of Function | Missense_Mutation |
| 1467 | TP53 | NM_000546 | 45896 | c.466delC | p.R156fs*14 | Loss of Function | Frame_Shift_Del |
| 1468 | TP53 | NM_000546 | 44439 | c.656C>T | p.P219L | Loss of Function | Missense_Mutation |
| 1469 | TP53 | NM_000546 | 43739 | c.467G>A | p.R156H | Loss of Function | Missense_Mutation |
| 1470 | TP53 | NM_000546 | 44076 | c.655C>T | p.P219S | Loss of Function | Missense_Mutation |
| 1471 | TP53 | NM_000546 | 10760 | c.467G>C | p.R156P | Loss of Function | Missense_Mutation |
| 1472 | TP53 | NM_000546 | 44301 | c.468C>G | p.R156R | Loss of Function | Synonymous_Mutation |
| 1473 | TP53 | NM_000546 | 44854 | c.655C>A | p.P219T | Loss of Function | Missense_Mutation |
| 1474 | TP53 | NM_000546 | 44921 | c.748_756delCCCATCCTC | p.P250_1_252delPIL | Loss of Function | In_Frame_Del |
| 1475 | TP53 | NM_000546 | 43848 | c.472C>T | p.R158C | Loss of Function | Missense_Mutation |
| 1476 | TP53 | NM_000546 | 45019 | c.471_472CC>TT | p.R158C | Loss of Function | Missense_Mutation |
| 1477 | TP53 | NM_000546 | 43831 | c.472_475delCGCG | p.R158fs*11 | Loss of Function | Frame_Shift_Del |
| 1478 | TP53 | NM_000546 | 43781 | c.472delC | p.R158fs*12 | Loss of Function | Frame_Shift_Del |
| 1479 | TP53 | NM_000546 | 11087 | c.472C>G | p.R158G | Loss of Function | Missense_Mutation |
| 1480 | TP53 | NM_000546 | 10690 | c.473G>A | p.R158H | Loss of Function | Missense_Mutation |
| 1481 | TP53 | NM_000546 | 10714 | c.473G>T | p.R158L | Loss of Function | Missense_Mutation |
| 1482 | TP53 | NM_000546 | 44096 | c.748C>G | p.P250A | Loss of Function | Missense_Mutation |
| 1483 | TP53 | NM_000546 | 43940 | c.474C>T | p.R158R | Loss of Function | Synonymous_Mutation |
| 1484 | TP53 | NM_000546 | 46393 | c.520_536del17 | p.R174fs*1 | Loss of Function | Frame_Shift_Del |
| 1485 | TP53 | NM_000546 | 44725 | c.522delG | p.R174fs*73 | Loss of Function | Frame_Shift_Del |
| 1486 | TP53 | NM_000546 | 44609 | c.748_749CC>TT | p.P250F | Loss of Function | Missense_Mutation |
| 1487 | TP53 | NM_000546 | 44476 | c.749C>A | p.P250H | Loss of Function | Missense_Mutation |
| 1488 | TP53 | NM_000546 | 45034 | c.748_749CC>AA | p.P250N | Loss of Function | Missense_Mutation |
| 1489 | TP53 | NM_000546 | 43957 | c.750C>T | p.P250P | Loss of Function | Synonymous_Mutation |
| 1490 | TP53 | NM_000546 | 44742 | c.523_540del18 | p.R175_E180delRCP | Loss of Function | Frame_Shift_Del |
| 1491 | TP53 | NM_000546 | 43680 | c.523C>T | p.R175C | Loss of Function | Missense_Mutation |
| 1492 | TP53 | NM_000546 | 10870 | c.523C>G | p.R175G | Loss of Function | Missense_Mutation |
| 1493 | TP53 | NM_000546 | 10648 | c.524G>A | p.R175H | Loss of Function | Missense_Mutation |
| 1494 | TP53 | NM_000546 | 44464 | c.749_750CC>AG | p.P250Q | Loss of Function | Missense_Mutation |
| 1495 | TP53 | NM_000546 | 43695 | c.748C>T | p.P250S | Loss of Function | Missense_Mutation |
| 1496 | TP53 | NM_000546 | 44566 | c.525C>G | p.R175R | Loss of Function | Synonymous_Mutation |
| 1497 | TP53 | NM_000546 | 45515 | c.525C>T | p.R175R | Loss of Function | Synonymous_Mutation |
| 1498 | TP53_ENST | ENST00000269305 | 99725 | c.832C>G | p.P278A | Loss of Function | Missense_Mutation |
| 1499 | TP53 | NM_000546 | 11090 | c.541C>T | p.R181C | Loss of Function | Missense_Mutation |
| 1500 | TP53 | NM_000546 | 43587 | c.832_833CC>TT | p.P278F | Loss of Function | Missense_Mutation |
| 1501 | TP53_ENST | ENST00000269305 | 129831 | c.833C>T | p.P278L | Loss of Function | Missense_Mutation |
| 1502 | TP53 | NM_000546 | 45046 | c.542G>C | p.R181P | Loss of Function | Missense_Mutation |
| 1503 | TP53 | NM_000546 | 43728 | c.543C>T | p.R181R | Loss of Function | Synonymous_Mutation |
| 1504 | TP53 | NM_000546 | 10705 | c.586C>T | p.R196* | Loss of Function | Nonsense_Mutation |
| 1505 | TP53 | NM_000546 | 45021 | c.585_586CC>TT | p.R196* | Loss of Function | Nonsense_Mutation |
| 1506 | TP53 | NM_000546 | 44757 | c.586delC | p.R196fs*51 | Loss of Function | Frame_Shift_Del |
| 1507 | TP53 | NM_000546 | 43814 | c.587G>C | p.R196P | Loss of Function | Missense_Mutation |
| 1508 | TP53_ENST | ENST00000269305 | 139044 | c.832C>T | p.P278S | Loss of Function | Missense_Mutation |
| 1509 | TP53 | NM_000546 | 44569 | c.588A>G | p.R196R | Loss of Function | Synonymous_Mutation |
| 1510 | TP53 | NM_000546 | 44615 | c.586C>A | p.R196R | Loss of Function | Synonymous_Mutation |
| 1511 | TP53 | NM_000546 | 45233 | c.884C>T | p.P295L | Loss of Function | Missense_Mutation |
| 1512 | TP53 | NM_000546 | 44750 | c.883C>T | p.P295S | Loss of Function | Missense_Mutation |
| 1513 | TP53 | NM_000546 | 45311 | c.898C>G | p.P300A | Loss of Function | Missense_Mutation |
| 1514 | TP53 | NM_000546 | 43766 | c.899C>T | p.P300L | Loss of Function | Missense_Mutation |
| 1515 | TP53 | NM_000546 | 44729 | c.898C>T | p.P300S | Loss of Function | Missense_Mutation |
| 1516 | TP53 | NM_000546 | 44753 | c.901C>T | p.P301S | Loss of Function | Missense_Mutation |
| 1517 | TP53 | NM_000546 | 11290 | c.625A>T | p.R209* | Loss of Function | Nonsense_Mutation |
| 1518 | TP53 | NM_000546 | 96575 | c.625_634del10 | p.R209fs*35 | Loss of Function | Frame_Shift_Del |
| 1519 | TP53 | NM_000546 | 45438 | c.626delG | p.R209fs*38 | Loss of Function | Frame_Shift_Del |
| 1520 | TP53 | NM_000546 | 13120 | c.626_627delGA | p.R209fs*6 | Loss of Function | Frame_Shift_Del |
| 1521 | TP53 | NM_000546 | 6482 | c.625_626delAG | p.R209fs*6 | Loss of Function | Frame_Shift_Del |
| 1522 | TP53_ENST | ENST00000414315 | 111722 | c.176_178delCTC | p.P59delP | Loss of Function | In_Frame_Del |
| 1523 | TP53_ENST | ENST00000545858 | 129858 | c.176C>T | p.P59L | Loss of Function | Missense_Mutation |
| 1524 | TP53 | NM_000546 | 10654 | c.637C>T | p.R213* | Loss of Function | Nonsense_Mutation |
| 1525 | TP53 | NM_000546 | 43807 | c.637delC | p.R213fs*34 | Loss of Function | Frame_Shift_Del |
| 1526 | TP53 | NM_000546 | 44358 | c.634delT | p.R213fs*34 | Loss of Function | Frame_Shift_Del |
| 1527 | TP53 | NM_000546 | 45777 | c.633delT | p.R213fs*34 | Loss of Function | Frame_Shift_Del |
| 1528 | TP53 | NM_000546 | 44102 | c.637C>G | p.R213G | Loss of Function | Missense_Mutation |
| 1529 | TP53 | NM_000546 | 43650 | c.638G>T | p.R213L | Loss of Function | Missense_Mutation |
| 1530 | TP53 | NM_000546 | 11860 | c.638G>C | p.R213P | Loss of Function | Missense_Mutation |
| 1531 | TP53 | NM_000546 | 10735 | c.638G>A | p.R213Q | Loss of Function | Missense_Mutation |
| 1532 | TP53 | NM_000546 | 45116 | c.742delC | p.R248fs*97 | Loss of Function | Frame_Shift_Del |
| 1533 | TP53 | NM_000546 | 11564 | c.742C>G | p.R248G | Loss of Function | Missense_Mutation |
| 1534 | TP53 | NM_000546 | 45543 | c.743_744GG>TT | p.R248L | Loss of Function | Missense_Mutation |
| 1535 | TP53 | NM_000546 | 6549 | c.743G>T | p.R248L | Loss of Function | Missense_Mutation |
| 1536 | TP53 | NM_000546 | 11491 | c.743G>C | p.R248P | Loss of Function | Missense_Mutation |
| 1537 | TP53 | NM_000546 | 10662 | c.743G>A | p.R248Q | Loss of Function | Missense_Mutation |
| 1538 | TP53 | NM_000546 | 44908 | c.743_744GG>AA | p.R248Q | Loss of Function | Missense_Mutation |
| 1539 | TP53 | NM_000546 | 46265 | c.224C>G | p.P75R | Loss of Function | Missense_Mutation |
| 1540 | TP53 | NM_000546 | 44287 | c.229C>G | p.P77A | Loss of Function | Missense_Mutation |
| 1541 | TP53 | NM_000546 | 43910 | c.245C>T | p.P82L | Loss of Function | Missense_Mutation |
| 1542 | TP53 | NM_000546 | 10656 | c.742C>T | p.R248W | Loss of Function | Missense_Mutation |
| 1543 | TP53 | NM_000546 | 6545 | c.741_742CC>TT | p.R248W | Loss of Function | Missense_Mutation |
| 1544 | TP53 | NM_000546 | 44916 | c.746delG | p.R249fs*96 | Loss of Function | Frame_Shift_Del |
| 1545 | TP53 | NM_000546 | 10668 | c.745A>G | p.R249G | Loss of Function | Missense_Mutation |
| 1546 | TP53 | NM_000546 | 44091 | c.746G>A | p.R249K | Loss of Function | Missense_Mutation |
| 1547 | TP53 | NM_000546 | 43871 | c.746G>T | p.R249M | Loss of Function | Missense_Mutation |
| 1548 | TP53 | NM_000546 | 45918 | c.253C>T | p.P85S | Loss of Function | Missense_Mutation |
| 1549 | TP53 | NM_000546 | 10785 | c.747G>C | p.R249S | Loss of Function | Missense_Mutation |
| 1550 | TP53 | NM_000546 | 10817 | c.747G>T | p.R249S | Loss of Function | Missense_Mutation |
| 1551 | TP53 | NM_000546 | 43665 | c.746G>C | p.R249T | Loss of Function | Missense_Mutation |
| 1552 | TP53 | NM_000546 | 43629 | c.745A>T | p.R249W | Loss of Function | Missense_Mutation |
| 1553 | TP53 | NM_000546 | 11392 | c.800G>C | p.R267P | Loss of Function | Missense_Mutation |
| 1554 | TP53 | NM_000546 | 43923 | c.800G>A | p.R267Q | Loss of Function | Missense_Mutation |
| 1555 | TP53 | NM_000546 | 43544 | c.260C>A | p.P87Q | Loss of Function | Missense_Mutation |
| 1556 | TP53 | NM_000546 | 11183 | c.799C>T | p.R267W | Loss of Function | Missense_Mutation |
| 1557 | TP53 | NM_000546 | 10659 | c.817C>T | p.R273C | Loss of Function | Missense_Mutation |
| 1558 | TP53 | NM_000546 | 44701 | c.817delC | p.R273fs*72 | Loss of Function | Frame_Shift_Del |
| 1559 | TP53 | NM_000546 | 43688 | c.265C>T | p.P89S | Loss of Function | Missense_Mutation |
| 1560 | TP53 | NM_000546 | 10660 | c.818G>A | p.R273H | Loss of Function | Missense_Mutation |
| 1561 | TP53 | NM_000546 | 10779 | c.818G>T | p.R273L | Loss of Function | Missense_Mutation |
| 1562 | TP53 | NM_000546 | 43896 | c.818G>C | p.R273P | Loss of Function | Missense_Mutation |
| 1563 | TP53 | NM_000546 | 43909 | c.817C>A | p.R273S | Loss of Function | Missense_Mutation |
| 1564 | TP53 | NM_000546 | 44390 | c.838A>T | p.R280* | Loss of Function | NonsenseMutation |
| 1565 | TP53_ENST | ENST00000545858 | 111723 | c.293_295delCTC | p.P98delP | Loss of Function | In_Frame_Del |
| 1566 | TP53 | NM_000546 | 44005 | c.835delG | p.R280fs*65 | Loss of Function | Frame_Shift_Del |
| 1567 | TP53 | NM_000546 | 11123 | c.838A>G | p.R280G | Loss of Function | Missense_Mutation |
| 1568 | TP53 | NM_000546 | 11287 | c.839G>T | p.R280I | Loss of Function | Missense_Mutation |
| 1569 | TP53 | NM_000546 | 10728 | c.839G>A | p.R280K | Loss of Function | Missense_Mutation |
| 1570 | TP53 | NM_000546 | 44568 | c.840A>G | p.R280R | Loss of Function | Synonymous_Mutation |
| 1571 | TP53 | NM_000546 | 44171 | c.840A>T | p.R280S | Loss of Function | Missense_Mutation |
| 1572 | TP53 | NM_000546 | 44233 | c.840A>C | p.R280S | Loss of Function | Missense_Mutation |
| 1573 | TP53 | NM_000546 | 10724 | c.839G>C | p.R280T | Loss of Function | Missense_Mutation |
| 1574 | TP53 | NM_000546 | 10992 | c.844C>G | p.R282G | Loss of Function | Missense_Mutation |
| 1575 | TP53 | NM_000546 | 44681 | c.293C>T | p.P98L | Loss of Function | Missense_Mutation |
| 1576 | TP53 | NM_000546 | 12296 | c.292C>T | p.P98S | Loss of Function | Missense_Mutation |
| 1577 | TP53 | NM_000546 | 44338 | c.845G>A | p.R282Q | Loss of Function | Missense_Mutation |
| 1578 | TP53 | NM_000546 | 44724 | c.846G>A | p.R282R | Loss of Function | Synonymous_Mutation |
| 1579 | TP53 | NM_000546 | 44918 | c.844C>A | p.R282R | Loss of Function | Synonymous_Mutation |
| 1580 | TP53 | NM_000546 | 10704 | c.844C>T | p.R282W | Loss of Function | Missense_Mutation |
| 1581 | TP53 | NM_000546 | 43585 | c.843_844CC>TT | p.R282W | Loss of Function | Missense_Mutation |
| 1582 | TP53 | NM_000546 | 45293 | c.407A>G | p.Q136R | Loss of Function | Missense_Mutation |
| 1583 | TP53 | NM_000546 | 45891 | c.847_866del20 | p.R283fs*16 | Loss of Function | Frame_Shift_Del |
| 1584 | TP53 | NM_000546 | 45188 | c.847delC | p.R283fs*62 | Loss of Function | Frame_Shift_Del |
| 1585 | TP53 | NM_000546 | 44850 | c.494A>T | p.Q165L | Loss of Function | Missense_Mutation |
| 1586 | TP53 | NM_000546 | 44851 | c.494A>C | p.Q165P | Loss of Function | Missense_Mutation |
| 1587 | TP53 | NM_000546 | 44308 | c.494A>G | p.Q165R | Loss of Function | Missense_Mutation |
| 1588 | TP53 | NM_000546 | 10743 | c.848G>C | p.R283P | Loss of Function | Missense_Mutation |
| 1589 | TP53 | NM_000546 | 43977 | c.849C>T | p.R283R | Loss of Function | Synonymous_Mutation |
| 1590 | TP53 | NM_000546 | 45679 | c.868C>T | p.R290C | Loss of Function | Missense_Mutation |
| 1591 | TP53 | NM_000546 | 45626 | c.501G>T | p.Q167H | Loss of Function | Missense_Mutation |
| 1592 | TP53 | NM_000546 | 45342 | c.500A>T | p.Q167L | Loss of Function | Missense_Mutation |
| 1593 | TP53 | NM_000546 | 10663 | c.916C>T | p.R306* | Loss of Function | Nonsense_Mutation |
| 1594 | TP53 | NM_000546 | 11071 | c.1009C>T | p.R337C | Loss of Function | Missense_Mutation |
| 1595 | TP53 | NM_000546 | 43882 | c.1010G>A | p.R337H | Loss of Function | Missense_Mutation |
| 1596 | TP53 | NM_000546 | 11411 | c.1010G>T | p.R337L | Loss of Function | Missense_Mutation |
| 1597 | TP53 | NM_000546 | 11073 | c.1024C>T | p.R342* | Loss of Function | Nonsense_Mutation |
| 1598 | TP53 | NM_000546 | 18597 | c.1024delC | p.R342fs*3 | Loss of Function | Frame_Shift_Del |
| 1599 | TP53 | NM_000546 | 43795 | c.1023delC | p.R342fs*3 | Loss of Function | Frame_Shift_Del |
| 1600 | TP53 | NM_000546 | 45639 | c.1024delC | p.R342fs*3 | Loss of Function | Frame_Shift_Del |
| 1601 | TP53 | NM_000546 | 45276 | c.1025G>C | p.R342P | Loss of Function | Missense_Mutation |
| 1602 | TP53 | NM_000546 | 43709 | c.500A>G | p.Q167R | Loss of Function | Missense_Mutation |
| 1603 | TP53 | NM_000546 | 45044 | c.576G>T | p.Q192H | Loss of Function | Missense_Mutation |
| 1604 | TP53 | NM_000546 | 44849 | c.575A>G | p.Q192R | Loss of Function | Missense_Mutation |
| 1605 | TP53 | NM_000546 | 45944 | c.318C>G | p.S106R | Loss of Function | Missense_Mutation |
| 1606 | TP53 | NM_000546 | 40942 | c.380C>T | p.S127F | Loss of Function | Missense_Mutation |
| 1607 | TP53 | NM_000546 | 45536 | c.1061A>G | p.Q354R | Loss of Function | Missense_Mutation |
| 1608 | TP53 | NM_000546 | 46115 | c.329G>A | p.R110H | Loss of Function | Missense_Mutation |
| 1609 | TP53 | NM_000546 | 44687 | c.379T>C | p.S127P | Loss of Function | Missense_Mutation |
| 1610 | TP53_ENST | ENST00000269305 | 99929 | c.329G>T | p.R110L | Loss of Function | Missense_Mutation |
| 1611 | TP53 | NM_000546 | 43970 | c.380C>A | p.S127Y | Loss of Function | Missense_Mutation |
| 1612 | TP53 | NM_000546 | 11508 | c.497C>A | p.S166* | Loss of Function | Nonsense_Mutation |
| 1613 | TP53 | NM_000546 | 44467 | c.497C>G | p.S166* | Loss of Function | Nonsense_Mutation |
| 1614 | TP53_ENST | ENST00000413465 | 99928 | c.329G>T | p.R110L | Loss of Function | Missense_Mutation |
| 1615 | TP53_ENST | ENST00000545858 | 242000 | c.359G>T | p.R120L | Loss of Function | Missense_Mutation |
| 1616 | TP53_ENST | ENST00000545858 | 99021 | c.464G>A | p.R155Q | Loss of Function | Missense_Mutation |
| 1617 | TP53 | NM_000546 | 10706 | c.548C>G | p.S183* | Loss of Function | Nonsense_Mutation |
| 1618 | TP53 | NM_000546 | 11717 | c.548C>A | p.S183* | Loss of Function | Nonsense_Mutation |
| 1619 | TP53_ENST | ENST00000545858 | 120006 | c.463C>T | p.R155W | Loss of Function | Missense_Mutation |
| 1620 | TP53 | NM_000546 | 46001 | c.466_486del21 | p.R156_1162delRVR | Loss of Function | In_Frame_Del |
| 1621 | TP53 | NM_000546 | 45314 | c.553delA | p.S185f5*62 | Loss of Function | Frame_Shift_Del |
| 1622 | TP53 | NM_000546 | 45154 | c.466C>G | p.R156G | Loss of Function | Missense_Mutation |
| 1623 | TP53 | NM_000546 | 43548 | c.467G>T | p.R156L | Loss of Function | Missense_Mutation |
| 1624 | TP53 | NM_000546 | 43744 | c.466C>A | p.R156S | Loss of Function | Missense_Mutation |
| 1625 | TP53 | NM_000546 | 44267 | c.472_477delCGCGCC | p.R158_A159delRA | Loss of Function | In_Frame_Del |
| 1626 | TP53 | NM_000546 | 44887 | c.644delG | p.S215f5*32 | Loss of Function | Frame_Shift_Del |
| 1627 | TP53 | NM_000546 | 43951 | c.643A>G | p.S215G | Loss of Function | Missense_Mutation |
| 1628 | TP53 | NM_000546 | 11450 | c.644G>T | p.S215I | Loss of Function | Missense_Mutation |
| 1629 | TP53_ENST | ENST00000269305 | 220779 | c.473G>A | p.R158H | Loss of Function | Missense_Mutation |
| 1630 | TP53 | NM_000546 | 44979 | c.645T>G | p.S215R | Loss of Function | Missense_Mutation |
| 1631 | TP53 | NM_000546 | 45122 | c.645T>A | p.S215R | Loss of Function | Missense_Mutation |
| 1632 | TP53 | NM_000546 | 46000 | c.643A>C | p.S215R | Loss of Function | Missense_Mutation |
| 1633 | TP53_ENST | ENST00000413465 | 220778 | c.473G>A | p.R158H | Loss of Function | Missense_Mutation |
| 1634 | TP53 | NM_000546 | 43615 | c.473G>C | p.R158P | Loss of Function | Missense_Mutation |
| 1635 | TP53 | NM_000546 | 44524 | c.521G>A | p.R174K | Loss of Function | Missense_Mutation |
| 1636 | TP53 | NM_000546 | 45671 | c.521G>T | p.R174M | Loss of Function | Missense_Mutation |
| 1637 | TP53 | NM_000546 | 44217 | c.718delA | p.S240f5*7 | Loss of Function | Frame_Shift_Del |
| 1638 | TP53 | NM_000546 | 43973 | c.718A>G | p.S240G | Loss of Function | Missense_Mutation |
| 1639 | TP53 | NM_000546 | 44518 | c.522G>A | p.R174R | Loss of Function | Synonymous_Mutation |
| 1640 | TP53 | NM_000546 | 44782 | c.520A>T | p.R174W | Loss of Function | Missense_Mutation |
| 1641 | TP53_ENST | ENST00000269305 | 99914 | c.524G>A | p.R175H | Loss of Function | Missense_Mutation |
| 1642 | TP53 | NM_000546 | 44838 | c.720T>C | p.S240S | Loss of Function | Synonymous_Mutation |
| 1643 | TP53_ENST | ENST00000413465 | 99022 | c.524G>A | p.R175H | Loss of Function | Missense_Mutation |
| 1644 | TP53 | NM_000546 | 10718 | c.524G>T | p.R175L | Loss of Function | Missense_Mutation |
| 1645 | TP53 | NM_000546 | 10709 | c.722C>G | p.S241C | Loss of Function | Missense_Mutation |
| 1646 | TP53 | NM_000546 | 45416 | c.524G>C | p.R175P | Loss of Function | Missense_Mutation |
| 1647 | TP53 | NM_000546 | 43931 | c.523C>A | p.R175S | Loss of Function | Missense_Mutation |
| 1648 | TP53 | NM_000546 | 10812 | c.722C>T | p.S241F | Loss of Function | Missense_Mutation |
| 1649 | TP53 | NM_000546 | 45017 | c.722_723CC>TT | p.S241F | Loss of Function | Missense_Mutation |
| 1650 | TP53 | NM_000546 | 43645 | c.721delT | p.S241f5*6 | Loss of Function | Frame_Shift_Del |
| 1651 | TP53 | NM_000546 | 44578 | c.721T>C | p.S241P | Loss of Function | Missense_Mutation |
| 1652 | TP53 | NM_000546 | 10738 | c.542G>A | p.R181H | Loss of Function | Missense_Mutation |
| 1653 | TP53 | NM_000546 | 10935 | c.722C>A | p.S241Y | Loss of Function | Missense_Mutation |
| 1654 | TP53 | NM_000546 | 44152 | c.542G>T | p.R181L | Loss of Function | Missense_Mutation |
| 1655 | TP53 | NM_000546 | 44599 | c.587G>A | p.R196Q | Loss of Function | Missense_Mutation |
| 1656 | TP53 | NM_000546 | 46074 | c.604C>T | p.R202C | Loss of Function | Missense_Mutation |
| 1657 | TP53 | NM_000546 | 43594 | c.605G>A | p.R202H | Loss of Function | Missense_Mutation |
| 1658 | TP53 | NM_000546 | 44925 | c.605G>T | p.R202L | Loss of Function | Missense_Mutation |
| 1659 | TP53 | NM_000546 | 43608 | c.605G>C | p.R202P | Loss of Function | Missense_Mutation |
| 1660 | TP53 | NM_000546 | 44074 | c.605_606GT>CG | p.R202P | Loss of Function | Missense_Mutation |
| 1661 | TP53 | NM_000546 | 44237 | c.904delG | p.S303f5*42 | Loss of Function | Frame_Shift_Del |
| 1662 | TP53 | NM_000546 | 44174 | c.604C>A | p.R202S | Loss of Function | Missense_Mutation |
| 1663 | TP53 | NM_000546 | 45995 | c.626G>A | p.R209K | Loss of Function | Missense_Mutation |
| 1664 | TP53 | NM_000546 | 45257 | c.626G>C | p.R209T | Loss of Function | Missense_Mutation |
| 1665 | TP53 | NM_000546 | 18610 | c.267delC | p.S90f5*33 | Loss of Function | Frame_Shift_Del |
| 1666 | TP53 | NM_000546 | 45500 | c.281C>A | p.S94* | Loss of Function | NonsenseMutation |
| 1667 | TP53_ENST | ENST00000269305 | 241998 | c.638G>T | p.R213L | Loss of Function | Missense_Mutation |
| 1668 | TP53_ENST | ENST00000413465 | 241997 | c.638G>T | p.R213L | Loss of Function | Missense_Mutation |
| 1669 | TP53_ENST | ENST00000269305 | 99602 | c.743G>A | p.R248Q | Loss of Function | Missense_Mutation |
| 1670 | TP53_ENST | ENST00000413465 | 99020 | c.743G>A | p.R248Q | Loss of Function | Missense_Mutation |
| 1671 | TP53 | NM_000546 | 85574 | c.291_295delCCCTT | p.S99fs*48 | Loss of Function | Frame_Shift_Del |
| 1672 | TP53 | NM_000546 | 44257 | c.301delA | p.T102fs*21 | Loss of Function | Frame_Shift_Del |
| 1673 | TP53 | NM_000546 | 44920 | c.742C>A | p.R248R | Loss of Function | Synonymous_Mutation |
| 1674 | TP53 | NM_000546 | 44303 | c.463A>G | p.T155A | Loss of Function | Missense_Mutation |
| 1675 | TP53 | NM_000546 | 44009 | c.463_470delACCCGCGT | p.T155fs*23 | Loss of Function | Frame_Shift_Del |
| 1676 | TP53 | NM_000546 | 44033 | c.464C>T | p.T155I | Loss of Function | Missense_Mutation |
| 1677 | TP53 | NM_000546 | 11218 | c.464C>A | p.T155N | Loss of Function | Missense_Mutation |
| 1678 | TP53 | NM_000546 | 10912 | c.463A>C | p.T155P | Loss of Function | Missense_Mutation |
| 1679 | TP53 | NM_000546 | 43670 | c.465C>T | p.T155T | Loss of Function | Synonymous_Mutation |
| 1680 | TP53 | NM_000546 | 45084 | c.744G>A | p.R248R | Loss of Function | Synonymous_Mutation |
| 1681 | TP53 | NM_000546 | 44384 | c.510G>A | p.T170T | Loss of Function | Synonymous_Mutation |
| 1682 | TP53 | NM_000546 | 45541 | c.510G>T | p.T170T | Loss of Function | Synonymous_Mutation |
| 1683 | TP53 | NM_000546 | 45735 | c.744G>C | p.R248R | Loss of Function | Synonymous_Mutation |
| 1684 | TP53 | NM_000546 | 44371 | c.631delA | p.T211fs*36 | Loss of Function | Frame_Shift_Del |
| 1685 | TP53 | NM_000546 | 43939 | c.632C>T | p.T211I | Loss of Function | Missense_Mutation |
| 1686 | TP53_ENST | ENST00000269305 | 120007 | c.742C>T | p.R248W | Loss of Function | Missense_Mutation |
| 1687 | TP53 | NM_000546 | 46211 | c.633T>C | p.T211T | Loss of Function | Synonymous_Mutation |
| 1688 | TP53 | NM_000546 | 45157 | c.688delA | p.T230fs*17 | Loss of Function | Frame_Shift_Del |
| 1689 | TP53 | NM_000546 | 44458 | c.688_698del11 | p.T230fs*6 | Loss of Function | Frame_Shift_Del |
| 1690 | TP53_ENST | ENST00000413465 | 120005 | c.742C>T | p.R248W | Loss of Function | Missense_Mutation |
| 1691 | TP53 | NM_000546 | 44625 | c.747G>A | p.R249R | Loss of Function | Synonymous_Mutation |
| 1692 | TP53 | NM_000546 | 44271 | c.688A>C | p.T230P | Loss of Function | Missense_Mutation |
| 1693 | TP53_ENST | ENST00000269305 | 131478 | c.747G>T | p.R249S | Loss of Function | Missense_Mutation |
| 1694 | TP53_ENST | ENST00000413465 | 131479 | c.747G>T | p.R249S | Loss of Function | Missense_Mutation |
| 1695 | TP53 | NM_000546 | 45784 | c.691delA | p.T231fs*16 | Loss of Function | Frame_Shift_Del |
| 1696 | TP53 | NM_000546 | 45706 | c.801G>T | p.R267R | Loss of Function | Synonymous_Mutation |
| 1697 | TP53_ENST | ENST00000269305 | 179804 | c.799C>T | p.R267W | Loss of Function | Missense_Mutation |
| 1698 | TP53 | NM_000546 | 44113 | c.693C>T | p.T231T | Loss of Function | Synonymous_Mutation |
| 1699 | TP53_ENST | ENST00000414315 | 220780 | c.77G>A | p.R26H | Loss of Function | Missense_Mutation |
| 1700 | TP53 | NM_000546 | 44460 | c.757_760delACCA | p.T253fs*91 | Loss of Function | Frame_Shift_Del |
| 1701 | TP53_ENST | ENST00000269305 | 99933 | c.817C>T | p.R273C | Loss of Function | Missense_Mutation |
| 1702 | TP53 | NM_000546 | 43843 | c.817C>G | p.R273G | Loss of Function | Missense_Mutation |
| 1703 | TP53_ENST | ENST00000269305 | 99729 | c.818G>A | p.R273H | Loss of Function | Missense_Mutation |
| 1704 | TP53 | NM_000546 | 44843 | c.759C>T | p.T253T | Loss of Function | Synonymous_Mutation |
| 1705 | TP53 | NM_000546 | 45843 | c.838_843delAGAGAC | p.R280_D281delRD | Loss of Function | In_Frame_Del |
| 1706 | TP53_ENST | ENST00000269305 | 129830 | c.839G>A | p.R280K | Loss of Function | Missense_Mutation |
| 1707 | TP53 | NM_000546 | 44470 | c.845G>T | p.R282L | Loss of Function | Missense_Mutation |
| 1708 | TP53 | NM_000546 | 44352 | c.850A>C | p.T284P | Loss of Function | Missense_Mutation |
| 1709 | TP53 | NM_000546 | 44835 | c.852A>T | p.T284T | Loss of Function | Synonymous_Mutation |
| 1710 | TP53 | NM_000546 | 44306 | c.845G>C | p.R282P | Loss of Function | Missense_Mutation |
| 1711 | TP53 | NM_000546 | 44417 | c.910delA | p.T304fs*41 | Loss of Function | Frame_Shift_Del |
| 1712 | TP53_ENST | ENST00000269305 | 99925 | c.844C>T | p.R282W | Loss of Function | Missense_Mutation |
| 1713 | TP53 | NM_000546 | 10911 | c.847C>T | p.R283C | Loss of Function | Missense_Mutation |
| 1714 | TP53 | NM_000546 | 46035 | c.847C>G | p.R283G | Loss of Function | Missense_Mutation |
| 1715 | TP53 | NM_000546 | 11483 | c.848G>A | p.R283H | Loss of Function | Missense_Mutation |
| 1716 | TP53 | NM_000546 | 10670 | c.469G>T | p.V157F | Loss of Function | Missense_Mutation |
| 1717 | TP53 | NM_000546 | 43710 | c.468delC | p.V157fs*13 | Loss of Function | Frame_Shift_Del |
| 1718 | TP53 | NM_000546 | 45111 | c.469_473delGTCCG | p.V157fs*22 | Loss of Function | Frame_Shift_Del |
| 1719 | TP53 | NM_000546 | 43903 | c.470T>G | p.V157G | Loss of Function | Missense_Mutation |
| 1720 | TP53 | NM_000546 | 44463 | c.848G>T | p.R283L | Loss of Function | Missense_Mutation |
| 1721 | TP53 | NM_000546 | 44017 | c.869G>A | p.R290H | Loss of Function | Missense_Mutation |
| 1722 | TP53 | NM_000546 | 43934 | c.471C>A | p.V157V | Loss of Function | Synonymous_Mutation |
| 1723 | TP53 | NM_000546 | 44526 | c.471C>T | p.V157V | Loss of Function | Synonymous_Mutation |
| 1724 | TP53 | NM_000546 | 44639 | c.869G>T | p.R290L | Loss of Function | Missense_Mutation |
| 1725 | TP53 | NM_000546 | 45278 | c.1025G>A | p.R342Q | Loss of Function | Missense_Mutation |
| 1726 | TP53 | NM_000546 | 44240 | c.514G>T | p.V172F | Loss of Function | Missense_Mutation |
| 1727 | TP53 | NM_000546 | 45906 | c.514delG | p.V172fs*2 | Loss of Function | Frame_Shift_Del |
| 1728 | TP53 | NM_000546 | 45047 | c.515T>G | p.V172G | Loss of Function | Missense_Mutation |
| 1729 | TP53_ENST | ENST00000414315 | 99023 | c.128G>A | p.R43H | Loss of Function | Missense_Mutation |
| 1730 | TP53 | NM_000546 | 44973 | c.516T>C | p.V172V | Loss of Function | Synonymous_Mutation |
| 1731 | TP53_ENST | ENST00000545858 | 220781 | c.194G>A | p.R65H | Loss of Function | Missense_Mutation |
| 1732 | TP53 | NM_000546 | 43732 | c.517delG | p.V173fs*1 | Loss of Function | Frame_Shift_Del |
| 1733 | TP53 | NM_000546 | 45583 | c.514_559del46 | p.V173fs*59 | Loss of Function | Frame_Shift_Del |
| 1734 | TP53 | NM_000546 | 43054 | c.518T>G | p.V173G | Loss of Function | Missense_Mutation |
| 1735 | TP53 | NM_000546 | 44383 | c.518T>G | p.V173G | Loss of Function | Missense_Mutation |
| 1736 | TP53 | NM_000546 | 43559 | c.517G>T | p.V173L | Loss of Function | Missense_Mutation |
| 1737 | TP53 | NM_000546 | 44057 | c.517G>C | p.V173L | Loss of Function | Missense_Mutation |
| 1738 | TP53 | NM_000546 | 11084 | c.517G>A | p.V173M | Loss of Function | Missense_Mutation |
| 1739 | TP53 | NM_000546 | 44517 | c.519G>A | p.V173V | Loss of Function | Synonymous_Mutation |
| 1740 | TP53 | NM_000546 | 44018 | c.214C>T | p.R72C | Loss of Function | Missense_Mutation |
| 1741 | TP53 | NM_000546 | 43905 | c.590T>G | p.V197G | Loss of Function | Missense_Mutation |
| 1742 | TP53 | NM_000546 | 45985 | c.215G>A | p.R72H | Loss of Function | Missense_Mutation |
| 1743 | TP53_ENST | ENST00000414315 | 241999 | c.242G>T | p.R81L | Loss of Function | Missense_Mutation |
| 1744 | TP53 | NM_000546 | 44845 | c.591G>A | p.V197V | Loss of Function | Synonymous_Mutation |
| 1745 | TP53_ENST | ENST00000545858 | 99024 | c.245G>A | p.R82H | Loss of Function | Missense_Mutation |
| 1746 | TP53 | NM_000546 | 45308 | c.607delG | p.V203fs*44 | Loss of Function | Frame_Shift_Del |
| 1747 | TP53 | NM_000546 | 44226 | c.380C>T | p.S127F | Loss of Function | Missense_Mutation |
| 1748 | TP53 | NM_000546 | 45015 | c.380_381CC>TT | p.S127F | Loss of Function | Missense_Mutation |
| 1749 | TP53 | NM_000546 | 44707 | c.609G>A | p.V203V | Loss of Function | Synonymous_Mutation |
| 1750 | TP53 | NM_000546 | 53285 | c.379T>A | p.S127T | Loss of Function | Missense_Mutation |
| 1751 | TP53 | NM_000546 | 44282 | c.496T>G | p.S166A | Loss of Function | Missense_Mutation |
| 1752 | TP53 | NM_000546 | 44274 | c.647T>A | p.V216E | Loss of Function | Missense_Mutation |
| 1753 | TP53 | NM_000546 | 44239 | c.647delT | p.V216fs*31 | Loss of Function | Frame_Shift_Del |
| 1754 | TP53 | NM_000546 | 43681 | c.647T>G | p.V216G | Loss of Function | Missense_Mutation |
| 1755 | TP53 | NM_000546 | 11210 | c.646G>T | p.V216L | Loss of Function | Missense_Mutation |
| 1756 | TP53 | NM_000546 | 10667 | c.646G>A | p.V216M | Loss of Function | Missense_Mutation |
| 1757 | TP53 | NM_000546 | 44289 | c.497C>T | p.S166L | Loss of Function | Missense_Mutation |
| 1758 | TP53 | NM_000546 | 44035 | c.496T>C | p.S166P | Loss of Function | Missense_Mutation |
| 1759 | TP53 | NM_000546 | 44300 | c.548C>T | p.S183L | Loss of Function | Missense_Mutation |
| 1760 | TP53 | NM_000546 | 44343 | c.547T>C | p.S183P | Loss of Function | Missense_Mutation |
| 1761 | TP53 | NM_000546 | 44714 | c.553A>G | p.S185G | Loss of Function | Missense_Mutation |
| 1762 | TP53 | NM_000546 | 44185 | c.555C>A | p.S185R | Loss of Function | Missense_Mutation |
| 1763 | TP53 | NM_000546 | 45198 | c.555C>T | p.S185S | Loss of Function | Missense_Mutation |
| 1764 | TP53 | NM_000546 | 44198 | c.653T>G | p.V218G | Loss of Function | Missense_Mutation |
| 1765 | TP53 | NM_000546 | 11307 | c.643A>T | p.S215C | Loss of Function | Missense_Mutation |
| 1766 | TP53 | NM_000546 | 44093 | c.644G>A | p.S215N | Loss of Function | Missense_Mutation |
| 1767 | TP53 | NM_000546 | 45511 | c.645T>C | p.S215S | Loss of Function | Missense_Mutation |
| 1768 | TP53 | NM_000546 | 13421 | c.814delG | p.V272fs*73 | Loss of Function | Frame_Shift_Del |
| 1769 | TP53 | NM_000546 | 44870 | c.815T>G | p.V272G | Loss of Function | Missense_Mutation |
| 1770 | TP53 | NM_000546 | 44175 | c.644G>C | p.S215T | Loss of Function | Missense_Mutation |
| 1771 | TP53 | NM_000546 | 43920 | c.680C>T | p.S227F | Loss of Function | Missense_Mutation |
| 1772 | TP53 | NM_000546 | 10891 | c.814G>A | p.V272M | Loss of Function | Missense_Mutation |
| 1773 | TP53 | NM_000546 | 44393 | c.821T>C | p.V274A | Loss of Function | Missense_Mutation |
| 1774 | TP53 | NM_000546 | 44448 | c.821T>A | p.V274D | Loss of Function | Missense_Mutation |
| 1775 | TP53 | NM_000546 | 10769 | c.820G>T | p.V274F | Loss of Function | Missense_Mutation |
| 1776 | TP53 | NM_000546 | 43945 | c.821T>G | p.V274G | Loss of Function | Missense_Mutation |
| 1777 | TP53 | NM_000546 | 44621 | c.718A>T | p.S240C | Loss of Function | Missense_Mutation |
| 1778 | TP53 | NM_000546 | 44443 | c.820G>C | p.V274L | Loss of Function | Missense_Mutation |
| 1779 | TP53 | NM_000546 | 45491 | c.822T>G | p.V274V | Loss of Function | Synonymous_Mutation |
| 1780 | TP53 | NM_000546 | 43660 | c.719G>T | p.S2401 | Loss of Function | Missense_Mutation |
| 1781 | TP53 | NM_000546 | 43684 | c.720T>G | p.S240R | Loss of Function | Missense_Mutation |
| 1782 | TP53 | NM_000546 | 44192 | c.272G>A | p.W91* | Loss of Function | Nonsense_Mutation |
| 1783 | TP53 | NM_000546 | 44492 | c.273G>A | p.W91* | Loss of Function | Nonsense_Mutation |
| 1784 | TP53 | NM_000546 | 44453 | c.309C>G | p.Y103* | Loss of Function | Nonsense_Mutation |
| 1785 | TP53 | NM_000546 | 11448 | c.321C>G | p.Y107* | Loss of Function | Nonsense_Mutation |
| 1786 | TP53 | NM_000546 | 45040 | c.321C>A | p.Y107* | Loss of Function | Nonsense_Mutation |
| 1787 | TP53 | NM_000546 | 46103 | c.319T>G | p.Y107D | Loss of Function | Missense_Mutation |
| 1788 | TP53 | NM_000546 | 45509 | c.321C>T | p.Y107Y | Loss of Function | Synonymous_Mutation |
| 1789 | TP53 | NM_000546 | 10862 | c.378C>G | p.Y126* | Loss of Function | Nonsense_Mutation |
| 1790 | TP53 | NM_000546 | 10888 | c.378C>A | p.Y126* | Loss of Function | Nonsense_Mutation |
| 1791 | TP53 | NM_000546 | 45261 | c.720T>A | p.S240R | Loss of Function | Missense_Mutation |
| 1792 | TP53 | NM_000546 | 44964 | c.719G>C | p.S240T | Loss of Function | Missense_Mutation |
| 1793 | TP53 | NM_000546 | 11517 | c.377A>G | p.Y126C | Loss of Function | Splice_Site |
| 1794 | TP53 | NM_000546 | 43900 | c.376T>G | p.Y126D | Loss of Function | Splice_Site |
| 1795 | TP53 | NM_000546 | 249845 | c.377_377delA | p.Y126fs*44 | Loss of Function | Frame_Shift_Del |
| 1796 | TP53 | NM_000546 | 44380 | c.376T>A | p.Y126N | Loss of Function | Splice_Site |
| 1797 | TP53 | NM_000546 | 44142 | c.377A>C | p.Y126S | Loss of Function | Splice_Site |
| 1798 | TP53 | NM_000546 | 43820 | c.489C>G | p.Y163* | Loss of Function | Nonsense_Mutation |
| 1799 | TP53 | NM_000546 | 45411 | c.489C>A | p.Y163* | Loss of Function | Nonsense_Mutation |
| 1800 | TP53 | NM_000546 | 10808 | c.488A>G | p.Y163C | Loss of Function | Missense_Mutation |
| 1801 | TP53 | NM_000546 | 44216 | c.487T>G | p.Y163D | Loss of Function | Missense_Mutation |
| 1802 | TP53 | NM_000546 | 45194 | c.487delT | p.Y163fs*7 | Loss of Function | Frame_Shift_Del |
| 1803 | TP53 | NM_000546 | 43846 | c.487T>C | p.Y163H | Loss of Function | Missense_Mutation |
| 1804 | TP53 | NM_000546 | 44623 | c.487T>A | p.Y163N | Loss of Function | Missense_Mutation |
| 1805 | TP53 | NM_000546 | 44224 | c.721T>G | p.S241A | Loss of Function | Missense_Mutation |
| 1806 | TP53 | NM_000546 | 44391 | c.489C>T | p.Y163Y | Loss of Function | Synonymous_Mutation |
| 1807 | TP53 | NM_000546 | 43928 | c.615T>A | p.Y205* | Loss of Function | Nonsense_Mutation |
| 1808 | TP53 | NM_000546 | 44924 | c.615T>G | p.Y205* | Loss of Function | Nonsense_Mutation |
| 1809 | TP53 | NM_000546 | 43947 | c.614A>G | p.Y205C | Loss of Function | Missense_Mutation |
| 1810 | TP53 | NM_000546 | 45168 | c.722_724delCCT | p.S241del | Loss of Function | In_Frame_Del |
| 1811 | TP53 | NM_000546 | 45548 | c.721_723delTCC | p.S241del | Loss of Function | In_Frame_Del |
| 1812 | TP53 | NM_000546 | 44067 | c.721T>A | p.S241T | Loss of Function | Missense_Mutation |
| 1813 | TP53 | NM_000546 | 45685 | c.613T>A | p.Y205N | Loss of Function | Missense_Mutation |
| 1814 | TP53 | NM_000546 | 146240 | c.806_808delGCT | p.S269_F270>I | Loss of Function | In_Frame_Del |
| 1815 | TP53 | NM_000546 | 44505 | c.660T>G | p.Y220* | Loss of Function | Nonsense_Mutation |
| 1816 | TP53 | NM_000546 | 10758 | c.659A>G | p.Y220C | Loss of Function | Missense_Mutation |
| 1817 | TP53 | NM_000546 | 45248 | c.805A>T | p.S269C | Loss of Function | Missense_Mutation |
| 1818 | TP53 | NM_000546 | 44585 | c.655delC | p.Y220fs*27 | Loss of Function | Frame_Shift_Del |
| 1819 | TP53 | NM_000546 | 44637 | c.658T>C | p.Y220H | Loss of Function | Missense_Mutation |
| 1820 | TP53 | NM_000546 | 43962 | c.805A>G | p.S269G | Loss of Function | Missense_Mutation |
| 1821 | TP53 | NM_000546 | 43850 | c.659A>C | p.Y220S | Loss of Function | Missense_Mutation |
| 1822 | TP53 | NM_000546 | 45114 | c.702C>A | p.Y234* | Loss of Function | Nonsense_Mutation |
| 1823 | TP53 | NM_000546 | 10725 | c.701A>G | p.Y234C | Loss of Function | Missense_Mutation |
| 1824 | TP53 | NM_000546 | 44236 | c.806G>A | p.S269N | Loss of Function | Missense_Mutation |
| 1825 | TP53 | NM_000546 | 44886 | c.807C>T | p.S269S | Loss of Function | Missense_Mutation |
| 1826 | TP53 | NM_000546 | 45507 | c.806G>C | p.S269T | Loss of Function | Missense_Mutation |
| 1827 | TP53 | NM_000546 | 43956 | c.700T>A | p.Y234N | Loss of Function | Missense_Mutation |
| 1828 | TP53 | NM_000546 | 43865 | c.701A>C | p.Y234S | Loss of Function | Missense_Mutation |
| 1829 | TP53 | NM_000546 | 43564 | c.708C>A | p.Y236* | Loss of Function | Nonsense_Mutation |
| 1830 | TP53 | NM_000546 | 44960 | c.708C>G | p.Y236* | Loss of Function | Nonsense_Mutation |
| 1831 | TP53 | NM_000546 | 10731 | c.707A>G | p.Y236C | Loss of Function | Missense_Mutation |
| 1832 | TP53 | NM_000546 | 43602 | c.706T>G | p.Y236D | Loss of Function | Missense_Mutation |
| 1833 | TP53 | NM_000546 | 44565 | c.907A>T | p.S303C | Loss of Function | Missense_Mutation |
| 1834 | TP53 | NM_000546 | 43986 | c.908G>A | p.S303N | Loss of Function | Missense_Mutation |
| 1835 | TP53 | NM_000546 | 43826 | c.706T>A | p.Y236N | Loss of Function | Missense_Mutation |
| 1836 | TP53 | NM_000546 | 44167 | c.908G>C | p.S303T | Loss of Function | Missense_Mutation |
| 1837 | TP53 | NM_000546 | 44132 | c.708C>T | p.Y236Y | Loss of Function | Synonymous_Mutation |
| 1838 | TP53_ENST | ENST00000269305 | 131534 | c.559+1G>A | p.? | Loss of Function | N/A |
| 1839 | TP53 | NM_000546 | 44832 | c.1096T>G | p.S366A | Loss of Function | Missense_Mutation |
| 1840 | TP53_ENST | ENST00000269305 | 179823 | c.528C>A | p.C176* | Loss of Function | Nonsense_Mutation |
| 1841 | TP53 | NM_000546 | 44048 | c.280T>A | p.S94T | Loss of Function | Missense_Mutation |
| 1842 | TP53 | NM_000546 | 44673 | c.284C>T | p.S95F | Loss of Function | Missense_Mutation |
| 1843 | TP53 | NM_000546 | 44447 | c.287C>T | p.S96F | Loss of Function | Missense_Mutation |
| 1844 | TP53 | NM_000546 | 44036 | c.296C>T | p.S99F | Loss of Function | Missense_Mutation |
| 1845 | TP53_ENST | ENST00000269305 | 118013 | c.592G>T | p.E198* | Loss of Function | Nonsense_Mutation |
| 1846 | TP53 | NM_000546 | 43678 | c.305C>T | p.T102I | Loss of Function | Missense_Mutation |
| 1847 | TP53 | NM_000546 | 44552 | c.509C>T | p.T170M | Loss of Function | Missense_Mutation |
| 1848 | TP53_ENST | ENST00000269305 | 126981 | c.880G>T | p.E294* | Loss of Function | Nonsense_Mutation |
| 1849 | TP53 | NM_000546 | 44238 | c.631A>G | p.T211A | Loss of Function | Missense_Mutation |
| 1850 | TP53 | NM_000546 | 44661 | c.632C>A | p.T211N | Loss of Function | Missense_Mutation |
| 1851 | TP53 | NM_000546 | 43868 | c.689C>T | p.T230I | Loss of Function | Missense_Mutation |
| 1852 | TP53_ENST | ENST00000269305 | 111498 | c.532delC | p.H178fs*69 | Loss of Function | Frame_Shift_Del |
| 1853 | TP53 | NM_000546 | 43806 | c.689C>A | p.T230N | Loss of Function | Missense_Mutation |
| 1854 | TP53 | NM_000546 | 45631 | c.688A>T | p.T230S | Loss of Function | Missense_Mutation |
| 1855 | TP53 | NM_000546 | 43980 | c.691A>G | p.T231A | Loss of Function | Missense_Mutation |
| 1856 | TP53 | NM_000546 | 44820 | c.692C>T | p.T231I | Loss of Function | Missense_Mutation |
| 1857 | TP53 | NM_000546 | 43889 | c.691A>T | p.T231S | Loss of Function | Missense_Mutation |
| 1858 | TP53 | NM_000546 | 45322 | c.757A>G | p.T253A | Loss of Function | Missense_Mutation |
| 1859 | TP53 | NM_000546 | 43683 | c.758C>T | p.T253I | Loss of Function | Missense_Mutation |
| 1860 | TP53 | NM_000546 | 45980 | c.757A>C | p.T253P | Loss of Function | Missense_Mutation |
| 1861 | TP53_ENST | ENST00000269305 | 117949 | c.574C>T | p.Q192* | Loss of Function | Nonsense_Mutation |
| 1862 | TP53 | NM_000546 | 43881 | c.757A>T | p.T253S | Loss of Function | Missense_Mutation |
| 1863 | TP53 | NM_000546 | 44544 | c.766A>G | p.T256A | Loss of Function | Missense_Mutation |
| 1864 | TP53 | NM_000546 | 44662 | c.766A>T | p.T256S | Loss of Function | Missense_Mutation |
| 1865 | TP53_ENST | ENST00000269305 | 99668 | c.586C>T | p.R196* | Loss of Function | Nonsense_Mutation |
| 1866 | TP53_ENST | ENST00000269305 | 99618 | c.637C>T | p.R213* | Loss of Function | Nonsense_Mutation |
| 1867 | TP53 | NM_000546 | 45728 | c.850A>G | p.T284A | Loss of Function | Missense_Mutation |
| 1868 | TP53 | NM_000546 | 46207 | c.910A>G | p.T304A | Loss of Function | Missense_Mutation |
| 1869 | TP53 | NM_000546 | 45128 | c.911C>T | p.T304I | Loss of Function | Missense_Mutation |
| 1870 | TP53 | NM_000546 | 44200 | c.242C>T | p.T81I | Loss of Function | Missense_Mutation |
| 1871 | TP53 | NM_000546 | 44329 | c.470T>A | p.V157D | Loss of Function | Missense_Mutation |
| 1872 | TP53 | NM_000546 | 45551 | c.469_471delGTC | p.V157del | Loss of Function | In_Frame_Del |
| 1873 | TP53_ENST | ENST00000269305 | 131480 | c.469G>T | p.V157F | Loss of Function | Missense_Mutation |
| 1874 | TP53_ENST | ENST00000413465 | 131481 | c.469G>T | p.V157F | Loss of Function | Missense_Mutation |
| 1875 | TP53 | NM_000546 | 43625 | c.469G>A | p.V157I | Loss of Function | Missense_Mutation |
| 1876 | TP53_ENST | ENST00000269305 | 99947 | c.916C>T | p.R306* | Loss of Function | Nonsense_Mutation |
| 1877 | TP53_ENST | ENST00000269305 | 99721 | c.1024C>T | p.R342* | Loss of Function | Nonsense_Mutation |
| 1878 | TP53 | NM_000546 | 45120 | c.469G>C | p.V157L | Loss of Function | Missense_Mutation |
| 1879 | TP53 | NM_000546 | 44996 | c.515T>C | p.V172A | Loss of Function | Missense_Mutation |
| 1880 | TP53 | NM_000546 | 44229 | c.515T>A | p.V172D | Loss of Function | Missense_Mutation |
| 1881 | TP53 | NM_000546 | 43955 | c.514G>A | p.V172I | Loss of Function | Missense_Mutation |
| 1882 | TP53 | NM_000546 | 44327 | c.518T>C | p.V173A | Loss of Function | Missense_Mutation |
| 1883 | TP53_ENST | ENST00000269305 | 121042 | c.517G>C | p.V173L | Loss of Function | Missense_Mutation |
| 1884 | TP53_ENST | ENST00000269305 | 99946 | c.378C>G | p.Y126* | Loss of Function | Nonsense_Mutation |
| 1885 | TP53_ENST | ENST00000269305 | 99641 | c.517G>T | p.V173L | Loss of Function | Missense_Mutation |
| 1886 | TP53_ENST | ENST00000413465 | 121043 | c.517G>C | p.V173L | Loss of Function | Missense_Mutation |
| 1887 | TP53_ENST | ENST00000413465 | 99638 | c.517G>T | p.V173L | Loss of Function | Missense_Mutation |
| 1888 | TP53_ENST | ENST00000413465 | 98964 | c.517G>A | p.V173M | Loss of Function | Missense_Mutation |
| 1889 | TP53 | NM_000546 | 44424 | c.590T>A | p.V197E | Loss of Function | Missense_Mutation |
| 1890 | TP53_ENST | ENST00000413465 | 131535 | c.559+1G>A | p.? | Loss of Function | N/A |
| 1891 | TP53 | NM_000546 | 46212 | c.589G>T | p.V197L | Loss of Function | Missense_Mutation |
| 1892 | TP53_ENST | ENST00000413465 | 179822 | c.528C>A | p.C176* | Loss of Function | Nonsense_Mutation |
| 1893 | TP53 | NM_000546 | 43779 | c.589G>A | p.V197M | Loss of Function | Missense_Mutation |
| 1894 | TP53 | NM_000546 | 44411 | c.608T>A | p.V203E | Loss of Function | Missense_Mutation |
| 1895 | TP53_ENST | ENST00000413465 | 118010 | c.592G>T | p.E198* | Loss of Function | Nonsense_Mutation |
| 1896 | TP53 | NM_000546 | 44365 | c.607G>T | p.V203L | Loss of Function | Missense_Mutation |
| 1897 | TP53 | NM_000546 | 43599 | c.607G>A | p.V203M | Loss of Function | Missense_Mutation |
| 1898 | TP53_ENST | ENST00000413465 | 111495 | c.532delC | p.H178fs*69 | Loss of Function | Frame_Shift_Del |
| 1899 | TP53 | NM_000546 | 44567 | c.647T>C | p.V216A | Loss of Function | Missense_Mutation |
| 1900 | TP53 | NM_000546 | 44607 | c.646_648delGTG | p.V216del | Loss of Function | In_Frame_Del |
| 1901 | TP53 | NM_000546 | 45110 | c.650T>C | p.V217A | Loss of Function | Missense_Mutation |
| 1902 | TP53 | NM_000546 | 44929 | c.650T>A | p.V217E | Loss of Function | Missense_Mutation |
| 1903 | TP53 | NM_000546 | 44375 | c.650T>G | p.V217G | Loss of Function | Missense_Mutation |
| 1904 | TP53_ENST | ENST00000413465 | 117946 | c.574C>T | p.Q192* | Loss of Function | Nonsense_Mutation |
| 1905 | TP53 | NM_000546 | 44334 | c.649G>T | p.V217L | Loss of Function | Missense_Mutation |
| 1906 | TP53 | NM_000546 | 44930 | c.653T>C | p.V218A | Loss of Function | Missense_Mutation |
| 1907 | TP53 | NM_000546 | 6496 | c.652_654delGTG | p.V218del | Loss of Function | In_Frame_Del |
| 1908 | TP53_ENST | ENST00000413465 | 99665 | c.586C>T | p.R196* | Loss of Function | Nonsense_Mutation |
| 1909 | TP53_ENST | ENST00000413465 | 99615 | c.637C>T | p.R213* | Loss of Function | Nonsense_Mutation |
| 1910 | TP53 | NM_000546 | 44317 | c.653T>A | p.V218E | Loss of Function | Missense_Mutation |
| 1911 | TP53 | NM_000546 | 44683 | c.652G>A | p.V218M | Loss of Function | Missense_Mutation |
| 1912 | TP53_ENST | ENST00000414315 | 131483 | c.73G>T | p.V25F | Loss of Function | Missense_Mutation |
| 1913 | TP53 | NM_000546 | 44294 | c.815T>C | p.V272A | Loss of Function | Missense_Mutation |
| 1914 | TP53 | NM_000546 | 44580 | c.815T>A | p.V272E | Loss of Function | Missense_Mutation |
| 1915 | TP53 | NM_000546 | 10859 | c.814G>T | p.V272L | Loss of Function | Missense_Mutation |
| 1916 | TP53 | NM_000546 | 45898 | c.814G>C | p.V272L | Loss of Function | Missense_Mutation |
| 1917 | TP53_ENST | ENST00000269305 | 99950 | c.814G>A | p.V272M | Loss of Function | Missense_Mutation |
| 1918 | TP53_ENST | ENST00000269305 | 165075 | c.820G>T | p.V274F | Loss of Function | Missense_Mutation |
| 1919 | TP53_ENST | ENST00000413465 | 99944 | c.378C>G | p.Y126* | Loss of Function | Nonsense_Mutation |
| 1920 | TP53 | NM_000546 | 43667 | c.820G>A | p.V274I | Loss of Function | Missense_Mutation |
| 1921 | TP53_ENST | ENST00000414315 | 121045 | c.121G>C | p.V41L | Loss of Function | Missense_Mutation |
| 1922 | TP53_ENST | ENST00000414315 | 99639 | c.121G>T | p.V41L | Loss of Function | Missense_Mutation |
| 1923 | TP53_ENST | ENST00000414315 | 98965 | c.121G>A | p.V41M | Loss of Function | Missense_Mutation |
| 1924 | TP53_ENST | ENST00000545858 | 131482 | c.190G>T | p.V64F | Loss of Function | Missense_Mutation |
| 1925 | TP53_ENST | ENST00000414315 | 131537 | c.163+1G>A | p.? | Loss of Function | N/A |
| 1926 | TP53 | NM_000546 | 45288 | c.217G>C | p.V73L | Loss of Function | Missense_Mutation |
| 1927 | TP53_ENST | ENST00000414315 | 179824 | c.132C>A | p.C44* | Loss of Function | Nonsense_Mutation |
| 1928 | TP53 | NM_000546 | 43787 | c.217G>A | p.V73M | Loss of Function | Missense_Mutation |
| 1929 | TP53_ENST | ENST00000414315 | 118011 | c.196G>T | p.E66* | Loss of Function | Nonsense_Mutation |
| 1930 | TP53_ENST | ENST00000414315 | 111496 | c.136delC | p.H46fs*>45 | Loss of Function | Frame_Shift_Del |
| 1931 | TP53_ENST | ENST00000545858 | 121044 | c.238G>C | p.V80L | Loss of Function | Missense_Mutation |
| 1932 | TP53_ENST | ENST00000545858 | 99640 | c.238G>T | p.V80L | Loss of Function | Missense_Mutation |
| 1933 | TP53_ENST | ENST00000545858 | 98966 | c.238G>A | p.V80M | Loss of Function | Missense_Mutation |
| 1934 | TP53_ENST | ENST00000269305 | 220766 | c.319T>G | p.Y107D | Loss of Function | Missense_Mutation |
| 1935 | TP53_ENST | ENST00000414315 | 117947 | c.178C>T | p.Q60* | Loss of Function | Nonsense_Mutation |
| 1936 | TP53_ENST | ENST00000413465 | 220765 | c.319T>G | p.Y107D | Loss of Function | Missense_Mutation |
| 1937 | TP53 | NM_000546 | 44405 | c.376_396del21 | p.Y126_K132delYSP | Loss of Function | In_Frame_Del |
| 1938 | TP53_ENST | ENST00000414315 | 99666 | c.190C>T | p.R64* | Loss of Function | Nonsense_Mutation |
| 1939 | TP53_ENST | ENST00000414315 | 99616 | c.241C>T | p.R81* | Loss of Function | Nonsense_Mutation |
| 1940 | TP53 | NM_000546 | 44774 | c.376_393del18 | p.Y126_N131delYSP | Loss of Function | In_Frame_Del |
| 1941 | TP53_ENST | ENST00000269305 | 220783 | c.376T>G | p.Y126D | Loss of Function | Missense_Mutation |
| 1942 | TP53_ENST | ENST00000413465 | 220782 | c.376T>G | p.Y126D | Loss of Function | Missense_Mutation |
| 1943 | TP53_ENST | ENST00000545858 | 99719 | c.380A>G | p.Y127C | Loss of Function | Missense_Mutation |
| 1944 | TP53_ENST | ENST00000545858 | 165074 | c.422A>G | p.Y141C | Loss of Function | Missense_Mutation |
| 1945 | TP53_ENST | ENST00000545858 | 116673 | c.428A>G | p.Y143C | Loss of Function | Missense_Mutation |
| 1946 | TP53_ENST | ENST00000545858 | 131536 | c.280+1G>A | p.? | Loss of Function | N/A |
| 1947 | TP53_ENST | ENST00000269305 | 129852 | c.488A>G | p.Y163C | Loss of Function | Missense_Mutation |
| 1948 | TP53_ENST | ENST00000413465 | 129853 | c.488A>G | p.Y163C | Loss of Function | Missense_Mutation |
| 1949 | TP53_ENST | ENST00000545858 | 179825 | c.249C>A | p.C83* | Loss of Function | Nonsense_Mutation |
| 1950 | TP53 | NM_000546 | 45025 | c.488A>C | p.Y163S | Loss of Function | Missense_Mutation |
| 1951 | TP53_ENST | ENST00000545858 | 118012 | c.313G>T | p.E105* | Loss of Function | Nonsense_Mutation |
| 1952 | TP53 | NM_000546 | 43844 | c.613T>G | p.Y205D | Loss of Function | Missense_Mutation |
| 1953 | TP53 | NM_000546 | 11351 | c.614A>T | p.Y205F | Loss of Function | Missense_Mutation |
| 1954 | TP53 | NM_000546 | 43642 | c.613T>C | p.Y205H | Loss of Function | Missense_Mutation |
| 1955 | TP53_ENST | ENST00000545858 | 111497 | c.253delC | p.H85fs*69 | Loss of Function | Frame_Shift_Del |
| 1956 | TP53 | NM_000546 | 44169 | c.614A>C | p.Y205S | Loss of Function | Missense_Mutation |
| 1957 | TP53_ENST | ENST00000269305 | 99720 | c.659A>G | p.Y220C | Loss of Function | Missense_Mutation |
| 1958 | TP53_ENST | ENST00000413465 | 99718 | c.659A>G | p.Y220C | Loss of Function | Missense_Mutation |
| 1959 | TP53 | NM_000546 | 11847 | c.658T>G | p.Y220D | Loss of Function | Missense_Mutation |
| 1960 | TP53_ENST | ENST00000545858 | 117948 | c.295C>T | p.Q99* | Loss of Function | Nonsense_Mutation |
| 1961 | TP53_ENST | ENST00000545858 | 99667 | c.307C>T | p.R103* | Loss of Function | Nonsense_Mutation |
| 1962 | TP53_ENST | ENST00000545858 | 99617 | c.358C>T | p.R120* | Loss of Function | Nonsense_Mutation |
| 1963 | TP53 | NM_000546 | 44672 | c.658T>A | p.Y220N | Loss of Function | Missense_Mutation |
| 1964 | TP53_ENST | ENST00000269305 | 165073 | c.701A>G | p.Y234C | Loss of Function | Missense_Mutation |
| 1965 | TP53_ENST | ENST00000413465 | 165072 | c.701A>G | p.Y234C | Loss of Function | Missense_Mutation |
| 1966 | TP53 | NM_000546 | 43768 | c.700T>G | p.Y234D | Loss of Function | Missense_Mutation |
| 1967 | TP53 | NM_000546 | 44953 | c.700_702delTAC | p.Y234del | Loss of Function | In_Frame_Del |
| 1968 | TP53 | NM_000546 | 11152 | c.700T>C | p.Y234H | Loss of Function | Missense_Mutation |
| 1969 | TP53_ENST | ENST00000269305 | 116674 | c.707A>G | p.Y236C | Loss of Function | Missense_Mutation |
| 1970 | TP53_ENST | ENST00000413465 | 116672 | c.707A>G | p.Y236C | Loss of Function | Missense_Mutation |
| 1971 | TP53 | NM_000546 | 44072 | c.706_708delTAC | p.Y236del | Loss of Function | In_Frame_Del |
| 1972 | TP53 | NM_000546 | 44326 | c.706T>C | p.Y236H | Loss of Function | Missense_Mutation |
| 1973 | TP53 | NM_000546 | 44693 | c.707A>C | p.Y236S | Loss of Function | Missense_Mutation |
| 1974 | TP53_ENST | ENST00000414315 | 129855 | c.92A>G | p.Y31C | Loss of Function | Missense_Mutation |
| 1975 | TP53_ENST | ENST00000545858 | 99945 | c.99C>G | p.Y33* | Loss of Function | Nonsense_Mutation |
| 1976 | TP53_ENST | ENST00000545858 | 220784 | c.97T>G | p.Y33D | Loss of Function | Missense_Mutation |
| 1977 | TP53_ENST | ENST00000545858 | 129854 | c.209A>G | p.Y70C | Loss of Function | Missense__Mutation |

TABLE 19

| | Driver | | | | | |
|---|---|---|---|---|---|---|
| | Gene | 5′ Gene Symbol | 3′ Gene Symbol | 5′ Entrez Id | 3′ Entrez Id | Source |
| 1 | ABL1 | BCR | ABL1 | 613 | 25 | 11289094, 21435002, ngs |
| 2 | ABL1 | BCR | ABL1 | 613 | 25 | 11289094, 21435002, ngs |
| 3 | AKT3 | MAGI3 | AKT3 | 260425 | 10000 | Banerji et al 2012, Nature |
| 4 | ALK | EML4 | ALK | 27436 | 238 | ngs |
| 5 | ALK | EML4 | ALK | 27436 | 238 | ngs |
| 6 | ALK | EML4 | ALK | 27436 | 238 | literature |
| 7 | ALK | EML4 | ALK | 27436 | 238 | literature |
| 8 | ALK | EML4 | ALK | 27436 | 238 | literature |
| 9 | ALK | EML4 | ALK | 27436 | 238 | |
| 10 | ALK | EML4 | ALK | 27436 | 238 | OncoNetwork |
| 11 | ALK | EML4 | ALK | 27436 | 238 | OncoNetwork |
| 12 | ALK | EML4 | ALK | 27436 | 238 | OncoNetwork |
| 13 | ALK | EML4 | ALK | 27436 | 238 | OncoNetwork; ngs |
| 14 | ALK | EML4 | ALK | 27436 | 238 | OncoNetwork; ngs |
| 15 | ALK | EML4 | ALK | 27436 | 238 | OncoNetwork |
| 16 | ALK | EML4 | ALK | 27436 | 238 | OncoNetwork |
| 17 | ALK | EML4 | ALK | 27436 | 238 | OncoNetwork |
| 18 | ALK | EML4 | ALK | 27436 | 238 | OncoNetwork |
| 19 | ALK | EML4 | ALK | 27436 | 238 | OncoNetwork |
| 20 | ALK | EML4 | ALK | 27436 | 238 | OncoNetwork |
| 21 | ALK | EML4 | ALK | 27436 | 238 | OncoNetwork |
| 22 | ALK | EML4 | ALK | 27436 | 238 | OncoNetwork |
| 23 | ALK | KIF5B | ALK | 3799 | 238 | OncoNetwork |
| 24 | ALK | KIF5B | ALK | 3799 | 238 | OncoNetwork |
| 25 | ALK | KIF5B | ALK | 3799 | 238 | OncoNetwork |
| 26 | ALK | KLC1 | ALK | 3831 | 238 | cosmic |
| 27 | ALK | TFG | ALK | 10342 | 238 | cosmic |
| 28 | ALK | TFG | ALK | 10342 | 238 | cosmic |
| 29 | ALK | TFG | ALK | 10342 | 238 | cosmic |
| 30 | ALK | ALK | PTPN3 | 238 | 5774 | Jung et al 2012, Genes Chromosomes Cance |
| 31 | BRAF | AGTRAP | BRAF | 57085 | 673 | cosmic |
| 32 | BRAF | AKAP9 | BRAF | 10142 | 673 | AY803272.1 |
| 33 | BRAF | SLC45A3 | BRAF | 85414 | 673 | cosmic |
| 34 | CDK4 | CDK4 | UBA1 | 1019 | 7317 | Asmann et al. 2012 Cancer Research |
| 35 | ERBB2 | WIPF2 | ERBB2 | 147179 | 2064 | Asmann et al. 2011 Nucleic Acids Research |
| 36 | ERG | TMPRSS2 | ERG | 7113 | 2078 | cosmic; ngs |
| 37 | ERG | TMPRSS2 | ERG | 7113 | 2078 | ngs |
| 38 | ERG | TMPRSS2 | ERG | 7113 | 2078 | cosmic |
| 39 | ERG | TMPRSS2 | ERG | 7113 | 2078 | ngs |
| 40 | ERG | TMPRSS2 | ERG | 7113 | 2078 | cosmic; ngs |
| 41 | ERG | TMPRSS2 | ERG | 7113 | 2078 | cosmic; ngs |
| 42 | ERG | TMPRSS2 | ERG | 7113 | 2078 | cosmic; ngs |
| 43 | ERG | TMPRSS2 | ERG | 7113 | 2078 | ngs |
| 44 | ERG | TMPRSS2 | ERG | 7113 | 2078 | cosmic; ngs |
| 45 | ERG | TMPRSS2 | ERG | 7113 | 2078 | cosmic |
| 46 | ERG | TMPRSS2 | ERG | 7113 | 2078 | cosmic |
| 47 | ERG | TMPRSS2 | ERG | 7113 | 2078 | cosmic |
| 48 | ERG | TMPRSS2 | ERG | 7113 | 2078 | cosmic |
| 49 | ERG | TMPRSS2 | ERG | 7113 | 2078 | cosmic; ngs |
| 50 | ERG | TMPRSS2 | ERG | 7113 | 2078 | cosmic |
| 51 | ERG | TMPRSS2 | ERG | 7113 | 2078 | cosmic |
| 52 | ERG | TMPRSS2 | ERG | 7113 | 2078 | cosmic; ngs |
| 53 | ERG | TMPRSS2 | ERG | 7113 | 2078 | cosmic |
| 54 | ERG | TMPRSS2 | ERG | 7113 | 2078 | cosmic |
| 55 | ERG | TMPRSS2 | ERG | 7113 | 2078 | cosmic |
| 56 | ERG | TMPRSS2 | ERG | 7113 | 2078 | cosmic |
| 57 | ERG | TMPRSS2 | ERG | 7113 | 2078 | cosmic |
| 58 | ERG | TMPRSS2 | ERG | 7113 | 2078 | cosmic |
| 59 | ETV1 | TMPRSS2 | ETV1 | 7113 | 2115 | ngs |
| 60 | ETV1 | TMPRSS2 | ETV1 | 7113 | 2115 | cosmic; ngs |
| 61 | ETV1 | TMPRSS2 | ETV1 | 7113 | 2115 | cosmic |
| 62 | ETV1 | TMPRSS2 | ETV1 | 7113 | 2115 | cosmic |
| 63 | ETV1 | TMPRSS2 | ETV1 | 7113 | 2115 | cosmic |
| 64 | ETV1 | TMPRSS2 | ETV1 | 7113 | 2115 | cosmic |
| 65 | ETV4 | TMPRSS2 | ETV4 | 7113 | 2118 | ngs |
| 66 | ETV4 | TMPRSS2 | ETV4 | 7113 | 2118 | ngs |
| 67 | ETV4 | TMPRSS2 | ETV4 | 7113 | 2118 | cosmic |
| 68 | ETV4 | TMPRSS2 | ETV4 | 7113 | 2118 | cosmic |
| 69 | ETV4 | TMPRSS2 | ETV4 | 7113 | 2118 | cosmic |
| 70 | ETV5 | TMPRSS2 | ETV5 | 7113 | 2119 | EU314929.1 |
| 71 | ETV5 | TMPRSS2 | ETV5 | 7113 | 2119 | EU314930.1 |
| 72 | ETV5 | TMPRSS2 | ETV5 | 7113 | 2119 | EU314931.1 |
| 73 | FGFR3 | FGFR3 | TACC3 | 2261 | 10 | cosmic; ngs |
| 74 | FGFR3 | FGFR3 | TACC3 | 2261 | 10 | cosmic |
| 75 | FGFR3 | FGFR3 | TACC3 | 2261 | 10 | cosmic |
| 76 | FGFR3 | FGFR3 | TACC3 | 2261 | 10460 | |
| 77 | FGFR3 | FGFR3 | TACC3 | 2261 | 10460 | |
| 78 | FGFR3 | FGFR3 | TACC3 | 2261 | 10 | ngs |
| 79 | FGFR3 | FGFR3 | TACC3 | 2261 | 10 | ngs |
| 80 | FGFR3 | FGFR3 | TACC3 | 2261 | 10 | ngs |
| 81 | FGFR3 | FGFR3 | TACC3 | 2261 | 10 | ngs |
| 82 | FGFR3 | FGFR3 | TACC3 | 2261 | 10 | cosmic |
| 83 | FGFR3 | FGFR3 | TACC3 | 2261 | 10 | cosmic; ngs |
| 84 | NTRK3 | ETV6 | NTRK3 | 2120 | 4916 | ARUP |
| 85 | NTRK3 | ETV6 | NTRK3 | 2120 | 4916 | ARUP |
| 86 | RAF1 | ESRP1 | RAF1 | 54845 | 5894 | cosmic |
| 87 | RARA | PML | RARA | 5371 | 5914 | 12032336, ngs |
| 88 | RARA | PML | RARA | 5371 | 5914 | 12032336, ngs |
| 89 | RARA | PML | RARA | 5371 | 5914 | ngs |
| 90 | RET | CCDC6 | RET | 8030 | 5979 | OncoNetwork; ngs |
| 91 | RET | ERC1 | RET | 23085 | 5979 | ngs |
| 92 | RET | ERC1 | RET | 23085 | 5979 | ngs |
| 93 | RET | ERC1 | RET | 23085 | 5979 | ngs |
| 94 | RET | GOLGA5 (PTC5) | RET | 9950 | 5979 | Klaugbauer et al. 1998, Cancer Research |
| 95 | RET | HOOK3 | RET | 84376 | 5979 | DQ104207.1 |
| 96 | RET | KIAA1468 (RFG9) | RET | 57614 | 5979 | Klugbauer et al 2000, Cancer Res |
| 97 | RET | KIF5B | RET | 3799 | 5979 | OncoNetwork |
| 98 | RET | KIF5B | RET | 3799 | 5979 | OncoNetwork |
| 99 | RET | KIF5B | RET | 3799 | 5979 | OncoNetwork |
| 100 | RET | KIF5B | RET | 3799 | 5979 | OncoNetwork |
| 101 | RET | KIF5B | RET | 3799 | 5979 | OncoNetwork |
| 102 | RET | KIF5B | RET | 3799 | 5979 | OncoNetwork |
| 103 | RET | KIF5B | RET | 3799 | 5979 | OncoNetwork |
| 104 | RET | KTN1 (PTC8) | RET | 3895 | 5979 | Salassidis et al 2000, Cancer Res |
| 105 | RET | NCOA4 | RET | 8031 | 5979 | ngs |
| 106 | RET | PCM1 (PTC4) | RET | 5108 | 5979 | Corvi et al 2000, Oncogene |
| 107 | RET | PRKAR1A | RET | 5573 | 5979 | Bongarzone et al. 1993, Molecular and cellu |
| 108 | RET | TRIM24 (PTC6) | RET | 8805 | 5979 | Klugbauer and Rabes 1999 Oncogene |
| 109 | RET | TRIM27 | RET | 5987 | 5979 | Saenko et al 2003, Mutat Res |
| 110 | RET | TRIM33 (PTC7) | RET | 51592 | 5979 | Klugbauer and Rabes 1999 Oncogene |
| 111 | ROS1 | CD74 | ROS1 | 972 | 6098 | OncoNetwork; lungrx; ngs |
| 112 | ROS1 | CD74 | ROS1 | 972 | 6098 | OncoNetwork; lungrx |
| 113 | ROS1 | CD74 | ROS1 | 972 | 6098 | lungrx |
| 114 | ROS1 | EZR | ROS1 | 7430 | 6098 | lungrx |
| 115 | ROS1 | EZR | ROS1 | 7430 | 6098 | OncoNetwork; ngs |
| 116 | ROS1 | GOPC | ROS1 | 57120 | 6098 | OncoNetwork |
| 117 | ROS1 | GOPC | ROS1 | 57120 | 6098 | OncoNetwork |
| 118 | ROS1 | LRIG3 | ROS1 | 121227 | 6098 | OncoNetwork |
| 119 | ROS1 | SDC4 | ROS1 | 6385 | 6098 | OncoNetwork |
| 120 | ROS1 | SDC4 | ROS1 | 6385 | 6098 | OncoNetwork |
| 121 | ROS1 | SDC4 | ROS1 | 6385 | 6098 | OncoNetwork |
| 122 | ROS1 | SDC4 | ROS1 | 6385 | 6098 | OncoNetwork |
| 123 | ROS1 | SLC34A2 | ROS1 | 10568 | 6098 | |
| 124 | ROS1 | SLC34A2 | ROS1 | 10568 | 6098 | |
| 125 | ROS1 | SLC34A2 | ROS1 | 10568 | 6098 | |
| 126 | ROS1 | SLC34A2 | ROS1 | 10568 | 6098 | OncoNetwork |
| 127 | ROS1 | SLC34A2 | ROS1 | 10568 | 6098 | OncoNetwork |
| 128 | ROS1 | TPM3 | ROS1 | 7170 | 6098 | OncoNetwork |
| 129 | ALK | CLIP4 | ALK | 79745 | 238 | Cazes et al. 2013, Cancer Research |
| 130 | ALK | GTF2IRD1 | ALK | 9569 | 238 | ngs |
| 131 | ALK | MEMO1 | ALK | 51072 | 238 | ngs |
| 132 | ALK | NCOA1 | ALK | 8648 | 238 | N/A |
| 133 | ALK | PRKAR1A | ALK | 5573 | 238 | N/A |
| 134 | ALK | STRN | ALK | 6801 | 238 | cosmic; ngs |
| 135 | ALK | TPM1 | ALK | 7168 | 238 | ngs |
| 136 | RET | AKAP13 | RET | 11214 | 5979 | ngs |
| 137 | RET | FKBP15 | RET | 23307 | 5979 | ngs |
| 138 | RET | SPECC1L | RET | 23384 | 5979 | N/A |
| 139 | RET | TBL1XR1 | RET | 79718 | 5979 | N/A |
| 140 | ROS1 | CEP85L | ROS1 | 387119 | 6098 | ngs |
| 141 | ABL1 | BCR | ABL1 | 613 | 25 | 11289094, 21435002 |
| 142 | ABL1 | BCR | ABL1 | 613 | 25 | 11289094, 21435002 |
| 143 | ABL1 | BCR | ABL1 | 613 | 25 | 11289094, 21435002 |
| 144 | ABL1 | BCR | ABL1 | 613 | 25 | 11289094, 21435002 |
| 145 | ABL1 | BCR | ABL1 | 613 | 25 | 11289094, 21435002 |
| 146 | ABL1 | BCR | ABL1 | 613 | 25 | 11289094, 21435002 |
| 147 | ABL1 | BCR | ABL1 | 613 | 25 | 11289094, 21435002 |
| 148 | ABL1 | BCR | ABL1 | 613 | 25 | 11289094, 21435002 |
| 149 | | PAX8 | PPARG | 7849 | 5468 | COSMIC COSF1223 |
| 150 | | PAX8 | PPARG | 7849 | 5468 | COSMIC, ngs COSF1215 |
| 151 | | PAX8 | PPARG | 7849 | 5468 | COSMIC, ngs COSF1217 |
| 152 | | PAX8 | PPARG | 7849 | 5468 | COSMIC CSOF1221 |
| 153 | | PAX8 | PPARG | 7849 | 5468 | COSMIC COSF1219, COSF1222 |
| 154 | RARA | PML | RARA | 5371 | 5914 | Ampang |
| 155 | RARA | ZBTB16 | RARA | | | Ampang |
| 156 | RARA | PML | RARA | | | Ampang |
| 157 | ABL1 | BCR | ABL1 | 613 | 25 | Ampang |
| 158 | ABL1 | BCR | ABL1 | 613 | 25 | Ampang |
| 159 | ABL1 | BCR | ABL1 | 613 | 25 | Ampang |
| 160 | ABL1 | BCR | ABL1 | 613 | 25 | Ampang |
| 161 | | ABL1 | BCR | 25 | 613 | Ampang |
| 162 | | ABL1 | BCR | 25 | 613 | Ampang |
| 163 | ABL1 | EML1 | ABL1 | | | Ampang |
| 164 | RARA | ZBTB16 | RARA | | | Ampang |
| 165 | | RARA | ZBTB16 | | | Ampang |

| | | | | | Cosmic IDs | Cosmic IDs | | NGS |
|---|---|---|---|---|---|---|---|---|
| | 5′ Exon | 5′ Exon | 3′ Exon | 3′ Exon | (Observed | (Inferred | | Breakpoint |
| | Number | Type | Number | Type | Sequence) | Breakpoint) | Cosmic Fusion Syntax | Label |
| 1 | 1 | cds | 2 | cds | | | | BCR_ABL1_23 |
| 2 | 14 | cds | 2 | cds | | | | BCR_ABL1_24 |
| 3 | 9 | cds | 2 | cds | | | | |
| 4 | 6 | cds | 18 | cds | | | | EML4_ALK_87 |
| 5 | 6 | cds | 17 | cds | | | | EML4_ALK_88 |
| 6 | 14 (with an | cds | 20 | cds | | | | |
| | additional | | | | | | | |
| | 11 | | | | | | | |
| | nucleotides | | | | | | | |
| | of | unknown | | | | | | |
| | origin) | | | | | | | |
| 7 | 14 | cds | 20 | cds | | | | |
| 8 | 15 | cds | 20 | cds | | | | |
| 9 | | N/A | see | N/A | | | | |
| | | | ‘NGSfusionsequences’ | | | | | |
| | | | tab | | | | | |
| 10 | 17 | cds | 20 | cds | COSF1366, | COSF1368 | | |
| | | | | | COSF1367 | | | |
| 11 | 6 | cds | 19 | cds | COSF1296 | COSF1297 | | |
| 12 | 13 | cds | 20 | cds | COSF408, | COSF463, | | |
| | | | | | COSF1062 | COSF489, | | |
| | | | | | | COSF1063, | | |
| | | | | | | COSF462, | | |
| | | | | | | COSF410, | | |
| | | | | | | COSF414 | | |
| 13 | 20 | cds | 20 | cds | COSF409 | COSF465, | | EML4_ALK_12 |
| | | | | | | COSF490, | | |
| | | | | | | COSF731, | | |
| | | | | | | COSF464 | | |
| 14 | 6 | cds | 20 | cds | COSF411, | COSF474, | | EML4_ALK_32 |
| | | | | | COSF412, | COSF734, | | |
| | | | | | COSF1296 | COSF476, | | |
| | | | | | | COSF493, | | |
| | | | | | | COSF1297 | | |
| 15 | 6 (plus 33 | cds | 20 | cds | COSF411, | COSF474, | | |
| | nucleotides | | | | COSF412, | COSF734, | | |
| | from | | | | COSF1296 | COSF476, | | |
| | exon 6b) | | | | | COSF493, | | |
| | | | | | | COSF1297 | | |
| 16 | 14 (with an | cds | 20 | cds | COSF477 | COSF491 | | |
| | additional | | (starting | | | | | |
| | 11 | | at | | | | | |
| | nucleotides | | nucleotide | | | | | |
| | of unknown | | 50 in | | | | | |
| | origin) | | exon 20) | | | | | |
| 17 | 2 | cds | 20 | cds | COSF478 | COSF480 | | |
| 18 | 2 | cds | 20 | cds | COSF479 | | | |
| | | (contains | | | | | | |
| | | an | | | | | | |
| | | additional | | | | | | |
| | | 117 | | | | | | |
| | | nucleotides | | | | | | |
| | | from intron | | | | | | |
| | | 19) | | | | | | |
| 19 | 13 | cds | 20 | cds | COSF1062 | COSF1063 | | |
| | | | (starting | | | | | |
| | | | at | | | | | |
| | | | nucleotide | | | | | |
| | | | 69 in | | | | | |
| | | | exon 20) | | | | | |
| 20 | 14 | cds | 20 | cds | COSF1064 | COSF1065 | | |
| | | | (starting | | | | | |
| | | | at | | | | | |
| | | | nucleotide | | | | | |
| | | | 13 in | | | | | |
| | | | exon 20) | | | | | |
| 21 | 15 | cds | 20 | cds | COSF413 | COSF475 | | |
| | (minus 19 | | (starting | | | | | |
| | nucleotides) | | at | | | | | |
| | | | nucleotide | | | | | |
| | | | 21 of | | | | | |
| | | | exon 20) | | | | | |
| 22 | 18 | cds | 20 | cds | COSF487 | COSF1376 | | |
| 23 | 15 | cds | 20 | cds | COSF1060, | | | |
| | | | | | COSF1381 | | | |
| 24 | 24 | cds | 20 | cds | COSF1058 | | | |
| 25 | 17 | cds | 20 | cds | COSF1257 | | | |
| 26 | 9 | cds | 20 | cds | 1276 | 1277 | KLC1{ENST00000389744}: | |
| | | | | | | | r.1_1530_ALK{NM_004304}: | |
| | | | | | | | r.4080_6222 | |
| 27 | 5 | cds | 20 | cds | 426 | | TFG{ENST00000240851}: | |
| | | | | | | | r.1_1029_ALK{NM_004304}: | |
| | | | | | | | r.4080_6222 | |
| 28 | 4 | cds | 20 | cds | 424 | 425 | TFG{ENST00000240851+56: | |
| | | | | | | | r.1_864_ALK{NM_004304}: | |
| | | | | | | | r.4080_6222 | |
| 29 | 6 | cds | 20 | cds | 428 | 429 | TFG{ENST00000240851}: | |
| | | | | | | | r.1_1170_ALK{NM_0 04304}: | |
| | | | | | | | r.4080_6222 | |
| 30 | **** Fusion contains exons 1 | | | | | | | |
| | and 2 of PTPN3 with part of | | | | | | | |
| | intron 9 followed by exons 10 | | | | | | | |
| | and 11 of ALK and the exons 3, | | | | | | | |
| | 4, 5, of PTPN3**** | | | | | | | |
| 31 | well | cds? | 8 | cds | 828 | 829 | AGTRAP{ENST00000314340}: | |
| | within | | | | | | r.1_598_BRAF{NM_004333}: | |
| | exon 5? | | | | | | r.1043_2513 | |
| 32 | 8 | cds | 9 | cds | | | | |
| 33 | 1 | utr5 | 8 | cds | 871 | 872 | SLC45A3{ENST00000367145}: | |
| | | | | | | | r.1_66_BRAF{NM_004333}: | |
| | | | | | | | r.1042_2513 | |
| 34 | Exons not specified. | | | | | | | |
| 35 | 1 | utr5 | 4 | cds | | | | |
| 36 | 1 | utr5 | 2 | utr5 | 23 | 123 | TMPRSS2{NM_005656.2}: | TMPRSS2_ERG_67 |
| | | | | | | | r.1_71_ERG{NM_004449.3}: | |
| | | | | | | | r.38_3097 | |
| 37 | 1 | utr5 | 3 | cds | | | | TMPRSS2_ERG_73 |
| 38 | 1 | utr5 | 3 | utr5 | 24 | 124 | TMPRSS2{NM_005656.2}: | |
| | | | | | | | r.1_71_ERG{NM_004449.3}: | |
| | | | | | | | r.140_3097 | |
| 39 | 1 | cds | 4 | cds | | | | TMPRSS2_ERG_62 |
| 40 | 1 | utr5 | 4 | cds | 38 | 138 | TMPRSS2{NM_005656.2}: | TMPRSS2_ERG_63 |
| | | | | | | | r.1_71_ERG{NM_004449.3}: | |
| | | | | | | | r.226-?3097 | |
| 41 | 1 | utr5 | 4 | cds | 25 | 125 | TMPRSS2{NM_005656.2}: | TMPRSS2_ERG_63 |
| | | | | | | | r.1_71_ERG{NM_004449.3}: | |
| | | | | | | | r.226_3097 | |
| 42 | 1 | utr5 | 4 | cds | 39 | 139 | TMPRSS2{NM_005656.2}: | TMPRSS2_ERG_63 |
| | | | | | | | r.1_71 + ?_ERG | |
| | | | | | | | {NM_004449.3}: | |
| | | | | | | | r.226-?_3097 | |
| 43 | 1 | cds | 5 | cds | | | | TMPRSS2_ERG_77 |
| 44 | 1 | utr5 | 5 | cds | 26 | 126 | TMPRSS2{NM_005656.2}: | TMPRSS2_ERG_61 |
| | | | | | | | r.1_71_ERG{NM_004449.3}: | |
| | | | | | | | r.444_3097 | |
| 45 | 1 | utr5 | 6 | cds | 36 | | TMPRSS2{NM_005656.2}: | |
| | | | | | | | r.1_71_ERG{NM_004449.3}: | |
| | | | | | | | r.596_3097 | |
| 46 | 1 | utr5 | 2 (no | utr5 | 41 | | TMPRSS2{NM_005656.2}: | |
| | | | exon 5) | | | | r.1_71_ERG{NM_004449.31: | |
| | | | | | | | r.38_443_ERG{NM_004449.3}: | |
| | | | | | | | r.596_3097 | |
| 47 | 1 | utr5 | 3 (no | utr5 | 40 | | TMPRSS2{NM_005656.2}: | |
| | | | exon 4) | | | | r.1_71_ERG{NM_004449.31: | |
| | | | | | | | r.140_225_ERG | |
| | | | | | | | {NM_004449.3}: | |
| | | | | | | | r.444_3097 | |
| 48 | 2 | cds | 2 | utr5 | 27 | 127 | TMPRSS2{NM_005656.2}: | |
| | | | | | | | r.1_142_ERG{NM_004449.3}: | |
| | | | | | | | r.38_3097 | |
| 49 | 2 | cds | 4 | cds | 28 | 128 | TMPRSS2{NM_005656.2}: | TMPRSS2_ERG_64 |
| | | | | | | | r.1_142_ERG{NM_004449.3}: | |
| | | | | | | | r.226_3097 | |
| 50 | 2 | cds | 5 | cds | 29 | 129 | TMPRSS2{NM_005656.2}: | |
| | | | | | | | r.1_142_ERG{NM_004449.3}: | |
| | | | | | | | r.444_3097 | |
| 51 | 2 | cds | 4 (with | cds | 216 | | TMPRSS2{NM_005656.2}: | |
| | | | repeat of | | | | r.1_142_ERG{AY204740.1}: | |
| | | | portion | | | | r.226_320_ERG{NM_004449.3}: | |
| | | | of 4) | | | | r.226_3097 | |
| 52 | 3 | cds | 4 | cds | 30 | 130 | TMPRSS2{NM_005656.2}: | TMPRSS2_ERG_68 |
| | | | | | | | r.1_365_ERG{NM_004449.3}: | |
| | | | | | | | r.226_3097 | |
| 53 | 4 | cds | 4 | cds | 18 | 118 | TMPRSS2{NM_005656.2}: | |
| | | | | | | | r.1_452_ERG{NM_004449.3}: | |
| | | | | | | | r.226_3097 | |
| 54 | 4 | cds | 5 | cds | 17 | | TMPRSS2{NM_005656.2}: | |
| | | | | | | | r.1_452_ERG{NM_004449.3}: | |
| | | | | | | | r.444_3097 | |
| 55 | 5 | cds | 4 | cds | 16 | 116 | TMPRSS2{NM_005656.2}: | |
| | | | | | | | r.1_572_ERG{NM_004449.3}: | |
| | | | | | | | r.226_3097 | |
| 56 | 4 (no | cds | 4 | cds | 202 | | TMPRSS2{NM_005656.2}: | |
| | exon 2 | | | | | | r.1_71_TMPRSS2{NM_005656.2}: | |
| | or 3) | | | | | | r.366_452_ERG{NM_004449.3}: | |
| | | | | | | | r.226_3097 | |
| 57 | 4 (no | cds | 5 | cds | 203 | | TMPRSS2{NM_005656.2}: | |
| | exon 2 | | | | | | r.1_71_TMPRSS2{NM_005656.2}: | |
| | or 3) | | | | | | r.366_452_ERG{NM_004449.3}: | |
| | | | | | | | r.444_3097 | |
| 58 | unknown | unknown | unknown | unknown | 21 | 121 | TMPRSS2{NM_005656.2}: | |
| | | | | | | | r.?_ERG{NM_004449.3}:r.? | |
| 59 | 2 | cds | 9 | cds | | | | TMPRSS2_ETV1_59 |
| 60 | 1 | utr5 | 7 | cds | 33 | | TMPRSS2{NM_005656.2}: | TMPRSS2_ETV1_53 |
| | | | | | | | r.1_71_ETV1{NM_004956.3}: | |
| | | | | | | | r.323_6158 | |
| 61 | 2 | cds | 7 | cds | 34 | 134 | TMPRSS2{NM_005656.2}: | |
| | | | | | | | r.1_142_ETV1{NM_004956.3}: | |
| | | | | | | | r.323_6158 | |
| 62 | 1 | utr5 | 6 | cds | 14 | | TMPRSS2{NM_005656.2}: | |
| | | | | | | | r.1_71_ETV1{NM_004956.3}: | |
| | | | | | | | r.269_6158 | |
| 63 | 2 | cds | 6 | cds | 15 | 115 | TMPRSS2{NM_005656.2}: | |
| | | | | | | | r.1_142_ETV1{NM_004956.3}: | |
| | | | | | | | r.269_6158 | |
| 64 | unknown | unknown | unknown | unknown | 22 | 122 | TMPRSS2{NM_005656.2}: | |
| | | | | | | | r.?_ETV1{NM_004956.3}:r.? | |
| 65 | 1 | utr5 | 2 | utr5 | | | | TMPRSS2_ETV4_83 |
| 66 | 1 | utr5 | 3 | cds | | | | TMPRSS2_ETV4_82 |
| 67 | 8 kb | intergenic? | 3 | cds | 214 | | TMPRSS2{NM_005656.1}: | |
| | upstream of | | | | | | r.(1-8013_1-8000)_ETV4 | |
| | start | | | | | | {NM_001986.1} | |
| | | | | | | | r.131-19_2212 | |
| 68 | 8 kb | intergenic? | 2 | cds | 213 | 212 | TMPRSS2{NM_005656.2}: | |
| | upstream | | | | | | r.(1-8047_1-8000)_ETV4 | |
| | of start | | | | | | {NM_001986.1}: | |
| | | | | | | | r.131-19_2212 | |
| 69 | unknown | unknown | unknown | unknown | 44 | 144 | TMPRSS2{NM_005656.2}: | |
| | | | | | | | r.?_ETV4{NM_001986.1}:r.? | |
| 70 | 1 | utr5 | 2 | utr5 | | | | |
| 71 | 3 | cds | 2 | utr5 | | | | |
| 72 | 3 | cds | 2 | utr5 | | | | |
| 73 | 17 | cds | 11 | cds | 1348 | | FGFR3{NM_000142}: | FGFR3_TACC3_3 |
| | | | | | | | r.1_2530_TACC3 | |
| | | | | | | | {ENST00000313288}: | |
| | | | | | | | r.2048_2781 | |
| 74 | 17 + | cds | middle | cds | 1350 | 1351 | FGFR3{NM_000142}: | |
| | extra? | | of 5? | | | | r.1_2530 + | |
| | | | | | | | 104_TACC3{ENST00000313288}: | |
| | | | | | | | r.541_2781 | |
| 75 | 17 | cds | 8 | cds | 1353 | 1355 | FGFR3{NM_000142}: | |
| | | | | | | | r.1_2530_TACC3 | |
| | | | | | | | {ENST00000313288}: | |
| | | | | | | | r.1751_2781 | |
| 76 | | N/A | see | N/A | | | | |
| | | | ‘NGSfusionsequences’ | | | | | |
| | | | tab | | | | | |
| 77 | | N/A | see | N/A | | | | |
| | | | ‘NGSfusionsequences’ | | | | | |
| | | | tab | | | | | |
| 78 | 16 | cds | 11 | cds | | | | FGFR3_TACC3_51 |
| 79 | 15 | cds | 11 | cds | | | | FGFR3_TACC3_29 |
| 80 | 16 | cds | 10 | cds | | | | FGFR3_TACC3_18 |
| 81 | 17 | cds | 6 | cds | | | | FGFR3_TACC3_11 |
| 82 | 17 + | cds | middle | cds | 1357 | 1358 | FGFR3{NM_000142}: | |
| | extra? | | of 9? | | | | r.1_2530 + | |
| | | | | | | | 63_TACC3{ENST00000313288}: | |
| | | | | | | | r.1877_2781 | |
| 83 | 17 | cds | 10 | cds | 1359 | 1360 | FGFR3{NM_000142}: | FGFR3_TACC3_19 |
| | | | | | | | r.1_2530_TACC3 | |
| | | | | | | | {ENST00000313288}: | |
| | | | | | | | r.1943_2781 | |
| 84 | 5 | cds | 13 | cds | COSF571 | COSF572, | | |
| | | | | | | COSF889 | | |
| 85 | 4 | cds | 13 | cds | COSF823 | COSF824 | | |
| 86 | 13 | cds | 6 | cds | 826 | 830 | ESRP1{ENST00000358397}: | |
| | | | | | | | r.1_1955_RAF1{NM_002880}: | |
| | | | | | | | r.975_3245 | |
| 87 | 6 | cds | 3 | cds | | | | PML_RARA_25 |
| 88 | 3 | cds | 3 | cds | | | | PML_RARA_26 |
| 89 | 4 | cds | 3 | cds | | | | PML_RARA_27 |
| 90 | 1 | cds | 12 | cds | COSF1271 | COSF1272 | | CCDC6_RET_44 |
| 91 | 7 | cds | 12 | cds | | | | ERC1_RET_10 |
| 92 | 12 | cds | 12 | cds | | | | ERC1_RET_85 |
| 93 | 17 | cds | 12 | cds | | | | ERC1_RET_86 |
| 94 | 7 | cds | Includes | | | | | |
| | | | RET | | | | | |
| | | | Kinase | | | | | |
| | | | domain | | | | | |
| 95 | 11 | cds | 12 | cds | | | | |
| 96 | 10 | cds | Not | | | | | |
| | | | specified | | | | | |
| 97 | 24 | cds | 8 | cds | COSF1236 | COSF1242 | | |
| 98 | 24 | cds | 11 | cds | COSF1262 | COSF1263 | | |
| 99 | 16 | cds | 12 | cds | COSF1231 | COSF1240 | | |
| 100 | 15 | cds | 11 | cds | COSF1255 | COSF1256 | | |
| 101 | 23 | cds | 12 | cds | COSF1234 | COSF1235 | | |
| 102 | 22 | cds | 12 | cds | COSF1253 | COSF1254 | | |
| 103 | 15 | cds | 12 | cds | COSF1232 | COSF1233 | | |
| 104 | 30 | cds | Includes | | | | | |
| | | | RET | | | | | |
| | | | Kinase | | | | | |
| | | | domain | | | | | |
| 105 | 7 | cds | 12 | cds | | | | NCOA4_RET_89 |
| 106 | 29 | cds | Described | | | | | |
| | | | as RET | | | | | |
| | | | breakpoint | | | | | |
| | | | is the | | | | | |
| | | | same as | | | | | |
| | | | RET/PTC | | | | | |
| | | | 1/2/3 | | | | | |
| | | | with | | | | | |
| | | | intact | | | | | |
| | | | Kinase | | | | | |
| | | | domain | | | | | |
| 107 | Exons not specified. | | | | | | | |
| 108 | Exons not specified. The fusion includes the | | | | | | | |
| | RET tyrosine kinase domain | | | | | | | |
| 109 | 3 | cds | The | | | | | |
| | | | fusion | | | | | |
| | | | includes | | | | | |
| | | | the RET | | | | | |
| | | | tyrosine | | | | | |
| | | | kinase | | | | | |
| | | | domain | | | | | |
| 110 | Exons not specified. The fusion includes the | | | | | | | |
| | RET tyrosine kinase domain | | | | | | | |
| 111 | 6 | cds | 34 | cds | COSF1200 | COSF1203 | | CD74_ROS1_30 |
| 112 | 6 | cds | 32 | cds | COSF1202 | COSF1201 | | |
| 113 | | N/A | see | N/A | | | | |
| | | | ‘NGSfusionsequences’ | | | | | |
| | | | tab | | | | | |
| 114 | | N/A | see | N/A | | | | |
| | | | ‘NGSfusionsequences’ | | | | | |
| | | | tab | | | | | |
| 115 | 10 | cds | 34 | cds | COSF1267 | COSF1268 | EZR_ROS1_43 | |
| 116 | 8 | cds | 35 | cds | COSF1139 | COSF1251 | | |
| 117 | 4 | cds | 36 | cds | COSF1188 | COSF1210 | | |
| 118 | 16 | cds | 35 | cds | COSF1269 | COSF1270 | | |
| 119 | 2 | cds | 32 | cds | COSF1265 | COSF1266 | | |
| 120 | 4 | cds | 34 | cds | COSF1280 | COSF1279 | | |
| 121 | 4 | cds | 32 | cds | COSF1278 | COSF1279 | | |
| 122 | 2 | cds | 34 | cds | not in | not in | | |
| | | | | | cosmic | cosmic | | |
| 123 | | N/A | see | N/A | | | | |
| | | | ‘NGSfusionsequences’ | | | | | |
| | | | tab | | | | | |
| 124 | | N/A | see | N/A | | | | |
| | | | ‘NGSfusionsequences’ | | | | | |
| | | | tab | | | | | |
| 125 | | N/A | see | N/A | | | | |
| | | | ‘NGSfusionsequences’ | | | | | |
| | | | tab | | | | | |
| 126 | 4 | cds | 32 | cds | COSF1198 | COSF1197 | | |
| 127 | 13 | cds | 32 | cds | COSF1261, | COSF1260 | | |
| | | | | | COSF1259 | | | |
| 128 | 8 | cds | 35 | cds | COSF1273 | COSF1274 | | |
| 129 | 11 | cds | 23 | cds | | | | |
| 130 | 7 | cds | 20 | cds | | | | |
| 131 | 2 | cds | 7 | cds | | | | |
| 132 | | N/A | see | N/A | | | | |
| | | | ‘NGSfusionsequences’ | | | | | |
| | | | tab | | | | | |
| 133 | | N/A | see | N/A | | | | |
| | | | ‘NGSfusionsequences’ | | | | | |
| | | | tab | | | | | |
| 134 | 3 | cds | 20 | cds | COSF1430 | COSF1431 | STRN{ENST00000263918}: | |
| | | | | | | | r.1_421_ALK{NM_004304}: | |
| | | | | | | | r.4080_6222 | |
| 135 | 8 | cds | 20 | cds | | | | |
| 136 | 36 | cds | 12 | cds | | | | |
| 137 | 25 | cds | 12 | cds | | | | |
| 138 | | N/A | see | N/A | | | | |
| | | | ‘NGSfusionsequences’ | | | | | |
| | | | tab | | | | | |
| 139 | | N/A | See | N/A | | | | |
| | | | ‘NGSfusionsequences’ | | | | | |
| | | | tab | | | | | |
| 140 | 8 | cds | 36 | cds | | | | |
| 141 | 6 | cds | 2 | cds | | | | |
| 142 | 8 | cds | 2 | cds | | | | |
| 143 | 13 | cds | 2 | cds | | | | |
| 144 | 19 | cds | 2 | cds | | | | |
| 145 | 1 | cds | 3 | cds | | | | |
| 146 | 13 | cds | 3 | cds | | | | |
| 147 | 14 | cds | 3 | cds | | | | |
| 148 | 2 | cds | la | utr5 | | | | |
| 149 | 7 | cds | 2 | cds | | | | |
| 150 | 8 | cds | 2 | cds | | | | |
| 151 | 9 | cds | 2 | cds | | | | |
| 152 | 9 (short - | cds | 2 | cds | | | | |
| | only the | | | | | | | |
| | first 102 | | | | | | | |
| | bases of | | | | | | | |
| | exon 9) | | | | | | | |
| 153 | 10 | cds | 2 | cds | | | | |
| 154 | 6 | cds | 3 | cds | | | | |
| 155 | 3 | cds | 3 | cds | | | | |
| 156 | 5 | | 3 | | | | | |
| 157 | 18 | cds | 2 | cds | | | | |
| 158 | 6 | cds | 3 | cds | | | | |
| 159 | 19 | cds | 3 | cds | | | | |
| 160 | 18 | cds | 3 | cds | | | | |
| 161 | 1 | | 14 | | | | | |
| 162 | 1 | | 15 | | | | | |
| 163 | 17 | | 2 | | | | | |
| 164 | 4 | cds | 3 | cds | | | | |
| 165 | 2 | | 4 | | | | | |

| | | | | | | | | NGS |
|---|---|---|---|---|---|---|---|---|
| | | 5′ NGS | | 3′ NGS | Reference | | | Sample |
| | 5′ Accession | Breakpoint | 3′ Accession | Breakpoint | Build | 5′ NGS Sequence | 3′ NGS Sequence | Count |
| 1 | NM_004327 | 23524426 | NM_005157 | 133729451 | hg19 | CACTGCCCGGTTGTCGTGTCCG | AAGCCCTTCAGCGGCCAGT | 1 |
| | | | | | | AGGCCACCATCGTGGGCGTCCG | AGCATCTGACTTTGAGCCT | |
| | | | | | | CAAGACCGGGCAGATCTGGCCC | CAGGGTCTGAGTGAAGCC | |
| | | | | | | AACGATGGCGAGGGCGCCTTCC | GCTCGTTGGAACTCCAAG | |
| | | | | | | ATGGAGACGCAG | GAAAACCTTCTCGCTGGAC | |
| | | | | | | | CCAGTGA | |
| 2 | NM_004327 | 23632600 | NM_005157 | 133729451 | hg19 | ATTCCGCTGACCATCAATAAGG | AAGCCCTTCAGCGGCCAGT | 2 |
| | | | | | | AAGATGATGAGTCTCCGGGGCT | AGCATCTGACTTTGAGCCT | |
| | | | | | | CTATGGGTTTCTGAATGTCATCG | CAGGGTCTGAGTGAAGCC | |
| | | | | | | TCCACTCAGCCACTGGATTTAAG | GCTCGTTGGAACTCCAAG | |
| | | | | | | CAGAGTTCAA | GAAAACCTTCTCGCTGGAC | |
| | | | | | | | CCAGTGA | |
| 3 | | | | | | | | |
| 4 | NM_019063 | 42491868 | NM_004304 | 29450442 | hg19 | GATGATAGCCGTAATAAATTGT | AAGTGATGGAAGGCCACG | 1 |
| | | | | | | CGAAAATACCTTCAACACCCAA | GGGAAGTGAATATTAAGC | |
| | | | | | | ATTAATACCAAAAGTTACCAAA | ATTATCTAAACTGCAGTCA | |
| | | | | | | ACTGCAGACAAGCATAAAGATG | CTGTGAGGTAGACGAATG | |
| | | | | | | TCATCATCAACC | TCACATGGACCCTGAAAGC | |
| | | | | | | | CACAAGGT | |
| 5 | NM_019063 | 42491869 | NM_004304 | 29451751 | hg19 | ATGATAGCCGTAATAAATTGTC | AGGCGGCAATGCAGCCTC | 1 |
| | | | | | | GAAAATACCTTCAACACCCAAAT | AAACAATGACCCCGAAAT | |
| | | | | | | TAATACCAAAAGTTACCAAAACT | GGATGGGGAAGATGGGG | |
| | | | | | | GCAGACAAGCATAAAGATGTCA | TTTCCTTCATCAGTCCACTG | |
| | | | | | | TCATCAACCA | GGCATCCTGTACACCCCAG | |
| | | | | | | | CTTTAAAA | |
| 6 | | | | | | | | |
| 7 | | | | | | | | |
| 8 | | | | | | | | |
| 9 | | | | | | | | |
| 10 | | | | | | | | |
| 11 | | | | | | | | |
| 12 | | | | | | | | |
| 13 | NM_019063 | 42552694 | NM_004304 | 29446394 | hg19 | GGAAGGTGCACTGGACATTCCA | TGTACCGCCGGAAGCACC | 1 |
| | | | | | | GCTACATCACACACCTTGACTGG | AGGAGCTGCAAGCCATGC | |
| | | | | | | TCCCCAGACAACAAGTATATAAT | AGATGGAGCTGCAGAGCC | |
| | | | | | | GTCTAACTCGGGAGACTATGAA | CTGAGTACAAGCTGAGCA | |
| | | | | | | ATATTGTACT | AGCTCCGCACCTCGACCAT | |
| | | | | | | | CATGACCGA | |
| 14 | NM_019063 | 42491870 | NM_004304 | 29448327 | hg19 | TGATAGCCGTAATAAATTGTCG | GTGTACCGCCGGAAGCAC | 1 |
| | | | | | | AAAATACCTTCAACACCCAAATT | CAGGAGCTGCAAGCCATG | |
| | | | | | | AATACCAAAAGTTACCAAAACT | CAGATGGAGCTGCAGAGC | |
| | | | | | | GCAGACAAGCATAAAGATGTCA | CCTGAGTACAAGCTGAGC | |
| | | | | | | TCATCAACCAA | AAGCTCCGCACCTCGACCA | |
| | | | | | | | TCATGACCG | |
| 15 | | | | | | | | |
| 16 | | | | | | | | |
| 17 | | | | | | | | |
| 18 | | | | | | | | |
| 19 | | | | | | | | |
| 20 | | | | | | | | |
| 21 | | | | | | | | |
| 22 | | | | | | | | |
| 23 | | | | | | | | |
| 24 | | | | | | | | |
| 25 | | | | | | | | |
| 26 | | | | | | | | |
| 27 | | | | | | | | |
| 28 | | | | | | | | |
| 29 | | | | | | | | |
| 30 | | | | | | | | |
| 31 | | | | | | | | |
| 32 | | | | | | | | |
| 33 | | | | | | | | |
| 34 | | | | | | | | |
| 35 | | | | | | | | |
| 36 | NM_005656 | 42880008 | NM_004449 | 39956869 | hg19 | GAGTAGGCGCGAGCTAAGCAG | GTTATTCCAGGATCTTTGG | 3 |
| | | | | | | GAGGCGGAGGCGGAGGCGGA | AGACCCGAGGAAAGCCGT | |
| | | | | | | GGGCGAGGGGCGGGGAGCGCC | GTTGACCAAAAGCAAGAC | |
| | | | | | | GCCTGGAGCGCGGCAG | AAATGACTCACAGAGAAA | |
| | | | | | | | AAAGATGGCAGAACCAAG | |
| | | | | | | | GGCAACTAA | |
| 37 | NM_005656 | 42880008 | NM_004449 | 39947671 | hg19 | GAGTAGGCGCGAGCTAAGCAG | CCGTCAGGTTCTGAACAGC | 1 |
| | | | | | | GAGGCGGAGGCGGAGGCGGA | TGGTAGATGGGCTGGCTT | |
| | | | | | | GGGCGAGGGGCGGGGAGCGCC | ACTGAAGGACATGATTCA | |
| | | | | | | GCCTGGAGCGCGGCAG | GACTGTCCCGGACCCAGC | |
| | | | | | | | AGCTCATATCAAGGAAGC | |
| | | | | | | | CTTATCAGT | |
| 38 | | | | | | | | |
| 39 | NM_001135099 | 42879877 | NM_004449 | 39817544 | hg19 | GGGGTCCGGGCTGGGGAGGGG | GAAGCCTTATCAGTTGTGA | 26 |
| | | | | | | AACCTGGGCGCCTGGGACCCGC | GTGAGGACCAGTCGTTGTT | |
| | | | | | | CGATGCCCCCTGCCCCGCCCGG | TGAGTGTGCCTACGGAAC | |
| | | | | | | AGGTGAAAGCGGGTGTGAGGA | GCCACACCTGGCTAAGAC | |
| | | | | | | GCGCGGCGCGGCAG | AGAGATGACCGCGTCCTCC | |
| | | | | | | | TCCAGCG | |
| 40 | NM_005656 | 42880008 | NM_004449 | 39817544 | hg19 | GAGTAGGCGCGAGCTAAGCAG | GAAGCCTTATCAGTTGTGA | 34 |
| | | | | | | GAGGCGGAGGCGGAGGCGGA | GTGAGGACCAGTCGTTGTT | |
| | | | | | | GGGCGAGGGGCGGGGAGCGCC | TGAGTGTGCCTACGGAAC | |
| | | | | | | GCCTGGAGCGCGGCAG | GCCACACCTGGCTAAGAC | |
| | | | | | | | AGAGATGACCGCGTCCTCC | |
| | | | | | | | TCCAGCG | |
| 41 | NM_005656 | 42880008 | NM_004449 | 39817544 | hg19 | | GAAGCCTTATCAGTTGTGA | 34 |
| | | | | | | GAGTAGGCGCGAGCTAAGCAG | GTGAGGACCAGTCGTTGTT | |
| | | | | | | GAGGCGGAGGCGGAGGCGGA | TGAGTGTGCCTACGGAAC | |
| | | | | | | GGGCGAGGGGCGGGGAGCGCC | GCCACACCTGGCTAAGAC | |
| | | | | | | GCCTGGAGCGCGGCAG | AGAGATGACCGCGTCCTCC | |
| | | | | | | | TCCAGCG | |
| 42 | NM_005656 | 42880008 | NM_004449 | 39817544 | hg19 | GAGTAGGCGCGAGCTAAGCAG | GAAGCCTTATCAGTTGTGA | 34 |
| | | | | | | GAGGCGGAGGCGGAGGCGGA | GTGAGGACCAGTCGTTGTT | |
| | | | | | | GGGCGAGGGGCGGGGAGCGCC | TGAGTGTGCCTACGGAAC | |
| | | | | | | GCCTGGAGCGCGGCAG | GCCACACCTGGCTAAGAC | |
| | | | | | | | AGAGATGACCGCGTCCTCC | |
| | | | | | | | TCCAGCG | |
| 43 | NM_001135099 | 42879877 | NM_004449 | 39795483 | hg19 | GGGGTCCGGGCTGGGGAGGGG | GAACTCTCCTGATGAATGC | 1 |
| | | | | | | AACCTGGGCGCCTGGGACCCGC | AGTGTGGCCAAAGGCGGG | |
| | | | | | | CGATGCCCCCTGCCCCGCCCGG | AAGATGGTGGGCAGCCCA | |
| | | | | | | AGGTGAAAGCGGGTGTGAGGA | GACACCGTTGGGATGAAC | |
| | | | | | | GCGCGGCGCGGCAG | TACGGCAGCTACATGGAG | |
| | | | | | | | GAGAAGCAC | |
| 44 | NM_005656 | 42880008 | NM_004449 | 39795483 | hg19 | GAGTAGGCGCGAGCTAAGCAG | GAACTCTCCTGATGAATGC | 5 |
| | | | | | | GAGGCGGAGGCGGAGGCGGA | AGTGTGGCCAAAGGCGGG | |
| | | | | | | GGGCGAGGGGCGGGGAGCGCC | AAGATGGTGGGCAGCCCA | |
| | | | | | | GCCTGGAGCGCGGCAG | GACACCGTTGGGATGAAC | |
| | | | | | | | TACGGCAGCTACATGGAG | |
| | | | | | | | GAGAAGCAC | |
| 45 | | | | | | | | |
| 46 | | | | | | | | |
| 47 | | | | | | | | |
| 48 | | | | | | | | |
| 49 | NM_005656 | 42870046 | NM_004449 | 39817544 | hg19 | GGCGGGGAGCGCCGCCTGGAG | GAAGCCTTATCAGTTGTGA | 24 |
| | | | | | | CGCGGCAGGTCATATTGAACAT | GTGAGGACCAGTCGTTGTT | |
| | | | | | | TCCAGATACCTATCATTACTCGA | TGAGTGTGCCTACGGAAC | |
| | | | | | | TGCTGTTGATAACAGCAAGATG | GCCACACCTGGCTAAGAC | |
| | | | | | | GCTTTGAACTCA | AGAGATGACCGCGTCCTCC | |
| | | | | | | | TCCAGCG | |
| 50 | | | | | | | | |
| 51 | | | | | | | | |
| 52 | NM_005656 | 42866283 | NM_004449 | 39817544 | hg19 | TCCCCCGTGCCCCAGTACGCCCC | GAAGCCTTATCAGTTGTGA | 1 |
| | | | | | | GAGGGTCCTGACGCAGGCTTCC | GTGAGGACCAGTCGTTGTT | |
| | | | | | | AACCCCGTCGTCTGCACGCAGC | TGAGTGTGCCTACGGAAC | |
| | | | | | | CCAAATCCCCATCCGGGACAGT | GCCACACCTGGCTAAGAC | |
| | | | | | | GTGCACCTCAA | AGAGATGACCGCGTCCTCC | |
| | | | | | | | TCCAGCG | |
| 53 | | | | | | | | |
| 54 | | | | | | | | |
| 55 | | | | | | | | |
| 56 | | | | | | | | |
| 57 | | | | | | | | |
| 58 | | | | | | | | |
| 59 | NM_005656 | 42870046 | NM_004956 | 13971374 | hg19 | GGCGGGGAGCGCCGCCTGGAG | ATTTCGCCGCCAGCTTTCT | 1 |
| | | | | | | CGCGGCAGGTCATATTGAACAT | GAACCCTGTAACTCCTTTC | |
| | | | | | | TCCAGATACCTATCATTACTCGA | CTCCTTTGCCGACGATGCC | |
| | | | | | | TGCTGTTGATAACAGCAAGATG | AAGGGAAGGACGTCCTAT | |
| | | | | | | GCTTTGAACTCA | GTACCAACGCCAGATGTCT | |
| | | | | | | | GAGCCA | |
| 60 | NM_005656 | 42880008 | NM_004956 | 13978871 | hg19 | GAGTAGGCGCGAGCTAAGCAG | TGGCTTTTCATGGCCTGCC | 1 |
| | | | | | | GAGGCGGAGGCGGAGGCGGA | ACTGAAAATCAAGAAAGA | |
| | | | | | | GGGCGAGGGGCGGGGAGCGCC | ACCCCACAGTCCATGTTCA | |
| | | | | | | GCCTGGAGCGCGGCAG | GAAATCAGCTCTGCCTGCA | |
| | | | | | | | GTCAAGAACAGCCCTTTAA | |
| | | | | | | | ATTCAG | |
| 61 | | | | | | | | |
| 62 | | | | | | | | |
| 63 | | | | | | | | |
| 64 | | | | | | | | |
| 65 | NM_005656 | 42880008 | NM_001986 | 41623036 | hg19 | GAGTAGGCGCGAGCTAAGCAG | GTCTCGGCCCCCGCTTGGG | 1 |
| | | | | | | GAGGCGGAGGCGGAGGCGGA | GCCCCGGCCGTGCGGCCG | |
| | | | | | | GGGCGAGGGGCGGGGAGCGCC | GAGGGAGCGGCCGGATG | |
| | | | | | | GCCTGGAGCGCGGCAG | GAGCGGAGGATGAAAGCC | |
| | | | | | | | GGATACTTGGACCAGCAA | |
| | | | | | | | GTGCCCTACA | |
| 66 | NM_005656 | 42880008 | NM_001986 | 41622735 | hg19 | GAGTAGGCGCGAGCTAAGCAG | AAATCGCCCGGAAATGGG | 2 |
| | | | | | | GAGGCGGAGGCGGAGGCGGA | AGCTTGCGCGAAGCGCTG | |
| | | | | | | GGGCGAGGGGCGGGGAGCGCC | ATCGGCCCGCTGGGGAAG | |
| | | | | | | GCCTGGAGCGCGGCAG | CTCATGGACCCGGGCTCCC | |
| | | | | | | | TGCCGCCCCTCGACTCTGA | |
| | | | | | | | AGATCTCT | |
| 67 | | | | | | | | |
| 68 | | | | | | | | |
| 69 | | | | | | | | |
| 70 | | | | | | | | |
| 71 | | | | | | | | |
| 72 | | | | | | | | |
| 73 | NM_000142 | 1808661 | NM_006342 | 1741429 | hg19 | GATCATGCGGGAGTGCTGGCAT | GTAAAGGCGACACAGGAG | 8 |
| | | | | | | GCCGCGCCCTCCCAGAGGCCCA | GAGAACCGGGAGCTGAGG | |
| | | | | | | CCTTCAAGCAGCTGGTGGAGGA | AGCAGGTGTGAGGAGCTC | |
| | | | | | | CCTGGACCGTGTCCTTACCGTG | CACGGGAAGAACCTGGAA | |
| | | | | | | ACGTCCACCGAC | CTGGGGAAGATCATGGAC | |
| | | | | | | | AGGTTCGAAG | |
| 74 | | | | | | | | |
| 75 | | | | | | | | |
| 76 | | | | | | | | |
| 77 | | | | | | | | |
| 78 | NM_000142 | 1808408 | NM_006342 | 1741429 | hg19 | GCTGGGGGGCTCCCCGTACCCC | GTAAAGGCGACACAGGAG | 1 |
| | | | | | | GGCATCCCTGTGGAGGAGCTCT | GAGAACCGGGAGCTGAGG | |
| | | | | | | TCAAGCTGCTGAAGGAGGGCCA | AGCAGGTGTGAGGAGCTC | |
| | | | | | | CCGCATGGACAAGCCCGCCAAC | CACGGGAAGAACCTGGAA | |
| | | | | | | TGCACACACGAC | CTGGGGAAGATCATGGAC | |
| | | | | | | | AGGTTCGAAG | |
| 79 | NM_000142 | 1808276 | NM_006342 | 1741429 | hg19 | CGACTACTACAAGAAGACGACC | GTAAAGGCGACACAGGAG | 1 |
| | | | | | | AACGGCCGGCTGCCCGTGAAGT | GAGAACCGGGAGCTGAGG | |
| | | | | | | GGATGGCGCCTGAGGCCTTGTT | AGCAGGTGTGAGGAGCTC | |
| | | | | | | TGACCGAGTCTACACTCACCAG | CACGGGAAGAACCTGGAA | |
| | | | | | | AGTGACGTCTGG | CTGGGGAAGATCATGGAC | |
| | | | | | | | AGGTTCGAAG | |
| 80 | NM_000142 | 1808408 | NM_006342 | 1739325 | hg19 | GCTGGGGGGCTCCCCGTACCCC | GTGCCAGGCCCACCCCCA | 1 |
| | | | | | | GGCATCCCTGTGGAGGAGCTCT | GGTGTTCCCGCGCCTGGG | |
| | | | | | | TCAAGCTGCTGAAGGAGGGCCA | GGCCCACCCCTGTCCACCG | |
| | | | | | | CCGCATGGACAAGCCCGCCAAC | GACCTATAGTGGACCTGCT | |
| | | | | | | TGCACACACGAC | CCAGTACAGCCAGAAGGA | |
| | | | | | | | CCTGGATG | |
| 81 | NM_000142 | 1808661 | NM_006342 | 1732899 | hg19 | GATCATGCGGGAGTGCTGGCAT | GAGAGAGCCTTGAACTCT | 1 |
| | | | | | | GCCGCGCCCTCCCAGAGGCCCA | GCCAGCACCTCGCTTCCCA | |
| | | | | | | CCTTCAAGCAGCTGGTGGAGGA | CAAGCTGTCCAGGCAGTG | |
| | | | | | | CCTGGACCGTGTCCTTACCGTG | AGCCAGTGCCCACCCATCA | |
| | | | | | | ACGTCCACCGAC | GCAGGGGCAGCCTGCCTT | |
| | | | | | | | GGAGCTGA | |
| 82 | | | | | | | | |
| 83 | NM_000142 | 1808661 | NM_006342 | 1739325 | hg19 | GATCATGCGGGAGTGCTGGCAT | GTGCCAGGCCCACCCCCA | 2 |
| | | | | | | GCCGCGCCCTCCCAGAGGCCCA | GGTGTTCCCGCGCCTGGG | |
| | | | | | | CCTTCAAGCAGCTGGTGGAGGA | GGCCCACCCCTGTCCACCG | |
| | | | | | | CCTGGACCGTGTCCTTACCGTG | GACCTATAGTGGACCTGCT | |
| | | | | | | ACGTCCACCGAC | CCAGTACAGCCAGAAGGA | |
| | | | | | | | CCTGGATG | |
| 84 | | | | | | | | |
| 85 | | | | | | | | |
| 86 | | | | | | | | |
| 87 | NM_002675 | 74325755 | NM_000964 | 38504568 | hg19 | CCCCACCTGGATGGACCGCCTA | CCATTGAGACCCAGAGCA | 8 |
| | | | | | | GCCCCAGGAGCCCCGTCATAGG | GCAGTTCTGAAGAGATAG | |
| | | | | | | AAGTGAGGTCTTCCTGCCCAAC | TGCCCAGCCCTCCCTCGCC | |
| | | | | | | AGCAACCACGTGGCCAGTGGCG | ACCCCCTCTACCCCGCATC | |
| | | | | | | CCGGGGAGGCAG | TACAAGCCTTGCTTTGTCT | |
| | | | | | | | GTCAGGA | |
| 88 | NM_002675 | 74315749 | NM_000964 | 38504568 | hg19 | GAGGAGCCCCAGAGCCTGCAA | CCATTGAGACCCAGAGCA | 7 |
| | | | | | | GCTGCCGTGCGCACCGATGGCT | GCAGTTCTGAAGAGATAG | |
| | | | | | | TCGACGAGTTCAAGGTGCGCCT | TGCCCAGCCCTCCCTCGCC | |
| | | | | | | GCAGGACCTCAGCTCTTGCATC | ACCCCCTCTACCCCGCATC | |
| | | | | | | ACCCAGGGGAAAG | TACAAGCCTTGCTTTGTCT | |
| | | | | | | | GTCAGGA | |
| 89 | NM_002675 | 74317268 | NM_000964 | 38504568 | hg19 | CCTCAGCTCTTGCATCACCCAGG | CCATTGAGACCCAGAGCA | 3 |
| | | | | | | GGAAAGATGCAGCTGTATCCAA | GCAGTTCTGAAGAGATAG | |
| | | | | | | GAAAGCCAGCCCAGAGGCTGCC | TGCCCAGCCCTCCCTCGCC | |
| | | | | | | AGCACTCCCAGGGACCCTATTG | ACCCCCTCTACCCCGCATC | |
| | | | | | | ACGTTGACCTG | TACAAGCCTTGCTTTGTCT | |
| | | | | | | | GTCAGGA | |
| 90 | NM_005436 | 61665880 | NM_020630 | 43612032 | hg19 | AGAGAACAAGGTGCTGAAGAT | GAGGATCCAAAGTGGGAA | 2 |
| | | | | | | AGAGCTGGAGACCTACAAACTG | TTCCCTCGGAAGAACTTGG | |
| | | | | | | AAGTGCAAGGCACTGCAGGAG | TTCTTGGAAAAACTCTAGG | |
| | | | | | | GAGAACCGCGACCTGCGCAAAG | AGAAGGCGAATTTGGAAA | |
| | | | | | | CCAGCGTGACCATC | AGTGGTCAAGGCAACGGC | |
| | | | | | | | CTTCCATC | |
| 91 | NM_178039 | 1250953 | NM_020630 | 43612032 | hg19 | GGATATGGCTGAAGAGAAGGG | GAGGATCCAAAGTGGGAA | 1 |
| | | | | | | GACACAAGCTGGAGAGATACAT | TTCCCTCGGAAGAACTTGG | |
| | | | | | | GACCTCAAGGACATGTTGGATG | TTCTTGGAAAAACTCTAGG | |
| | | | | | | TGAAGGAGCGGAAGGTTAATGT | AGAAGGCGAATTTGGAAA | |
| | | | | | | TCTTCAGAAGAAG | AGTGGTCAAGGCAACGGC | |
| | | | | | | | CTTCCATC | |
| 92 | NM_178039 | 1346070 | NM_020630 | 43612032 | hg19 | GAAGCACAAGGAACAGGTGGA | GAGGATCCAAAGTGGGAA | 1 |
| | | | | | | AAAAAAGAAGAGTGCACAAAT | TTCCCTCGGAAGAACTTGG | |
| | | | | | | GTTAGAGGAGGCGCGACGACG | TTCTTGGAAAAACTCTAGG | |
| | | | | | | GGAGGACAATCTCAACGACAGC | AGAAGGCGAATTTGGAAA | |
| | | | | | | TCTCAGCAGCTACAG | AGTGGTCAAGGCAACGGC | |
| | | | | | | | CTTCCATC | |
| 93 | NM_178039 | 1553916 | NM_020630 | 43612032 | hg19 | CCCCCTGATCCTCCGTGGACTCA | GAGGATCCAAAGTGGGAA | 1 |
| | | | | | | CTCCACCAGCTTCCTATAACTTG | TTCCCTCGGAAGAACTTGG | |
| | | | | | | GACGATGACCAGGCGGCTTGG | TTCTTGGAAAAACTCTAGG | |
| | | | | | | GAGAATGAGCTGCAGAAGATG | AGAAGGCGAATTTGGAAA | |
| | | | | | | ACCCGGGGGCAG | AGTGGTCAAGGCAACGGC | |
| | | | | | | | CTTCCATC | |
| 94 | | | | | | | | |
| 95 | | | | | | | | |
| 96 | | | | | | | | |
| 97 | | | | | | | | |
| 98 | | | | | | | | |
| 99 | | | | | | | | |
| 100 | | | | | | | | |
| 101 | | | | | | | | |
| 102 | | | | | | | | |
| 103 | | | | | | | | |
| 104 | | | | | | | | |
| 105 | NM_005437 | 51582939 | NM_020630 | 43612032 | hg19 | CCTTGGAAGCAAACCTGCCAGT | GAGGATCCAAAGTGGGAA | 2 |
| | | | | | | GGTTATCAAGCTCCTTACATACC | TTCCCTCGGAAGAACTTGG | |
| | | | | | | CAGCACCGACCCCCAGGACTGG | TTCTTGGAAAAACTCTAGG | |
| | | | | | | CTTACCCAAAAGCAGACCTTGG | AGAAGGCGAATTTGGAAA | |
| | | | | | | AGAACAGTCAG | AGTGGTCAAGGCAACGGC | |
| | | | | | | | CTTCCATC | |
| 106 | | | | | | | | |
| 107 | | | | | | | | |
| 108 | | | | | | | | |
| 109 | | | | | | | | |
| 110 | | | | | | | | |
| 111 | NM_004355 | 149784243 | NM_002944 | 117645578 | hg19 | ATAGACTGGAAGGTCTTTGAGA | ATGATTTTTGGATACCAGA | 1 |
| | | | | | | GCTGGATGCACCATTGGCTCCT | AACAAGTTTCATACTTACT | |
| | | | | | | GTTTGAAATGAGCAGGCACTCC | ATTATAGTTGGAATATTTC | |
| | | | | | | TTGGAGCAAAAGCCCACTGACG | TGGTTGTTACAATCCCACT | |
| | | | | | | CTCCACCGAAAG | GACCTTTGTCTGGCATAGA | |
| | | | | | | | AGATT | |
| 112 | | | | | | | | |
| 113 | | | | | | | | |
| 114 | | | | | | | | |
| 115 | NM_003379 | 159191796 | NM_002944 | 117645578 | hg19 | GAAACCGTGGAGAGAGAGAAA | ATGATTTTTGGATACCAGA | 1 |
| | | | | | | GAGCAGATGATGCGCGAGAAG | AACAAGTTTCATACTTACT | |
| | | | | | | GAGGAGTTGATGCTGCGGCTGC | ATTATAGTTGGAATATTTC | |
| | | | | | | AGGACTATGAGGAGAAGACAA | TGGTTGTTACAATCCCACT | |
| | | | | | | AGAAGGCAGAGAGAG | GACCTTTGTCTGGCATAGA | |
| | | | | | | | AGATT | |
| 116 | | | | | | | | |
| 117 | | | | | | | | |
| 118 | | | | | | | | |
| 119 | | | | | | | | |
| 120 | | | | | | | | |
| 121 | | | | | | | | |
| 122 | | | | | | | | |
| 123 | | | | | | | | |
| 124 | | | | | | | | |
| 125 | | | | | | | | |
| 126 | | | | | | | | |
| 127 | | | | | | | | |
| 128 | | | | | | | | |
| 129 | | | | | | | | |
| 130 | NM_005685 | 73935627 | NM_004304 | 29446394 | hg19 | | | 1 |
| 131 | NM_015955 | 32168371 | NM_004304 | 29543748 | hg19 | | | 1 |
| 132 | | | | | | | | |
| 133 | | | | | | | | |
| 134 | NM_003162 | 37143221 | NM_004304 | 29446394 | hg19 | | | 2 |
| 135 | NM_000366 | 63354844 | NM_004304 | 29446394 | hg19 | | | 1 |
| 136 | NM_006738 | 86284726 | NM_020630 | 43612032 | hg19 | | | 1 |
| 137 | NM_015258 | 115932802 | NM_020630 | 43612032 | hg19 | | | 1 |
| 138 | | | | | | | | |
| 139 | | | | | | | | |
| 140 | NM_001042475 | 117641193 | NM_002944 | 117641193 | hg19 | | | 1 |
| 141 | NM_004327 | 23613779 | NM_005157 | 133729451 | hg19 | | | |
| 142 | NM_004327 | 23615961 | NM_005157 | 133729451 | hg19 | | | |
| 143 | NM_004327 | 23631808 | NM_005157 | 133729451 | hg19 | | | |
| 144 | NM_004327 | 23654023 | NM_005157 | 133729451 | hg19 | | | |
| 145 | NM_004327 | 23524426 | NM_005157 | 133730188 | hg19 | | | |
| 146 | NM_004327 | 23631808 | NM_005157 | 133730188 | hg19 | | | |
| 147 | NM_004327 | 23632600 | NM_005157 | 133730188 | hg19 | | | |
| 148 | NM_004327 | 23596167 | NM_005157 | 133710831 | hg19 | | | |
| 149 | | | | | | | | |
| 150 | | | | | | | | |
| 151 | | | | | | | | |
| 152 | | | | | | | | |
| 153 | | | | | | | | |
| 154 | | | | | | | | |
| 155 | | | | | | | | |
| 156 | | | | | | | | |
| 157 | | | | | | | | |
| 158 | | | | | | | | |
| 159 | | | | | | | | |
| 160 | | | | | | | | |
| 161 | | | | | | | | |
| 162 | | | | | | | | |
| 163 | | | | | | | | |
| 164 | | | | | | | | |
| 165 | | | | | | | | |

What is claimed is:

1. A method to determine an actionable treatment recommendation for a subject diagnosed with cancer, comprising:
obtaining a biological sample from the subject
detecting at least one variant using a set of probes that hybridize to and amplify i) the variants of at least one gene in Tables 13-17 and 19; or ii) EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS genes to detect at least one variant,
determining, based on the at least one variant detected, an actionable treatment recommendation for the subject.

2. The method of claim 1, wherein the cancer is an adenocarcinoma lung cancer or a squamous cell carcinoma lung cancer.

3. The method of claim 1, wherein the sample is tissue comprising tumor tissue.

4. The method of claim 3, wherein the sample is an FFPE tumor tissue sample.

5. The method of claim 1, wherein the at least one variant is selected from ALK fusion, ROS1 fusion (EZR, SLC34A2, CD74, and/or SDC4), or MET gene amplification, EGFR (L858R, Exon 19 del, G719X and/or T790M), KRAS (G12C/V/D/A/S/R/F, G13C, G3D and/or G12F), BRAF (L597R, D594H/N, V600E), ERBB2 exon 20 ins, PIK3CA (E545K, E545G, E545a, H1047R, E542K, and/or H1047L), and a combination thereof.

6. The method of claim 1, wherein the actionable treatment recommendation is a course of treatment, refraining from a treatment, enrollment in a clinical trial, or a combination thereof.

7. The method of claim 6, further comprising performing the actionable treatment recommendation.

8. The method of claim 1, wherein the set of probes are in the same reaction mixture.

9. A method to determine an actionable treatment recommendation for a subject diagnosed with lung cancer, comprising:
detecting in a sample from a subject, at least one variant using a set of probes that hybridize to and amplify i) the variants of at least one gene in Tables 13-17 and 19; or ii) EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS genes to detect at least one variant,
determining, based on the at least one variant detected, an actionable treatment recommendation for the subject.

10. The method of claim 1 or 9, wherein the actionable treatment recommendation is selected from
a. Crizotinib when the variant detected is an ALK fusion, ROS1 fusion (EZR, SLC34A2, CD74, and/or SDC4), or MET gene amplification;
b. EGFR tyrosine kinase inhibitor (TKI) when the variant detected is EGFR (L858R, Exon 19 del, and/or G719X);
c. A non EGFR TKI when the variant detected is EGFR T790M;
d. A MEK inhibitor when the variant detected is KRAS G12C/V/D/A/S/R/F, G13C, G3D and/or G12F;
e. Vermurafenib when the variant detected is BRAF V600E;
f. An irreversible pan-erb inhibitor when the variant detected is ERBB2 exon 20 ins; and
g. A PIC3CA inhibitor when the variant detected is PIK3CA (E545K, E545G, E545a, H1047R, and/or H1047L).

11. The method of claim 1 or 9, further comprising treating the subject with the recommended actionable treatment.

12. The method of claim 1, wherein detection of a variant in at least one of the genes provides an actionable treatment recommendation for i) a cancer in Table 18; or ii) at least 50% of all primary lung adenocarcinoma.

13. A method to determine the likelihood of a response to a treatment in an individual afflicted with cancer, comprising:
determining the presence or absence of at least one gene variant in a sample obtained from the individual, wherein the at least one variant is i) a gene variant in Tables 13-17 and 19 or ii) in EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS genes,
wherein the presence of at least one variant indicates the individual is likely or unlikely to respond to the treatment.

14. The method of claim 13 wherein the treatment is selected from:
a. crizotinib when the variant detected is an ALK fusion, ROS1 fusion (EZR, SLC34A2, CD74, and/or SDC4), or MET gene amplification;
b. EGFR tyrosine kinase inhibitor (TKI) when the variant detected is EGFR (L858R, Exon 19 del, and/or G719X);
c. a non-EGFR TKI treatment when the variant detected is EGFR T790M;
d. a MEK inhibitor when the variant detected is KRAS G12C/V/D/A/S/R/F, G13C, G3D and/or G12F;
e. vermurafenib when the variant detected is BRAF V600E;
f. an irreversible pan-erb inhibitor when the variant detected is ERBB2 exon 20 ins; and
g. a PIC3CA inhibitor when the variant detected is PIK3CA (E545K, E545G, E545a, H1047R, E542K and/or H1047L).

15. A method of detecting a nucleic acid variant in a sample, comprising
obtaining a biological sample,
a) amplifying at least one gene selected from i) the genes in Tables 13-17 and 19 or ii) EGFR, ALK, ROS1, KRAS, BRAF, ERBB2, ERRBB4, MET, RET, FGFR1, FGFR2, FGFR3, DDR2, NRAS, PTEN, MAP2K1, TP53, STK11, CTNNB1, SMAD4, FBXW7, NOTCH 1, KIT/PGDFRA, PIK3CA, AKT1, and HRAS, using primers that specifically hybridize to the genes;
b) amplifying at least one variant selected from i) the variants in Tables 13-17 and 19, or ii) EGFR (L858R, Exon 19 del, G719X, T790M and/or Exon 20 ins), KRAS (G12C/V/D/A/S/R/F, G13C, G13D and/or G12F), BRAF (L597R, D594H/N, V600E), ERBB2 exon 20 ins, PIK3CA (E545K, E545G, E545a, H1047R, and/or H1047L), c) detecting at least one nucleic acid variant present in the sample.

16. A method of treating lung adenocarcinoma in a patient, comprising testing for the presence of variants in at least one of ALK, ROS1, KRAS, BRAF, ERBB2, MET, RET, FGFR1, and KIT/PDGFRA genes in a lung tumor sample from the patient and administering a therapeutically effective amount a treatment to the patient, wherein the treatment is
a. Crizotinib when the variant detected is an ALK fusion, ROS1 fusion (EZR, SLC34A2, CD74, and/or SDC4), or MET gene amplification;
b. EGFR tyrosine kinase inhibitor (TKI) when the variant detected is EGFR (L858R, Exon 19 del, and/or G719X);
c. A MEK inhibitor when the variant detected is KRAS G12C/V/D/A/S/R/F, G13C, G3D and/or G12F;
d. Vermurafenib when the variant detected is BRAF V600E; and
e. An irreversible pan-erb inhibitor when the variant detected is ERBB2 exon 20 ins.

17. A method of identifying patients with lung cancer eligible for treatment with crizotnib, an EGFR TKI, or a treatment other than an EGFR™, a MEK inhibitor, vermurafenib, or an irreversible pan-erb inhibitor, comprising testing a lung tumor sample from the patient for the presence of a variant comprising an ALK fusion, ROS1 fusion (EZR, SLC34A2, CD74, and/or SDC4), EGFR (L858R, Exon 19 del, and/or T790M), KRAS (G12C/V/D/A), wherein the presence of at least one of said variants indicates the patient is eligible for treatment with at least one of said treatments.

18. The method of claim 17, wherein the ALK gene fusion isoform is an EML4-ALK gene fusion isoform.

19. A kit comprising a set of probes, wherein the set of probes specifically recognize the genes i) in Tables 13-17 and 19; or ii) AKT1, ALK, BRAF, ERBB2, EGFR, FGFR1, HRAS, KIT, KRAS, MET, PIK3CA, RET and ROS, and wherein the set of probes can recognize and distinguish one or more allelic variants of the genes in i) Tables 13-17 and 19; or ii) AKT1, ALK, BRAF, ERBB2, EGFR, HRAS, KRAS, MET, PIK3CA, RET and ROS.

20. The kit of claim 19, wherein the allelic variants include one or more of the polynucleotides encoding AKT1 (E17K), BRAF (L597R, D594H/N, V600E), EGFR (L858R, G719X, T790M), HRAS (Q61L/K/R, G12C/D), KRAS G12A/C/D/F/R/V) and PIK3CA (E545A/G/K, H1047L/R).

21. The kit of claim 19, wherein the allelic variants include one or more of the polynucleotides encoding EGFR (L858R, G719X, T790M) and KRAS G12A/C/D/F/R/V).

22. The kit of claim 19, wherein two or more of the probes are primer pairs.

23. The kit of claim 19, wherein one or more of the probes are detectably labeled.

24. The kit of claim 19, wherein the set of probes comprises at least 4 amplification detection assays, wherein the at least 4 amplification detection assays are specific for the genes ALK, EGFR, KRAS and ROS.

25. The method of claim 1, wherein the method is performed using probes from the kit of claim 19.

26. A composition comprising a set of probes, wherein the set of probes specifically hybridize with a plurality of genes i) in Tables 13-17 and 19; or ii) the genes AKT1, ALK, BRAF, ERBB2, EGFR, FGFR1, HRAS, KIT, KRAS, MET, PIK3CA, RET and ROS, and wherein the set of probes can recognize and distinguish one or more allelic variants of the genes i) in Tables 13-17 and 19; or ii) AKT1, ALK, BRAF, ERBB2, EGFR, HRAS, KRAS, MET, PIK3CA, RET and ROS.

27. The composition of claim 26, further comprising a sample.

28. The composition of claim 27, wherein the sample comprises nucleic acids from tumor cells.

29. The method of claims 1, 11, 15 or 16, wherein the gene variants are selected from an A1 and prevalence selected from AI1+Prevalence>1%, AI2+Prevalence>1%, AI3+Prevalence>1%, AI1+Prevalence 0.1%-1%, AI2+Prevalence 0.1%-1%, AI3+Prevalence 0.1%-1%, and combinations thereof.

30. The kit of claim 19, wherein the gene variants are selected from an A and prevalence selected from AI1+Prevalence>1%, AI2+Prevalence>1%, AI3+Prevalence>1%, AI1+Prevalence 0.1%-1%, AI2+Prevalence 0.1%-1%, AI3+Prevalence 0.1%-1%, and combinations thereof.

31. The composition of claim 26, wherein the gene variants are selected from an A1 and prevalence selected from AI1+Prevalence>1%, AI2+Prevalence>1%, AI3+Prevalence>1%, AI1+Prevalence 0.1%-1%, AI2+Prevalence 0.1%-1%, AI3+Prevalence 0.1%-1%, and combinations thereof.

32. A method to determine an actionable treatment recommendation for a subject diagnosed with lung cancer, comprising:
obtaining a biological sample from the subject;
detecting at least one variant using a set of probes that hybridize to and amplify EGFR, ALK, ROS1, KRAS, BRAF, ERBB2, MET, RET, FGFR1, KIT/PGDFRA, PIK3CA, AKT1, BRAF, and HRAS genes to detect at least one variant;
determining, based on the at least one variant detected, an actionable treatment recommendation for the subject.

33. A method of reporting an actionable index
obtaining a biological sample
amplifying a plurality of genes selected from the genes in Tables 13-17 and 19,
amplifying at least one variant selected from the variants Tables 13-17 and 19,
detecting at least one nucleic acid variant present in the sample,
determining the actionable index of the nucleic acid variant present,
reporting the actionable index.

34. The method of claim 33, wherein the biological sample comprises cancer cells.

35. The method of claim 33, wherein the actionable index is a treatment index.

35. The method of claim 33, wherein actionable index is selected from category A1, A2, A3, A4 or A5.

36. The method of any one of claims 1, 9, 13, wherein the nucleic acid variant is detected with one or more sequencing methods.

37. The method of any one of claims 1, 9, 13, wherein the nucleic acid variant is detected with one or more method selected from Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry, sequencing by hybridization, next generation sequencing (NGS), and a combination thereof.

38. The method of claim any one of claims 1, 9, 13, wherein the nucleic acid variant is detected with NGS.

39. The method of claim 38, further comprising confirming the detection of the nucleic acid variant with one or more methods selected from Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry, and sequencing by hybridization.

40. The method of any one of claims 1, 9, 13, wherein the at least one variant is associated with a cancer in Table 18.
